SEQUENCE LISTING (242 sequences)

SEQ ID NO: 1 - protein, 316 aa, Rhipicephalus microplus
MKIENLLIRQLINEFLGTMILITIGDSIMAIIIAGDNESLAACVGPLGWGVAIYVAVQISGGVSSHLNPAVTLAQASVRKFPIAKVPLYFAAQYLGGFVGAALVFATYKDAIEHFDQGIRQVTGEKATAGIFATYPRPHVSTLTCFIDQVIATGIMMVCVEAIGDTRNFGGIPPHIHPICLGLMIMAIIFSFAYNCMCPLNPARDISPRLFTLMAGWGPETFTLRGWNYVWVPLLGPHIGAILGVWLYKVAIGDHWPEKPKPAISMDHPVAVVVAKKAPSRTPPALSPSAAPTTSTSPQPAAPQATDVNAGSPAKA

SEQ ID NO: 2 - protein, 293 aa, Rhipicephalus microplus
MKPNTVTRAWRQVTGCCIENTLARQALAEMVGTLVLTLVGDCVLASLAVFQLGSVGLAAAPLGWGLAVFLGVLVAGGVSGAHLNPAVTVALATIGKLGWCNVLAYVTAQYLGAFLASGLVYLVYADALSQVDVNLAIVYGTNATAPVFSCFPAPGVSTLTCLLDQTVSTAVLLLGICAITDGRNMAVSRGQQPLLVGLTVSACMYAFSYNCGNPLNPARDLAPRIFTAMCGWGSAVFSFRSYNWFWVPVVGPHLGAVIGVWIYKLAVDNHWKDEDEVDEDEKRPLLSNAKICA

SEQ ID NO: 3 - protein, 650 aa, Rhipicephalus microplus
MRGIALFVAAVSLIVEGTAESSICSDFGNEFCRNAECEVVPGAEDDFVCKCPRDNMYFNAAEKQCEYKDTCKTRECSYGRCVESNPSKASCVCEASDDLTLQCKIKNDYATDCRNRGGTAKLRTDGFIGATCDCGEWGAMNMTTRNCVPTTCLRPDLTCKDLCEKNLLQRDSRCCQGWNTANCSAAPPADSYCSPGSPKGPDGQCINACKTKEAGFVCKHGCRSTGKAYECTCPSGSTVAEDGITCKSISHTVSCTAEQKQTCRPTEDCRVHKGTVLCECPWNQHLVGDTCISDCVDKKCHEEFMDCGVYMNRQSCYCPWKSRKPGPNVNINECLLNEYYYTVSFTPNISFDSDHCKWYEDRVLEAIRTSIGKEVFKVEILNCTQDIKARLIAEKPLSKHVLRKLQACEHPIGEWCMMYPKLLIKKNSATEIEEENLCDSLLKDQEAAYKGQNKCVKVDNLFWFQCADGYTTTYEMTRGRLRRSVCKAGVSCNENEQSECADKGQIFVYENGKANCQCPPDTKPGEIGCIERTTCNPKEIQECQDKKLECVYKNHKAECECPDDHECYREPAKDSCSEEDNGKCQSSGQRCVIENGKAVCKEKSEATTAATTTTKAKDKDPDPGKSSAAAVSATGLLLLLAATSVTAASL

SEQ ID NO: 4 - protein, 436 aa, Rhipicephalus microplus
MELIVLPLALLLTCALGMPQQNESGSTTSTTSPAASSSASANATTARTGSGAHPGPAPVVCYYSAWANSRPHPANYGIKDVSGDLCTHVNFAYAGVNPQTWELKSEVPEFERNRDLFKNFTAIKTQYPQLKTLLSVGGWQHETGIFSQMVASADRRALFIESVLLWMKEYNLDGVDMAWRFPGVSYRGGSPRDKENYASLIRELAGAFQGKDLLLTVVVPVADEFLDPGYDVAEISKYVDWINAQAYDLRGTWNGVTDVHTPLFPRSIDLGSQATLNVKDGLARIVSLGAPKSKVVMGIAFFGRGFTLLDPQQHGLHALINRDVPPHAGPFVRSSEIFAYYEICLNLKGNWKREFDDEGKCPYVYYRDQWIGYDDAVSIQHKIDFLLQEGYRGVYVFNNDLDDFRGFCGEPNILLKTIKNGLTEKNELDARLNQKE

SEQ ID NO: 5 - protein, 259 aa, Rhipicephalus microplus
MMFHPFHLVEKSPWPLTSSISALSLTLGFVSFFQNLNFFLLMLSILMIFMSSFQWWRDISREGSFQGFHTYWVLNGLKMGMILFILSEIFFFISFFWAFFHSSLSPNIEIGGQWPPKNIFPFNPFEIPLLNSTILISSGITITWSHHSIMNKNYISTLNSLLITIILGVTFTMFQGFEYFQAQFSISDGIFGSTFFLTTGFHGIHVLIGSIFLLVSFFRIKKQLISSNHFFGFEASAWYWHFVDVVWLFLFTFMYWWIY

SEQ ID NO: 6 - protein, 216 aa, Rhipicephalus microplus
MPVLLYNLEGSPHCSFIRCLAKEIGVQLSIKEINCAKGEHRSAEFIKKNPFHKVPTIDDGGLIVYESNAIAYYLLRKYAPESQLYPNCIKTRARIDQVLAAANGNISPILGTYLAARCLKRTKPSADEVKECEEKVLKCLEHLLGESKFAAGDKITLADLCLIGHVAVYLELPCVNRAKYPKLAAYYDRVKGALPYFNEVFGPAIAQAKKLWDMVH

SEQ ID NO: 7 - protein, 559 aa, Rhipicephalus microplus
MVSVIIVLVLAIAGEAMQQPRLVYPRLLEERSADGRMVMHLHDQLTLNLRKASVAARNFRVLEQEDGREVTHFFNGEDLEQDLHEDEKQFASVHVRRRGGGVEVEGVVGPRHRIEPVHTMERSEEGLIAHMIHEIEMKDMHDIVMAPIGKGAPTVSERNYGSTQNVPPSVTIELFVVSDRPHHQHFQTTIQLIQYLCVMVNSMNLRYADTDSPRVIFLLTGVEKDEHSPYRNGNDKYLESASSLEQFRSYAYGKRHQFGNPDVTFLITGYDVYSTASGGSKSTSVLGIGYVGGLCTEYFVALGEDSAGLYTGMHTLTHECGHVLGAAHDESRPVSWIKGDPGSMACLWKEGHIMSYVDGGVKHHHFSKCSLAQIRNVVILRGLVCWQLGNTGHTYDGIYPGMEVAPNAYCRHAFPDKENVTADMESDSMKKCMVKCQYPEYRRVCYSSRCFTYVTTYSLQLHAQDYMPCGGNKVCIQGVCGEGKIVMPSQKPTTVTVTADTEAKTTTTTTTTTTPTTPTTATTECRCDCSTTASTTQKRPWQRSAPRWPYRSGNRWEEK

SEQ ID NO: 8 - protein, 380 aa, Rhipicephalus microplus
MAVNQLGDSVLNFAIGLYGQLKPKDGRKKNIFFSPFSISAALSMALGGARNKTAKELSTVLRVPDDAQIHRHFSDFFSKLSSYAADVKLHIANRMYCEQTFPVLESYLSLLRDSYGATVESVDFKNKSETIRLQANEWVERETESKIKDLLPGGSVNDNTTLILINAIYFKGLWASQFEPDATHPSYFHLDSKSKKQVDMMFQKDRYCTGRSKELGVEALEIPYRGGKTSMVILLPDELGGLSKLEKRLTASKLGNLLENLRGFFDVELYLPKFELEQTINLKEVLKQMGINDFFSFDADLSAISKKDKLAASEVVHKAFVEVNEEGTEAAAATAVMMAACCLSSAPPQTYKFIVDRPFMFVIRSRDPDIVLFMGSVRDL

SEQ ID NO: 9 - protein, 404 aa, Rhipicephalus microplus
MGSRITAILLCAAVIMAPPASAQDIQHEQARANNALGVALFKELCSNSADRNVFFSPASLSIALGMLYAGAGGKTLQELSTVLGLADAGLVDRDAVLSAYKSLLETKSANATLDIANTVLIQKDFEVLEQYKKDVVEYFQAEACSVDFVRDAHKVVAEINDWVNKKTRGKISKLLDDAPPMNTVAFLINAIYFKGTWVTKFKARRTKPLPFYNHGRREAKVATMSVRHRFGYASVEELDARALEVPYAGDRFSMVVVLPRSRTGLSTVETRLTTDLVEKITNSLTDTDVQLWLPKFKLQTDYDLVTPLRKLGLESAFGKGADLSGISARNNLEVSDVKHKALIEVSEQGTVAAAVTAIRVSVKSGKSAGPLPPISFRVEHPFAFFIWDKVSKQALFMGAIRSLA

SEQ ID NO: 10 - protein, 380 aa, Rhipicephalus microplus
MAVNQLRDSVLNFAVDLYGQLKPEGRDKRNIFFSPFSISAALSMALGGARNKTAEELCTVLRVPDDVQIHRHFSDFFSKLKSYAADVKLHIANRMYCEQTFPVLASYLSLLRDSYGATIESVDFKGDHEAVRLQANAWVERETEYKIKDLLPGGSVNAGTTLILINAIYFKGLWASQFKPDATRPSDFHLDSKSKKKVEMMFHKDRYSTARCQELDVEALEITYQGNKTSMVILRPNDVEGLSKLEERLTASKLASLLDNLCGFADVELYLPKFKLEQVISLKEVLQEMGIKDFFSSDADLSGISEKKKLAASEVVHKAFVEVNEEGTEAAAATAVMMVACCMSSLPPRTYKFIVDRPFMFVIRSRDPDLVLFMGSVRDL

SEQ ID NO: 11 - protein, 161 aa, Rhipicephalus microplus
MACATLKRTHDWDPLHSPSGRSPKRRRCMPLSPPPTRAHQIDPSPFGDVPPKLTSEEIAANIREEMRRLQRRKQLCFQGADPESQHTSGLSSPVHRDQPLFTFRQVGLICERMMKERESKIREEYDHVLSTKLAEQYDTFVKFTYDQIQKRFEGATPSYLS

SEQ ID NO: 12 - protein, 273 aa, Rhipicephalus microplus
MAPPCYADLGKQARDLFNKNYHFGVVKLDCKSTTQTGVEFNVSGTSLNDTGKVNASLETKYKVPEYGLTLKEKWNTDNTLSTEICSEEKLARGLKVAFHANFAPQTGKKSGALKAAYKFDNVHLNGDVDLGPPGPLVHGAAVLHYQGWLAGGQVSFDTTKNRLSKTNFAVGFQAGDFVLHTNVNDGQEFAGSVFQRVNPQLQTGVQLAWTAGTNATRFGLGCVYDLDMETSVRAKVNNSGQIGLGFTHRLRPGISLTLSTMLDGKNFNQGGHK

SEQ ID NO: 13 - protein, 1783 aa, Rhipicephalus microplus
MRVLCGLPLLLLAAAANGVLFDPVPPTQSGGVVYKVRATVTLKSPELSVTEGAGLQYAGDLALYSAGQDTYLAQFLNFSVVKHEKLYGGLDNLTAVPLNESVPLFNESSAVDYSQKFRLPVKFVLSAGKVVKYEVSPEVPVWALNVYKSVLTLLQNQVETPQEVPVSAAYYEDGVSGHCRVTYEVHSLTKFLPTEGKVYNVTKTKYLDDCKLPRPVHSLSGAYRKGYPAQCAKHLPNNFLPGFQEDTSAYESRPLVGCPEGLSPVDSVVTAHEVSYYNVSGSLLEGALTDSLVVLPLVTSKVVVRTRLQLELASLSVPPTNPVAFNGELRTSLEHQLPEVAEYLDLPVYSYLVGSPELVKPEVFTQVLEEVAEELVSLELDTETKKTPALMLKLVHMVSLLNTDQLKQALPQSLLTNTSELEPKQQVLKSLYVELLGKAGSKSAVEVAVHLVKQQVLSLYDATRLFRDLSVYQAYVDKDTVELLLELCKTTETYLHPLVKVAVCDALGEAVQKACPTKVHYEAVPYPTYKSSWRRIPKLKSRYEQTPVVPKHLLPYESVERLVLPQEKQCTVADLYEYVTALEEALKVTKDFKVLVGYLNALGKVAKPEVLPVLMSYLNGTAENLYELAEQEEDVTETVYFVKKAVLLALSHVVEYFPKEVNPLVRAVLLNVSEPVDVRTLAFDVWLKSVPTKWDLQTVSLVVKLDRSLELKSYVYSALKYVVKDKHPANHVLASRVRGVFVHLDALNVGPRYSKYVKKTYYNVAKHFGVESVFKHVANNVSFFPTHLHAGLKYNLGPYVKTLLEGKLLLKGGEKFLAEVFSTYGPSTLRSVFSRNIESTYEGGAKYSVVKELNIETRKVEFPKAVLFAKFLGGDVVLPVDKELFDTLKTELVEYLKKVSSSGPVTTHLVRVLLPKKYVHVEPTVVGLPVVQTVLRPVVVSLKLKDVSVLAETSVDSLLPVTLNVTGTVQPLVLSVKQNRVFVPADLTEESPSVLHTSVKHLQGKAHLTLGYDRKYEKKVRVAVKPTFGTTLLSAISTEHVVETPNPFLLLEKPVVDYKKVLDSLVKPFYYHKVYEAKLVNVEVESVSHGPLLPLPLYGKLYQQPSERMTKFVEYLTHTATKQHLLKVKVLPYEKDPVTEWVTTFKYEHNLDFLAKTTLKKVYTPTKPLAYDESQETPYERMHYKPATPGYETYESEESYDYYPEYNQWNKTLVKHVLNVTLEGKHEGSLKKLTKLDFAYYHTLNKTLKHYYVDVKTLTRPLVSVFLNLTHPVPPSPFWYQPSFVGKDLVNATLYVTVGNEPEPFVLTYNATKTVEQLKGLHAYDATPLLHWFVPQCLADQYAGNTVSYACRLATVHDAHLNKQVVAFKVPTQVSPKVKSVALKFLSYLKFRMFPYATFYALPLTEQEEYYVVFKLNKTDVNPYVTVAHGELLLPGEKVVLTDLKLSKYLVPNLVLSGLDRLKHSVFEGHPHPPCSVGKNWVRTYDNVSYPLEVRPNCKYLVTSDCSAKHDFAVVAQPLDLTVGTKKLIVQLGPTVVELPPPDLYKAEVLLTVNGTYYVANTTQDVVLPYKWDRKLLVTVYPTSGPHDPPVVKLTNSLKTFKLLFDGVNFFVWVNPLYQGKTCGLCSNYDNEPYHEFVTPENWLVSNYSEFVASYGFGLPQCKEPLVYTLPVHYLEELKKPLGYESSYPTHREYPTHHKYPTHREYPTHREYPTRHEYPTRHEYPTRHEYPTHHKYPTHHEYPTRHEYPTRHEYPTRHEYPTHHKYPPHHESYPYTANLQREG

SEQ ID NO: 14 - protein, 1799 aa, Rhipicephalus microplus
MKFTACAVLAVVALYLVGNVFSECPQAWFDCGNGRCIAMFWRCDGQNDCGNHKDETGCSATHSRCPADKFACRDSSYCVPQIWVCDGEADCHDSSDEQDCHSSNCTGFRCHNNECIPAHWRCDQTEDCADASDELDCGGVQNSSTTTPTPRCDVDQGRFPCLDGQCLLPSKVCDGRKDCSDGADEGSFCKVNECSQKKCSQGCFVATNGSTCYCNAGFRLLADHISCADVDECAEDPHVCSHGCINSPGSYSCHCLEGYQLTDNSFCKARDPEPLLVFSTTKEIRGLWLRSNRYFEIHPAEAQAVGVEFDSDQHRVFWTDVSTRRSSIHSCRLDGSDFKTLFSAEKTLLEDLSLDWVANNLYITDSLVKRILVCTTDGASCSALITDSVDSPRAIIVNPPQKVVYWTDWGSRPAIMHASMDGTNIQQLVSTDLGWPNGLTLDHTTNRLYWCDAKLSSLEYLELATLKRDVVMHEEVFHPFALAVFEDTVYWSDWASYSLDSSNKRTGKQHHRVLRENGHHIMGVHVYHPVLRQRGIQNPCWDNPCDHICVLSADSYMCLCRIGYKLSANKHSCAVTKDFSFVIVAEEDLVYKIDLNKVGAPVPVTLPVHNLGIISALTFDWSNQTLHYSDNRHSVLSAINVKSFEQWTVHDHIGSVFGIDFDVTHQLLYWVDADKYTLEACHANGSGHVIIRDDLHRPVGVALYPFAGVLFVLSAGDKPTITSYTMDGQNPRVLPLSTLLLPVSISVDLVARTLVWADAVRGTIESLDLQKVFTGTPFIVQQVKAHISSVCAAHNEIHWTSRDKASLEYIDRSSEPSVHRHVSLRSTRNGTFSRRVIVAAQVPPFAPGPCGLNNGGCSHTCLPVRTTDRSCFCPPGMALNADNRTCRVETSTCRPHELPCAGSCIAAVNWCDGHKDCSDNADEESCGSATCPATDFSCSNGRCIEKEWQCDGYNDCGDSSDERNCTVTTCASHQYTCRGGVCLPLYWRCDGSEDCPDGDDELNCSSVRCPSAHSRCDNGQCIPQDWTCDGHSDCSDSSDEKNCTEQPSCFEDDFHCANGQCVDKRLRCDHDNDCEDSSDEVGCDYAKANRTKCSTGMVDCGDGQCIYTHDMCDGYIDCHNSRDERNCSAPICHSAEFFCTGTKRCILQNWLCDGDDDCGDGMDETLPRCHPTTKVSTTSVPACASNEFKCGSRECIAWSRVCDGRTDCADFSDEGTHCVSYCGTTNGGCAHLCRESPTGPLCSCHPGYRLNTDRKSCDDIDECATPGHCSHFCQNSKGSYKCTCADGYALGADRRYCKVQYGEPFLLYMLPNQIRSFSMHGHAQHLLAEDSLSDMHGMDYRVTDKSIFWTEMDEGVINVMTLGNGKQFTLLEDIHKPYHIAVDWVAGNIYFTDGWVHIQACEPTFKHCTDVIDTAYSHLNTFALSANDGLMFWAVWHEVVQKDHGLIERSNMDGTARVVLLTDKILWPCSITVDAVHKRIYWSDANKNVIESATYDGKDRKLVRGTGLSSPFSIALFEDWLYWSDWGSDSLMACNRYTGTHVGLVHHGTAKATVLKVLHAVHQPSGVNRCARNQCAHICLLNPNAYTCACSHGYTLAEDAHKCVESDERYHINSSDILGQFCNPVCLNGGRCISGNDSYFCKCADGFKGPSCTDTSVVSMLSQKSTSSSTALASILVSALCVALLVLGYVLYRRNRDKLAALDFSVSFKKPTFKKRQGLLEDEHPIAADEDYHAMNTTPGFINPAFNTRKTELLSEDGELKRWASSDSLQSSSSKEQSSCVLAGDMAAKQDKVFFFRKH

SEQ ID NO: 15 - protein, 140 aa, Rhipicephalus microplus
TSPMLIAKDLMQSGGGNKTYCISINLAVLKFTTNAGSLSFANSSHCDSEDEVAYSELCQMSNAVHVYDMNWMTASLKASRKLYTFEKAEMLLSKVLFLKAWFPSLCVAAFHAELDTVDGEACPQSEQYARLLSVRKGLPS

SEQ ID NO: 16 - protein, 318 aa, Rhipicephalus microplus
MVREDKTTWKSNYFLRLVQLLDEYPKCFIVGVDNVGSKQMQTIRVSLRKHAVLLMGKNTMIRKAIRGHLDNNPALEKLLPHIKGNVGFVFTKEDLTEVREKIIDNKVKAPARAGALAPLDVMIPAQNTGLGPEKTSFFQALQIPTKISKGTIEILNEIHLIKKDDRVGASEATLLNMLNISPFSYGLKILQVYDSGTVFSPDILDITPEDLRSAFVEGVRNVAAVSLSIGYPTVASVPHSIVNGLKNLIAIAVETDITFKEAEMAKEYLKDPSKFAAAAAPAAGGGAAAAKPEESKKEEAKKEESEEEDDDMGFGLFD

SEQ ID NO: 17 - protein, 391 aa, Rhipicephalus microplus
MKIIICLAGLLALCYGQEEHKVTAANNHFGFKLLNAIPVSPQTNLFYSPYSVSTAMAMAYVGARGETQRDLHETLGYSSVGLTPDHVPSAHAQHTHLLRAPSNSTLRVANAAVVQENYTVTDEYLGLLRQSFDAEVSVSSLHDEQSVRAINDWVKNKTEQKIEKLLSEPLPPNTRLVLLNAIYFKGVWNVPFDARGTQKRPFFNAGTEIVEVDTMYEQISAGYARDDETNADVLDLPYAGLDYSLTIILPRERTGVDALRQNLSWPIFQRLLSKLNMNSPMEVSLPKFKIEGSYKLKAPLSALGASKAFDERYADFSGISGARDLTIYDVVHKAVVEVNEEGSEAAGATAVIFYTKSAAVGIPFVVDHPFLFFIRNRHTGDVLFAGQVNHL

SEQ ID NO: 18 - protein, 189 aa, Rhipicephalus microplus
MLRIVLVVLAASVALAGNNLNDQVNKYILPDRCRAGLQEQLNLELHASLVYMQMAAHLANNKVARGGFARFFRDQSSEEREHAQKIIDYLNLRGGTVSAVNVDMPPTAIWMSVLDALQAALALEHRVTNRLYELHRLAEEYDAQMADFLEQEFLAEQVRSIDQLQRLITQLQNMETGLGEFLLDQQLRA

SEQ ID NO: 19 - protein, 610 aa, Rhipicephalus microplus
MKFPVVFVVAFGFICLTVTDADEETAQGPLKHKPCGIHEEFKECQSSSCGEWRCGEVRGALRPCTADCRNDCFCSEEYNRHPNGTCVPQDHCPPSAVDQSEEPRDGDSGVPVFKFPPDPADCGTNEVWKQCVSSTCAETTCTKRIVGPGCTYDCRYGCFCADGFYRNAEGNCVTVDQCPSADPDLGSSQQCGTNEEWKVCVSSSCAETTCEKKTIGPACTADCQRGCYCSDGFHRNKEGACVTADQCPTVSATSAVPRRAEKKLEPRSTRIICSERVRRRTSAMKCYVVLLAVAVAVCVADTDGDSGVPALKFPREAADCGSNEVWKQCVSSTCAETTCKKRFVGPSCTYDCIYGCYCADGFYRNAEGNCVTSDQCPPEEQNLGNHCCSKRFHMPYIVRRRRRQRRNRSEKGVNPDEVKDPAELQPEMAEGSVAKRRFSCRGGSRSRGSPRSRGSPRSRGHPRSRGHPRPTGHPRAIGYSRSSGLSCSRSQLRSRSMSAVDGAGPLIWRGQAPAPGSLTPNKKVTWADTIGDSGSATTTPTTSKQGSSQQCGPNEEWKVCVSSSCAETTCEKRTIGPACTADCQRGCYCSDGFHRNKEGACVTADQCPTV

SEQ ID NO: 20 - protein, 156 aa, Rhipicephalus microplus
MAIVRQAVVLVALVGACWAHKDGAPESACATMIPGHKDSTHEEAGPSTPFHLVQDKRDFKAGDVVAVTLSSSGTPFKGFFVKAFNENNQEVGQFEASSDAKAVTKCSGVTHTSPTDKTAVKVLWKAPEGAAGKVHFRATAVIDYHKSVTGIQSTVA

SEQ ID NO: 21 - protein, 219 aa, Rhipicephalus microplus
MGDTDADRRRKDAEDAHRRLKEEEKRKMEEKERKKAEVRKRLEEAAKAKKAGGKRGFMTPERKKKLRNLLRKKAAEELKKEQERKAEQRRKIIAERVGQPKPLDGANEATLQGILKQYHARIAALEDAKYDLEYEVRQKDFVINELTIQVNDLRGKFVKPALKKVSKFDKLKMVAKSTSEVDFRSNLKAVKKDAFKLDEEKQADKKPEWALGSKKENEE

SEQ ID NO: 22 - protein, 860 aa, Rhipicephalus microplus
MPRRKTSARGHLTSPKPSKTCGVMGFVGTKHVLAVMVACFAAHVWIQGSSAIDSVDPDISRNVSQIIADKGYPVEEYEVETRDGFVLSMQRLPRGVKPAPKKPAGAKKVAFLLHYLLGSSADWVINYPEQSLPYLLADAGYDVWLGNVRGNTYSRHLRYKRYQKEFWDFCFDEMISYDLPAMLDFVLNATGQQKLFYVGHSQGCLVLFGLLAERPEYNDKIELFAAMAPVTTVTYMRSPIRHLAPFVESAGALFALFGEYDFLPSSPLMRLVAGTLCQFEESRSLCENAIFFICGPEGPQLNKTRLPVLVSHTPAGTSVRNMAHYAQLVLSEKFQKFDFGRTRNRALYGQAEPPEYNLTRVTAPVALYWSVGDWLADPKDVSQLRDKLPRLVLDFRVRDRSFTHLDFSVGVGARHLVYEPLMKAMASVRALVNHFYPDSEQDRMKATVAFGGKAFHKLGVLVAILASILWLRPPSAGNVDPDAERNVAEIIADKGYPVQEFAVTTRDGYVLPLQRIPYGREDLWSTHPRPFRPGPPVILMHALLFSSNVWVVNGPDQSLPYVLADAGFDVWLGNVRGNSQSRHVNYTFYQKELWDFSFEEMGEFDLPDTIDFVLNATGHNETFVIGHSMGAAMTFALLSEKPEYNDKILLFSAMAPAVRIDSMTSQIRHILSLMDDLAPVAALFGRYDFLYHDRMIDFTAKTLCRYPVCRAFCPWFISFASGQPTQPINSTRLPVFLKDIPSGTSYKNMIHMAQLYETGRFAKFDYGAKRNLEVYGKTTPPEYDLRRVSAPVALFWTQGDALVPAANLGFLRDTLPNLVLDFRVEDDRFSHQEFAIGVTAKEAVYDGLVDVMRRFSGQAS

SEQ ID NO: 23 - protein, 412 aa, Rhipicephalus microplus
MHPTLPKGFIVPCALLLLVASGWLTVANAASTSIFSVTSPNASSTTAAAVVVNGSCDERVCMRPNCSCESDQPPAGLRPSQVPQFVTLTFDDAVNVGNMAFYRELLNGTRKNKNNCRIAATFFVSHEYTDYEAVNQLYSWGNEIALHSISHRTDSAYWQTINTTQWEREVAAQKEMMETFAKVPASNVTGVRGPFLYTGGDDGFRMLQKHFRYDCTLVHQRERDREDPVYPYTMDYGFRRNCMVWPCPKDTYAGLWVVPMNVLFKERQGQDYPCAMADACLPQPLTADDTFEYLRSNFEDYYNRSRAPFPLFLHEAYLRHPGRKQGYLQFVDWLLTKDDVYLVTITEMLRYMQNPQPLTTYARQACPTAQQSTCTKPKTCTYKNTSLGGDRYMRTCSRCPSNYPWVGNPMGI

SEQ ID NO: 24 - protein, 519 aa, Rhipicephalus microplus
MKGVALFIALLCASSTLCLAQTTSVAKDVTSSERPVFFCYWGTWSHYRSGAGKFSVGQIDPSLCTHLVYAFAKLENGVIAAFDAYLDLKDNYGLGMYEKVNKLKVAHPHLKTLLAIGGWNEGSVKYSQMASTPEGRQRFAQSVLSFLDKHGFDGLDLDWEYPAARGGVPQDKQNFVLLIQELRRVLGPGRLLTAAVSAGESIVDGAYDVPAITRYLDYVSVMAYDFFGAWNSYTGHCSPLGIRQNASEEEKKLNVETAMKMWLNKGAEPNKLLLGMPLYGRTFTLANPSNDGFLAPTVGPGQAGPVTGESGYLGYIEICAQLKASKDWKITRDPRVVAPVAVKGNLWIGYDDAQSLTAKVLFARSLGLAGAMVWSIETDDFSGTCGGAKNPLLRAIRDALRSNTTISLPTTVTSLPSTTPAKSTPAETVSTKETNLQSTTSEASKSGSDTNVSVVTTSVPTSTTAAPELTCPADGFYPYPNDLHRFYRCVLLAGGTYAIYVFDCPAGTVFNPSVSVCTF

SEQ ID NO: 25 - protein, 129 aa, Rhipicephalus microplus
MARMAVSIALLVTALIAVSLAIPGGWSTKEPSSSPKYKELAHFVVAQRVEGLQKYDTVLKLTKVETQIVAGVNYRLTFTITTTDCIIAEVEYNAERCPPKNNQAKATCTAVVYERPWENVRSLTSLRCA

SEQ ID NO: 26 - protein, 1548 aa, Rhipicephalus microplus
MRVLWLSLLVAAASGFEVGKEYVYKYKGTLRVENPEQPLQSSGIAFKSRLVLQPKPDGTHFKLLNFEADSFNSEHIDVAHHEFNYVANENFAGVLEHPFAAKFDEGKLEEFVIGKNEPLFVRNLKKGLLSLFQLDLVKGRNDHHNDKQYHVKEDGLHGPCDTLYIVHEEAHGGIEVSKVKNLEKCDQHHYSFYGHEKGKRCLKCKEVATNPHTATSEVYYELKGTAQQYVIGHAWAESEQLFKPHGEGKQFHVILNRTLDLLEAHDAATTDTALPDGGEKEHSLAQEFPETHDLQNPEQLKHPNRLVAHFGLTPNKEDFVQGLQKLAQLEYTDEDIKEVDNKESGSLLFLMLFHNFLTFSYEDINDVYQNHVLTAPEDVKESMRRVFLDLLAAAGLNPHVTFGLNLIKSNELSADAADSFYHKLHLNLKEVSPALLQEIADSCKSEAVKSHREIWTTCKLAATTIAGGKGCKRAHDEHEKDHGLCAPELISHMFNYSVTPLDIENEPEYESTVFIRSAGNLGTRKALRYLERFIYPKWHANEPKRMAALWALKQAARLHPELARSIALPVFHNTSEPSEIRIAAFLVNVMTNPDLFVLRHIALEVLTDPSDQVVAFVVSAFRSLANSKYPCHKAIAQKLKYVLPLWETNPRFRKPLNKASSHLLISSGYNPKYDYGGLTLVEMIRSHDSYLPRNLYIVMKDYVAGHSTETVAFSFESWGLDKLLNRLVGPQPGSSKSLWNFMGRRRFPRDASAKERKEIEDALHIHEREYDPVYARLSLSLFGKAVDSWDFDESIFEAVKGKGAPEKTVEKLLGKEIRKKQFYISQDMTYLHPTELGVPVFFDFKQADFVYAHRQKIDIAHGDNAEINLNIKRHYLYETRLQQMVGFAWTYSRSSLGSGYDARTVVSWPLDLKATIAPLEGKLTLNRPLHLPWNAMNHHFHPFTFNTPYDLTRSHSNAIAEFTAKAKPLYRPDELLQFDRHYFGEIFGVAMKVKGHLVKRGLSQAMDEFYHKMDWRQRFYYLQVNPHWHPRNVKVYFEPAGDSPTKEMDIDIAYKFLEPDDERHSHFKANDQIGEDPEVPSTHVLNFNVNFKGDAKERKVAGELRYSFNHDLFNHKFQFFYERTPFRSNDNEGFKICLGATAKFPHPDWTRVNELATFYQGKHIDADLDIHYGSSCDEGQSSVHLHGQYTHTDSDEAQLVNAAAGKPITGNLRYNGLHRMALKCQAGREQGIPFNYYCLKFMRHSSRLAKLTADVEWKNYKPLFDKVFPVHAKYLAFKPEHGGFFSVIRSHFTGENGKLHVVSQVPWWDLKEEPHTDMVITTEDGKNYRHWGVPTFSHMLEPRVFSSLGYSNMAEYAKQYRHRYCDLQSLSLRTFDGTLVKLPETDCYKVVSRDCSPNKRFLILARSTNNPSLTKALKVFIHTTKLEILPVTADSGLIVRVDGNKVEATPERPYSHTDHDVELFEVKTHDKWFEVTSKPYGLYLTFNGNLLFVQTAPFYRGKLCGLCGDYNLDRNHELSGPDGHLYNNTLEFAKSYVVPSPECQAPAH

SEQ ID NO: 27 - protein, 873 aa, Rhipicephalus microplus
MSSRSSKYMYKSSGGAGDISIEYGTDLGALTRLEDKLRLLQEDLESERELRQRIEREKSDLTVQLMQLSDRLEEAEGSSETVVEMNKKRDTELSKLRKLLEDVHLESEETAHHLRKKHQEAVAEMQEQMDLMTKAKSKAEKERQKFQAEVYELLAQVENTNKEKITIQKTVEKLEHTVYELNIRIEELNRTVTEVTAQRTRLSAENAEYLKEVHELKVSLDNVNHIKSQLATQLEDTRRRLEDEERKRASLESSMHTLEVEIESLKVQLEEESEARLEVERQLVKANADAAAYKTKYETEVQAHADEVEELRRKMAQKISEYEEQLEALLTRCSNLEKQKSRLQSEVEVLIMDLEKATAHAQNLEKRVAQLEKLNIDLKSKVEELTILLEQSQRELRQKVAEIQKLQHEYEKMREQRDALQRENKKLVDDLSEAKSQLADAIRRLHEYELEIKRLENERDELAAAYKEAETLRKQEEAKCQRLTAELAQVRHEYERRLQIKEEEIEALRKQYQLEVEQLNMRLAEAEAKLKTEIARIKKKYQAQITELEMSLDAANKQNMDLQKIIKKQAIQITELQAHYDEVHRQLQQCADQLAISQRRCQGLQAELDEQRVALESALRSKRAAEQSLEESQARVNELTTINVNIAAAKNKLESELSALQADYDELHKELRVVDERCQRTIVELKSTKDILVEEQERYIKVESIKKSLEVEVRNLQVRLEEVEANALAGGKRVIAKLEARIRDVEIELEEEKKRHAETQKILRKKDHRAKELLLQTEEDHKTITMLNDAVEKLNEKVKVYKRQLNEQEGLSQQNLTRVRRFQRELEAAEDRADSAESNLSLIRAKHRSWVTTSQVPGGTRQVFVTEESSQNF

SEQ ID NO: 28 - protein, 289 aa, Rhipicephalus microplus
MAPRNWKRPLTLIYNDNYKYGNSLYSGALDDIEKRYQASLSRTTFRADRGDLGLSTFSSSNIGSAPEPSAGRPATTVTNESDHALLSPSFSAPSRSPVRSMLDTDHDLRKARSQGDFGRLRSSALRPNSWSDDEALMDDHSYKRRQRRRRPDRNDFLLSSPTVAAGENIDYYFDKCRELKSELEALHGRVLDAEARARSEAGTLRNKVQTEIAELSVQLEDTTSQNNELQKAVKRQAKQLLDLQAYCDEMQRHLQGAVDDLSTANKRCLDLQNEVDSLRSNLESGMRHI

SEQ ID NO: 29 - protein, 299 aa, Rhipicephalus sanguineus
MKIESLLVRQLISEFLGTMILITIGDAIMAIIIAGDNESLAACVGPLGWGVAIYVAVQIAGGVSSHLNPAVTLAQASVRKFPIAKVPLYFAAQYLGGFVGAALVFVTYKDAIEHFDQGVRQVTGEKSTAGIFATYPKPHVSTLTCFIDQVIATGIMMVCCEAIGDPRNFGGIPPHIHPICLGLLIMALIFSFAYNCMCPLNPARDIAPRLFTLMAGWGPETFTLRGGNYVWVPLLGPHIGAILGVWLYKVAIGDHWPDRPKPASTNGKETKEDLVETLYKVDGDKMVLELEPAQQHQRL

SEQ ID NO: 30 - protein, 291 aa, Rhipicephalus sanguineus
MKPNAVTRAWRRVSGCCIEKPLARQALAEMVGTLVLTLVGDCVLASLAVFQLGSVGLAAGPLGWGLAVFLGVLVAGGVSGAHLNPAVTVALATIGKLGWCNVLAYVAAQYLGAFLASGLVYLVYADALAQVDANLDTVYGINATAPVFSCFPAPGVSTLTCLLDQTVSTAVLLLGICAITDTRNMAVPRGQQPLLVGLTVSACMYAFSYNCGNPLNPARDLAPRIFTAMCGWGSAVFSFRAYNWFWVPVVGPHLGAVIGVWIYKLAVDNHWRDEDEIDDEKKPLLGNGKIA

SEQ ID NO: 31 - protein, 704 aa, Rhipicephalus sanguineus
MRSLALFVAAVSLIGGCAAQSSVCSDFGNEFCGNAVCEVVPGAEHDFVCKCPGDDMYYNAAEKHCEYKSTCKTKECSYGRCAQISPRKAVCGCDGVDSLTQRCNIQGWYADECRRKGGTAKLRTDGFLGAKCDCGEWSVMDRNKRKCVPTTCLRPDLTCKDLCEKNLLEKDSRCCQGWNSANCSTAPEEGSYCSPGNLKGKDGSCKDACSAKEGTLVCKYGCKGSTKPMRAYECECQSGDEVAEDGITCKRVPYTGGCSDEQKKTCRPSEYCLAKKENVVCECPMRQHLVNDECTSDCVDDKCHENFTECGVYMWKQNCYCPWRTRKPLPNVSINECVLNEYYYTVSFTPNISLDSRSCDSYAARVLDAIRTSIGNDVFKVEILNCTHDIKARLIASKPLSKHVIKRLQACEHPVDDLCMLYPKLPIKKGSATEIEEENLCDSILKTQENGYKGQNKCVKVDNFFWFQCADGYRAVDEITRGRLRRSVCEAGVSCSAKEQLECANKGQICVYKNEKANCQCPPGTVAGQAGCTARTTCNPKEIRECQDQKRKCVYKDQKAQCKCPEGTVDDGDGCSIEPAKEPCSEYGVGKCRSKGQSCVMENGKPVCKEISDATTAATATTKAKDKDQEPCSEEDSGTCRSSGQRCVMEYGNPVCKKISDATTATTTTTKAKDKDQDRGKSSAAAVSATWLLLLVVATSVVSA

SEQ ID NO: 32 - protein, 2680 aa, Rhipicephalus sanguineus
MKTRLLHLIVACSLLLGTVTCSNSTDDGSSLRTQRQNNGSSSRTNPGRIVCYYSAWALYRPEPMNYDIEDIPLDKCTHLIYAFVGLSNQTWELFSIDPEYDFNKGGYRRFTALRKKNPQLKTLLAVGGWAEGGKKYSEMVSTTGRRHTFINSALKWVQTYGFDGFDLDWEYPGAYDRGGAYSDKANFLLLVRELRSVFNQYRLLLTAAVPVAKFRLQEGYEVAELGELLDWINVMTYDLRGNWAGFTDVHSPLFRRSFDEWAYEKLNVHDGLQLWMSLGAPREKLVVGVPFYGRTYTLSDKSNTGLRAYINKEKNGGIPGPYTNATGFLAYYEVCPHVNSGTWTKKFDDVGKCPYAYYDNQWIGYEDPESISIKMDYIMGQGYGGAMIWAIDMDDFQGVCGRKNALINVIYEKLADYDVPLPEPTTTPVRSTFDDGQWKPTTTPLPSTEPTTPKIAVTKSYPRDCDSPTISFIPHENDCTKYYWCVYGTPMVMFCEGGTVWNQDNGNCDWPERVDRPECKHITRKPKRPHKDIENESAYRATADTRLEDSVSLRTWRTTEENMWFTVPLLICLPAVFLQPAAAVSKGSDAIQGIKIPRNKFEVPVVCYYYGWASRRPSPMNYQVEDVPLDMCTFVVLAFAGIDDKTFELKSMIPEYQEDSQRYLNFTNLKTQHLYLKTMLAVGGWHLGGEVFSKMVSTPESRAAFVWSALRWMLDYKFDGLEIIWKYPGYEKRGGRPQDKENFALLLKELKEAFAAHPLYLTAAVPLEENYLDDGYDIPKMQRHVDWFNVLAYDLRGPWNRFTDMHSILYKRAADSPYFQDLTIAEGMKRLVRMGAPKQKLMLGMAYYGRTYVLRDSKKNGPKARIIPNGKPEVGPYVGSDELKGYYEICQDVKSNGWTRKFDNEAKCPYAFKGDQWVGYEDEESVANKMDFILREKYRGVMVFNNDLDDFRGVCGPKNPLMTVIFNKVGEKALREIRANQIRSTPNKETATRRCLRLSFKCIEHLQVGSHGSFTRCFGERERNGVTTLWLRGSTRTNAIYPLRNMREVLRGFLDSFLRNLESIENTRRHRVALLILLLAALQQPASAVTSGRDAVEGVTIPRNKFEVPVVCYYYGWASQRPSPANYSIEDVPLDMCTFVVLAFAGIDNETFEIKSMIPEYEEDERKYLGFTDLKNRHLYLKTMLAVGGWDLGGEVFSDMVSRPERRAAFVESAVRWMRDHEFDGLEILWKYPGYEKRGGKPRDKRNFVLLLKELKEAFAKYPLYLTTAVPLEENYLDGGYDIANMERYVDWFNVLAYDLRGTWNRFTDVHSILYKRRNDPPYFQDLTIAQGMKRLVRMGAPKRKLMLGIAFYGRSYVLLDAGDHGVKARIKFDEQAEAGPYVNSTELKAYYEICMDLKKGGWTREFDNVAKCPYAYKGDQWIGYEDEESVGHKMDFILREGYRGVMVFNNDLDDFQGLCGPKNPLMTVIFNKVGQKALQELRNQTRSQQPEVTTTTTTMLHIFFCGFLLSHVLQTVRPDDDFIRNGGSPVVCYYAGVAADRFSPMNYRIRDIPGDLCTHVNLNYAGVDKDTLGVKDLIPAYTNNSHYKDFTAIKKKFLFVKTLISLGGWEHGGESFSYVAADRSRRCNFTRNLYKFLKDNDFDGVDIDWRFPASADKNGKPEDKENYVLFLKAQGLKRLVELGAPKKKLVLGIPFFGRSYVLADKEKHNVGDPIRDIPAAPGPFIGSTEILAYYEICTNIEDGIATRAFDNESMCPYMYYEDQWVGYEDEESVGIKVDFVIKEGYAGVMVFNNDMDDFNGVCGTTHPLLKLLQLGVLLRMFQAAVFIENFSHGRMLPVVCYYEKGQALRPFPMNYDIDDVPGHLCSHVNLAYITLNHTTYTIHEDNTIKFADFAKLKTNFTRLKTFISIGGWNNQTQVISAMASMADRRKAFINDTVRLLVDHNFDGLDIFWLFPGSVDRGGSIEDKVNFVRLLEELYAAFRVHKLLLTAAVPINQQILDNGYDIPEINRCLDWMNVIGYDLRGYWNGRTDVHGPLHRRSIDGNDTADLNVETGLQALLDRGASKRKLVLGVAFYGRVYKLANPKEAGLHAPIDFINKPERGAFLMSDNIHAYFEVCKNIKFGSWNRSFDSEGMCPYASNGRDWVGYEDAESISKKVEFVRREGYAGIMVLSCDMDDFRGVCGTRNILLQTINDAIPRLRNTVILRNVEQERALVRSILVEANHAAFRTASALNSGQRIAMELLTLPLALLLTCALAMPQQNGSGSTSSTTSTASSSSSTSASANATTARTRSDTHSAPAPVVCYYYAWANARPHPANYGVKDIPGDLCTHVNFAYAGVNPQTWELRSEVPEFESNRDLFKNFTAIKTQYPQLKTLLSVGGWQHENGVFSQLVASADRRALFIESVLLWMKEYNLDGVDMAWRFPGVSYRGGSPRDKENYASLIRELAGAFQGKDLLLTVVVPVSDEFLDPGYNVAEMSKYVDWINAQAYDLRGAWNGVTDVHTPLFPRSIDFGPQATLNVKDGLARIVSLGAPKSKVVMGIAFFGRGFTLLDPQQHGLHALINREVPPHAGPFVRSNEIFAYYEICLNLKGNWKREFDDEGKCPYVYYRDQWIGYDDAESIQHKIDFLLQEGYRGVYVFNNDLDDFRGFCGEPNILLKTIKNGLTEKNELDARVNEKV

SEQ ID NO: 33 - protein, 257 aa, Rhipicephalus sanguineus
MMFHPFHLVEKSPWPITSSISALSLTLGFVSYFQNLSMYLILLAVLMITISSFQWWRDISREGSFQGFHTHWVLNGLKMGMILFILSEVFFFVSFFWAFFHSSLSPNIEIGSQWPPKGIYPFNPFEIPLLNSTILISSGITVTWSHHAIMNKNYISALNSLLITILLGVAFTMFQGFEYFQAQFKISDGIFGSTFFMTTGFHGLHVLIGSIFLLVSYFRIKNQLISSDHFFGFEASAWYWHFVDVVWLFLFTFMYWW

SEQ ID NO: 34 - protein, 216 aa, Rhipicephalus sanguineus
MPVLLYNLVGSPPCGFIRCLAKEIGVQLSLKDLDFGKGEHRSAEFLKINPFHKVPAIDDSGFIVYESNAIAYYLLRKYAPESQLYPNCIKTRTRIDQVLAAVNGNINPSLGAFLRPLCLQSTKHGADELKACEENVVKTLERLVGESKFAAGDKITIADLCLIGHVIFCLEIPCVNKAKYPKLTAYYDRVKSALPYFDEIYGPVLTLIKKLWDRMK

SEQ ID NO: 35 - protein, 549 aa, Rhipicephalus sanguineus
MLRIIVVSVLVISSRGMQQPRLVYPRLLEERSEDGRMVMHLHDKLTLNLRKASVAARNFRVLEHEDGHEVTHFFDGRDLEQNLHEDEKQLATVHVTRRDGGIEVEGVVGPHHRIEPVLTMERSEEGLIPHVIHEIEKKEMVDIERAIPEKDAWTVNERSSGTGNVPEEVTVEIFIVSDVLHFSHFKSNVELITYLCIHVNSVNMRYADTKGPRVKFLLIGVERDQFSTYRNGTGKLIESQSSLDNFRQYADMKRYEYGYPDVTYLMTGYDAYSLNADGTKNMNVLGLGFVGGLCTKFFVALGEDVAGTYSGMHTLTHEGGHVLGASHDQSSPKSWIPGDPGSLSCPWSDGHIMSYVDGGVRHHRFSRCSLEQIRNLVILRGYVCWKVGITNTGYTEPGKYPGMAVAPNEFCKSVFPNERNVTADMKSDRMAQCMIKCQYPVVRQHCFSYGYCTTYTSVRYTHEHALDYMPCTADKVCVRGVCGGSTINVPQRTVTTSTSTRRTTTSVADTSECKCDCSSTTTAPVSTYPPRRSYPWWARRNGIRRSRGQ

SEQ ID NO: 36 - protein, 380 aa, Rhipicephalus sanguineus
MAVNQLGDSVLNFALDLYGQLKPKDGRKGNVFFSPFSISAALSMALGGARNKTAKELSTVLRVPDGAQIHNHFSDYFSKLQFYAADVKLHIANRMYCEQTFPVLESYLSLLRDSYSATIESVDFRNDYETVRLKANAWVERATESKIKDLLPGGSVNAKTTLILINAIYFKGLWASQFRTDATQPSDFHLDSKNKKQVDMMYHKDRYCTGRSKELDVEALEIPYRGGKTSMVILLPNEVEGLSKLEKRLTASKLGNLLENLRGFFDVELYLPKFKLEQAINLKGVLKEMGIKDFFSSDAELSAICEKDKLTASDVVHKAFVEVNEEGTEAAAATAVMMAACCLSSTPPQTYKFIVDRPFMFVVRSHDPDVVLFMGSVRDL

SEQ ID NO: 37 - protein, 403 aa, Rhipicephalus sanguineus
MASFVAALLLSAAASLAPTFPQDAHLRHARASNAFGVALFKELCSKTTDRNVVFSPASVSIALGMLYAGASGKNLGELSTVLGLSDAGLVDRDAVLSAYKSLVETKSANATLDIANIVLIQKDFEVLEQYRKDVAKYFHAGALSVDFFRDAQRVAAEINDWVKKKTRGEIPKLLDVAPPMNTVAFLINAIYFKGMWETTFQDRDTKPLAFYNHGQDEVKVATMSVRRRFGYAVVEELEARALEVPYAGDRFSMVVMLPNSRTGLSTVETRLTTDLVEKIANRLTLRDVQLWLPKFKLQTEYDLVEPLRRLGLISAFGKGADFSGISARNDLQVSDVVHKAVVEVSEEGTVAAAVTAVRVSFKSGGFVRLPPPTPFRVEHPFAFYIWDKVDKRALFMGAIRSLQ

SEQ ID NO: 38 - protein, 380 aa, Rhipicephalus sanguineus
MAVNLLGDSVLNFAVDLYGHLKPKDGRKGNVFFSPFSISAALSMALGGARNKTAKELSTVLRVPDGAQIHNHFSDFFSKLPSYAADVKLHIANRMYCEQTFPVLENYLSLLRDSYGATIESVDFRNGYETVRLQANAWVERETESKIKDLLPGGSVNERTSLILINAIYFKGLWASQFRPDATHPSDFHLDSNSKKQVDMMFHKDSYSRARCEELDVEALEIPYQGDKTSMVIILPNDVEGLSKLEERLTASKLANLLENLCGFADVELYLPKFKLEQAICLKEVLQEMGIKDFFSSDADLSAICEKDKLAASDVVHKAFVEVNEEGTEAAAATAVIMLTCCMSSLPPRTYKFIVDRPFMFIIRSRDPDLVLFMGSVRDL

SEQ ID NO: 39 - protein, 161 aa, Rhipicephalus sanguineus
MACATLKRTHDWDPLHSPSGRSPKRRRCMPLSPPPTRAHQIDPSPFGDVPPKLTSEEIAANIREEMRRLQRRKQLCFQGTDPECQPTSGLSSPVRRDQPLFTFRQVGLICERMMKERESKIREEYDHVLSTKLAEQYDTFVKFTYDQIQKRFEGATPSYLS

SEQ ID NO: 40 - protein, 282 aa, Rhipicephalus sanguineus
MAPPCYADLGKQARDLFNKNYHFGVVKLDCKSTTHTGVEFNVSGTSLNDTGKVNASLETKYKVPEYGLTLKEKWNTDNTLSTEICTEEKLARGCKVAFHANFAPQTGKKSGALKAAYKLENVHLNGDVDLGPPGPVVHGAAVLHYQGWLAGGQVSFDTTKNRLSKTNFAVGYQAGDFVLHTNVNDGQEFAGSVFQRVNPQLQTGVQLAWTAGTNATRFGLGCVYDLDMETSVRAKVNNSGQIGLGFTHRLRPGISLTLSTMLDGKNFNQGGHKLGLGLDLSA

SEQ ID NO: 41 - protein, 1782 aa, Rhipicephalus sanguineus
MRVLCGLPLLLLAAAANGVLFDVVPPTQSGGVVYKVRATVALKSPDLSVTEGAGLQYAGDLALYSSGQDTYLAQFLNFSVVKHEKLYGGLDNLTAVPLNESVPLFNESSAVDYSQKFRLPVKFVLSAGKVVKYEVSPEVPVWALNVYKSVLTLLQNQVETPQEVPVAAAYYEDGVSGHCRVTYEVHSLTKFLPTEGKVYNVTKTKYLDDCKLPRPVHSVSGAYSKGYPAVCAKHLPNNFLPGFQEDTSAYESKPLVGCPEGLSPVDSVVTAHEVSYYNVSGSLLESALSDSLAVLPLLTTRVVVRTRLQLELASLSVPPTNPVAFNGEPHTSLEHQLPEVAEYLDLPVYSYLVGSPEQVKPEVFTQVLEEVAEELVSVELDTETKKTPALMLKLVHMVSLLNTDQLKQALPQSLLANTSVLEPKQQVLRALYVDLLGKAGSKSAVEVAVHLVKQQVLSLYEATRLLRELSVFQAYVDKDTVELLLELCKATETVLHPLVKVAVCDTLGEAVKKACPTKVHLEAVPYPTYKSSWRRIPKLKTRYEQTPVVPKHLLPYESVERLVLPQEKQCTVADLYEYVNALEEALKVTKDFKVLVGYLNALGKVAKPEVLPVLMSYLNGSAENLYELAEQEEDVTETVYFVKKAVLLALSHVVEYFPKEVSPLVRTVLLNVSEPVDVRTLAFDVWLKSVPTKWDLQTVSLVVKLDRSLELKSYVYSALESVVKDKHPANHVLASRVRGVFVHFEALNVGPRYSKYVKKTYYNVAKQFGVESLFKHVANNVSYFPTHLHAGLKYNLGPYVKTLLEGKLLLKGGEKFLAEVFSTYGTSTLLKSVPTRKIGSTYESGAKYSVVKELNLEARKVEFPKAVLFAKFLGGDAVLPVDKELFEQLKSELVEYLKKVASSGPVTTHLVRVLLPKKYVHVEPTVVGLPVVQTVLHPIVVSLKLKNVSLLAETSVDSLLPVTLNVTGTVQPLVLSVKQNHVFVPADLTEESPSVVHTSVKHLHAKAHLTLGYDKKYEKKVHVAVKPTFGSVLLSAVSTEHVVETPSPFLLLEKPVVDPKKVVDSLVKPFTYHKVYEAKLVNVAVDAVSHGPLLPLPLYGKLYHLPTERMTKFVEYLTHTGSKQHLLRVSVKPYEKDPVNEWVATFKYEHNLDYLAKATLKKVYTPTKSSAYNESDETSYEHMSYRPSTPAYESYEPEESYDYYPEYHQWNKTLVKHVLNVTLEGKQEGSLKKLTKLDFAYYHTVNKTLKHYYVDVKTLTKPLVSVFLNLSHPVPPSPFWYEPSFVGKDLVNATLYVTYGNEPEPFVLTYNATKTVEQLKGLHAYDATPLLHWFVPQCLADQYAGNTVSYACRLATVHDAHLNKQVVAFKVPTQVSPKVKSVALKVLSYLKFKLFPYASFYALPLTEQEEYHVVFKLNKTDVNPYVTVAHGELLLPGEKVVLTDLKLSKYLVPNLVLSGFDRLKHSLYENHPHPPCSVGKNWVRTYDNVSYPLEVRPNCKYLVTSDCSAKHDFAVVAQPLDLTVGTKKLVVQLGPTVVELPPPDLYKAEVLLTVNGTYYVANATQDVVLPYKWDRKLLVTVYPTSGPHDPPVVKLTNSLKTFKLLFDGVNFFVWVNPLYQGKTCGLCSNYDNEPYHEFVTPENWLVSNYSEFVASYGFGLPQCKEPLVSAVPVHYLEQLKKPVGHQSVYPTHHEYPSHHQEYPSHHDKYPSHHQKYPSHHHKYPSHHEYPTHHEYPTHHEYPTHHEYPSHHEYPTHHKYPTHHKYPPHHEPYPHSANLQREG

SEQ ID NO: 42 - protein, 1799 aa, Rhipicephalus sanguineus
MKATACAVLAIAALYLVGDVSSECPQAWFDCGNGRCIAMFWRCDGQNDCGNHKDETGCSATQSRCPADKFACHDSSYCVPQIWVCDGEADCHDSSDEQDCHSSNCTGYRCHNNECIPAPWRCDQTEDCIDASDELDCGGAKNSSATTLAPRCDVDQGRFPCLDGQCLLPSKVCDGRKDCSDGADEGAFCKVNECSQKKCSQGCFVATNGSTCYCNAGFRLMADHISCADIDECVEDPHVCSHGCINSPGSYSCHCLEGYQLADKSFCKARDPEPLLVFSTTKEIRGLWLRSNRYFEIHPAEAQAVGVEFDSDQHRVFWTDVSTWKSSIHSCRLDGSDFKTLFSAEKTLLEDLSLDWVTNNLYITDSLVKRILVCTTDGVSCSALITDSVDSPRAIIVNPPQKAVYWTDWGPRPAIMHSSTDGTNIQQLVSTDLGWPNGLTLDHTTNRLYWCDAKLSRLEYLELATLKRDVVMDEDVFHPFALTVFEDTVYWSDWASYSLDSSNKRTGKQHHRILRENSHHIMGVHVYHPVLRQRGIQNQCWDNPCDHICVLSGDSYMCLCRIGYKLAANKHSCTVTKDFSFVIVAEEDLLYKIDLNKVGAPVPKTLPVTNLGMISALAFDWSNQTLHYSDNRHSVLSAINVNTFEQWTVHDHIGSVFGMDFDVTHQLLYWVDADKYTLEACHANGSGHTIIRDDLHRPVGVALYPFAGILFVLSAGDKPTITSYTMDGQNPRVLPLSTLLLPVSLSVDLVARTLVWADAVRGTIESLDLQRVSTGTPFIVQQVKAHISSVSAAHNEIHWASRDKASLEYIDLSSEPSVRRHVSLRSTRNGTFSRRVIVAAQVPPFAPGPCGQSNGGCSHTCLPVRTTDRSCFCPPGMALNADNITCRVENGTCRPHELPCAGRCIAATNWCDGHKDCSDNADEESCGSTACPSTDFTCSNGRCIEKEWQCDGYNDCGDSSDERNCTRQTCASHQYTCRSGVCVPLYWRCDGSEDCPDGDDELNCRSVRCPSGHNRCANGQCIPQDWTCDGHADCTDSSDEKNCTESPSCFEDDFHCANGQCVDKRLRCDHDDDCEDSSDEVGCDFAKVNRTKCSTGMVDCGDGQCIYTHDMCDGYVDCHTGRDERNCSAPICRSAEFFCTGTKRCILQSWFCDGDDDCGDGMDEHLERCHPTTKVSATPTPACGSNEFKCGSRECISWSRVCDGRTDCADFSDEGSHCVNYCGTTNGGCAHLCRESPAGPLCSCHPGYRLNTDKKSCDDIDECATPAHCSHFCQNSKGSYKCTCADGYALGADRRYCKVQYGEPFLLYMLPNQIRSFSMHGHAQHLLAEDSLSDMHGMDYRVTDKSIFWTEMDEGVINVMTLGNGKQFTLLEEIHKPYHIAVDWVAGNIYFTDGWVHIQACEPTFKHCTDVLDTAYSHLNTFALSANDGLMFWAVWHEVVNKDYGLIERSNMDGTARVALLTDKILWPCSITIDAVHKRIYWSDANKNVIESATYDGKDRKVVRGAGLSSPFSIALFEDWLYWSDWGSDSLMACNRHTGTHVGLVHHGTAKATVLKVLHAVHQPSGVNRCARNQCAHICLLNPSAYTCACSHGYTLAEDSHSCVESDEHYHLNSSDILGQFCNPVCLNGGRCISANDSYFCTCAGGFQGPSCADAVVVSMLPQLSNSSSTALASILISVLCVALLVLGYVLYRRNRDKLAALDFSVSFKKPNFRKRQGLLEDEHPIAADEDYHAMNTAPGFINPAFDTRKGQLLSENGEFKRWASSDSLQSSSSKEQSSCVLAGDSAAKQDQVFFFRKH

SEQ ID NO: 43 - protein, 967 aa, Rhipicephalus sanguineus
MRLSYARGVAFTDIRIASTEVAQLALGIQALVEQLPKDTSLILGLEITDYDEAGQLLAQRLAPIYEYADVVVLQTHLRSGHNWPFCRTTFASLFDHPSDDSCLQTPPAVTALSWIKLVQPFSVPTCLSLNMAAVKFKASKNATPEASCENAVETNDVCTAQAWPSAEESSSVLAAYRYKDGFSVTMETAELLREKLSRAHRLYPRFCVALYNVDVDAEPCEGSDLAFARVAQIADAAQYKHRKPTTEQPRGRDSGGSQDDESGPRTAVQNATSSNALVCILSASALNMETFPMRLCSYVVYTRRVTPGAPPQPFVPQNDAKFYKFVRRCEETESKGLVAVTPPMLATLFKVNATLDVDALNSVMRRLAASRVAGLAVFATPSTDLQALAVMTQETFKVLKDTFKMVVGVHVPSFDAPVLKELSKNSHLLVLFTHSMLRGSTCTVTAPSSLSLTPTHYELMKNLPTLEGDRQSSTCISVNFAVMGFKTRSSNASLGDGCVNRRLESYDHTCWAAANLWQTTSQLVHRHNSSWVQTFETEKSVGSAVRPFLEAYPQGCAAAVYADFEDASGTCRPRYSRLRALSALMATKAAEASLAANSASTTAETVADSLPTGLPEDMISTHVTKRNKMVCFVTPSTHIVTDVPADSCDYLVLSTQNFTAVENVTDQFGRWKELMTQLSQKTRLALGMNARTLVSLYKGDYAAGVLLVESVPSWLKPNGGRALALLAGSLASQDELLLFRILKDLRSALNQAGSPAPEIILAVSSDSELFHKELIELSDVFVLVDHHIPREGGSPSCKVAFPSVSPRTSSLTSPMLIAKDLMQKGADNKTYCISINLAVLKFATNTGSLSLSNGSHCDSEDEVAYSEVCQMSNAVPVYDMNWMTASLKASRKLYTFEKAEMLLSKVLFLKAWFPSLCVAAFHAELDTVDREACPQSEQYARLLSVRRGLDSAVHGESHWRRDAADGA

SEQ ID NO: 44 - protein, 318 aa, Rhipicephalus sanguineus; identical to SEQ ID NO: 16

SEQ ID NO: 45 - protein, 391 aa, Rhipicephalus sanguineus
MKIIICLAGLLTLCSGQVEHKVTAANNQFGFKLLNEIPVSPQTNLFYSPYSVSTAMAMAYIGARGETQRDLHETLGYSSVGLTPDHVPSAHAQHTHLLRAPSNSTLRVANAAVFQENYAVTPEYVEILRQSFEAEVRAASLNDEQSVRVINDWVKNKTEQKIEELLSEPLPPDTRLVLLNAIYFKGLWNVPFDARGTLKKPFFNAGTERVEVDTMYEQVTAGYAEDDETNADVLDLPYAGLDYSLTIILPKERTGVDSLRQNLSWPVFQRLLSKLNMNAPMEVSLPKFKIEGSYKLKAPLSALGASKAFDERHADFSGISGARDLVISDVVHKAVVEVNEEGSEAAGATAGIFYTKSAPVGIPFVVDHPFLFFIRNRHTGDVLFAGQVNHL

SEQ ID NO: 46 - protein, 191 aa, Rhipicephalus sanguineus
MLRVVLLTLAATSAWAGNSLNEQANQNRYFLNDRCRVGLQEQVHLELHASLVYMQMAAHLANNKVARGGFARFFRDQSSEEREHAQKIIDYVNLRGGTVSAVHVDMPPTASWMSVLDALQAALALEHRVTNRLYELHRLAEEHDPQMADFLEQEFLAEQVRSIDQLQRLITQLQNMDTGLGEFLLDQQLRT

SEQ ID NO: 47 - protein, 96 aa, Rhipicephalus sanguineus
MKTSFVIVLFLCSAALTVATDEESDSAGSWRPEPQPCAEGETWKECVSGSCAEATCDQPVVGPACTEDCNFGCYCADGFYRDQHKACVTRDKCAEH

SEQ ID NO: 48 - protein, 156 aa, Rhipicephalus sanguineus
MAFVRQAVLLVVLVGACWAHKDGAPDSACATMIPQHNDSTHDVAGPSTPFHLVQDKRDFKAGDVVAVTLSSSGTPFKGFFVKAFNENNQEIGQFETSPEAKAVTKCSGVTHTNPAEKTTVKVLWKAPEGAAGKVHFRATVVIDYHKSVGGIQSTVA

SEQ ID NO: 49 - protein, 219 aa, Rhipicephalus sanguineus
MGDTDAERRRKEAEDAHRRLKEEEKRKMEEKERKKAEVRKRLEEAAKAKKAGGKRGFMTPERKKKLRNLLRKKAAEELKKEQERKAEQRRKIISERVGQPKPLDGANEATLQGILKQYHARIAALEDAKYDLEYEVRQKDFVINELTIQVNDLRGKFVKPALKKVSKFDKLKMVAKSTSEVDFRSNLKAVKKEAFKLDEEKQAEKKPEWALGSKKENEE

SEQ ID NO: 50 - protein, 441 aa, Rhipicephalus sanguineus
MPRRRTGARGQSTSPKPPPSKTGGVMGFVSTKHVLAVMVACFATHVWIQGSSAVDSVDPDVSRNVSQIIADKGYPVEEYEVETSDGFVLSMQRVPRGLKSGSKRLPGAKKVVFLLHYLLGSSADWVVNYPEQSLPYMLADAGYDVWLGNVRGNTYSRHLRYNRREKDFWDFCFDEMIAYDLPAMLDFVLNKTGQQKLFYVGHSQGCLILFGLLAERPEYNDKIELFAAMAPVTTVTYMRSPIRHLAPFVENAGALFALFGEYDFLPSSPLMRLVAGTLCQFEESRSLCENVIFFICGPEGPQLNKTRLPVLVSHTPAGTSVRNMAHYAQLVLSEKFQKFDFGRIRNRARYGQPNPPEYNLTRVTAPVALYWSVGDWLADPKDVSQLRDKLPRLILDFRVRDRSFTHLDFSVGVGARRLVYEPLMKAMASVRALVHHFYPDS

SEQ ID NO: 51 - protein, 445 aa, Rhipicephalus sanguineus
MTTQLSSPLSIVLLCFWQTVAAGSATTCNLSECRSRDNCACMSTRPPENLNASDMPQFVMLTFDDAINEKNMDFYRQLLDPGNRRNRANGCNMAATFFVSAGYTDYSLVHELHSVGSEIAIHSITHRDNLTYWRALDVAGWKEEFVGDRDLLRDYAAIPERDMVGARAPYLEVGNGSAYTMMRQNGFVYDSSVCIDYMNETDKLPFFPYTLDYGLGLTCTVPSCPSRGHRGLWLVPLNSYYMTTTSGGDGSRTIVTSCAMPDTCAPKPTTKDDTLDFLRSNFERYYHTNRAPFPVFIHETWLWTPGRRQGYLSFVDWLLTLEDVFVVTVAEVVRFMRDPKPLGEYVQASCSRASEFQRCPQVHACSFPNSPIEETRHLVGCRPCPESYPWLRDVARTAKHDAPFHVAEKQNFCAGCVILFCLAIIVCLGYFATKPLERRSKTHTL

SEQ ID NO: 52 - protein, 539 aa, Rhipicephalus sanguineus
MKGIALFIVSLCASSTLLLAQKTSSLTKDVTSSKRPVFCYWGTWSHYRSGAGKFSVGQIDPSLCTHLVYAFAKLENGVIAAFDAYLDLKDNYGLGMYEKVNKLKVTHPHLKTLLAIGGWNEGSQKYSQMASSPEGRQRFAQSVLSFLDKHGFDGLDLDWEYPAARGGVPQDKQNFVLLIQELRKVLGPGRLLTAAVSAGESTVDGAYDVPAITRYLDYISVMAYDFFGAWNSYTGHCSPLGVRENASEEEKKLNVETAIRMWLNKGADPNKLLLGMPLYGRTFTLANPSNNGFLAPTVGPGPAGPVTGESGYLGYNEICTQLKTSKDWKITRDPRVVAPVAVKGNLWIGYDDAQSLTAKVLFARSLGLAGAMVWSIETDDFSGTCGGTKNPLLRAIRDTLESNATIPLPTTVTKLPSTTPAKSTPAKTVSTTGTNLPSTTPAKSTAAETVSTTGTNLQSTTSAASKPGTDAGVSVLTTSVPTTTATPELACPADGFYPYPNDPHRFYRCVSLPDGTYAIYVFDCPAGTVFNPSVSVCTF

SEQ ID NO: 53 - protein, 129 aa, Rhipicephalus sanguineus
MAHLTGSIAVLVTALVSVSLAIPGGWSTKEPSSSPKYKELAHFAVAQRVEGLQNYDTVLELTNVETQVVAGVNYRLTFSIAATDCIIAEVEYNAERCPPKDDQAKATCTAVVYEKPWENLLSLTSLDCA

SEQ ID NO: 54 - protein, 1548 aa, Rhipicephalus sanguineus
MRVLWLSLLVVAASGFEVGKEYVYKYKGTLHVANPEQPLQSSGIAFQSRLVLQPKPDGTHFKIVNFEADSFNSEHIDVAHHEFNFAANENFAGVLEHPFAGKFDEGKLEEFAIGKNEPLFVRNLKKGILSLFQLDLVKGRHEHHDDKEYHVKEDGLHGPCDTLYIVHEEEHGEIEVTKVKNLEKCDHHHYSFYGHEKGKRCLKCKEVATNPHTATSEVYYELKGTAQHYVIDHAWAESDQLFKPHGEGKTFHVILNRTLDLVEEHDAPTTDTTLLEAGEKEHSLAQEFPETHDLKNPEELKHPNRLVAHFGLTPNKEDFVQGLQKLAHIEYTDEDIKEIDNKESGSLLFMMLFHNLLTFSYDDINDVYQNHVLTAPEDIKESIRSIFLDLLAAAGLNPHVTFGLNLIKNNELSVDAADSFYTKLHLNLKEVSPALIQEIADSCKSEAVKSHREIWTSCKLAASAIAGGKGCKHAHDDHEEDHGTCGPEIISHMFNYSVTPSFVEHEPEYVSTVYIRSAGNLGTRKAMHYLERFIYPKWHANEHKRMAALWALKQAAKQHPELARSIALPVFHNTSEPSEIRIASFLVVVVTDPDLFVLRHIGLEVLTDPSDQVVSFVISAFRSLANSKYPCHKQLAQKLQYVLPLWETNQRFRKPIDKASSHLQISSGYNPKFDYGGLTLVEMIRSHDSYLPRNLYITMKDYVAGHSTDTVAFSFESWGLDKLFNRLVGPQPGSTKNLWNFMGRRRFPRDASAKERKEIEDALHIHEREYDPVYARLSLSVFGKAVDSWDFDESIFEAVKTKDAPEKTVEKILGKQLRKKSFYVSQDMTYLHPTELGVPVFFDFKQAEFIYANREKIELTHGDNAEIRLGIKRHYLYETRLQQMVGFAWTYTRSSLGSGYDARTVISWPLDLKATIAPLEGKLSLNRPLHLPWNAMNHHFHPFTFNTPYDLTRSHSNAISEFTAKVKPLYRNDELLEFDRHYFGEIFGVGMNVKGHLIKRGLSQAMHEFYHEMSWRERFYYMQVNPNWHPRSVKLYFEPAGDAPTKEMDIDIAYKFLEPDDERHSHFKVHDQIGEDPEVPSTHVLNIDVNFKGDAKERKVAAELRYSFNHDLFNHKFQFFYERTPFRSDDHEGLKICLAASAKFPHPDWSRVNELATFYQGKHIDANLDIHYGSSCDESQSSIHLHGQYTHTDSDEVQLVNAAAGKPITGNLRWNGLHRMALKCQAGREHGIPFNYYCLKFLRHSSRLAKLTADVEWKNYKPLLNKLFPLHTKYQALRPKHGGFFGVIRSHFTGENGKLHVVSQVPWWDLKEEPHTDMVITTEDGQRFQHWGVPTFSHMLEPRVFSSLGYSNMGEYAKQYKHRYCDLQSLSVRTFDGTLVKLPETDCYKVVSRDCSPNKRFLILARSTNNPSLTKALKVFIHTTKLEILPVTEDSGLIVRVDGNKVDVAPEHPYSHTDHDVELFEVRTREKWFEVISKSYGLYLTFNGNLLFVQTAPFYRGKLCGLCGDYNLDRNHELSGPDGHLYNNTLEFAKSYVVPSPDCHAPAH

SEQ ID NO: 55 - protein, 873 aa, Rhipicephalus sanguineus
MTSRSSKYMYKSSGGAGDISIEYGTDLGALTRLEDKLRLLQEDLESERELRQRIEREKSDLTVQLMQLSDRLEEAEGSSETVVEMNKKRDTELSKLRKLLEDVHLESEETAHHLRKKHQEAVAEMQEQMDLMTKAKSKAEKERQKFQAEVYELLAQVENTNKEKITIQKTVEKLEHTVYELNIRIEELNRTVTEVTAQRTRLSAENAEYLKEVHELKVSLDNVNHIKSQLATQLEDTRRRLEDEERKRASLESSLHTLEVEIESLKVQLEEESEARLEVERQLVKANADAAAYKTKYETEVQAHADEVEELRRKMAQKISEYEEQLEALLTRCSNLEKQKSRLQSEVEVLIMDLEKATAHAQNLEKRVAQLEKLNIDLKSKVEELTILLEQSQRELRQKVAEIQKLQHEYEKMREQRDALQRENKKLVDDLSEAKSQLADAIRRLHEYELEIKRLENERDELAAAYKEAETLRKQEEAKCQRLTAELAQVRHEYERRLQIKEEEIEALRKQYQLEVEQLNMRLAEAEAKLKTEIARIKKKYQAQITELEMSLDAANKQNMDLQKIIKKQAIQITELQAHYDEVHRQLQQCADQLAISQRRCQGLQAELDEQRVALESALRSKRAAEQSLEESQARVNELTTINVNIAAAKNKLESELSALQADYDELHKELRVVDERCQRTIVELKSTKDILVEEQERYIKVESIKKSLEVEVRNLQVRLEEVEANALAGGKRVIAKLEARIRDIEIEFEEEKKRHAETQKILRKKDHRAKELLLQTEEDHKTITMLNDAVEKLNEKVKVYKRQLNEQEGLSQQNLTRVRRFQRELEAAEDRADSAESNLSLIRAKHRSWVTTSTVPGGTRQVFVTEESSQNF

SEQ ID NO: 56 - protein, 289 aa, Rhipicephalus sanguineus
MAPRNWKRPLTLIYNDNYKYGNSLYSGALDDIEKRYQASLSRTTFRADRGDLGLSTFSSSNIGSAPEPSAGRPATTATNESDHALLSPSFSAPSRSPVRSMLDTDHDLRKARSQGDFGRLRSSALRPNSWSDEEALMEDHSYKRRQRRRRQDRNEFLLSSPTVAAGENIDYYFDKCRELKNELEALHGRVLDAEARARSEAGTLRNKVQTEIAELSVQLEDTTSQNNELQKAVKRQAKQLLDLQAYCDEMQRHLQGAVDDLSTANKRCLDLQNEVDSLRSNLESGMRHI

SEQ ID NO: 57 - protein, 299 aa, Ixodes scapularis
MKIESRLIRELINEFIGTMILVLIGDSILAIIIAGDNEPLAPIVGPVGWGTAIFVAVTIAGGVSSHLNPAVTLALASTKKFPINKVPLYFAVQYLGAFVGAALVYLLYHDQIAHFDGGVRQITGKHGTAPIFATYPREQISTLTCFFDQLVGTGILMLTAEAITDPRNFGGMPKHLHPLALGFMIMALIFGFSYNCMAPLNPARDIGPRIFTAIAGWGTEVFTYRNWNYIWVPIFGPHIGAIIGAWIYKVGIGDNFPDDEPKLNGDLERGKVVELQEAAYRPIDDKLNLEAHTNEALKL

SEQ ID NO: 58 - protein, 309 aa, Ixodes scapularis
MKPNSVARVWSRICNCRLESRSCRDVLAEFLGTLILTLIGDSVLASLTASRLGYFGIAAGPLGWGLAVYLGIIVAGGVSGGHLNPAVTLGLASAGKFKWAKVLPYIAAQYLGAFAAAGLIFLLYQDALYKVDGGMRVVYGPNATASVFSCFPAPEVRTLTCLLDQVVSTAVLLVSICAIVDPKNMAVLKGHQPLLIGFAVAACMYAFSYNCGNPLNPARDLAPRIFTAMAGWGCDVFSIRGYNWFWVPVVGPHVGGVVGVWVYKFAVENHWATDAAAVVGKTPEEEHDYEGKRRLLRQDTHYIEADIAS

SEQ ID NO: 59 - protein, 597 aa, Ixodes scapularis
MRPLIVFALAVFAWDARTSPVLGLALQNEANEDQTIHEDPKGICKRVGSALCGDLAKCVALPAIDSFQCSSPKGGFYDANDKTCKHWQSCGPLPCKIGYCDDGNGSKKRHCKCGGIPGLTEFCKINKEAKFKCQEYGATAVLKGDEVSCDCGPAEKFINKKCVPVACLNFNMTCEELCQKKLLDKDDRCCQGWNIENCSKTPDETRYCKPGYISRNDSCVDACTAKVADPLCPDGCKSSATDRPFQCICKKGFQLAEDGITCTERRVCNEEEKKRCRKDQICGIVNEEVTCSCKSHQQEKDGVCTNECFVKKCSDPFANCEVYLGTEKCFCARPLFPAGNNTCGLEKYSYILTFRTNDTSPYTDDFCESKKEDIQKALKTLFGAQLVDDTLLSCKEDFVIRLAFSQKQDPAVLKRIETCQYLRGDVCIFPPRLYIKEGSAKLEEEDLCTEFFQKQLNQSNGMYVCQKVGDNYSFKCRDGLFSYNEVTSGRLTRWSCADKREDIPPASSESPKLVTDKEPQTGTEGTVTKTTPVSDDPCSPSPCVNGGVCKPGTGKTFTCECPKGFKGNLCEEKNSGVSVQAPVTAILGIAMSLLLLR

SEQ ID NO: 60 - protein, 420 aa, Ixodes scapularis
MKIAILMLACLAHTVLGQSQRNGRQTNSTISNVNSTAPSSARTMPIVCYYYGWAATRPDPASYDVDKIPGDLCTHVNFAYAGVAPNTWELDSQVPRYDADRGLYKKFTAIKDDYPKLKTLLSVGGWQHPNGVFSQMSSTPQNRQKFITSVLQWMKEYNLDGVDLAWPFPGVDYRGGGPQDKENYVQLAKELSKELRANGLMLTMEVPLSDQHLNPGYDIAELTKYVDWFNTHAYDLRGKWNGVTDVHSPLFPRDIDVGDQQKLNVKDGLENLVSRGAPKSKLVMGIPFFGRGFTLLDSSQHGLHAIMNGNVPPNPGPFVRSSDVYSYFEICLFLKSGWTREFDDQGKAPYAYYNNQWIGYEDTESIKHKMNFLLREGYAGVYVFNNDMDDFRGLCGEPNVLLKTIRNSLNQDKNDIAETL

SEQ ID NO: 61 - protein, 259 aa, Ixodes scapularis
MMFHPFHMVEKSPWPFSSCISSIMITMSTILMLHFSFSYMISLALIILMLTLFQWWRDVSREAALQGLHSSYVLWGLKFGMILFIISEIFFFISFFWAFFHSSLSPNIEIGAMWPPLNIFPFNPFEIPLLNTTILISSGISISWSHHSIINNNYKEALYSLLITILLGFTFSILQMWEYIQAQFSMSDSIFGSSFFMTTGFHGIHVIIGTTFLIVSFFRMNKNLMSSTHHFGFEAAAWYWHFVDVVWLFLFTFMYWWIF

SEQ ID NO: 62 - protein, 216 aa, Ixodes scapularis
MPVDLYNLYGSPPCGLVRMLAKHIGVELNVKNVNLAKGENRTPEFLKMNPFHKVPTIDDDGFVLYESTAICYYLLNKYAPDSELYPKCPRGRARVDQVLATMTSTIQPPFMAFFRPRFFTQSKPTDEEVKALEENVLQGIETLVGDGNYALGDKLTLADLSLMAHLSLLAEIPVFDSGKFPKVAAYYERLKAELPYYEEINRPGISALVNLWPVLK

SEQ ID NO: 63 - protein, 470 aa, Ixodes scapularis
MIVILAFTAFVKPAATGYVSEELVFPKVIQERIASSEKVVFIKPGIFLNLEKSHILADQLILTDSEDDALVHTIINGTKAEENLYHDKYKGSSLFLHQTGDAVYLKGMLTPEWQIEPSITEERSPNNGVAHRVAKIPKAPLYCKQIAAEANHIHHNVVTESRQLPELVTPEVYVISDYRHNQAFTHSDLVAYMAALFNAVNLRFASISKPSVRLRLVGIQRGTPETEIYMKKHLGHTLGTESLLRLRNYVNYGYVHGNPDMVYLMTGKKIATLKNGRMSTRVSGLAYVGGLCWSTKVAIGEDIPLSYKGVHIAAHEIAHLLGAVHDGSGPISYIPGHPGAKKCPWSDGYIMSYIDSGEKNFRFSVCTNEQIRILLRNRDERCIGLSSEQDLTSKSSKLPGQTISGSTFCEARYPSWEDVKYRVLPGMEKKCLFHCCGPTDENGAAVCQQHPALDGMSCEKEKICKNGICT

SEQ ID NO: 64 - protein, 377 aa, Ixodes scapularis
MEASLSNHILNFSIDLYKRLKPSGKDTAGNVFFSPFSIAAALSMALAGARGNTAMQIAAILHSNDDKIHNHFSSFLCKLPSYAPDVALHIANRMYSEQTFQPKAEYTTLLQKSYDSTIKAVDFAGNADRVRQEVNAWVEEVTRSKIRDLLAPGTVDASTSLILVNAIYFKGLWDSQFKPSATQPKDFHLTPHTSKTVDMMHQKGDFKMGHCSDLKVTALEIPYKGNKMSMIILLPEDVEGLSVLEEHLTALKLSALLGGMIVTPDVNLRLPKFKLEQSIGLKDVLMAMGVKDFFTSLADLSGISSTGDLYASDVIHKAFVEVNEEGTEAAAATAVPMMFGCALFPEVVNFFVDHPFMFLIHSHDPDVVLFMGSIREL

SEQ ID NO: 65 - protein, 400 aa, Ixodes scapularis
MFAPTALFFLMAVASCYSQSDEQNLAKANNQFSVSLLKQLIAEKPDDNIFFSPTSISTALAMIYAGAGGSSEEELSTVLGQAGFGLTDRDAVLAAYKKLLAGTNSEKVTLDIANSVLIQNGLPVLDSFKKALTESFGAELRSADFANDGPKVQSEVNKWVSQKTRGKIPSIVDDGFPMNTVMFLLNAVYFNGTWHNQFPQKNTVPRPFFNRGSEEVHVKTMALRGSIRHTRLDDLKARAVELPYQGEEYSMVIVLPNAKMGLSQLREGLTVSMLQKITNQMSAKTVSLTLPKFELQTNYDLIPTLKQMGLKSVFDEKSDFSGITGDKSLYVSGVRHKAMVEVNEKGTVAAAVTSISVALRTSLIRPKIVHFHVDRPFLFYIRSRATGRLLFMGEVHQLQA

SEQ ID NO: 66 - protein, 377 aa, Ixodes scapularis; identical to SEQ ID NO: 64

SEQ ID NO: 67 - protein, 184 aa, Ixodes scapularis
MACATLKRTHDWDPLHSPNGRSPKRRRCMPLSVTQAATPPTRAHQINPSPFGEVPPKLTSEEIAANIREEMRRLQRRKQLCFSSPLESGSPSATPPAADCGPASPTGLSPGGLLSPVRRDQPLFTFRQVGLICERMMKERESQIRDEYDHVLSAKLAEQYDTFVKFTYDQIQKRFEGATPSYLS

SEQ ID NO: 68 - protein, 282 aa, Ixodes scapularis
MAPPCYADLGKQARDLFNKNYHFGVVKLDCKTTTKTGVEFNVSGTSLNDTGKVNASLETKYKVSDLGLTLKEKWNTDNTLATEISCDDQLARGLKLAFNANFAPQTGKKSGALKTAYKVDNIHVNSDVDLGIGGPIAHAGAVLHYQGWLAGAQLSYDANKSRLSKTNFAVGYQGGDFVLHTNVNDGQEFAGSIYQRVNDCLETGVQLSWTAGTNATRFGLGCVYQLDRETSVRAKVNNSGQVGLGFTHRLRPGISLTLSTMLDGKNFNQGGHKLGLGLDLSA

SEQ ID NO: 69 - protein, 2225 aa, Ixodes scapularis
MRVLCGLPLLLVAATANALLFELVPTSRGSGSAVYKVNGTVTLKTLELDVTEGPALTYEGDLAVQKLTETDYVAKFLNFTLVKFDKVLGDVHHFEPHYESSLFGQEVDYFQYVQYPVWFSLKQGKVVEFGVAQEVREGALNVYKAVLTLLQSQPETFQELPAVVSYYEDGVSGYCRVNYELQSLDAHVYTGVNVLNVTKTKYLDDCKKTRPVYTVDSVEVQGYPPLCNKHLPNNFLPGYQEDTAEYEASPTVGCPVGYKPFDTLVTAHEVSYYNLSDNVLESAYTESLDVLNVFTGKVVVKTLLKVWLAHVDGPQLEEFTPVQTYQTLELVLPETAHHFDLPVYSLLVETPEEVSVEVFQRALTTVVDELVSLEVEDETVEPKQTPGLLLQLVKTVAVLNLEQLKQTVPEFLQRPVLELAPHEQVHRSLWVDLIGKAGTKSSLDLVLYLLQQNLLTRNEAARVLQDVAAFKAYPEKETIEKFLEFALGQSQVLPPLVFSTLLHTLGELVNEACPSEVEYPSYEEGYLVEVEQHAPLHRLYLPVGAQCTVQDLKQYVLTISEALKQTDDFKKVVAYLHGLGKFAKPEVLPVLLAYVNGTAENLYRLVSQDEDYLESVYFVKKAALLALDHVVKYYPKEVSPLVRTLVLNTTEPTDLRTLAFDIWLKTVPAKWDLQQVVLAAKTDLSLEFGTYVYTALKSIVKDKRPVNSLLASRVRGVFTHLKPHDFGLRFAHLYKHTYYNAEHLFGLESLFKKVATNTSFFPSYLSHGIKYNLGPYFKTLLSGKLLLKGGEKVLDDVFGHDGLLTKVVLALQGELDTSPRHFEHHTRETSLFKELNVEPRKVETPKAVLLWQFLTGEAVLPVDEELFHELQKELLELVHKVGQEGLTYHFVRLVLPKQLYHVEPSPVGLPVVHTIQHPVLLSLKLDKLALSLQTAPGSLLPVSAKFTGTVHPSLFTTNQVRVFVSDLVGTPSPTVVLTDTKELNFKHTVEVEYSKEDAQQLQVSVKPHYGRVFLSAKCTEHVLTTNKIFEPLTTPFLEYSKCIKSMAVPYTYEKTYGGEFLNVDVSAVSHQPWSKPPLYKVLPEGTTRLDALLQWISSKTYKHTVLSVDVKPYAADPVTEWTATFKYQNNLKELVEATLHQEEGLYPEVNAAKEASWFARVNPFHGKKHHHEQEHHEEEEHHWEFPAQYVEAIEDYEEISGMPILSKVRKAHHTHHHTRHHTHHKSHHKTPKTHRTQYKWTVPTFLNKTHTKHALTVTLEGKDGGAVKKGVQLNYVGLRTLDKSSRKLYLDVETTFLPKLSLFLNVTHPVVPSPFYYVPSFLGKDVANATLYIRYGDDLTKPIVVTYNATKTEEQVLGVHGYDGAPLLKWFVPQCLEDQHNGFAVSYACRLATTFDAHLNHQVVHVKVPQHFAEDLVLKKYAFKSLNYLKYKLFPYVTFQALPHLTHQVEPEFTLYFNKSHVNPFVTVGSGVVVLPGEKLTVKNLKVSEFYSPNLLLSGYDRFLHSHYNGFPYLPCGVGKNYLRTYDNVSYPLELKDECKYLLTGDCTTRSDFSVVLEPLDLEVGTKKLTLQLFDTVVVLPPPDLYKAEVVLTVNGTTYVATEYKDVVLPYKAYHKLFITVYPTSGPHDPPVVQLTNTLRTFKVVFDGVNAYVWVHPLYQSKTCGLCGNYDNEPAFEFFTPTNELVSNYSEFVASYGFGGAQCHEVVPSVLSTLSQLQVLKGAFLATWAARPAHLGRSWFNVMGHHQHHHHELPEENSDEVKDGVLPWFHPEHLLPHHKYPQHHKTTPYPYATPFTPRYHHHSTQYHYATPFTPRHYEHSKHHHYATPEYYESEEYRRATPFTPRHYGSTKYNYAATPFTPRHYESEEELRATPFTPRYHHYKATPFTPRYHHQAKKFTPLYYESEEDLRATPFTPRYHHYKATPFTPRYHHATPFTPRHYESEEELGATPFTPRYHHYRATPFTPRYHHATPFTPRHYESEEELGATPFTPRYHHYKATPFTPRYHHATPFTPRHYESEEELGATPFTPRYHHYKATPFTPRYHRATPFTPRHYESDEELGATPFTPRYHHSTPYPYATPFTPRHHHSKQHHYATPFTPRYPSEDLQRSEISEELKSGQMMKQYYEDTEESKCFQERTIMRDHEGMICYSQVPVPSCKPGCKKGKGSVQLSVPFVCLDRSSSMAKELTKKVRKLGNVGKLPIDLETTPHVEQVRVPEKCVPYEK

SEQ ID NO: 70 - protein, 1805 aa, Ixodes scapularis
MTTAAYSVLLATAFYLVSSASAECPQGWFDCENGRCIAMFWKCDGQNDCGNHNDEKNCRDEPEECPVEKFNCHDRSYCIPKVWLCDGEADCGDSSDEVGCTVNCTGFSCKSNECIPLNWRCDESEDCLDGTDELGCANATAAKLVQQSPKCDVDSGHFPCLDGKCLLPEKVCDGTKDCGDGADEGSFCKVNECSSKKCSQGCFVSANGSSCYCNTGFRLMDDGISCIDNDECTERPHVCSQMCTNTPGSYHCSCLNGYLLTDSTFCKAKDPEPLMIFSDSKQVRGLWLRTNRYFEIHSASGQAVGVDFDNSARRVFWTDVTSKKSAIHSCLLDGTGFKVLISEDYSLMEDVALDWLANNLYITDSALKRILVCKSDGSACATVVTAFLDSPRAIILNPSKRLMYWTDWGKSPAVMAAGMDGSNVRALVSENLGWPNGLAYDHSTDRLYWCDAELAKIEYMDLLTMKRNVVIQDAVFHPFSLTVFEDTLYWSDWLAYSLDSSNKFDGKHHHRILREDSKHIMGVHIYHPLLRSKDLVNPCWDNPCEDICVLAADGYECLCRLGYEPSPKDKHTCTVTRLSSYAVVSEENVLYRVRHESIGGESVSKMPVNRLIMVGAMTLDWATQTLFLSDNPREMILAVDMNTYNVSTIREHHVGSIFGMDFDGQNRNLYWVDADKKTIEVCRSNGSGHIILRSDLRRPVDLALHPKAGVMYVLSAGDTPSIVRYTMDGLSPIRVQLTSMLLGLSLSTDLVAERLFWGDSVRATIEFIDLKNGSSTPFIVEQIHGHITSVSASHGLVHWTTREDGALYYVDLHVKPTSVHSVSLPPRNGTVARKVIYAAQIPDYAPGPCSVKNGGCSHVCLPALTSRSCLCPPRMVLSANNVTCKDENGTCSAHELSCAGTCIPATYWCDGYQDCSDNSDENNCGTVTCPANGFRCGNGKCVENAWLCDGYNDCGDHSDEINCTRRNCTSEEYQCRANYCIPVYWRCDGEKDCPDNDDEGNCSLIECPSGYDRCRDGQCFSRDWRCDGQADCKDSSDELDCGATTGCLPDDFRCTNKECIDPRFRCDRRVDCEDGSDENGCDSSYTANTTSQCPRGMVNCGDGQCIYEHDICDGYVDCHLGQDERNCTSAMCSSKEVFCINTKRCILEAWLCDGEDDCGDNMDETLPRCHPTTTPSTTTTEAACWSDEFRCGSHECVPWSRVCDRHLDCMDASDEGSHCDTHCGKDNGGCAHICRESPTGPKCSCHPGYYLTGDFKTCEDLDECQKPGHCSHFCTNTKGSFKCTCAEGYAIAADRQYCKAQSGEASLLYMLPNQIRSFSMRGHAQHLLAQDDLADMHGLDYHFDDKAIYWTEMKEGTINVMLLAKKTPQTVLEDVHRPFHVAVDWIAGNIYFTDGWVHIQACDYSFKHCTDVLETTYSHVNSFTLAANHGIMFWCVWSDVLGKSDGIIERSNMDGSGRVILVSDKILWPCSLTVDHVHKLLYWSDSNKNVIESATFDGKDRKTIVMGAISSPFSIALYEDWLYWADWGSDSLMACNRHTGRDKSVVHHGDVKATVLKIVHQVHQPSGTNRCARNQCEHVCLLTPLSYTCACAHGYKLMEDSHSCFDMENETAYSLQSSDVLKQPCDPECVNGGICVHNKNKLYCQCPKEFEGLSCEDPVVLALPVADQASNSMWLASVLIFIVCVALLAFGYLFYRRNRHKLSAIDFTVGFKTPTFAAKDEGLLENEHPIAEDYHVVAASHPGFKNPLFLDGHKSHLLTENGEFQRWSSNESVVSSVGVDDSQATLAATEDETKQKKQVFFFRKV

SEQ ID NO: 71 - protein, 221 aa, Ixodes scapularis
MYSILKDLKDSLSSSTGQPFQLIIAVASGSELFHAELVQLADVFVYADYHMPNNDGSCKIQFPSVSSQTASLTSSLLRTKELMEKGSGNKTFCVTLNLAVLKFNASPERLSLGAQCGAESLAGYSEVCSLPSSRTVFDMNWMSASLRSGDVLYTFEKEGVLLSKVLFLKTLHPSLCVAAFHTELDSTSCTQPFARLVDIRMALDSAVHGEGATVRAQPVDV

SEQ ID NO: 72 - protein, 319 aa, Ixodes scapularis
MVREDKTAWKSNYFLRLVQLLDEYPKCFIVGVDNVGSKQMQTIRVSLRKHAVLLMGKNTMIRKAIRGHLDNNPALEKLLPHIKGNVGFVFTKEDLGEVREKIIENKVKAPARAGALAPLDVMIPAQNTGLGPEKTSFFQALQIPTKISKGTIEILNEIHLIKKDDRVGASEATLLNMLNISPFSYGLKILQVYDSGTVFSPEILDITPEDLRSAFVEGVRNVASVSLAIGYPTVASAPHSIVNGLKNLIAVALETDITFKEAEMAKEYLKDPSKFVAAAAAAAPAAGGGAAAKPDAKKEEAKKEESEEEDDDMGFGLFD

SEQ ID NO: 73 - protein, 392 aa, Ixodes scapularis
MMLGRLAALLVLVVSCKAQEELKLTLANNRFGLRLFHALPSAAEVNVFFSPYSVSTAMGMTYAGARRDTAEELFRGLGYSASGLTAPQVLDSYARHTQRLLSSESQSTLKVANGAAIQENLTLLDSFRNTLESSFNAEVHQVDFVHRQQDAFEFINRWVKQKTSDKIDKLFDEPLSPATKLVLLNAIYFKGVWNTQFDRALTRRRQFWNGGVSPILVDTMMQNMHVGYGSFGDLDVDVAELPYRGRDYSMVILLPKRNDSVDKLKGNLTVEILRSLLSELRERDVDVFLPKFKLESTYQLEGPLTALGIRQIFSNGADLSGISDRNLRVSAVVHKAVLEVSEEGSEASSATGVVILPGSAMPVAFVADHPFLLFIRNTRTNDIIFAGQVNNL

SEQ ID NO: 74 - protein, 196 aa, Ixodes scapularis
MKQFVVLLALIGAATSGNNLFENLDKYPLQDECQAALQEHINVEMHASLVYMQMAAHFDNNKVARKGFSTFFAENSKEEREHAQKIIDYINKRGSTVSLVNIDMPQITTWKSVLQALRDAISLENKVTNKLHAVHKTADEECKDPQLMDFIESEFLEEQVTSIDKLQRMITVLSNMDSGTGEYLLDRELLGDKKEF

SEQ ID NO: 75 - protein, 91 aa, Ixodes scapularis
MKTYVLQALLLTLAVAVVRAANTGIEWPPKPQKCAEGETWKECVGSSCAELTCEHPEPSLGCTYDCNYGCYCAPDFFRNANKECVKKDKCP

SEQ ID NO: 76 - protein, 161 aa, Ixodes scapularis
MMALKVTTSLILVLLSAVICTGHPDGADDEACKDLYPSHGYTAKSAAEGHAEGYRLVQDTEDYKPGDVITVTLSSEASPFMGFLVKAFDENEKDVGSFRSTGPDSRAFSHCAGITHTWRDLKKRVVVQWLAPEDRSGKVHFKVTVVKQIKDFYHAIPSTLA

SEQ ID NO: 77 - protein, 220 aa, Ixodes scapularis
MGDESETERRRREAEEAHRRLKEEEKRKMEEKERKKAEVRKRLEEAAKAKKSGGKRGFMTPERKKKLRTLLRKMAAEELKKEQERKAEQRKKIIAERIGAPKPVDNCNEATLQGILKQYHSRIAALEDAKYDLEYEVRQKDFLINELTIQVNDLRGKFVKPALKKVSKFDKLKMVAKSTSEVDFRSNLKSVKKETFKLDEEKGPVDKPEWALGAKKEAEE

SEQ ID NO: 78 - protein, 411 aa, Ixodes scapularis
MESHRYTRAGLVLFVVMVFAGARHTEQDSGDAIDMDCYRNASSLIASKGYQVEEYEVTTSDGYILAVQRIPEGRSNALRNQDSPKKVVFLLHGLLGSSADWVLNYPPQSLGFILADAGYDVWLGNVRGNTYSRHVKYNRRSKEFWNFSVDEMIERDLPETLDFVLKRTGRQKLFFVGHSQGTSIMFGLLSLRPEYSEKIKLFCALGPVSAITNTRSPMRYMSPFGKYIGAFVNSLGRYEFLPNNFVMKLLVDAVCRHEGPRDVCGNIVFLIYGPETMELNVTRLPVFLCHVPAGTSVRTMVHYSQILISGRFQKFDFGENRNQLVYGASTPPEYDVSRVAVPVALFWSEGDWMADPRDVALLRRRLPNVVLDFKVSQPKFSHIDFAAGIHAKALVYEPMMKLMASYKSAGE

SEQ ID NO: 79 - protein, 394 aa, Ixodes scapularis
MRAVLVVPFLLAAAVAAPASGGEEQCDPAKCKGSENCMCASIKPPNGIEAKDMPQLVMLAFEGAVNTVNMPFYRELMDTTDRKNKQSGCRIGTTFFVNHEYLDYSAVHELHNRGSEIALRSITLNGTMAYWSDLDTDGWKAEIVGERDLLATQAAIPASDIYGMQAPLLTTGGDKSFKMIKEAGLLYDASIPHNRVKDSGRIMFPYTLDYGLQTPCVIEPCPEDKYPGVWAIPLNVWFKENQIEHLKIDFPCSTIDSCTPPPASADEAYEFLMANFKEFYENNKAPFPMFLHEGWLREGDRKAGFLRFIDWLLAKDDVFLVTVKEVIEFMKDPKPAKSYKESRCVTEVKPSDKCKNPEVCVYPRVKIGYNIGDRVMRSCVKCAPEYPWVRAENN

SEQ ID NO: 80 - protein, 494 aa, Ixodes scapularis
MRAFDPATVFLSLAAIACSADAYRTSRQRVFLCYFASWSYYRVGDGKFSVAQIDATPCTHLIYAYAKPVNGSIAPMDTRLDTNVHKMYRKFNALKRTRRGLKTLISIGGWNEGSERFSKIASSERERRAFVESVVAFLDLHGFDGIDIDWKYPTQRDGSPNDKKNFALLLKDLRKALDRKDHILTASVSALKAIIDAAYDVKEISRHVHFMSIMGYDLFGPWNYHTGHPAPLRGRIGGSSDESTLNVETSIASWISEGADVGKLVLGMPLYGRTYTLKDPSEHGFNAPTWGPGESGPFTREPGLLGYNEICTLISTGGWKVTRDPNVDAPVAVKGNLWIGFDDAESLANKVQLALDKGLAGAMVWSIETDDFRGKCGRGKNPLQEAIRKALLLNASEPTMTTSESTMTASEPTMTTSEPTMTTSQRLTRSPVTNSPGLFTCVSEDRFPDPDTDTGYIWCVRRGSDFQLFKMRCPRGTFFRKHTRRCDHVYKNAL

SEQ ID NO: 81 - protein, 133 aa, Ixodes scapularis
MSLLNAALVCSVVVSLSTAFPGIWRRHHPDVDPRYKEWAHFAISSQVEGRTNYDTLLKLLSVESQVIAGVDYKLKMMVAESTCVVGKDWYSRTRCHPKVDAPHMFCTAVVNYIPWEHTKSIKYYSCGEPVNNS

SEQ ID NO: 82 - protein, 1537 aa, Ixodes scapularis
MRVPYVLPVLFLAAASAFEVGKDYVYHYNGKMQVYNPEQPLQSSGFAFRSKVVAQPRPDHTHFKIIDFEVDSFNGDHVHVGEHEFNYHSTEALKQFIERPFAGKFSEGKLEEAELSKSEPKWARNLKKGVLSIFQLDLVKGRHDHPHAKQFHVREEGLHGNCDTLYVVAEEEGHLKVTKIKNLEKCDKEHYAVYGRIKGHECVDCEAQETHPFVATSQVKYRLDGTPEHYVINHACATSENVFRPFGQGKTFVAQLNRTLDLEEVHDANTDTQLPEDLEKVHHIAQTFPESDEVESLEELKHVNRYVTTFDLSTDKDKFISGLNHLAALEYEDSDIKDVHSKESGGLNFLILFGSLASMPFEDIAHVYEQAVANAPEASKSQVRKVFLDLLSAVGNNPHAAFGLQLVKEDKLTDEEAEHFLAKLALNLKENSPALLTELAEVCEHVKPKRPVWVNCQLALSSLAGQEGCVRAKTDKEHDEGFCKPSIVSHFFNYEIKPEDKKDQPEYKRTVYMKAAGNLATRGAVHYLERYVSDTNQPEYRRSAALWAMVRAAPHHHELVRDVALPVYKNKSETAYLRIGAFVNVLMTKPDLYLLKYIGHNIIDDPSDQLASYVTSAFRSLVKSKYPCHQELAQHLRYVVPMWNDVSRFAKPLDHTKSQVILSSGYDPKFDYGGSSVFAQVRADDSYLPREVYFSAKDYFHGRSFESVSLKFESWGLDTLLNHVVGPQPGSTKNLWNVFGRRRFTRDASAKDLQEVEAALPITDRDYDHVYGRLSLDLYGHAFDTWEFDESILQELSKGEGEEAPGDKLKNLLGESKRKKAFILSKDICIIVPSELGVPVFYDIKQVEYMYINPKKFAFDQTQDAKFAMDVERHYVHESRAFSMFGVALTFNKTSLGTGSDSQTIINLPIDLKITLDPIHKKLSMKRPLSLPWNVLNHHFRPFTFVMSYDINEDVTTAETKMTQAMYPLYNKEELTEFDRNYFGEVLGYGLNVKGSLLSKGLKKGLHDYWYESDDRQKFYYATLNPEWHPRELQFTVVPAAQDATTEVEVDLGYKFLEPDDPRESHFEAHDNVGDDAEVPSTHVLNLDYTLKGTKERKVSAELRYSFTKDLLKHKVQLFYDRTPFHPKEQDHTKVCLDVISKFPEPDWERLSNLAVFHEGKDIESQLNLHYGSNCVDQSSITFKGKYTHTDDEEKQIRENAEGKPLGINRMKKYTLRRPYKKCTEHQKRGVPLNFECMKYLYYTSRLGKLTTDVEYKNLKPLFPMLLKYYKKVHKEGGFLSTLASHVHGPTGKLHVVSQVPPVEKYSDIVVTTEDGHSFHHDHVPIYSHLLEPRIFSVLGYSNMVNYISYYKQKHCDLQGKSLRTFDNVVVNLPETDCFKVVAKDCSPNKRFTVLARATGNAALPKALKAFIQSTKVELLPVSADSGLVLRVDGNKVDLTPGVPYSHTAHDVELFTVTQHNKYFEVMSQPYGVFIGFDGNVLFVQTANFYRGKLCGLCGDYNYDRQHELVGPNLHHYNDTLEFAKSYVVPASDCTAP

SEQ ID NO: 83 - protein, 873 aa, Ixodes scapularis
MSARSSKYMYKSSGGAGDISIEYGTDLGALTRLEDKLRLLQEDLESERELRQRIEREKSDLTVQLMQLSDRLEEAEGSSETVVEMNKKRDTELSKLRKLLEDVHLESEETAHHLRKKHQEAIAEFQEQLDGMTKLKSKAEKERQKFQAEVYELLSQVENSNKEKLTIQKTVEKLEHTVYELNIRIEELNRTVIEVTAQRTRLSAENAEYLKEVHELKVSLDNVNHLKGQLATQLEDTRRRLEDEERKRASLESSLHTLEVEIESLKVQLDEESEARLEVERQLVKANADAAAYKTKYETEVQAHADEVEELRRKMAQKISEYEEQLEALLTRCSSLEKQKSRLQSEVEVLIMDLEKATAHAQNLEKRVAQLEKLNIDLKSKVEELTIMLEQSQRELRQKQADLQKLQHEYEKLREQRDALQRENKKLVDDLADAKSQLQDCVRRLHEYEIEIKRLENERDELAAAYKEAETLRKQEEAKCQRLTAELAQVRHEYERRLQAKDEEIEALRKQYQLEVEQLQMRLAEAEAKLKTEIARLKKKYQVQITELEMSLDAANKQNLDLQKIIKKQAIQIQELQSHYDEIHRQLQQTADQLAVSQRRCQGLQAELDEQRVALESALRSKRVAEQSLEESQVRVNELTTINVNIAAAKNKLESELSALQADYDELHKELRVVDERCQRTIVELKSTKDILIEEQERYIKVESIKKSLEIEVRNLQVRLEEVEANALAGGKRVIAKLESRIRDVEIEVEEEKKRHAETQKILRKKDHRAKELLLQTEEDHKTITMLNDAVEKLNEKVKVYKRQLNEQEGMSQQNLTRVRRFQRELEAAEDRADSAESNLSMIRAKHRSWVTTSQVPGGTRQVFVTEESSQNY

SEQ ID NO: 84 - protein, 286 aa, Ixodes scapularis
MAPRNWKRPLTLIYNDNYKYGNSLYSGAIDDIEKRYQASMSRTAFRSDRGDLGLTTFSSSQIGSAPEPSSSRPTTNASEPEPTLLSPSFSAPSRSPARSVLDIDTDLRKARSSGDFGRLRSSYRPTSWSDDEDLGLLDDPGYSRRRRRRRQDRNDYLLTSPSAAGGDFYLEKCRELKGELEALHGRVMDAESRARNEAGNLRSKVQAEIAELSLQLEDTTLQNNELQKAVKRQAKQLLDLQAYCDEMQRHMQGAVDDLSMANKRCLDLQNEVESLRSNLESGVRHI

SEQ ID NO: 85 - protein, 305 aa, Ixodes pacificus
MLDGFKIKNAVIRESLAEMLGTFILVLFGNTVLAILTFEKASTDGLAACFWGWGLALTLGVLVAGGASGAHLNPAITVAVTTIGKFPWRKIVPYVLAQYIGAFIASVVLFITYRGAMDNFDGGNRIVTGVNGTAGIFASYPKEFLSTGNGLVDQIVGTALLMLCILAITDARNMAVPQGVQPLFIGFALAAIILSFGYNCGAPLNPARDLAPRVFTAMAGWGGEVFSFRDYNWFWVPILGPHIGAILGAWIYTLAVELHWPGATYEMDGGNPVSTKDDIHGPDGMYSEFENSVQMRGIKLGDSKN

SEQ ID NO: 86 - protein, 309 aa, Ixodes pacificus
MKPNSVARVWSRICKCRLESRSCRDILAEFLGTLILTLVGDSVLASLTASRLGYFGIAAGPLGWGLAVYLGVIVTGGVSGGHLNPAVTLGLASAGKFKWAKVLPYITAQYLGAFAAAGLIFLLYQDALYKVDGGMRVVYGPNATASVFSCFPAPEVRTLTCLLDQVVSTSVLLVSICAIVDPKNMAVLKGHQPLLIGFAVAACMYAFSYNCGNPLNPARDLAPRIFTAMAGWGCEVFSIRGYNWFWVPVVGPHVGGVVGVWIYKFAVENHWATDAAVVGGKRPEEEHDYEGNRRLLRQDTHHIEADIAS

SEQ ID NO: 87 - protein, 510 aa, Ixodes pacificus
MRSRFLFVWSAHFACCVIVTCSSQTGIMRAREKQNHRFQGDDFKDICTEGSVGYETCHKTGGFCSLIQAKKTFQCDCWREDLYYDANETMCKHWQSCEPNPCRYGKCNDQGGKKPRSCSCPRMKDLYEDCQIKSEKKVSCEVAEAKARIDANGRVYCDCGPAKTYDNGKCELTGCLNYSKTCQELCNENILDKDDRCCEGWNVSNCSRPPIASTFCPTGYVRVHGQCQDACTANLTSPICPVGCTPMTNDRRPFHCKCKSGFELAEDGLTCREKVACNEEEKKKCHESEICTLIDTKAVCTCAEGLQRLNGVCTDKCTTKCDHKFAKCVIRDRYERCSCIYPLSKTSNGDHMCTLEFYSYILVFQTNDTEPYTSSFCDKKLPDINSALKVVFGRELLKVETVSCKNEFVLRLVFENKQHPAVLRRMSNCQYPQGDVCIFPPKLNVKATSIKTIEEENLCAGILQELFNVSDASSECTKEEDNYTIRCREGFTAYDEVKSGRLTRWSCKGK

SEQ ID NO: 88 - protein, 403 aa, Ixodes pacificus
SSTGHGRRTNSTISNVNSTAASSTHTMPIVCYYYGWAATRPDPASYDVDKIPGDLCTHVNFAYAGVAPNTWELDSQVPRYDADKGLYKKFTAIKEDYPKLKTLLSVGGWQHPNGVFSQMSSTPQNRQKFITSVLQWMKEYNLDGVDLAWPFPGVDYRGGGPQDKENYVQLAKELSKELRANGLMLTMEVPLSDEHLNPGYDITELAKYVDWFNVHAYDLRGKWNGVTDVHSPLFPRDIDVGDQQKLNVKDGLENLVSRGAPKSKLVMGIPFFGRGFTLLDRSQNGLHAIMNGNVPPNPGPFVRSSDVYSYFEICLFLKSGWTREFDDQGKAPYAYYNNQWIGYEDTESIKHKMNFLLREGYAGVYVFNNDMDDFRGLCGEPNVLLKTIRNSLNQDKNDIAETL

SEQ ID NO: 89 - protein, 259 aa, Ixodes pacificus
MMFHPFHMVEKSPWPFSSCITSIMITMSTIMLLHFSFSHMISLALIILMLTLFQWWRDVSREAALQGLHSSYVLWGLKFGMILFIISEVFFFISFFWAFFHSSLSPNIEIGTLWPPSNILPFNPFEIPLLNTTILISSGISVSWSHHSIINNNFKEAFYSLFITILLGLMFSILQMWEYMQAQFSMSDSVFGSTFFMTTGFHGIHVIIGTSFLIVSLLRMNKNLMSSSHHFGFEAAAWYWHFIDVVWLFLFTFMYWWIF

SEQ ID NO: 90 - protein, 216 aa, Ixodes pacificus
MPVDLYNLYGSPPCGLVRMLAKHIGVELNVKNVNLAKGENRTPEFLKMNPFHKVPTIDDDGFVLYESTAICYYLLNKYAPDSELYPKCPRGRARVDQVLATMTSTIQPPFMAFFKPRFFTQSKPTDEEVKALEENVLQGIETLVGDGNYALGDKLTLADLSLMAHLSLLAEIPVFDSGKFPKVAAYYERLKTELPYYEEINRPGISALVNLWPVLK

SEQ ID NO: 91 - protein, 488 aa, Ixodes pacificus
MSGLCLILCTVAFFSFCLAEKEHGIVYPRMLESRAVTGERMLKINDDLTLTLQKSKVFADDFLFSTTDENGPIDYYIKAEDAERDIYHDSTHMASVRVTDDDGVEVEGILGERLRVKPLPAMARTSDGLRPHMLYEVDAHQNHLPNDYGTPNTSNTPVESRAGSTTHSMYKIPMEIFPEVHIVVDSTFASAFKFKVYKVTRYFAVLTNAANLRYASFVFPKVQLRITGITMNKKPSDEPYIRIVKGHEKYRNILFKETLEDFNKQMKSKPFYHKSDIVFLVTAKNMSEWVDGKLQSWTGGYAYVGTACSEWRVGMCEDRPTSYYGAYVFAHELAHNLGCQHDEDGPNDWVKGHIGSADCPWDDGYLMSYKMQDERQYKFSYCCQREVRNLYNRPEFKCLRERSTTKTIKRSSKLPGRMTTMSNYCQRVYMFEEGMHADKTYGVRDCRVKCTGKTNYWTLGVVDGTPCGNKKACILGECVDAIKISKTD

SEQ ID NO: 92 - protein, 377 aa, Ixodes pacificus
MEASLSNHILNFSVDLYKRLKPSGKDTAGNVFFSPFSIAAALSMALAGARGNTAKQIAAILHSNDDKIHSHFSSFLCKLPSYAPDVALHIANRMYSEQTFHPKAEYTTLLQKSYDSTIKAVDFAGNADRVRLEVNAWVEEVTRSKIRDLLAPGTVDASTSLILVNAIYFKGLWDSQFKPSATKPGDFHLTPQTSKKVDMMHQKGDFKMGHCRDLKVTALEIPYKGNKTSMVILLPEDVEGLSFLEEHLTAPKLSALLGGMYVTSDVNLRLPKFKLEQSIGLKDVLMAMGVKDFFTSLADLSGISATGNLCASDVIHKAFVEVNEEGTEAAAATAIPIMLMCARFPQVVNFFVDRPFMFLIHSHDPDVVLFMGSIREL

SEQ ID NO: 93 - protein, 397 aa, Ixodes pacificus
MKASTVSSLLAAVVVCVVQGDEVQLARANNEFALNLLKELSSRKPGGNVIFSPTGIYEALAMVYAGSRGESQAELSKVLGLDGAGLKNRDAVLSAYKKLFAFKNDSEMNVTLNVANAVLVQEELRVLDGYKTELAEVFNAKLKTADFFNESSRVSSEINQWVRQKTGGKIQSVLPDGIPMNTVMLLLNAVYFKGTWLIKFDPEQTVQRPFYNHGTKFVAKDTMVLRGEIKHAWLLELDAQAVELPYKDERFSMVIVLPNSKTGLPKLRDSFSLKLMDEIDEELDVEKVHLKLPKFELKADYDLVPSLQRLGLKSVFTSDADLSGISGSRDLAVSDVKHAAVVEVNEEGTAAASATSVGIVAKSIPQTANFYVDHPFLFYVQDVESRRVLFMGEVHEL

SEQ ID NO: 94 - protein, 377 aa, Ixodes pacificus; identical to SEQ ID NO: 92

SEQ ID NO: 95 - protein, 184 aa, Ixodes pacificus
MACATLKRTHDWDPLHSPNGRSPKRRRCMPLSVTQAATPPTRAHQINPSPFGEVPPKLTSEEIAANIREEMRRLQRRKQLCFSSPLESGSPSATPPAAECGPASPTGLSPGGLLSPVRRDQPLFTFRQVGLICERMMKERESQIRDEYDHVLSAKLAEQYDTFVKFTYDQIQKRFEGATPSYLS

SEQ ID NO: 96 - protein, 195 aa, Ixodes pacificus
MAPPCYADLGKQARDLFNKNYHFGVVKLDCKTTTKTGVEFNVSGTSLNDTGKVNASLETKYKLSDLGLTLKEKWNTDNTLATEISFDDQLARGLKLAFNANFAPQTGKKSGALKTAYKVDNIHVNSDVDLGIGGPIAHAGAVLHYQGWLAGAQLSYDTNKSRLSKTNFAVGYQGGDFVLHTNVNDGQEFAGSLYQ

SEQ ID NO: 97 - protein, 837 aa, Ixodes pacificus
MAVPYTYEKTYGGEFLNVDVSAVSHQPWSKLPFYKVLPEGTTRLDALLQWIASKSYKHHVVSVDVKPYAADPVTEWTATFKYQNNLKDLVEATLHQEEGLYPEVSATKEASWFARVNPFHGKKHHHEQEHHDEEEYHWEFPAQYYKWTVPTFLNKTHIKHALTVTLEGKDGGAVKKGVQLNYVGLRTLDKSSKKLYLDVETTFLPKLSLFLNVTHPVVPSPFYYVPSFLGKDVANATLYIRYGDDRTKPIVVTYNATKTEEQVLGVHGYDGAPLLKWFVPQCLEDQHNGFAVSYACRLATTFDAHLNQQVVHVKVPQRFADLVLKKYAFKSLNYLKYKLFPYVTFQALPHLTHQVEPEFTLYFNKSQVNPFVTVGSGVVILPGEKLTVKNLKVSEFYSPNLLLSGKDRVLYSYYNGFPYLPCGVGKNYLRTYDNVSYPLELKDECKYLLTGDCTTRSDFSVVLEPLDLEVGTKKLTLQLSDTVVVLPPPDLYKAEVVLTVNGTTYVATEYKDVVLPYKAYQKLFVTVYPTSGPHDPPVVQLTNTLRTFKVVFDGVNAYVWVHPLYQSKTCGLCGNYDNEPAYEFFTPANELVSNYSLFVASYGFGGSQCQEPVPSVLTGALQKQILHGNLGIIAPPERRHNTVVDYHHQHHKAPEDQDPSLQHFHGYKHPHHHPQHPGHSHPPHHPYPGHHYPQHSYPPHHPPHHHPGHLYPEQPYPPHHQFPHHPYPGHPYPEQHYPGHPYPGHHQQPHHYPSYPGHTSPPYGAQYVPHYYVTPHPLPPSFYGHPGENHGHYKWSKEESWETGGVEKYGPKTGVRESKQIEAGPMIRQFPGESKAT

SEQ ID NO: 98 - protein, 1773 aa, Ixodes pacificus
MFWKCDGQNDCGNHNDEKNCRAGSEPEECPVEKFNCHDRSYCIPKVWLCDGEADCGDSSDEVGCTVNCTGFSCKNNECIPLNWRCDESEDCLDGTDELGCANATAAKLVQQPPKCDVDSGHFPCLDGKCLLPEKVCDGTKDCGDGADEGSFCKVNECSSKKCSQGCFVSANGSSCYCNTGFRLMADGISCIDNDECTERPHVCSQMCTNTPGSYHCSCLNGYLLTDSTFCKAKDPEPLMIFSDSKQVRGLWLRTNRYFEIHSASGQAVGVDFDNSARRVFWTDVTSKKSAIHSCLLDGTGFKVLISEDYSLMEDVALDWLANNLYITDSALKRILVCKSDGSACATVVTAFLDSPRAIILNPSKRLMYWTDWGKSPAVMAAGMDGSNVRALVSENLGWPNGLAYDHSTDRLYWCDAELTKIEYMDLLTMKRNVVIQDAVFHPFALTVFEDTLYWSDWLAYSLDSSNKFDGKHHHRILREDSKNIMGVHIYHPLLRSKDLINPCWDNPCEDICVLATDGYECLCRLGYEPSPKDKHTCTVTRLSSYAVVSEENVLYRVRHESIGGESVSKMPVNRLIMVGAMTLDWATQTLFLSDNPREMILAVDMNTYNVSTIREHHVGSIFGMDFDDQNRNLYWVDADKKTIEVCRSNGSGHVILRSDLRRPVDLALHPKAGVMYVLSAGDTPSIVRYTMDGLSPIRVQLTSMLLGLSLSTDLVAERLFWGDSVRATIEFLDLKNGSSTPFIVEQIHGHITSVSASHGLVHWTTREDGALYYVDLHVKPTSVHSVPLPPRNGTVARKVVYAAQIPDYGKFLPPGPCSVKNGGCSHVCLPALTSRSCLCPPRMVLSANNVTCKDENGTCSAHELSCAGTCIPATYWCDGYQDCSDNSDENNCGTVTCPASGFRCGNGKCVENTWLCDGYNDCGDHSDEMNCTRRNCTSEEYQCRANYCIPVYWRCDGEKDCPDNDDEGNCSLVECPSGYDRCRDGQCFSRDWRCDGQADCKDSSDELDCGATTGCLPEDFRCTNKECIDPRFRCDRRVDCEDGSDENGCDSSYTANTTSQCPRGMVNCGDGQCIYEHDICDGYVDCHLGQDERNCTSAMCSSKEVFCINTKRCILEAWLCDGEDDCGDNMDETLPRCHPTTTPSTTTTEAACWSDEFRCGSHECVPWSRVCDRHLDCMDASDEGSHCDTHCGKDNGGCAHICRESPTGPKCSCHPGYYLTGDFKTCEDLDECQKPGHCSHFCTNTKGSFKCTCAEGYAIAADRQYCKAQSGEASLLYMLPNQIRSFSMRGHAQHLLAQDDLADMHGLDYHFDDKAIYWTEMKEGTINVMLLAKKTPQTVLEDVHRPFHVAVDWIAGNIYFTDGWVHIQACDYSFKHCTDVLETTYSHVNSFTLAANHGIMFWCVWSDVLGKSDGIIERSNMDGSGRVILVSDKILWPCSLTVDHVHKLLYWSDSNKNVIESSTFDGKDRKTIVMGAISSPFSIALYEDWLYWADWGSDSLMACNRHTGRDKSVVHHGDVKATVLKIVHQVHQPSGTNRCARNQCEHVCLLTPLSYTCACAHGYKLMDDSHSCFDMENETAYSLQSSDVLKQPCDPECVNGGICVHNKNKLYCQCPKEFEGPSCEDPVVLALPVADQANNSMWLTSVLIFILCVALLAFGYLFYRRNRHKLSAIDFTVGFKTPTFAAKDEGLLENEHPIAEDYHVVAASQPGFKNPLFLDGHKSHLLTENGEFQRWSSNESVASSVGRDDSNAPFSVTEEETKQKKQVFFFRKV

SEQ ID NO: 99 - protein, 383 aa, Ixodes pacificus
DETSRKSLLDSTRVFTSQETSEKKATGAPRSPPPKVIGPARYPEVSHPLLCTVSSLAVEELRYPHQYCSHIIYTHAEYNFTKKSFVAPMSSLSFSTFLRSLHKRSNGTRGPRYLVSLSQAFVERNLTEMNDPHTPGAMLAWIVEHELSGMAFMEHVIVPENVHFYTSFSWAFKTAQPKSWEMIIGFSSPDLAVLKSKLGRLSNLVDYIVLETHVKQFPDNCIAMFPTAFNESRNPKVLQLTKNLFPRGALMFMPHLPSGKAKTCLSILMAVLMYVVPKGLLHAPSQDCIDFSWCTYSEVCHEKPAFRYYEWAEAVVQSTQSRMYMFDDETTVVNKVRKVHQLSPKTCISLYRADLDDYSGTCNRGTPFLRVAAVRRAVDGKMA

SEQ ID NO: 100 - protein, 280 aa, Ixodes pacificus
MQTIRVSLRKHAVLLMGKNTMIRKAIRGHLDNNPALEKLLPHIKGNVGFVFTKEDLGEVREKIIENKVKAPARAGALAPLDVMIPAQNTGLGPEKTSFFQALQIPTKISKGTIEILNEIHLIKKDDRVGASEATLLNMLNISPFSYGLKILQVYDSGTVFSPEILDITPEDLRSAFVEGVRNVASVSLAIGYPTVASAPHSIVNGLKNLIAVALETDITFKEAEMAKEYLKDPSKFVAAAAAVAPAAGGGAAAKPDAKKEEAKKEESEEEDDDMGFGLFD

SEQ ID NO: 101 - protein, 392 aa, Ixodes pacificus
MMVGRLAALLVLVVSCKAQEELKLTLANNRFGLRLLHALPSAADVNVFFSPYSVSTAMGMTYAGARRDTGEELFRGLGYSASGLTAAQVLDSYARHTQRLLSSESHSTLKVANGVAIQENLTLLDSFRNTLESSFNAEVHQVDFVQRPQDAFEFINRWVKRKTSDKIDKLLDEPLPAAAKLVLLNAIYFKGEWNRQFDRAHTRKRQFWNGGVSLTLVDTMMQIMHVGYGSFDDLDVDVAELPYRGRDYSMVILLPKRNDGVDRLKGNLTVEIMRSLLSELRERDVDVFLPKFKLESTYQLKDSLTALGIRQIFSNGADLSGISDRNLHVSAVVHKAVLEVSEEGSEASSATGVVIMPGSAMPVAFVADHPFLLFIRNTRTNDIIFAGQVNKL

SEQ ID NO: 102 - protein, 196 aa, Ixodes pacificus
MKHFVVLLALIGAATSGNNLFENLDKYPLQDECQAALQEHINVEMHASLVYMQMAAHFDNNKVARKGFSTFFAENSKEEREHAQKIIDYINKRGSTVSLVNIDMPLITTWKSVLQALRDAISLENKVTNKLHAVHKIADEECKDPQLMDFIESEFLEEQVTSIDKLQRMITVLSNMDSGTGEYLLDRELLGDKKEF

SEQ ID NO: 103 - protein, 91 aa, Ixodes pacificus
MKTSVLQALLLTLAVAVVRAANTGIEWPPKAQKCAAGETWKECVGSSCAELTCEHPEPSLGCTYDCNYGCYCAPDFFRNANKECVKKDKCP

SEQ ID NO: 104 - protein, 160 aa, Ixodes pacificus
MALKLATSLILVLLSALICTGHPDGADDEACKDLYPSHGYTAKSAAEGHAEGYRLVQDKEDYKPGDVITVTLSSEASPFMGFLIKAFDENEMDVGSFRSTGADSRAFSHCAGITHSWRDLKKRVVVQWLAPEDRSGKVHFKVTVVKQVKDFYHAIPSTLD

SEQ ID NO: 105 - protein, 186 aa, Ixodes pacificus
MCAEWEANVRKAYEKSTSIKERCQFLTLFPNEMTKQQILNAVPAATKYLIDKSRKLKATNGVWTLPDPYTSNKLKESDVATALRNFLEDEMDCSIQSLNKKDVVTVNILGNQGVCCQTLHDSLYSRIVCSLDESSSRHQAWSNKILYVAPKMGQDCSVPGPMCVCTLCELRTAVSINKHHLINTFH

SEQ ID NO: 106 - protein, 214 aa, Ixodes pacificus
QIVLFCALGPVSAITNTRSPMRYMSPFGKYIGEFVNFLGKYEFLPNNFVMKLLADAVCRYEGPRDVCGNIVFLICGPETMELNVTRLPVFLCHVPAGTSVRTMVHYSQILISGRFQKFDFGENRNQLIYGTSTPPEYDVSQVVVPVALFWSEGDWMADPRDVALLRRRLPNVVLDFKVSQPKFSHIDFAAGIHAKALVYEPMMKLMASYKSAGE

SEQ ID NO: 107 - protein, 394 aa, Ixodes pacificus
MRTVLVVPFLLVAAVAAPASDGEEECDPVKCKGSQNCMCASIKPPNGLEAKDMPQLVMLAFEGAVNVVNMPFYRELMDTPDRKNKQSGCKIGTTFFVNHEYLDYSAVHELHNRGSEIALRSITLNGTMAYWSNLDTDGWKAEIVGERDLLATQAAIPASDIFGMQAPLLATGGDRSFKMIKEAGLLYDASIPHNRVKDSGRITFPYTLDYGLQTPCVIEPCPKDKYPGVWTIPLNVWFKENQIEHLKIDFPCSTIDSCTPPPASADEAYQFLMANFKEFYENNKAPFPMFLHEGWLREGDRKAGFLRFIDWLLTKDDVFLVTVKEVIEFMKDPKPAKSYKESRCVTEVKPSDKCKEPETCVYSKVKIGYNIGDRVMRSCVKCAPEYPWVRAKNN

SEQ ID NO: 108 - protein, 360 aa, Ixodes pacificus
MRAFHPAIVFLSLAAIACSTDAYRTSRQRVFLCYFASWSYYRVGDGKFSVEQIDATPCTHLIYAYAKPVNGSLAPMDTRLDTDVHKMYRKFNTLKRARRGLKTLISVGGWNEGSERFSKIASSERGRRAFAESVVAFLDLHGFDGIDIDWKYPTQRDGSPNDKKNFALLLKDLRKALDHRDHILTASVSALKETIDAAYDVKEISRHVHFMSIMGYDLFGPWNDHTGHPAPLRGRTGGSSVESTLNVETSIGSWISKGADVGKLVLGMPLYGRTYTLEDPNEHGFNAPTVGPGESGPFTREPGLLGYNEICTLISTGGWRVTRDPNVNAPVAVKGNLWIGFDDAESLANKVSFFAVLVRK

SEQ ID NO: 109 - protein, 151 aa, Ixodes pacificus
MEPNKYRYCIARHRTEAEMSLLSAALVCSVVVSLSTAFPGIWRRHHPDVDPRYKEWAHFAISSQVEGRTNYDTLLKLLSVESQVVAGVDYKLKMMVAESTCVVGKDWYSRTRCHPKVDAPHMICTAVVNYIPWEHTKSIKYYSCGEPVDNS

SEQ ID NO: 110 - protein, 1536 aa, Ixodes pacificus
MIVPYVLPVLFIAVAAAYEVGKDYVYHYNGKLQVQNPEQPLQSSGFAFRSKVVAQPRPDHTHFKIIDFEVDSFNGDHIHLSDHQFHYHSTDKLKQFIERPFAGKFSQGKLVTAEIGKNEPKWSMNLKKGVISVFQVDLIKGRHDNPHAKQFYLKEESPEGNCDTLYIVGEKEGNLEVTKIKNLQKCDEEIYTFGRIKGHGCVNCETLETHPGLATSQIKYRLDGTPEHYVVDHACATSDTEFKPFGPGGKTIRVQINRTLDLEEVHDANTDTQLPEDIEKVDHLWLSFPESGDAESLEDLKKVNHLVIDYDLTNDKEQFIAGFNQLAATEYEDDDIKDVHSKESVALRFLILHSLFAVMPFEDVSQMYDQAVANAPEASKSNVRRLFLDLLSAAGTNPHVAFGLQLIKEDKLSDDEAEHFLSKLALHLKENSPALMTELSEACEHVKPRRQVWVNCQLALSSLAGQEGCVRAKTDNEHDEGLCKPSLVSHFFNYQIKPEDKKDQPEYKRTVYMKAAGNLATRGAVHYLERYVSDTNQPEYRRSAALWALKRSSKHHPELVRDVALPVYVNTSESSYLRIAAVTIVLTTNPDLYLLKHIGHNIIDDPSDQVASFVTSMFRSLEKSKYPCHQELAQHLRYVVPMWNDVARFDKPLDHTKSQLLISSGYEPKYDYGGATTFGQIRADDSYLPREVYLGFKHYYSGASYETASMWFESWGMDKLLNRVFGPHPGSTTNMWNVFGRRRFTRDASAKELKEIEDALPVTDREYDHVYGRLSLDLFGHAIDTFEFDESLYEEAANGKENPLDNLKNLLGNVQRTKSFYLSKDIATGMPSELGVPIFIEHKQVGFTYINRQKFAVDTTEDGKFSLDFKRHYVHESGGYSMVGVGLTFNKTFVGVGADIQTVFNLPIDLKITVDPIHKKLSLKRPLSLPWNIVNHHFRPYTFAMPYDLATDVSKAVTTLSKPQYPLYNKDEVTSFDRTYFKYLLGYGLNVKGSLLSKGLKKGLHDFLYHSDCNKRLLYAVMNPEWHPRELQLTVVPDEKDATTEVDVELGYEFLEPDDPRESHFPVDDNVHGNVEVPSTHVLNLNYALKGTKERKVSAELRYSYTKDNLKHKVQFFYDRTPFHWMDKDHSKICLDVTTKFPEPDWDRLKNLAIFHEGKDLEFQMNIHQGSNCVDQASVSFTGKYTHTDDEAKQIRENAEGKPLGANRMKKYNLRVPYNKCTEHQKQGAPLNFDCIKYLYYTSRLGKLTVDIVYKNRKDLFSPMLDYYKRNNPEGGFVSTLSEYYRRATSGKLHVVSQVPPVEKYADIVVTTEDGHIYNHFHVPIYTHLLEPKIFPMLGYSNMANYISYYKHKYCDLQGKSVKTFDNVVVQLPETDCYKVVAKDCSPNKRFTVLARATGNAALPKALKAFIQSTKVELLPIAEDSALVLRVDGNRVPLTSGVPYSHTAHDVELFTVKMEQEYFEVTSKPYGLYLGFNGNILGVQTANFYRGKLCGLCGDYNYERQHELVGPDLHHYNDTLEFAKSYVVPSSDCTPP

SEQ ID NO: 111 - protein, 775 aa, Ixodes pacificus
QDKLRLLQEDLESERELRQRIEREKSDLTVQLMQLSDRLEEAEGSSETVVEMNKKRDTELSKLRKLLEDVHLESEETAHHLRKKHQEAIAEFQEQLDGMTKLKSKAEKERQKFQAEVYELLSQVENSNKEKLTIQKTVEKLEHTVYELNIRIEELNRTVIEVTAQRTRLSAENAEYLKEVHELKVSLDNVNHLKGQLATQLEDTRRRLEDEERKRASLESSLHTLEVEIESLKVQLDEESEARLEVERQLVKANADAAAYKTKYETEVQAHADEVEELRRKMAQKISEYEEQLEALLTRCSSLEKQKSRLQSEVEVLIMDLEKATAHAQNLEKRVAQLEKLNIDLKSKVEELTIMLEQSQRELRQKQADLQKLQHEYEKLREQRDALQRENKKLVDDLADAKSQLQDCVRRLHEYEIEIKRLENERDELAAAYKEAETLRKQEEAKCQRLTAELAQVRHEYERRLQAKDEEIEALRKQYQLEVEQLQMRLAEAEAKLKTEIARLKKKYQVQITELEMSLDAANKQNLDLQKIIKKQAIQIQELQSHYDEIHRQLQQTADQLAVSQRRCQGLQAELDEQRVALESALRSKRVAEQSLEESQVRVNELTTINVNIAAAKNKLESELSALQADYDELHKELRVVDERCQRTIVELKSTKDILIEEQERYIKVESIKKSLEIEVRNLQVRLEEVEANALAGGKRVIAKLESRIRDVEIEVEEEKKRHAETQKILRKKDHRAKELLLQTEEDHKTITMLNDAVEKLNEKVKVYKRQLNEQ

SEQ ID NO: 112 - protein, 286 aa, Ixodes pacificus
MAPRNWKRPLTLIYNNNYKYGNSLYSGAIDDIEKRYQASMSRTAFRSDRGDLGLTTFSSSQIGSAPEPSSSRPTTNASEPEPTLLSPSFSAPSRSPARSVLDIDTDLRKARSSGDFGRLRSSYRPTSWSDDEDLGLLDDPGYSRRRRRRRQDRNDYLLTSPSAAGGDFYLEKCRELKGELEALHGRVMDAESRARNEAGNLRSKVQAEIAELSLQLEDTTLQNNELQKAVKRQAKQLLDLQAYCDEMQRHMQGAVDDLSMANKRCLDLQNEVESLRSNLESGVRHI

SEQ ID NO: 113 - protein, 302 aa, Amblyomma americanum
MKIENLLVRQLIHEFLGTAVLIVIGDSIMAIIIAGGNESIAAIVGPVGWGVAIFCAVSVAGGVSAHLNPAVTLANASIRKFPLAKVPLYFAAQYMGAFVGAALVFLTYKDLIDHFDNGERHVLGEKGTGGIFATYPKEHVSTLTCFIDQVIATGVMVITAESIVDERNFGGLPKFLHPTALGLMIMAIIFSFAYNCMCPLNPARDLSPRLFTLMAGWSAETFTLRNWNYVWVPILGPHIGAILGAWIYKVAISDNWPDAKPRPAASNETKDPKEDLVETLYKADGEKLVLELEPPPPQAQRL

SEQ ID NO: 114 - protein, 335 aa, Amblyomma americanum
MRGPFVIRTRYPIVRGKRHLALDVPLRGRGENRLFPENWTGNKRSMCRRILKLWSPAVAEMLEKLKITNATLREALSEFVGTFVLVLLGTAVLASLTFDKAGVVSYSAGFWGWGLALTLGVLVAGGASGAHLNPAVTVAFATVRKFSWRKILPYIVAQYLGAFLAAVVVFITYLGALHQYDGGIRSVTGVNATAAIFASYPKDFVGTGNCLVDQIVGTALLMVALLAILDARNMNVAPGLQPLFIGLALAAIALCFGYNCGAPLNPARDLSPRIFTAIAGWGVEVFSFRSYNWFWVPIVGPHIGAIVGAWFYTLTVELHCPAYDHDSQGSSRVPL

SEQ ID NO: 115 - protein, 498 aa, Amblyomma americanum
MRHCIVFVVGALLIGKTAGDETATEKVPQADICNEFGNKFCKGEKCNASLDVKGTFTCQCERDTMYFDAVKEMCQYKKTCVTTECSVGQCKEIGINLAKCGCENKAYVTLTCKVTDFYANECQKKGGTAVVNMDSLDGARCSCGEWAVMNSNKTKCIPTTCLYPALSCKDLCEKNLLDKDPRCCEGWDQKDCSKAPPIGTYCSPGTIRKGADCLSACIAGEGKLLCKNGCRASKAPGRAYECQCNSGYVVAEDGIQCKVDYTGLACPQEEEQKCLPGQYCIMRNKTAVCECPLHQHVVDGQCTGECSENKCHEDFTDCRVHFGKQRCSCPWALRKHPKSSVTKECILNEYYYTVSFKPNISLEARDCDTHKNRVLQAMQTTIGPEIFRVEILNCTDDITARLITGKPLSPYLLRKLQICEYPEGNICRIYPRLPIQKGSATEIQEENLCGSLLKEQETATKDTNECVRDGEYFWFKCKSGFREVDLKTRGRLRRSVCE

SEQ ID NO: 116 - protein, 443 aa, Amblyomma americanum
MKLIALPLALLLTCALGMPQQDGDATTPSTSSTSLSTSAASSVSTSQAPANATSSTTTGAASSGRSPVVCYYYGWANTRPNPANYGVDDIPGDLCTHVNFAYAGVDPQTWELKSEVPEYESNRELFKNFTAIKTRYTQLKTLLSVGGWQHENGVFSAMAANSNRRALFIESVLRWMKEYNLDGVDMAWPFPGVSYRGGSPRDKENYASLIRELAGAFEGRGLLLTVVVPLPEEFLEAGYDIPEISKHVDWINAQAYDLRGVWNGVTDVHTPLYSRSIDIGPQKTLNVKDGLARIVSSGAPKSKVVMGIAFFGRGFTLLDPAQHGLHALVNRDVPPRPGPFVRSNEVFAYYEICLNLKGNWKREFDEEGKCPYVYYRDQWIGYDDAVSIRHKINFLLQEGYRGVYVFNNDLDDFRGFCGETNILLKTIKEGLNGNKNEITTSPA

SEQ ID NO: 117 - protein, 259 aa, Amblyomma americanum
MMFHPFHLVEKSPWPLTSSISAFSLTLGFVNYFNNNNFYLVSIAILTLILSSYQWWRDVSREASFQGNHTIYVLQGLKLGMILFILSEVFFFISFFWAFFHSSLSPNIEIGSQWPPKNIIPFNPFEIPLLNSTILISSGISVTWTHHAIMNGDYNSCLLSLKITVMLGVLFTLFQIFEYYQAQFSITDSIFGSTFFLTTGFHGIHVLIGSLFLSISYFRIKNNLISTSHFFGFEASAWYWHFVDVVWLFLFTFMYWWIF

SEQ ID NO: 118 - protein, 250 aa, Amblyomma americanum
MPVASCNGQRGRLSSFSPFHRPFFGRGTSSVVFTMPIVLYYLVGSPPCGFIWSLAKEIGVQVCLKELDFAKREHLGEDFLKLNPFHKVPTIDDDGFVVYESNAIAYYLLRRYAPGSVLYPTCVKERTRIDQVLACASCNIHPDLATFLRPRVFDSTKPTAEEVTALEKNVIKGIENLIGDGKFAVGDKLTIADLCLIGHVVLALENGSVNKDNYPKLAGYYEHVKSELPYFDQVYGPAIDSAKRVWARLK

SEQ ID NO: 119 - protein, 531 aa, Amblyomma americanum
MKLVGLLVLLVAHVRGSPKPKLVYPRLLEERSSDGRMVMHLHDDLTLNLRKASVAAPALRVLTEENGRPVTLFYNGKDIDKDLYEDEEKIASVAVTRNENGLHINGLVGPQHRIEPLPIAGRSEDGVVPHAIYEIEQDEMLDKALAHSEEREKAVIIELFFVLDTRHHSHFSTNEKLLIYLCVMINSVNLRYGPAKNPRINFMVTGMDKSTNESYAVLAKQNDKYIFDEGTLSNLKGYVHERETKYGQPDVVYLMTGRDVYTVINGKASVNGLGIGYVASICTYSKVALGEDRPGLYTGTHTLTHELAHVLGAHHDGEASTTYGHPGASQCSWNDGHIMSYINKGPSHHRFSWCSLNQIQYVVRRAGQDCWTIKGKGYIVQGHYPGSVVATADFCKDAIKDNQAVIDNVTVSERTCKVRCMYYKRDTSYVNGRLTWRYYQEVDALDYMSCGGNKVCIQGVCVKKPAAEPEATTRGSVVYTTKAPTEAPTTIPRVTDPRCRCDCNNTVPARTSLRPYYRPWPGNRNPGYRSG

SEQ ID NO: 120 - protein, 374 aa, Amblyomma americanum
MAANPLGESLLSFSIDLYKQLISESGRSGNVFYSPFSISAALSMALAGARNTTAKQLTEVLHVNSDDIHKHFSSFFSQLSGFSPDVKLHVANRMYADRAFPVLDTYLSLLRDSYGATIESVDFQSNYEKVRQEVNAWVEKVTESKIKNLLPEGTLDASTVLILVNAIYFKGLWSSQFDPKSTHRSHFHLDSKNKKEVEMMYQQSDYKMSRSDDLEVTALEIPYRGGKTSMVVILPDSVEGLSKLEDSLTASNLSDLLKNFRNGSDVELYLPKFKLEQAIYLKETLKAMGIKDFFTAAAELRGISDRRNLVVSEVIHKAFVEVNEEGTEAAAATAVVMFDTLLMTTRFVVDRPFMFLIRSLDPELVLFMGSVRQL

SEQ ID NO: 121 - protein, 402 aa, Amblyomma americanum
MALYAVKVLLLCAALVVSASAQDVVLRHARANNDFGISLFKEICFGNRDKNVFFSPASISIALGLLYAGASSKTLSELSAVLGLTDAGLVDRDVVLPAYKSLIETRSEKATLDIANTVLIQHSTEILDQYRKDLADFFHAEVRSVDFLRDGSKVAAEINSWVSAKTRGKIPKILDGPLPMNTVAFLINAVYFKGTWLTKFKASQTKPLPFYNHGRDEVKVATMSLRRSLRYAFVDELGARAVEVPYAGDRFSMIIVLPRTRTGLSTVESQLTVDALEKITNELASRNVNLWLPKFKLETDYDLVTLLRKLGLESAFGGGADFSGISARNNLQVSDVKHKALVEVNEEGTVAAAVTSIRMRMKSSRRAQPPPPVEFRVEHPFAFIIWDRIDKRALFMGAVRKL

SEQ ID NO: 122 - protein, 375 aa, Amblyomma americanum
MAANPLGESLLSFSIDLYKQLISESGSSGNVFCSPFSISAALSMALAGARNTTAKQLTEVLHVNSDDIHKHFSSFFSQLSGFSPDVKLHVANRMYADRAFPVLDTYLSLLRDSYGATIEPVDFQSNYEKVRKEVNAWVEKVTESKIKNLLPEGTLDASTVLILVNAIYFKGLWSSQFDPKSTHRSHFHLDSKNKKEVEMMYQQSDYKMSRSDDLEVTALEIPYQGGKTSMVVLLPDSVEGLSKLEDSLTTSNLSDLLKNFRNGSDVELYLPKFKLEQAIDLKETMKAMGIKDFFTAAADLRGISDKRNLVASEVIHKAFVEVNEEGTEAAAATAVVMMCYSLPMTTRFVVDRPFMFLIRSLDPELVLFMGSVRQL

SEQ ID NO: 123 - protein, 149 aa, Amblyomma americanum
MPFSVNPPAPPTRAHQINPSPFGDVPPKLTSEEIAANIREEMRRLQRRKQLCFQGAECSSPPEGCSPIGSPSQHTGGLLSPVRRDQPLFTFRQVGLICERMMKERESQIREEYDHVLSTKLAEQYDTFVKFTYDQIQKRFEGATPSYLS

SEQ ID NO: 124 - protein, 282 aa, Amblyomma americanum
MAPPCYADLGKQARDLFNKNYHFGVVKLDCKSTTQTGVEFNVSGTSLNDTGKVNASLETKYKIPEYGLTLKEKWNTDNTLSTEVCSEEKLARGLKVAFHANFAPQTGKKSGALRTAYKLDNVHLNGDVDLGPPGPIVHGAAVLHYQGWLAGGQVSFDTTKNRLSKTNFAVGFQAGDFAVHTNVNDGQEFAGSVFQRVNPNLQTGVQLAWTAGTNATRFGLGCVYDLDQETSVRAKVNNSGQIGLGFTHRLRPGISLTLSTMLDGKNFNQGGHKLGLGLDLSA

SEQ ID NO: 125 - protein, 1906 aa, Amblyomma americanum
FAGRFKSQLATQVVWTFDSRHADPVPSAYPSGVVYKVRATVSLKSPELTVTEGAGLQYAGDLALYPVGQDQYEARFLNFSVVKHEQLYGDLDNLTALSLNESVPLLNETFAVDYSQQFRYPVRFVFSAGKVVKYEVSPEVPTWAHNVYKALLTLLQNQVETPQEVPTVASYYEDGVSGYCRVTYEVHSLTKNLYTEGKVYNVTKTKYLDDCKVTRPVHSVDDALRTGYYGVCSKHLPDNFLPGYQEDTSAYESKPLTGCLEGLRPVDSVVTAHEVSYYNVSESLLESALFDSLAVLPVLNAGEVVVRTRLQLELASLEAPPVNPTAFQGEEHTSLQLELPEATQYLDLPVYSVLVGTVDQVKPEVFVQVLEQVAEELVNLELDTEVKKTPALVLKLVHMVSLLNTEQLKQALPQALQSKTYELEPKEQVLRALYVDLLGKAGSKSAVEVAVYLVKEQLLTRYESSRLFRDLSVFKSYVDKDTVELLLDLCKSTEVSLEPFVKVAVCDALGEALNKACPTKVLYHSLPVSYYKASWRRIPKIKSRYDETLPKHKLPYEPVQRLVLPEEKKCTVTDLYEYVKVLEEALKSTKDFKVLVAYLNALGKVAKPEVLPVLLSYLNSTAENLYELVSEEEELYEAVYFVKKLVLLALDHVVEYFPKEVNPLVRAVLLNASEPVDVRTLAFDVWVKSVPTKWDLQEVSLTVKLDPSLELKSYVYSAFKALLKDKHPANSVLASRLRGVFVDVESLNVGPRYSKYYKKTDYETSKNLGVEAVFKHVASNVSFVPTYLHAGLKYNLASYVKTLVEAKLLVKGSERFVDEVFSSNGGLLQQFVQALAGNTETPVEKTPYETTTFEHSVVKELNLETRKVEFPKAVLFAKLLSGDLVLPVDKELLEVVKRELVQLAVKATKEAPLNKHYVRVLLLKKVAHVEPTAVGLPVVQTVLHPVVVSLKLKDVYLQAKASPKSLLPYTFNVSGTVQPLLLSVKQSRVFVPLEPTEEAPTVVLTNVKSAHFKTELALGYDSTVEKSLRLVVKPTFGTALLSAVCSDHVVEPYNPFLLLEKPIVDPKKCLDKVEKPYSYHRVYDAKLVDVEVDAYSHGPFVPLPLYSKLHKSLSGLLDKSYEYLVHMGTKQHLYRVKVQPNEKDPVTEWVATFKYHSNLNFLVKETLKKVYTETQRGSYDESVEKPYEQMRYKPVSPSLKPYESYDSSSSSESSYEYPEEVLHLWNRTFVKHVLNVTFESLQSGTVKKLTTLDLAYYHSLNKTLKHYYADLKTTTKPLVSLFLNVSYPVPPSPFWYVPSYVGNDLVNATLYLHYSDMPYPFVVTYNATKTQEQLLGVHAYDATPLLHWFVPQCLADQHAGNTVSYACRLATVFDAHLNHQVLAFKVPRDVSPKLKNAAVKVLSFLKYKLYPYASFFALPRTEEEELEVVVRLNKTDVNPYATYATGEVFLPGEKVVVSDLRVSKYSVPNLLLSVYDRLKHGLLKSYPLQPCSVGKNWVRTYDNVSYPLEVRQSCKYLVTSDCSKKHDFAVVVEPLDLTVGTKKLLVQLGHTVVELPPPDLYKAEVLLTVNGTQYVANTTQDVVLPYKWNRKVLVTVYPTSGPHDPPVVELTNNLHTFRLLFDGVNFFVWVNPLYQGKTCGLCSNYDNEPYYEFLTPTNELVSNYSEFVASYGFGQPVCKELVLPYAYPLHYLEELKKPEGVEGYYSKHHEVPRLYPAPKYPTHHQYPTHHKYPTHHQYPTHHQYPSHHQYPTHHQYPTHHQYPSHHQYPTHHQYPTHHQYPTHHEYPSHHQYPSHHYSQHEQYPHFPSLQEELDKCVENRTILRDRKGEICYSVFPVPTCKSDCRKTKGSTQLTVGFTCLPRNSSQAKEWTRKVREVGTVGVLPPTYPVTLSEKVRVPEKCVPLVA

SEQ ID NO: 126 - protein, 1801 aa, Amblyomma americanum
MAAVTRALLAVLLLQIAHQASSECARGWFDCGNGRCIAMFWRCDGQNDCGNHKDESGCTNAHRTCPVEKFACQDSSYCVPEIWVCDGEADCHDSSDELDCHSSNCSGHRCHNNECIPSHWRCDGTEDCADASDELGCAATGAASTTTTVSPRCDVDQGRFPCLDGQCLLPGKVCDGKKDCTDGADEGTFCKVNECSQKKCSQGCFVATNGSTCYCNPGFRLMADHTSCADIDECAEHPHLCSHGCTNSPGSYKCSCVEGYQLTDNSFCKARDPEPLLLFSTTNEIRGLWIRSNRYFEVHPAEAQAVGVEFDSEQLRLFWTDVSTKQSSVYSCRLDGSGFKTLFTAERTLLEDLSLDWVTNNLYITDSLGKRILVCSTDGLSCGAVVTTELDSPRAIIVNPTQKLMYWTDWGVKPSITRSGMDGSSIQRLVTTALGWPNGLALDHPTNRLYWCDAKLSRLEYLDLTTMERTVVLEESLFHPFALSVFEDTVYWSDWASYSLDTSNKLTGKQHHRLLRENGRHIMGVHVYHPILRQRGISNPCWDNPCDHICVLSGDSYMCLCRLGYKLSSNKHSCTVTKDFSFAIVAEEDCLYKIDLRTVGAPFPTKVPLSDVGMIAALAFDWSNQTLYFSDNQREILAAINMNTFEQRAVYDHIGSVFGIDYDMTHGLLYWVDADKYTLEVCRANGSGHAIVRDDLRRPVGIALYPFAGVLFVLTAGDRPAITCYTMDGQNPKPLPLTSLLVPVSIAVDLVARKLVWADATRGTIESLDLVTVFTGTPFIVHQVKSHVSSVSAAHNEIHWTSRDNSSLEYIDLSAEPHVRRSVPLRTGRNGTYSRRVLAASQVPDFEPGLCGRSNGGCSHTCLPVRTTERACFCPPGMALRTDNTTCRVENGTCRPHELPCAGGCIAAAYWCDGHKDCADNADEATCGTTCPSKDFACQNGKCVEMAWRCDGYDDCDDQSDEANCPHQTCASHQFTCKSGACLPFYWRCDGANDCPDGDDELDCRTLRCPNGHDRCANGQCIPRDWSCDGHADCSDSSDETNCTETVSCFEDDFHCANGQCIDKRLRCDHDEDCEDNSDESGCDYAPANNSKCVKGMVGCGDGQCVYTHDMCDGYADCHNGWDEHNCSAPVCQSAEFFCPGTKRCILQSWLCDGDDDCGDAMDELLARCRPATIPPSTNAPCWSDQFQCGSHECIAWSSVCDGRTDCADFSDEGSHCEHHCATANGGCAHICRESPSGPLCSCRPGYRLNSDRKSCDDVDECLSPGHCSHFCQNSKGGYKCTCAAGYALGADRRYCKVQYGEAFLLYMLPNQIRSFSMHGHVQHVLAEDTLSDMHGMDYRISDKSIFWTEMEEGVINVVTLGNSKQFTLLDDIHRPFHIALDWVAGNIYFTDGWVHIQACESTFKHCTDVVDTAYSHVNTFALAANDGLMFWGVWHEIVNKDYGVIERANMDGTDRRILLTDKILWPCSITVDAVHLRIYWSDANKNVIESATYQGKDRKLVRGTGLASPFSIALFEDWLYWSDWGSDSLMACNKYTGRNVALVHHGMSKASVLKVLHAVHQPSGVNRCARNSCGHICLLKPSSYKCACGHGFTLAKDSQNCVESDERSYNLTSADVLGQLCNPVCLNGGHCISEKSSYYCRCTKDFQGPSCQDPVVVYMAPHRASSRSTWLAPILITLLCISLLVLGYILYQRNKNKLAALDFSVSFKKPTFGKREGLLQHEHPVGADEDYHAMTTQEPGFGNPVFAPPKSQLLLEDGQFKRWASSDSLQSSDSQEKSTIAFAGQFSSAKQDKVFFLRKS

SEQ ID NO: 127 - protein, 2189 aa, Amblyomma americanum
MFCVVLGLAVAFALGSRPSSPHDEGDDGARPNFAAVVDKQPRPAASAIKPEQPAAPAGRQAAQGAAGNVNFNKAAFVNVSVVQSAPVRSPANGHLPQSADHSEEDESPLVCVLSERTESARTVTTSYCTHVVFSLRHFDNADLSVLPEGYLAQLQESGVRVLVAVHERLLQLSSLRESAETTAGQLRNRKLDGLALLYVSRSTRQLPLLARKMKELHDVYQSNGLCVLLSLQMVDYDKPAAHTATQLSALYENVDFLVVNTHYSGDYGPCRATPASTFNQPLSTCIPQVPVNLALGWMQNMTGGSSYLCFSVDMRAFAYQMYGPMVVDEACYSEEHLHYEQVCNKDEWTYKQDDYTLTNIGQLSNVLHSFETPATLKGKVLAASRSVRAPCVAAFNYDYEDSSGRCHGTPYARMKSLRQWLDEAAPAGKKGYASVQKGFLEPHETEDSTTTTSSSFEDYPSERPLVCVVSKTVHDEAAVPQGYCTHLVFSGALFNQVTKTVVVPRGIWRWRELRHDMALFLGFDDDKLIDYLIKSGSEAGMAFGTLAAKVLRGQGFNGIALLNVNRTSHGIEKLSSALPVIRELFANDLQIVISLDVVDISAPPTMMALRLNDIFKFSHLVVFQTHYRHSLGYCEVALPSTFDVPNHSPTVPVETALRWLGRMGAGKQACFSVNLAVIEFLESDDSYLCRKKNLLNYVEACDTKNWVEVGQPRGALCTLRQKGKLWQAYETEDQLSAKISKAFELSTGACVALFNVDYENYNSACFQKFGRMTSVVQSYLSYQHQRPRPTRRRGLLKLPFNRTKSSSEHNHEGLIRNTGITHPKLPRGNLMSLRVPATSRPLLCILSEQWSEIADVPDAYCSHFVLDVAHLNFKTGMSKDPVTLLKEKFGKRRQYLVKISKLPLRNLTQYAQELSTSIVKLQLHGIAIVNQHVPSPLLSTFNERLQLFRSSLKKELILALGLEIPDFDQKPSDVAARLAPLTRTADIVILQTHFRRLWKFCRTAYPSIVQESNDTCQQSVPMMNALKWIKLVEANRHVCLSVNLATLRFHYVDAPGPGSSCTKSREIINYCDSGPWNESTDTAYSMSAHRHKEGLWDTFEEEELIKAKVKKIEVARPKSCWAVFNVDYDRDTVPCSMHKESFARLKALGKAAGYSTEWTEGIQPDKDSSKRNRIGGGPYKGTQTGGSLVCVFGGTVSESLLPPAGFCSVAIYTSVLYDEAKRTLVAKDADSLHHFSTLAKHTSLVGAGVAPNILAANKTRDFALESAAWLRVKGFNTLALLTSKWPREDIFAAFKELRSVLKMSEGFGKVSIILGIPPQKNIKDLLFLQKEHVDKVIFMNHYEPGSGPCHVMHPSSSSKTMSAMPGLVENAEIIKAVSSQPNITTEICVSMNLAVLTFTLSRQNKFRLGSQCTHETMTSYSEVCPSHSGEDILYDSALSCYKQNSTHVQTFENEGTLEMKVEFLKKQLHSPCVAAFHAEYEDTEGVCENRTAFSRIAAIRYSLDGASSHKESSGKTEATKRNVSSIPGTPEPSEILLCVFSEDSMGLSALPVGLCTHVIYTGLSYVPKSKELVPFDESAFKHFLNLRIDARLLAAVPFAAILALQKPRDIESFSSAVRSWLIKHDLAGLALIATRNADFDQVYAVTKVLRWQFMNSKSRHLQLVFGAPIHVVTPALIHIADTVNYFIITHHDAMEQSCQLETMPAKQFGKRDMVETINLANKMALEVPSAIFCMSLSLAVVNVPLISVSQQGLVSCSNRTLTTYDKVCPHSQVGTVVEKDFAGVYQRNDNATFYFENEESLLEKVHVAQKIYPLKCIATFYTDYEDVAGVCKIRAPFSRLKQISKALGYHETKKEPVPVLETRNTSSSSRPLVCVIADHYPDFDKFPVQYCDYLLYVDPHVRIRRNSFHDFMGGTFQTFLRYSSQPNLLAVLGFLVTDETWKLQLSDSISYATSLTAWLKEKKLKSLAFIMPVVTEAIAEHLLVFVNLLKKSNPDLKLVLACPVTEKPTALIPFVESVDMLIFMTHRKAPESPCRITLPSTRSILQDENNDLAGSSLFMQAFGDKKHQPLMCFSLNFATLKFTVKDNRTEFGAPCEQETWVNYYETCPRRGVFEGFFTGFKTMVQFSEKDIWTYEDEHTVSWKLLSYLQSNPLTCVAAFNLDYEDRRGVCRQPYSRLARLSNALGRVDL

SEQ ID NO: 128 - protein, 318 aa, Amblyomma americanum
MVREDKTTWKSNYFLRLVQLLDEFPKCFIVGVDNVGSKQMQTIRVSLRKHAVLLMGKNTMIRKAIRGHLDNNPALEKLLPHIKGNVGFVFTKEDLTEVREKIIENKVKAPARAGALAPLDVMIPAQNTGLGPEKTSFFQALQIPTKISKGTIEILNEIHLIKKDDRVGASEATLLNMLNISPFSYGLKILQVYDSGTVFSPDILDITPEDLRSAFVEGVRNVASVSLAIGYPTVASAPHSIVNGLKNLIAIAVETDITFKEAEMAKEYLKDPSKFAAAAAPAAGGGAAAAKPEEAKKEEAKKEESEEEDDDMGFGLFD

SEQ ID NO: 129 - protein, 710 aa, Amblyomma americanum
MAMAYVGARNETQRDLHETMRYESAGLTPDDVHSVYARHTRLLRAPSNSTIRVANAAVVQNGYSVNEQYLAVLTRSFKAEISFASLSGEGSLNDINNWVSNKTEGKIKKLLSEPLPLDAKLVLLSAIYFKGLWMIPLLATKRSKAQFFNAGTQRVEVGTMYARGLLAHAEDNETNSDVIDLPYAGNGYSMTIVLPRQRTGADALRQSLTWPVFQRLRSKLEIKLVFMALPKFKLEGEYKLKRPLSDLGASKAFDESQADFSGITGSRDLAIYDVVHKAVVEPSGSTRRAVLDERTNLQARCPCFHRPRLFSSGGRLQCKTMKSAIWLAWLLALCVAQEEHKVTDANNQFGFRLLHKIPVSSEVNLFFSPYSVSTAMAMAYVGSRGETQHDLHQALSYSTTGLTPDHVPSAHAQHTHLLRAPSNSTVRVANAAVVQNEYSVLREYLDLLSQSFGAEVNRAGLNDELSLNSINDWVKNKTEGKIEKLLSQPLPPNSKLVLLNAIYFKGLWSTPFEPAATSKAPFFNAGAHSVEVDTMHAQLQAGYAEDEETNSDVVDIPYAGLDYSMTIVLPKQRTGAEALRRSLTWPVFQRLLSKLSNTVVDVALPKFKLEGEYLLKAPLSELGASKAFDEEHADFSGITGNRDLVIYDVVHKAVVEVNEEGSEAAGATGVIFYTKSAIVGVPFVVDHPFLFFIRNRQTGDVLFAGQVNHF

SEQ ID NO: 130 - protein, 181 aa, Amblyomma americanum
MLRLLVVASALAAAGSNNLDEQASQNKYFLHDRCRLGLQEQINLELHASLVYTQMAAYLANNNVARAGFAHFFRHQSDEEREHAHKLIDYVNLRGGTVSTVNLQMPTTATWMSVLDVLQRALALEHDVTNRLYELHRLADDHCRDPQWQRLINIPPEVSRILRYNGKTSKPHMVVCPPFLF

SEQ ID NO: 131 - protein, 94 aa, Amblyomma americanum
MNGTLVVVLCFVALGLAADEESDSVLRWPPTPQECAEGETWKECVSGSCAEATCERPVVGPACTDDCNYGCYCADGFYRNQHKACVTRDKCSQA

SEQ ID NO: 132 - protein, 193 aa, Amblyomma americanum
MAKKNWAERFGEKAIVCEHDESVASSESSSPRALSLRRDSLRFGHSAKKNSGVPMAFLRQAVLLVVLVGACWAHPDGAPASACATMNPEHNDSKHEEAGPSTPYTLVQDKKDFKAGDTVAVTLSGAPFKGFFIKAFDDKNKEIGQFEASGESKPVTECSGVTHTNSAEKTAVKVLWKAPAGASGHVQFRQVAF

SEQ ID NO: 133 - protein, 359 aa, Amblyomma americanum
MKRRARQGEEERPPPLESRATSLPAPALRHSQRPALGWEDLAQRFPSPGRRRRPLPRVLRFQTSGGVGVETQGACGQDLRLRSPRVAPHPRRSFVPHLQAPSTSEPVNTSKETKLATWETPTPNAGGGTLRMPTGDSRKTEHTRWQFLYRPWDQVCYYLKKKEERRKMEEKERKKAEVRKRLEEAAKAKKAGGKRGFMTPERKKKLRNLLRKKAAEELKKEQERKAEERRKIIAERIGQPKPLDGANEATLQGILKQYHARIEKLEDIKYDLEYEVRQKDFVINELTIQVNDLRGKFVKPALKKVSKFDKLKMVVKSTSEVDFRSNLKAVKKEAFKLDEEKQAEKKPEWALGAKKEDEE

SEQ ID NO: 134 - protein, 431 aa, Amblyomma americanum
MGLVGTKRALAVFVACFVTHVWIQGSSAVVSVDPDISRNVSQIIVDKGYPVEEYEVETSDGFVLSVQRLPSGRKGPPKKAPHGRDKKAVFLLHYLLGSSADWVMNYPDQSLPYILADAGYDVWLGNVRGNTYSRHVRYRRDQKEFWDFCFDEMISYDLPAMLDFVLNKTGQEKLFFVGTSQGAMILFGLLADKPEYNEKIELFTAMGPVTTVTHMRSPIRYLAPFVENAGAVFALFGEYDFLPSNALMKLIAGTLCSFEESRGLCENVMFFICGPEGPQLNKTRLPVLVSHTPAGTSVRNMAHYAQLVLSERFQKFDFGTTRNQVLYGQDTPPEYNLSRVSAPVALYWSRSDWLADPQDVSQLRNRLPRLVLDFQVQEPSFTHLDFTVGVGARRLVYEPVMKAMASVRTLVHHFYRAPRDSCSLCSSGSTA

SEQ ID NO: 135 - protein, 379 aa, Amblyomma americanum
MTTTNPTSQASTTAAPARGLYAGCDDNVCKRPDCVCARDLPPAGLRNDQMPQFVMLSFDGAVNVVNMKFYRELLEGARRKNKANGCGIAATFFVSHEYLDYVAVNELYSWGNEIALHSISHQTNWDYWHTINSTQWEREVVDQKVMLQTFANISGFDITGVRGPFLVSGGDQGFRKLSQHFRYDSTLVHPRRSDEKPFFPYTMDFGFQLSCNVDPCPNGTYPGLWLLPVNVLFRQQMPCAVADACMPQPTSAKETFEFLRSNFEYFYTTNRAPFPVFLQEGYLRDPGRKQGYLQFIDWLLQKDDVYLVTASEVLDFMQDPEPLGVYNQLVCPGGGAVVNTCPKPTTCSYKRTQLGSDRYMRICSRCPRKYPWVGNPLGN

SEQ ID NO: 136 - protein, 568 aa, Amblyomma americanum
MEAVTVLVVVLCALSLPHGEAASLAQDGSRVFFCYWGSWSHYRDGAGKFSVGQIDASLCTHLVYAFAKLDNGVIAAFDPYLDLKDNYGLGMYEKVNKLKTTHPQLKTLLAIGGWNEGSEKYSRMASTPEGRKRFAQSVSVFLSKYGFDGLDLDWEYPAARGGGSQDKQNFVLLLEELRSVLNPKGLLLTAAVSAGKATVDAAYDVPGIMRYLDYVSVMAYDFFGSWNSYTGHCSPLGAREHGSEEEKTLNVESSIRMWLDKGADRAKLVLGMPLYGRTFTLADPRNDGFLAPTVGPGPAGPITREAGYLGYNEPTGLQQSSEDKSQTGKEERHSRPRKRSSSGARGDNKSRDRHSSFPPLPGPERGHGGEQGSSHGRSPSATRKTICSQLQSSKDWKITRDPRVVAPVAVKGNLWIGYDDAQSLTAKVEFVRSLGLAGAMVWSIETDDFQGACGTTKNPLQRAIREALASNATVTTTTLAPTTQSAGTSTTAALPSTVSTTTLVTSATTLTTTPQPVLTCTQEGFYAYPNNPRKFYRCVLRPDGSYATYVFDCAPGTVFNATLSTCTF

SEQ ID NO: 137 - protein, 143 aa, Amblyomma americanum
MTTVRQAALVLFATATTALICCHNSTSVLVGGWTRQEPGSNPKYVDLAHYAISQQREGRKIYDTLVNLTEVYTQVVAGVNYRLVFTTAPSNCTIGKDTYSAQRCAPVEEVNGRCTAVVYEVPWTNTTGLTSYACSPTGQEQTS

SEQ ID NO: 138 - protein, 143 aa, Amblyomma americanum; identical to SEQ ID NO: 137

SEQ ID NO: 139 - protein, 874 aa, Amblyomma americanum
MSSSRSSKYMYKSSGGAGDISIEYGTDLGALTRLEDKLRLLQEDLESERELRQRIEREKSDLTVQLMQLSDRLEEAEGSSETVVEMNKKRDTELSKLRKLLEDVHLESEETAHHLRKKHQEAIAEFQEQLDVMTKAKSKAEKERQKFQAEVYELLAQVENTTKEKITIQKTVEKLEHTVYELNIRIEELNRTVTEVTAQRTRLSAENAEYLKEVHELKVSLDNVNHLKTQLATQLEDTRRRLEDEERKRSSLESSLHTLEVEIESLKVQLEEESEARLEVERQLVKANADAAAYKTKYETEVQAHADEVEELRRKMAQKISEYEEQLEALLTRCSNLEKQKSRLQSEVEVLIMDLEKATAHAQNLEKRVAQLEKLNIDLKSKVEELTILLEQSQRELRQKVAEIQKLQHEYEKMREQRDALQRENKKLVDDLSEAKSQLADAIRRLHEYELEIKRLENERDELAAAYKEAETLRKQEEAKCQRLTAELAQVRHEYERRLQAKDEEIEALRKQYQLEVEQLNMRLAEAEAKLKTEIARIKKKYQAQITELEMSLDAANKQNLDLQKIIKKQAVQITELQAHYDEVHRQLQQCADQLAVSQRRCQGLQAELDEQRVALESALRSKRAAEQSLEESQARVNELTTINVNIAAAKNKLESELSALQADYDELHKELRVVDERCQRTIVELKSTKDILVEEQERYIKVESIKKSLEVEVRNLQVRLEEVEANALAGGKRVIAKLEARIRDVEIEMEEEKKRHAETQKMLRKKDHRAKELLLQTEEDHKTITMLNDAVEKLNEKVKVYKRQLNEQEGLSQQNLTRVRRFQRELEAAEDRADSAESNLSLIRAKHRSWVTTSQVPGGTRQVFVTEETSQNF

SEQ ID NO: 140 - protein, 298 aa, Amblyomma americanum
MAPRNWKRPLTLIYNDNYKYGNSLYSGALDDIEKRYQASLARTTFRADRGDLGMSTFSSSNIGSAPEPSAARPAASTNEPDIALLSPSFSAPSRSPVRSMLDADHDLRKARSQGDFGRLRSALRPSSWSDEEALMDDQSYSRRRRRRRQDRNEFLISSPTVAAGEGVEYYFDKCRELKSELESLHGRVLDAEARARSEATTLRNKVQTEIAELSVQLEDTTSMNNELQKAVKRQAKQLLDLQAHCDEMQRHLQGAVDDLSTANKRCLDLQNEVDSLRSNLESGLGLNRYASLYGMRHI

SEQ ID NO: 141 - protein, 430 aa, Haemaphysalis longicornis
MLRQIALTSKVAPAVTCVVGAHFSAPQFVAPSVATPSNLGNQKRKRLQHVFKRHLRGAICCDAFKSRKPKAKAPPTRVQAAPSALKSSCQMRALGNGPSAVEGARDRKPAAGEVYLPRNSERLESVQICGATATKSFNMKIQNPLIREFISEFIGTMVLILIGDSVLAVIIAGDNESIAAVVGPLGWGVAIFVAVSVAGGVSAHLNPAVTLSLASARKFPLAKVPLYFAAQYLGAFVGAALVFITYRDQIAHFDKGQRMVTGKTATAGIFATYPKEHVSTLTCFIDQVIATGLLSLTAEAITDERNFGGIPKQMHPICLGLMIMALIFGFANNCMCPLNPARDLSPRIFTLLAGWSTETFTLRNWNYVWVPIVGPHIGAILGCVAVQGGHRRPLAQPAATRRLQYVSGRALPAVFFFPSHPHPRIRSVAL

SEQ ID NO: 142 - protein, 298 aa, Haemaphysalis longicornis
MKPNAFIRAWQRATGCSIGKPLVRQSLAELVGTLVLTLVGDCVLASLTVSRLGAVGLAAGPLGWGLAVFLGVLVAGGVSGAHLNPAVTVAMATVGKISWRKVLPYVASQYAGAFLASCLVYMVYADALRLVRWTALLTAVYGENATAPVFSCFPAPGVSTFTCFLDQVVSTAVLLLGICAVTDNRNMSVPRGQQPLLIGLTVSACMYAFSSNCGNPLNPARDLAPRIFTAISSWGSAVFSFRSYNWFWVPVVGPHVGGIVGVWTYKLAVDNHWESEDDTDSCERKLLRSSRGSNGPAA

SEQ ID NO: 143 - protein, 431 aa, Haemaphysalis longicornis
MAHENRHFINHSHKKFLPGDTVTPSSTTRGSTSSSSPNATSATTSTTTRSGATTERAPVVCYYYGWANTRPNPANYGVEDIPGDLCTHVNFAYAGVEPGTWELKSEVPEYERNRELFKNFTAIKTRYTQLKTLLSVGGWQHENGVFSALARNEQNRARFVESVMQWMKDYNLDGVDMAWPFPGVDYRGGSPRDKENYVALIRELANAFEGKGLLLTVKVPISEEQLNAGYNISEISRYVDWINAQAYDLRGVWNGVTDVHTPLYSRSIDVGDQKTLNVKDGLALLVRSGAPKSKVVMGIAFFGRGFTLLDPESHGLHALINRDVPPRAGPFVRSNEVFAYYEICLNLKGRWTREFDDEGKCPYAYYNDQWIGYEDPVSIRQKVEFLRQEGYRGVYVFNNDLDDFRGFCGETNALLKTIKESLYGEKNEVSA

SEQ ID NO: 144 - protein, 258 aa, Haemaphysalis longicornis
MFHPFHLVEKSPWPLTSSISALCLTLGFINYFNNYNYILSLMALIILILSSFQWWRDISREASYQGFHTMWVLKGLKLGMLLFILSEVFFFVSFFWAFFHSSLSPNIEIGGIWPPKNIFPFNPFEIPLLNSTILISSGISVTWSHHSIMMGNMSNSLLSLKITIALGILFTMFQLFEYYQAQFSISDGIFGSTFFLTTGFHGIHVIIGTIFLIVSAQRIKNSLISCEHFFGFEAAAWYWHFVDVVWLFLFTFMYWWIY

SEQ ID NO: 145 - protein, 308 aa, Haemaphysalis longicornis
MSRNARNRPPSPTPALYISVCINGRERCAEVAGIAGRRRPCKTPLGGGSSSPSRGREAPLKKHAPEATHRSVASLTCVVVELRSRSRLAAAMPAVLYNLIGSPPCGFVRSVAKHIGVELELKNLDLAKKEHLTEDFLKINPFHKVPTLDDDGFVVYESNAIVYYLLRKHAPESELYPACPRGRTRVDQVLAAVSSTVQPHLVAFFRPRFWHNTKPTEEELKNFEENVLKGFEHLIADDKKFAVGDKLTLADLTIVANLVLALECGSTDLSKFPKLAAYYERVKPEVPYFEEIYRPAIAHIQQRWSELK

SEQ ID NO: 146 - protein, 376 aa, Haemaphysalis longicornis
MAPNPLGDSLRSFSVDLYKQLLPKGGDAGNIFYSPFSISAALSMALAGARNNTAKQLSEALHANSDEIHKHFSSFLSHIPGFAPDVKLHVANRMYSEKTFVVMESYLDLLRDAYGATIESVDFQNQSEKVRQQVNAWVEEATASKIKDLLPGGSVDSSTSLILVNAIYFKGLWNSQFDPKSTHRSDFHLSTKNKKEVDMMYQKNDYRMGRCGDLGVTALEIPYRGGKTSMVVLLPDELEGLSNVERRLTSAKLGNLLKDLHMTSDVMLYLPKFKLEQTIDLNDTLKELGIRDFFSSSADLTGISAVGQLVASKVIHKAFVEVNEEGTEAAAATGIAMMCYSALMPDTRFVVDRPFMFLIRSQDPDVVLFMGSVRQL

SEQ ID NO: 147 - protein, 315 aa, Haemaphysalis longicornis
MLNLSKRQDVLAAYKLLLDNFDNGFDTVNTILIRKGAEILDQYKEDLKECFAAEARSVDFARDGTKVAEQVNEWAHAKTRGTISQLLDGELQANTAVLLLSAAYFKGVWLTKFKADQTKELPFYNYGRDEVRVPTMWLQRGLRYAALEGLKAAAVEVPYAERQFSMIILLPDDKEGLPALESELGVDVIDEIIDGFAYLNVELWFPKFSFSSNHDLLPALREMGLQNVFTDDADLSGITSGNDLRVSDVKHKAVIEVNEDGTVAAAVTWLSLILRSSFRARPVTPVTIRVDHPFCFVIWDKFNQRAVFMGTVQKL

SEQ ID NO: 148 - protein, 376 aa, Haemaphysalis longicornis; identical to SEQ ID NO: 146

SEQ ID NO: 149 - protein, 207 aa, Haemaphysalis longicornis
MACATLKRPANDWATLASARSAKRPRRGAPPSSAKASRLGASRAAPKRTHTWHLDDPDGTCSKIRRCGQPPDTDQPSPFPDVPLKTSEEIAADVRDELRRLQRRGQLVGSDSDGCEGYYYPSEATTPPGFVGLAGSSASRKEQPLFTFEQVGLICERAMRERDGEIRAEYDRVLRDRLSEQYDAFVKFTHDQIRKRFEGGVAPSYLS

SEQ ID NO: 150 - protein, 282 aa, Haemaphysalis longicornis
MAPPCYADLGKQARDLFNKNYHFGVVKLDCKSTTKTGVEFNVSGTSLNDTGKVNASLETKYKVPEYGLTLKEKWNTDNTLATEICSEEKLARGLKVAFHANFAPQTGRKSGALRTAYKLDNLHFNGDIDLGPPGPVAHGAAVLHYQGWLAGGQVSFDTTKNRLSKTNFAVGFQGGDFVLHTNVNDGQEFQGTVFQRVNPNLQAGVQLAWTAGTNTTRFGLGCVYELDGETSVRAKVNNSGQIGLGFTHRLRPGISLTLSTMLDGKNFNQGGHKLGLGLDLSA

SEQ ID NO: 151 - protein, 1221 aa, Haemaphysalis longicornis
MVPHKMGRSHAGNSNGDIQSRDRFLRFSRTVTAAEPVPQTSDSVVYKVRATVSLKSPEFTVTEGAALQYAGDLALHKVAQDTYVARFLNFSVAKYEHLYGDLENLTAVPLNESVLLLDEVEPVDLSQQLSYPVKFELSSGKVVKYEVSKEVSTWALNVYKSLLTLLQSQVELPQEVPTVVNRFEDGVSGYCRVTYEVHSLTKNLFTEGRVFNVTKTKYLDDCKLTRPVHSVVGNLHKGYHAVCRKHLPDNFLPGFQEETSAFEARPFVGCPEGFSPTDSPVTAHEVSYYNVSESVLEGALSDSLTVLPLLTGEVVVRTRLQLQLATLYGPPTTPVETLGEPHTSLEQHLPEAKEILDLPVYSLLVSGPEEVKAEVFTQVLEQVTDELVSLELEVETKKTPALLLKLVHMVSLFNTEQLKQVMPQALHQKATELEPKDQVLRALYVDLLGKAGSKSAFEVAAYLVKENVLTRFEVTRLFRDLSVYKAYVDKDTLDLLLDLCKATEVALEPLLKVAVCETFGHAVHKACSSKVFVRPVPLKSNWRRIPKIKSRYEEDPELKVRKEDLVYEPVQRLVLPEEKRCTFADLDQYVQALEEALKTTKDFKLLVAYVNALGKMAKPEVLPVLVSYLNGTAENLYDFVDEEEEPTEAVYFVKKAVLLALHHVVEYFPKEVSPLVRAVLLNESEPIDVRTLAFDAWVKSVPTKWELQHVSIAVKLSRSLELKSYVYTAFTHLLKDKHPASSLLVSRLRGIFVHVEALNVGPRYSTFLQKTYYDAHKHLGLEFLLKHIANNVSFFPTFAHAAFKYNLGPLVQTVLETKVLLKGSEKLLDEVFSRNGGLLQKVTQALAGQATPYEKYERNYHETPTYTVVKELGIVTRKVESPKAVLFAKFLDGESVLPLDREVLEDLKKEVLELAVKVGKEGVLNKHYVRLVLPKKVAHVEPTVVGLPVVHTLLHPVLVSLKLKDVSVQATAQPLSVLPETLTVTGTVQPFLLSVKQSRVFVPVEVTEEAPSVLLTNVHHLHLKGLLSLEYDTKVQKKVKVSVKPSFGSVLLSAVCSEHVLDKHSPFVQLEKPLLDVNKCVETIEKPFVYHRTYGAKVFNLEVDAVSHGPYPPLPLYGKLYQYKSELVSNLVKYLTHMGTKQHVLRLKVLPTVEDPVTEYTATFKYSDNLDYLMKTTLDKVYVPYTQHDSSEEEYREQMLFSRKTERRRFRPSWPFKPNPK

SEQ ID NO: 152 - protein, 1731 aa, Haemaphysalis longicornis
MFWKCDGENDCGNHKDETGCGSAHNACRSDKFACHDSSYCVPKIWLCDGEADCHDSSDELDCHGTNCTGFRCQNHECIPQNWRCDSTEDCADASDELHCNVSASTPSPRCDIDQGRFPCLDGQCLLPEKVCDGKKDCTDGADEGTFCKVNECSQKKCSQGCFVSSNGSTCYCNSGFRLLADHVSCSDIDECAEQPHLCSHTCTNSPGSYKCSCLDGYQLADASFCRARDPEPLLLFSDSKQVRGIWLRSNRYFEVHPAEGQSVGVEFDSDQRRVFWTDVSTQKSSIHSCLLDGTGFKTLFTSEHTLLEDLSLDWVTSNLYVTDSFGKRILVCTLDGLSCSAIVKDKVDAPRAIVVHPENRTMYWTDWGSAPAVLRSNMDGTNVQAIVSTDLGWPNGLSLDSPANRLYWCDAKHSKLEYIDLATMKRTSVLNEALYHPFALAVFEDTIYWSDWSSFSLDSSHKLTGKQHHRILRENDKHIMGVHVYHPVLRPRVPSSLCFLCSCRSSINLPSCCLPTPLSCVMLTLSPYFNLVMLIAVTKDSSFAIIAEEDTIFKIDLDRVGAPYPVLLPVAGAVMIGALAFDWPNQTLYFSDNQKETISAVNVNNFELKILHDHLGSVFGMDFDVAHQLLYWVDADKFTLEACRANGSGHVIVLDDLRRPVDVALYPFAGILFVLTVAEKPEITSYTMDGRNPKTLPLSTLLRPVSLEVDLVARKLVWADAVQTTFCPQNGFPAVFFSTPFIVQQVKEHVSSMSAAHNEIHWTCRDNASLHYIDLSQQPPVHRYVPFRSVRNGTFSRRVLFAALVPPFEPGPCGVNNGGCSHTCLPVLATRRSCFCPPGMALNADNVTCRVESGTCRPHELPCAGVCIAAIYWCDGRQDCPDNADEKACDAATCPSDDFSCANGHCIGKAYHCDGYDDCGDHSDEKNCTRQTCTPNQFTCTSGGCTPFLWRCDGEKDCPAGEDELNCGNIRCPNGHDRCANGQCIPHDWYCDGHADCTDSSDERNCTEPSDCFPDDFHCKNGQCLDKRLRCDHDEDCEDSTDELGCDYGKSNKSKCVHGMIDCGDGHCIYAHDLCDGFADCHSGRDESNCTSAICHSAQFFCPYTKRCILQSWLCDGDDDCGDNMDELLPICHPTTPSPIATTDSACWSDEFRCGSKECIPWSRVCDMHLDCADYSDEGSHCETHCGTANGGCAHICRESPLGPQCSCHPGYRLNADSKACDDVDECGTPGHCSHFCQNSKGSYKCTCADGYSLAADHRSCKVQRTTLSPLLIFACSHALLLIHQLFDQYAGQTYGRWSPKLVSLASPRTSIFFLPFFQGTINVMTLGNGKHFTLLEQIYKPFHIAVDWVANNVYFTDGWVHIQACEPTFKHCADVVDTTYPHVNSFTLAANDGLMFWAVWMDVVRQPHGLIERANMDGTARTILLTDKILWPCSVTVDAVHKLIYWADANKNILESATYDGKNRNTVLGVGISSPFSIALFQDWLYWSDWGSDSIMACNKHTGADVTLVHHGTAKATVLKVFHAVHQPSGVNRCAHNPCGHICLLNPTSYICACAHGFTLAKDSHTCVESDDRSFNLSSSDVLAQPCNPVCLNGGRCLSENSSYFCKCSDDFQGPSCELPVVVAMPHVQSSSRSTALAAILLTVLCVALLVLGYILYRRHKNKLAALDFSVSFKKPAFGKREGLLDNEHPVAAADECATASPDSGFMNPAFGGRKAIRVIRVRLGSSPAR

SEQ ID NO: 153 - protein, 320 aa, Haemaphysalis longicornis
MVREDKTTWKSNYFLRLVQLLDEFPKCFIVGVDNVGSKQMQTIRVSLRKHAVLLMGKNTMIRKAIRGHLDNNPALERLLPHIKGNVGFVFTKEDLTEVREKIIENKVKAPARAGALAPLDVMIPAQNTGLGPEKTSFFQALQIPTKISKGTIEILNEIHLIKKDDRVGASEATLLNMLNISPFSYGLKILQVYDSGTVFAPDILDITPEDLRSAFVEGVRNVASVSLAIGYPTVASAPHSIVNGLKNLIAIALETDITFKEAEMAKEYMKDPSKFAAAAAAAPAAGGGAAGAKPAEAKKEEAKKEESEEEDDDMGFGLFD

SEQ ID NO: 154 - protein, 159 aa, Haemaphysalis longicornis
MALLGLPLLVLLSLSATMAFPDGAPELTCNPLFDMRPVHSETRPLNAKNSPYRLVQDKVDFAPKERVKVTLYTVGKPFRGFKVKSVDEQGQEVGRFIPGGGYKPLSECAAATHQSRAEKEHVEMHWLAPADRSGRVHFNATVVHRYYEFYMNLTSTLQQ

SEQ ID NO: 155 - protein, 228 aa, Haemaphysalis longicornis
MDSHSSMGLYGTLWRAFHLLLMLFFVFSFGGRVQEEKRKMEEKERKKAEVRKRLEEAAKAKKAGGKRGFMTPERKKKLRNLLRKKAAEELKKEQERKAEQRRKIIAERIGQPKPLDNCNEATLVGILKQYHARIAQLEDAKYDLEYEVRQKDFVVRPLVENGTRRTSLRFADDSTAPSHPQYNHRIENALGHILPRQYLTFENKTALKKLPATEMILTKGDFASNNEF

SEQ ID NO: 156 - protein, 380 aa, Haemaphysalis longicornis
MQSEIIIDKGYPVEEHEVETSDGFVLSVQRLPWGRKGRSNKRDDVRKKPVFLLHYLLGSSADWVINFPEQSLPYILADAEYDVWLGNVRGNTYSRHLRYSRHEKEFWDFSFDEMIAYDLPAMLDHVLNETGRERLLYVGHSQGTLILFGLLAERPEYNDKIELFAAMGPVTTVTYMRSPIRYLAPFVENAADLFALFGEYDFLPSSALMKLIAGTVCQFEESRSLCESVIFFICGPEGQQINKTRLPVLVSHTPAGTSVRNMVHFAQLVLSERFQKFDFGKTRNQILYGQETAPVYDLTRVTAPVALYWSEGDWLADPRDVSQLRDQLPRLVLDFRIHDPKFTHLDFTLGMGARRLVYEPLMKALSRARALLHYFHGAPA

SEQ ID NO: 157 - protein, 389 aa, Haemaphysalis longicornis
MGVAPAGLLYPIFLCFVAIASEAEKCDSSKCRPEDNCLCMSRQPPGNLSVEEMPQFVMLTFDDHVNAENMDFYRRLLAPGRRKNRANGCNVASTFFVSAVRTDFSFVHELHSAGNEIALHSITHRYNYTYWKTLDAAGWEAELFGEREILRDYALIPEQDMVGARAPFLEAGKGDSLFVMRQHGLLYDSSVTVNYMEEPDKLRVFPYTLDYGLQTLCVIEPCLEGTYKGLWTVPLNGYYQTFQDDTGSLVQSPCAMADSCEPQPLTKADTFAYLRSNFEFSYHYNKAPFPIFIHAVWLDEPERAKGLVAFVDWLLTMDDVFFVTVEEVVRFMQNPKPVKEYVQSKCSKATAFIRCSEIHTCEFAETQINGTRWLRSCRRCPKAIRGFAK

SEQ ID NO: 158 - protein, 615 aa, Haemaphysalis longicornis
MLPRECYLLAGRSAPLLGTSSDEVKMLPRLLLESAVLLALMAGSALATPIMGYSYAYRPRYADAHDYKVVCYYGSWAVYRPGDGKFPVENIDPFICTHIVYGFAGLGSDFKIRSLDSWNDLEDNYGKGAFKRFTALKKQNPKLKALIAIGGWNEGLGQPAARKIFADSVVDFCKKYNFDGLDLDWEYPAARGGKPEDKANFVTLLRELSQEFKPYGLLLTAAVSAGKHFMDPAYDVPQVSKYLDLINVMAYDFHGGWETRAGHHAPMYSRPEERPDERILNVNFSVNYWIEKGAPRNKLVLGMGLYGRSFTLRRAEDHMPGDEAPQKGQAGPYTREPGSLGYNEICEKFAREKWTVVKDPYFMAPYAFRDRQWVGYDDMESITAKVEFAKAMGLAGGMVWSIETDDFQGKCHGLKYPMLSTINQVFAIGGPGVIATPPPTQATPATVPTPSTEKTWWPRPSTPGKCADTPTTPSTPKESTPESDTSGTTPTSSTAKVWWPPSRSSTPSSTPSTTTSTTPTTTTSEAPSSSSMTPGGSGFECQSSGSFAHPSDCQRFYDCVAQGGKLVAFEKTCPPRTVFNPENKLCVWPESVPKCENYYENEAYEALPDKSQTSL

SEQ ID NO: 159 - protein, 1408 aa, Haemaphysalis longicornis
MRIVGLLLLAAAAAGFEVGKEYVYKHKGTLNVINPDHPQQLTGVAFRSKVLVQVKPDHTHFKILNFEADTYNAEHVDLGEHEFAYKSNENLVGALEHPFAGKYHEGKLEEVEIGKNEPMWVRNVKKGILSLFQLDLVKGRHEHPHSKEYHVKEDGLHGVCDTLYIVHEEEHDYMEVTKVKNLEKCEHRPYIFYGRVRGKPCIHCEAEDTHPYAESSQVYYELKGTPEHYVIQLAWAESDNQFKAFGEAKEHHIVLNRTLELEAEHDAPTVETALPEDAAKEHSLLQEFATSEHLQNPEELKHANPLVAQFGLAPAKEIFVQGLDKLARLEYTDDDIKEIDQKPSGGLSFLMLFQSMMTLSYDDLNDIYRNHVVTAPEDIKDSMSHLFMDLLAATGLNPHVTLGIDLIKNNELHVLDAERFFMKLHLNLKEVSPAMIKEIRSGVKSARAVWTACKLAASAITSGADCHRSHNDTDEDHGTCAPELVSHLFNYTVTPHDVHDEPEYETTVYLRMAGNLATRKSMHYLGRFICPGQHASEPKRMAALWALKQAAKYHKELARSIALPVFFNETEPSEIRIAAFLVVVVTDPDLYLLRHIALEMITEPSDQVTHFVASAFHALAKSQYPCHWTIAQHLKYVLPLWQNTPKLNKRVDYASSHMQIDSGYNESPEQTAQKLLGTARNKKVFYMTQDMTFLAPTELGVPVFFDYKQAEFMYANIQQVEITHGDMAEINLNLKRHYLYEVRSHKFIGTALTFDKSSLGSGIDQRTVVSWPLELKATLSILEGKLKLHRPMTMPYNVGNHYAHPFTFKMPYDLTKDLADPITAFALATKPLYHTEDLREFDRSYFGELLGMTLNVKGHFIDEGLEDGLREFFHEMTWRERIYYLALNPHWHPRNVKAYLLPAAEDATKAVDVDITYKLLEPEDPRESHFPIHDQFPGDNEVPQTHVLNLDVHLRGDTKDRKAESEFRYSFSHDLFKHKMQFFYDRSQFSKDPHSALKICLDASAVFPKPDWSRMKDMATFYQGKHVDAKLDMHYGDTCESGSTITINGQYTHTDDDERQLTNAAAGKPLRYTHAKPNGLQKFYAYCQRDRASGVFYNYWCLKFLRHSSRLAKLTADVEYKNYKPLLKCLLPSYGSELARKTKLRGFFGAIRSHFHGENGKLHVVSQVPWWTRDEKPHADIVVTTEDGHKHHHWNVPIFSHMLEPRVFSSLGYTNFGEYSKNYRHRHCDLQALSVRTFDGSVVQLPETDCYKVVSRDCSPNKRFLLLARATGNPSFAKAVKLFIGTTKVEILPVTDASAPIVRVDGTKVDVTPERPYSHTSHDAELFEVRTENKWFELVSKPYGISLDFNGNVLFVQTAPFYRGKLCGLCGDYNLDRSTDLSGPDGHLYNNTLEFASSYVVHSPDCHA

SEQ ID NO: 160 - protein, 453 aa, Haemaphysalis longicornis
MHLSMKRPDDLAEAKNQLADAIRRLHEYEMEIKRLENERDELAAAYKEAETLRKQEEAKCQRLTAELAQVRHEYERRLQAKDEEIEALRKQYQLEVEQLNMRLAEAEAKLKTEIARLKKKYQAQITELEMSLDAANKQNLDLQKIIKKQAVQITELQAHYDEIHRQLQQCADQLAISQRRCQGLQAELDEQRVALESALRSKRAAEQSLEESQVRVNELTTINVNIAAAKNKLESELSALQADYDELHKELRVVDERCQRTIVELKSTKDILVEEQERYIKVESIKKSLEIEVRNLQVRLEEVEANALAGGKRVIAKLEARIRDVEIEMEEEKKRHAETQKMLRKKDHRAKELLLQTEEDHKTITMLNDAVEKLNEKVKVYKRQLNEQEGLSQQNLTRVRRFQRELEAAEDRADSAESNLSLIRAKHRSWVTTSQVPGGTRQVFVTEETSQNF

SEQ ID NO: 161 - protein, 176 aa, Haemaphysalis longicornis
MAPRNWKRPLTLIYNDNYKYGNSLYSGALDDIEKRYQASLSRTTFRSDRGDLGLSTFSSSNIGSAPEPSSGRAATATNESDHTLLSPSFSAPSRSPVRSMLDTEHDLRKARSSGDFGRLRSALRPSSWSEEDDRALLDDQTFPRRRRHRRQDRSDYLMTSPTVAAGESVEYYFDKW

SEQ ID NO: 162 - protein, 283 aa, Dermacentor variabilis
MKIENLLVRQLINEFIGTLVLVLIGDSVLAVIIAGENEPIAAVVGPLGWGVAIYVAVQIAGGVTAHLNPAVTLALASVRKFPIAKVPLYFAAQYLGAFVGAALVFVTYKDCIEHYDQGVRAVVGKKATAAIFATYPREHVSTLTCFIDQVISTGILMLTAEAITDPRNFGGIPVSIHPICLGLMIMALIFSFAYNCMCPLNPARDISPRLFTLMAGWGPETFTLRGWNYVWVPVVGPHIGAILGAWLYKVAIADHWPDIKPKPTTSTEHLVASPSPGQLAKEA

SEQ ID NO: 163 - protein, 293 aa, Dermacentor variabilis
MKPNAVTRAWRRVCGCCIERPLARQALAEMVGTLVLTLVGDCVLASLAVFQLGSTGLAAGPLGWGLAVFLGVLVAGGVSGAHLNPAVTVAMATVGKLAWCKVLAYVAAQYVGAFLASCLVYVVYTDALSQIDANLTTVYGINATAPVFSCFPAPGVSTLTCLVDQIVSTAVLLVGICAITDTRNMDVSRGQQPLLVGLTVSACMYAFSYNCGNPLNPARDLAPRIFTAMSGWGSAVFSFRAYNWFWVPVVGPHLGAVIGVWIYKLAVDNHWRADDEIDAAERKPLLGSPKLVA

SEQ ID NO: 164 - protein, 2881 aa, Dermacentor variabilis
MRSLTLFVAAISLIGGCASKDITHDGLQDPPAPPTPDVCSHFGNKFCGTEKCEVVPDKTDAFVCRCQRNDTYYDAAEGTCLFKRTCKTTECTYGACIENGVNNARCGCLKVESLSVNCKIRDWFIDECKRKGATAVLNKEWSFGAKCDCGEWAAMDRSKKKCVPTTCLRPDLTCKELCEKNWLEKDNRCCQGWSSADCSAAPPSNTYCSPGSIMAADGKCQDACTAKEAKLVCPDGCRKSESSAGAYECRCKYNEVVAEDGITCKAAWTAQACSEEQKKTCLPSQTCLVEKDKIICACPRNHQLVNGLCTSRCTENKCHENFTDCDVYMGKQSCSCPWTTRKPGQIYIRECTLKEYYYTVSFTPNISLDANNCNLYEDRVLEAMKTSIGTEVFKVEILNCTQKIKARLIAAKPLSKYLLKKLQTCEHPDGDLCMLYPKLPIKKHTATEIEEENLCDSLLKGQEEAYNGQNECVKFEDFFWFKCAAGFREVDRVTRGRLRRSVCEPGVSCTQDKELECASKGQICVYENEEPKCQCPPGMVTGQGGCSARTTCNSKERRECEDKKRECVYKDQKAECKCPQGTVDDGHGCSDVCSHFGNKFCGTEKCEVVPDKTDAFVCRCQRNDMYYDATEGTCLFKRTCKTTECTYGVCIEKGVNNARCGCPKVESLSVNCKIRDWFIDECKRKGATAVLNKEWSFGAKCDCGEWAAMDRSKKKCVPTTCLRPDLTCKELCEKNWLEKDNRCCQGWSSADCSAAPPSNTYCSPGSIMAADGKCQDACTANEAKLVCPDGCRKSESSARAYECRCKYNEVVAEDGITCKAAWTAQACSEEQKKTCLPSQTCLVEKDKIICACPRNHQLVNGLCTSRCTENKCHENFTDCDVYMGKQSCSCPWTTRKPGQIYIRECTLKEYYYTVSFTPNISLDANNCNLYEDRVLEAMKTSIGTEVFKVEILNCTQKIKARLIAAKPLSKYLLKKLQTCEHPDGDLCMLYPKLPIKKDTATEIEEENLCDSLLKGQKEAYNGQNECVKFEDFFWFKCAAGFREVDRVTRGRLRRSVCEPGVSCTPDKELECASKGQICVYENEEPKCQCPPGMVTGQGGCSARTTCNSKERRECEDKKRECVYKDQKAECKCPQGTVDDGHGCSDVCSHFGNKFCGTEKCEVVPDKTDAFVCRCQRNDTYYDATEGTCLFKRTCKTTECTYGACIENGVNNARCGCPKVESLSVNCKIRGWFIDECKRKGATAVLNKEWSFGAKCDCGEWAAMDRSKKKCVPTTCLRPDLTCKDLCEKNWLEKDNRCCQGWSSANCSAAPPSNTYCSPGSIMAADGKCQDACSAREAKLVCPDGCRESESSARAYECRCKYNEVVAEDGITCKAAWTAQACSEEQKKTCLPSQTCLVEKDKIICACPRNHQLVNGLCTSRCTENKCHEDFMDCDVYMEKQGCSCPWTTRKTGQIFIMECTLKEYYYTVSFTPNISLDANNCNLYEDRVLEAMKTSIGTEVFKVEILNCTQKIKARLIAAKPLSKYLLKKLQTCEHPDGDLCMLYPKLPIKKDTATEIEEENLCDSLLKGQKEAYNGQNECVKFEDFFWFKCAAGFREVDRVTRGRLRRSVCEPGVSCTPDKELECASKGQICVYENEEPKCQCPPGMVTGQGGCSARTTCNSKERRECEDKKRECVYKDQKAECKCPQGTVDDGHGCSDVCSHFGNKFCGTEKCEVVPDKTDAFVCRCQRNDTYYDAAEGTCLFKRTCKTTECTYGACIENGVNNARCGCLKVESLSVNCKIRDWFIDECKRKGATAVLNKEWSFGAKCDCGEWAAMDRSKKKCVPTTCLRPDLTCKELCEKNWLEKDNRCCQGWSSADCSAAPPSNTYCSPGSIMAADGKCQDACTAKEAKLVCPDGCRKSESSARAYECRCKYNEVVAEDGITCKAAWTAQACSEEQKKTCLPSQTCLVEKDKIICACPRNHQLVNGLCTSRCTENKCHENFTDCDVYMGKQSCSCPWTTRKPGQIYIRECTLNEYYYTVSFTPNISLDANNCNLYERRVLEAMKTSIGTEVFKVEILNCTQKIKARLIAAKPLSKYLLKKLQTCEHPDGDLCMLYPKLPIKKHTATEIEEENLCDSLLKGQEEAYNGQNECVKFEDFFWFKCAAGFREVDRVTRGRLRRSVCEPGVSCTQDKELECASKGQICVYENEEPKCQCPPGMVTGQGGCSARTTCNSKERRECEDKKRECVYKDQKAECKCPQGTVDDGHGCSDVCSHFGNKFCGTEKCEVVPDKTDAFVCRCQRNDTYYDATEGTCLFKRTCKTTECTYGACIENGVNNARCGCPKVESLSVNCKIRGWFIDECKRKGATAVLNKEWSFGAKCDCGEWAAMDRSKKKCVPTTCLRPDLTCKDLCEKNWLEKDNRCCQGWSSANCSAAPPSNTYCSPGSIMAADGKCQDACSAREAKLVCPDGCRESESSARAYECRCKYNEVVAEDGITCKAAWTAQACSEEQKKTCLPSQTCLVEKDKIICACPRNHQLVNGLCTSRCTENKCHEDFMDCDVYMEKQGCSCPWTTRKTGQIFIRDCTLNEYYYTVSFTPNISLDANNCNLYERRVLEAMKTSIGTEVFKVEILNCTQKIKARLIAAKPLSKYLLKKLQTCEHPDGDLCMLYPKLPIKKHTATEIEEENLCESLLKGQKEAYNGQNECVKFEDFFWFKCAAGFREVDRVTRGRLRRSVCEPRTTCNSKERRECEDKQRECVYKDQKAECKCPQGTVDDGHGCSAAVVPESCNEEETNKCRSSGQRCVMENQKAVCKEASNTPATEVVDPAPSQCDEEMRKKCTKKGAECVTVDGKAICKCPEGKVDTSQGCSDPGPSSAVSVSSTTLLLLAAMTAAAAAAA

SEQ ID NO: 165 - protein, 437 aa, Dermacentor variabilis
MKLLALPLALLLTCAFGMPQQDGSGTGISTASTAAVSSSTPASANATTSTTTRSGARSGGPAPIVCYYYGWANTRPHPANYGVNNIPGDLCTHVNFAYAGIDPETWELRSEVPEYESNRELFKNFTAIKTKYPQLKTLLSVGGWQQEHGVFSALVSKADRRALFIESVLLWMKEYNLDGVDMAWRFPGVSYRGGSPRDKENYASLIRELAGAFEGKGLLLTVVMPVTDEFLNPGYNIPEISKYVDWINAQAYDLRGAWNGVTDVHTPLYSRSIDVGPQVTLNVKDGLARIVSGGAPKSKVVMGIAFFGRGFTLLDPQQHGLHALINRDVPPHAGPFVRSNEVFAYYEICLNLKGNWKREFDDEGKCPYVYYRDQWIGYEDAVSIQHKIDFLQQEGYRGVYVFNNDLDDFRGFCGEPNILLKTIKNGLSEKNEIPVSA

SEQ ID NO: 166 - protein, 259 aa, Dermacentor variabilis
MMFHPFHLVEKSPWPFSSSIAALALTLGFISYFHNLDFYLLMMAYIMIITSAFQWWRDISREASFQGFHTKWVLKGLKLGMILFILSEIFFFISFFWSFFHSSLSPNIEIGGLWPPKNIYPFNPFEIPLLNSTILISSGITVTWSHHAIMNKNFISALTSLKITISLGILFTMFQIFEYFQAPFSISDSIFGSTFFMTTGFHGFHVLIGSIFLIVSYYRVKNQLISSNHFFGFEASAWYWHFVDVVWLFLFTFMYWWVY

SEQ ID NO: 167 - protein, 216 aa, Dermacentor variabilis
MPVVLYNLVGSPPCGFIRCLAKHIGVELNLNNLDFAKGEHRTEQFLKVNPFHKVPAIDDDGFIVYESNAIAYYLLRKYSPESELYPACIETRTRIDQVLAAASSNIHPQLGAFFRPRYFKSTKPSAEEVKAFEENVVKNLENLIGDSKFAVGDKLTAADLCLIGHVTVCLEFPCFDKAKYPKLTAYYELVRNTLPYYQEIFGPVTAQTKQLWDRLK

SEQ ID NO: 168 - protein, 540 aa, Dermacentor variabilis
MIRIIIVSVLVISSKGIQPPRLVYPRLLEERSADGRMVLHLHDQLTLNLRKASVAARNFRVLEHEDGHEVTHFFNGEDIEQNLHEDEKQFATVHVTRRDSGVEVEGVVGPYHRIEPMPEMERSDQGLIPHMIHKIEKKEMLDIEVAPSGKDAWIVNERTYGAESVPMEVTIEIFMVSDYPHFKYFKTTLQLINYLCVQINSVNMRYIDTSEPRVKFLFVGVEKDQFSTYKNGSGILMESSSSLDEFRVYADSRRYEYGYPDMVYLITGHEVYSEDTAGTKSMTVLGIGFVGGLCTQFFVALGEDSAGLYTGMHTLTHEAAHVLGAAHDESQPKSWIPGDPGSLACLWSEGHIMSYVDGGTKYHRFSTCSLRQIRNLVILRGSACWRVSNTGHTNKGRYPGMEVAPNQFCKSVFPDKDNVTADMDSDRMKYCMVKCQYPIFKQYCSNKYGRQRCYTHTTIKYLYKHALDYMPCAVDKVCVRGVCGGSTLTVPQAPATESTTPEATKTPTTDGTTECRCDCDSTTPTISTAYPPRPTTPSKG

SEQ ID NO: 169 - protein, 379 aa, Dermacentor variabilis
MAVNMLGDSVLNFAVDLYHQLSPKGAHKGNVFFSPFSISAALSMALGGARNRTAKEMSAVLRVDGEQMHNHFSDFLSKLPSYAADVKLHVANRMYCEQTFPVLESYLALLRDSYGATIESVDFRNDYENIRRQVNAWVERATESKIRDLLPGGSVHSFTTLILVNAVYFKGFWESQFNPDATRRSDFHLDSKNKKLVDMMYHEGRYSMASSEELGVTALEIPYRGGKTSMVVLLPNDVEGLSNLEELLTAPKLAKLLNNLGGFVNVELYLPKFKLEQTVGLKETLQEMGINDFFSSEADLSGISEKENLSASDVVHKAFVEVNEEGTEAAAATAVVMVACCMSSMPLQTYKFVVDRPFMFLIRSCDPDLVLFMGSVRDM

SEQ ID NO: 170 - protein, 402 aa, Dermacentor variabilis
MMASVVAAVLLCGAFLAPTCAQDAELRHARANNGFGVALFKELCSKTVDQNVFFSPASVSIALGMLYAGAGGGTLEELSSVLGLANAGLVDRDAVLTAYKSLLETKPANATLDIANTVLIQKDFEILDQYRNDVVKYFQAEVRSVDFVRDGSRATAEINEWVKAKTEGKIPKLLDEALPMNTAAFLINAVYFKGTWVTKFQARDTKPLPFYNHGRDEVKVATMSIRRRFGYAVVDELEARALEVPYAGDRFSMIIVLPKSRTGLPTVEARLTAGLVDKIATRLTRGDVQLWLPKFELRTDYDLVKPLRRLGLESAFGGRADFSGISGRNDLAVSDVKHKAMVEVGEEGTVAAAVTSVSLRWKTTSVRRLPPIALRVEHPFAFFIWDKVSMQALFMGAVRSLN

SEQ ID NO: 171 - protein, 379 aa, Dermacentor variabilis; identical to SEQ ID NO: 169

SEQ ID NO: 172 - protein, 165 aa, Dermacentor variabilis
MACATLKRTHDWDPLHSPNGRSPKRRRCMPLSVSPPAPPTRAHQINPSPFGEVPPKLTSEEIAANIREEMRRLQRRKQLCFQGSDPESQPTSGLLSPVRRDQPLFTFRQVGLICERMMKERESQIREEYDHVLSTKLAEQYDTFVKFTYDQIQKRYEGATPSYLS

SEQ ID NO: 173 - protein, 282 aa, Dermacentor variabilis
MAPPCYADLGKQARDLFNKNYHFGVVKLDCRSTTQTGVEFNVSGTSLNDTGKVNASLETKYKVPEYGLTLKEKWNTDNTLSTEICTEEKLARGVKVAFHANFAPQTGKKSGALRTAYKLENVHLNGDVDLGPPGPVVHGAAVLHYQGWLAGGQVSFDTTKNRLSKTNFAVGFQTGDFVLHTSVNDGQEFTGSVFQRVNPQLQTGVQLAWTAGTNATRFGLGCVYELDQETSVRAKVNNSGQIGLGFTHRLRPGISLTLSTMLDGKNFNQGGHKLGLGLDLSA

SEQ ID NO: 174 - protein, 1843 aa, Dermacentor variabilis
MRVLCGLPLLLLAAAANGVLFDPVPPTQSSGGVVYKVRATVTLKSPELTVTEGAGLQYAGDLALYPAGQDTYEARFLNFSVVKHEHLYGGLDNLTAVPLNVSVPLLNETFAVDYSQHFRYPVKFVLSAGKVVKYEVGPEVSTLAHNVYKSLLTLFQNQVETPQEVPTSAAYYEDGVSGHCRVTYEVHSLTKNLYTEGPVYNVTKTKYLDDCKQTRPVHTTRGPAHKGYYAVCSKHLPNNYLPGYQEDTSAYESKPLAGCPEGLGPYDYVVAAHEVSYYNVSGSLLESALTDSLTVLPLLSGSVVVRTRLQLELASLEVPPTTPVQYQGEPHTSLELHLPEAAEYLDLPVYSYLVGSSEQVKPEVFTQVLDQVAEELVSLELETEAKKTPALMLKLVHMVSLLNTDQLKQALPQSLLTKTSELEPKEQVLRALYVDLLGQAGSKSAAEVAVHLVKEEVLSLYEATRLFRQLSVFSAYVDKETVELLLELCKTTETVLKPLVKVAVCDALGEAVKKACPTGVHYDTVSTTSYKSSWRRIPKIKSRYEQTPSVPKHLLPFVPVERLVLPLEKQCTVADLVEYVKVLEEALKTTTDFKVLVGYLNALGKVAKPEVLPLLVSYLNGTAENLYQLVEEGEEYYETVYFVKKAVLLALSHVVEYFPKEVSPLVRTVLLNVSEPVDVRTLAFDVWLKSVPTKWDLQQVSLVVKLDRSLELKSYVYTALKSVLKDKHPANHVLASRVRGVFVHYEALNVGPRYSTYAKKTYYDVARNLGFESVVKHVANNISYFPTYLHAGLKYNLGPYVKTLLEGKLLLKGGEKFLNEVFSTEGTTTFLRRVTEAFTRKVKSPYNTTPYETSTKYSVVKELNLKTRKVESPKAVLFAKFLGGDAVLPLDKELFEQLKNELVELAQKASSTGPLTTHLVRVLLPKKVAHVEPTVVGLPVVQTVLHPIVVSLKLKDVSLQVDSTVDSLLPVTLNVTGTVQPLVLSVKQHRVFVPAELTEESPSVVHTSVKHLHFKTHLSLGFNKYEKKVKVTVKPTFGPALLSAVSTEHVVETPSPFLLLEKHAVDPKKVMDTIVKPFVYHRVYETKVVNLEVDAVSHGPFAPLPLYGSLYHLSSERMPKLLQYLSHQASKQHLLRINVKPYEKDPVSEWVATFKYENNLDYLMKTTLKKVYTAARTTTYDENQETPYDYMLSEHVTPRPTTYAAYEEEDSYEYYPEYPYLLNKTLVKHVLNVTLEGKLEGAVKQLTKLDLAYHHSLNKTLKHYYVDVKTLTKPLVSLFLNVSSPVPPSPFWYVPSYLGNDLMNATLYLTYGDEPEPFVVTYNATKTEEQLLGLHAYDATPLLHWFVPQCLADQHAGHTVSYACSLATVFDAHLNQQVVAFKVPAQVSPKVKNLALKVLSFLKFKLFPYASFYVLPFVQKEEYEVVFRLNKTDVNPYVSVGYGELVLPGEKVVLNDLKLSKYSVPNLLLSVYDRLKHGLFKSYPHQPCSVGKHWVRTYDNVSFPLEVRPHCKYLVTSDCSAKHDFAVVALPLDLAVGTKKLIVQLGPTVVELPPPDLYKAEVLLTVNGTYYVANTTQDVVLPYKWDRKLFVTVYPTSGPHDPPVVELTTSLKTFKLLFDGVNFFVWVNPLYQGKTCGLCSNYDNEPYHEFVTPENYLVSNYSEFVASYGFGLPQCKEPLVPVYPLHYLDELKKPVGYEAGYPSHPEYPSHSKYPSHHKYPSHKEYPTHHKYPSHEEYPTHHKYPSHEEYPTHHKYPSHEEYPTHHKYPSHEEYPTHHKYPSHEEYPTHHKYPTHKKYPTHKYPTHHEYPTHHKYPTHHKYPSHHEYPPSREHYPYSPSLQREG

SEQ ID NO: 175 - protein, 1797 aa, Dermacentor variabilis
MKSVACVVLAAVALYLVGDAACECQQGWFDCGNDRCITMFWRCDGQNDCGNHKDETGCSDHAHRCPSDKFACRDGSYCVPEIWVCDGEADCHDSSDELDCHSSNCTGYRCHNNECIPNHWHCDETEDCADASDELNCHAATNSSTTTVAPRCDVDQGRFPCLDGQCLLPSKVCDGRKDCGDGADEGAFCKVNECSQKKCSQGCFVATNGSTCYCNTGFRLLADHISCADVDECAEEPYVCSHGCTNSPGSYSCHCLEGYQLADKSFCKARDPEPLLLYSNTKEIRGLWLRSNRYFEIHPAEAQAVGVEFDSDQRRVYWTDVSTLKSSVYSCRLDGSGLKTLFSAEKTLLEDLSLDWVTNNLYITDSLVKRILVCTTDGLSCSALITDSIDSPRAIIVNPAQKTMYWTDWGAQPAVMHSNMDGTNVAQLVSTDLGWPNGLTLDHTTNRLYWCDAKLSRLEYLELSTLRRAVVMAEDVFHPFALAIFEDTVYWSDWASYSLDSSNKRTGKHHHRVLRENGNHIMGVHVYHPVLRLQGIHNPCWANPCHHICVLSGNSYTCLCRIGYKLLADKRSCTVTKDFRFVVVAEEDLLYKIDLDRVGPPLPVALPFSNLGMISALAFDWSNQTLHYSDNRHEILSAINVNTFNQWTVHDHIGSVFGMDFDVAHQLLYWVDADKYTLEVCRANGSGHAIIRDDLRRPVGLALYPFAGILFVLSAGDAPTITSYTMDGQSPQILPLATLLLPVSLSVDLVARKLVWADATRGTIESLDLRNLFSATPFIVQQVKAHVSSVSAAHNEIHWISRDHASLEYIDLSVEPNLRHVPLPSKRNGTYSRRVIIASQVPPFAPGPCGQNNGGCSHTCLPVRTTDRSCFCPPGMALNADATTCRVENGTCRPHELPCAGRCIAATYWCDGHKDCSDNADEASCGPATCPSTDFTCSNGRCIENEWRCDGYNDCGDLSDEKNCTRQTCATHQYTCRSGVCVPLYWRCDGSEDCPDGDDELNCSGVRCPSGHDRCANGQCIPHDWTCDGHADCTDSSDEKNCTEPLTCLVDDFRCTNGQCLDKRLRCDHDNDCEDSSDEVGCDYAKVNRSKCSTGMVDCGDGHCIYAHDMCDGYVDCHNGRDERNCSAPICQSAEFFCTGTKRCILQSWLCDGDDDCGDGMDELLPRCHPTTQVATTTVAACWGNEFQCGSHECIAWTSVCDGRTDCADFSDEGSHCVKHCGTANGGCAHICRESPSGPICSCQPGYRLNSDHKSCDDVDECATPGHCSHFCQNSKGGFKCTCADGYALGVDRRYCKVQYGEAFLLYMLPNQIRSFSMHGHAQHLLAEDSLSDMHGMDYRVADKSIFWTEMDEGIINVMTVGNGKQFTLLEDIHKPHHIAVDWVAGNIYFTDGWVHIQACEPTFKHCTDVLDTTYSHLNTFALSANDGLMFWAVWHEVVNKDHGLIERSNMDGTTRVVLLTDKILWPCSITVDAVHKRIYWSDANKNVIESATYDGKQRKLVRGAGLSSPFSIALFEDWLYWSDWGSDSLMACNKYTGSNIGLVHHGTAKATVLKVLHAVHQPSGVNRCARNQCAHVCLLNPSAYTCACSHGYSLAHDAHTCIESDEHYNLTSSDVLGQFCNPVCLNGGRCISANGSYFCKCLNGFNGPSCKDAMVISTLPQQSSSHSTALASILVSTLCVALLVLGYVLYRRNRQKLAALDFSISFKKPTFRKRQGLLEDEHPVAADEDYHAMTPAPGFVNPAFSNRKSQLLTDDGQFKRWSSSESLQSSSSKDKSTCSLATDSAAKNEVFFFRKH

SEQ ID NO: 176 - protein, 2188 aa, Dermacentor variabilis
MADGAEQCQDSPVEKQEADDDGQSGLLSCRLFAVIAGAVFMALVVLFGVPKDEQRRGLSTAARHDHPAMHNVHQPGAWSNFSGSFSLADGRNAADRQNKTPGTQKGQPTEPRPLLCVVSRSLNNPIQFPRKHCTHIVYKDVAFNPTTGKFTPVNSATFVALRMLVEVTRQRALMAVANLEQHRAPDDARRLAAALVKWTPASGFHGVAALGIDTASDKLPDLGPLFKELHNALKPEKLEFLAAVHIRDWDIPSGLVVGRLAAIAPHLDYLVLETHFGNRYNSCHIAYSSVFYSTGAQPSPSVPIGQALSWMTSLLMEQRQYVTTCFSINMGAVVFRDATEMRGSCSGSDVVSYMQVCKGGEWRPSTVANLDALSVLRLGHGRVESHEDDPLLSSKVSHALANYPRACVVAYDVDLDDYEGQCDAEPFARLRNLRRAEVRNDPQMAAQSLAKSPNDELKKRCMPPGSGNKRPLVCVLSDRIDVDRNVSKLFCTHIVLSLRRYGRLDALKDITKEYVAQLRGAAPPGVCVLVAPHERALEQSHEGEAVRLAEATAVQLKGKQLDGLAFLHVARTSAALVELGPALEVLHETYKAANLCLALDLQILDPNTPAEEVGKSLSLVAKHVDLLVVNTHYPGRMGPCRAVPLASAVQPARSCIPTVATACTGGSWTQVHNNSRDSPMWQDGYNLQTYETASSLTQRVRSLLPLPNAGVAVFNFDYEDYAGVCNGGVPQARLMALRAALDEENITAQTTLTLETPDRPLVCVLSATVRNESRVPSSHCTHLVHEGARYSVTEEQLVIPENTVSLAANRSKQRHLVGLNGNEMVELLMTANDRDRHEIAARMASAVLAAKMQGLAILNFNRTSKTIESFGSVLEVIRSVFAKELTIIFGAEVLDFSLPAEIMGQRLTKAAKHVDIFILQTHYRRPRGYCRISYPSTYRGDTPLVTLSAATAWARAMETTNRVCVSYTLGVLQFQKGNAMGPVACDNVTLLDRADACDEKDWEVDANNTKTLSVVRKKDKFIQTFESPEFLAEKVSRTFSENPGSCVALFNVDLEDATGNCGSTHARKFARLAVVAEKYIELDSRTKSKHQLLPMLTKVVPNDTAANLAGYLPHFAPERLVGFKARTTVYRPDILGMPEKRPFVCLFSPSWTNASVSPVPSRCTHVVLSAESLDAPNLTATTSDRGAALERLSRGLGRGVKLYVRLQGWRRDIAAKAQAVQDAMRLSYAQGVALTDIRIASTEAAQLALGIQALMEQMPKDMSLILGLEMSDYDEAGQLLSQRLAPLYEYADVVVLQTHLRTGHSWPFCRTTYASLFDHPSDDSCLQTPPAVTALSWMKLLQPFSVPTCLSLNMAAVKFRASKNATPEASCEDALETNDVCTTQEWPSAAESKSKLAAYRYKDGFSVTMETAELLREKLSRAHRLYPRFCVALYNVDVDAEPCEGSDLSFARVAQIADAAQYKHRKPTTHEPRGRDSGGSQDDDSGPRTAVQNATSSRALVCILSASALNMETFPMRLCSYVVYTRRVTPGAPPQPFVPQNDPKFSKFMRRCVETESNGLVAVTPSMLATLFTNNSTLVADALNGVMRRLADSKIAGLAVFATPSADLQALASMTQEAFKLLKDKFKMVVGVHVMKFDAPVLKELSKSSHLLVLFTHSMLRGSNCTVTAPSSLPLTPSHYELMKQISTLEGGRQPSTCISVNFAVMGFKTRSSNASLGDGCVNRRLESYDHTCWAAGDLWQPTSLLVHRHNSSWVQTFETERSVGSAVRPFLEAYPQGCAAAVYADFEDASGTCRPRYSRLRALSALMTAKAADTSPAANSLPTEAESVTGSLPTAPPEDMISTHITKRNKMVCFVTPSSHIMTDLPADSCDYLVLSTQNFTAVENVTALFGRWKELMTPLSRKTRLALGMNAHTLVSLYKGDYAGGVLLVESIPSWLKPNGGSALALLTGSLASQDELLLFRILKDLRSALNQAGNPAPEVILAVSSDSELFHKELIELSDVFVLVDHHIPREAGSSSCKVSFPSVSPRTSSLTSPMLIAKDLMHRGTENKTYCISINLAVLKFGTSADNLADGSHCDSEDEVAYSEVCQRNNAIPVYDMNWMTASLRDSKKIYTFEKAEMLLSKVLFLKAWFPSLCVAAFHAELDAVDREACPQSEQYARLLSVRRGLDSSVHGESHWRRNAADGA

SEQ ID NO: 177 - protein, 318 aa, Dermacentor variabilis
MVREDKTTWKSNYFLRLVQLLDEYPKCFIVGVDNVGSKQMQTIRVSLRKHAVLLMGKNTMIRKAIRGHLDNNPALEKLLPHIKGNVGFVFTKEDLTEVREKIIDNKVKAPARAGALAPLDVMIPAQNTGLGPEKTSFFQALQIPTKISKGTIEILNEIHLIKKDDRVGASEATLLNMLNISPFSYGLKILQVYDSGTVFSPDILDITPEDLRSAFVEGVRNVAAVSLSIGYPTVASVPHSIVNGLKNLIAIAVETDITFKEAEMAKEYLKDPSKFVAAAAPAAGGGAAAAKPEASKKEEAKKEESEEEDDDMGFGLFD

SEQ ID NO: 178 - protein, 421 aa, Dermacentor variabilis
MRLLGTIKLVGKAPSPGSAELHALRRKLHAMNFLICLAGLLALCAAQEEHKVTGANNQFGFKLLDKIPVSPQVNLFYSPYSVSTAMAMAYVGARGETQHDLHETLGYSSVGLTPDHVPSAHAQHTHLLRAPSNSTLRVANAAVVQDGYVVTEEYLGLLRTSFGAEVNRAGLNDEQSVRGINDWVKNKTEGKIEQLLSEPLPPNTRLVLLNAIYFKGLWNTPFEVRGTSKAQFFNAGTERVEVDTMHAQLNAGLAEDDETNADVLDLPYAGLDYSMTIVLPRVRNGVDALRQNLSWPVFQRLLSKLNAHAVVEVALPKFKIEGSYKLKAPLSALGASKAFDERLADFSGISGSRDLVVYEVVHKAVVEVNEEGSEAAAATGVITYTKSATFGIPFVVDHPFLFFIRNRQTGDVLFAGQVNHL

SEQ ID NO: 179 - protein, 192 aa, Dermacentor variabilis
MLRLALLIALASAAWAGNNLNEQVNQNRYFLHDRCRIGLQEQVNLELHASLVYMQMAAHLANNKVARNGFARFFRDQSSEEREHAQKLVDYVNLRGGTVSAVSVDMPATATWMSVLDALQAALALEHRVTNRLHELHRLADDSQDPQMADFLEQEFLAEQVRSIDQLQRLITQLQNMDTGLGEFLLDQQLRA

SEQ ID NO: 180 - protein, 106 aa, Dermacentor variabilis
MPLLAALRSKRTSMKAFVVALLISFVALALGVDEETPVTIEWPPRPRTCAEGETWKQCVSGSCAEATCARPRVGPQCTSDCRYGCYCADGFYRNEQKVCVTRDKCP

SEQ ID NO: 181 - protein, 156 aa, Dermacentor variabilis
MAFVRQAVLLVALVAACWAHPDGAPESACATMIPEHKDAVGEVAGPATPFKLVQDKRDFKAGDVVAVTLSSSGTPFKGFFVKAFDEKNQEIGQFEASPEAKAVTKCSGVTHTGNAPKTAVKVLWKAPAGATGKVHFRATVVIDYHKHVSGLQSTVA

SEQ ID NO: 182 - protein, 219 aa, Dermacentor variabilis
MGDTDADRRRKDAEDAHRRLKEEEKRKMEEKERKKAEVRKRLEEAAKAKKAGGKRGFMTPERKKKLRNLLRKKAAEELKKEQERKAGERRKIIAERVGQPKPVDGANEATLQGILKQYHARIAALEDAKYDLEYEVRQKDFVINELTIQVNDLRGKFVKPALKKVSKFDKLKMIAKSTSEVDFRSNLKAVKKEAFKLDEEKQTEKKPEWALGSKKENEE

SEQ ID NO: 183 - protein, 443 aa, Dermacentor variabilis
MLRRRTAARGRTQSTMPPPSKAGGVMGFVGTKHVLAVLFACFATHVWIQGSSAVDSVDPDISRNVSQIIADKGYPVEEYEVETSDGFVLSVQRVPWGIKRRSDKLPGGGAKKVVFLLHYLLGSSADWVINYPEQSLPYLLADSGYDVWLGNVRGNTYSRHLRYRKDEKEFWDFCFDEMIAYDLPAMLDFVLNETAQQKLFYVGHSQGCLILFGLLAERPEYNDKIELFTAMAPVTTVTYMRSPIRYLAPFVEDAGAVFAMFGEYDFLPSSPLMRLVAGTLCLFEESRSLCENTIFFICGPEGPQLNKTRLPVLVSHTPAGTSVRNMAHYAQLVLSEKFQKFDFGKTRNRVRYGQETPPVYNLSRVMAPVALYWSSGDWLADPKDVSQLRDQLPRLVLDFRVRDPSFTHLDFSVGVGARRLVYEPLMKAMASVRTLLHHFYPES

SEQ ID NO: 184 - protein, 441 aa, Dermacentor variabilis
MTRYVLSLLFIVPCAWETTVAGVATTCDPRVCSLRDNCFCLGTRPPTNLSASSMPQFVMLTFDDAVNEQNIDFYRQLLSPGRRRNRANGCNLAATFFVSAGYTDYSLVHELHSVGSEIAIHSITHRNNLSYWRTLDIAGWEAEFVGDRDLLRDYALIPERDMVGARAPYLEAGNGEAYRMMRQNGFVYDSSICIDYMNKTDKLPVFPYTLDYGLATECTVPSCPSGSHRGLWLVPLNSFYRTTSRADGRLGSSCAMPDTCTPQPNTEDDTLDFLRSNFERYYHINKAPFPLFIHETWLQNPRRRQGYLSFVDWLLTMDDVFVVTVAEVVRFMRDPKPLGEYAQTGCSRASEFRRCARVHSCSFPDAPIDETRQLVGCKPCPESYPWLRDVTRRKAEDEHDSRVVRKQQYFGSGCVLVFCSALVVCLYLAARRLGRRKTHCS

SEQ ID NO: 185 - protein, 520 aa, Dermacentor variabilis
MKGIALFVVSLCASSILCLQAKASLLTEDEAASKRRVFFCYWGSWSHYRSGAGKFSVGQIDPSLCTHLVYAFAKLDNGVIAAFDAYLDLKDNYGLGMYENVNKLKVAHPHLKTLLAIGGWNEGSEKYSRMASTPEGRQRFARSVLDFLDKHGFDGLDLDWEYPAARGGLSQDKQNFVLLLQELRKVLGPGRLLTAAVSAGESTVDGAYDVPAITRHLDYISVMAYDFFGAWNSYTGHCSPLGVRENASEDEKKLNVVQAVKMWLDKGADPNKLVLGMPLYGRTFTLANPKNNGFLAPTVGPGPAGPATGEAGYLGYNEICMQLLASKDWKIMRDAGVVAPVAVNGNLWIGFDDAQSLTAKVLFARSLGLAGAMVWSIETDDFSGTCGGVKNPLQRAIKDALEANATIPLPTMATNLPSTTPAEPKPETTSTTGINSPSTLPATLKPENETSVSLLTTTGFPTMTTSAPELKCSNDGFYAYPNDPHRFYRCVSQPDGTYIIYVFDCPANTVFNPSVSVCTF

SEQ ID NO: 186 - protein, 129 aa, Dermacentor variabilis
MSQSAGMIAVLLTALFAVTLAIPGGWSTKQPSSSPKYKELAHFAVAQRIEGLQKYDTVLELTKVETQVVAGVNYRITFTIAGSDCIIGEVEYNAERCPPKDNQAKATCTAVVYERPWEHVRTLTSLNCA

SEQ ID NO: 187 - protein, 1547 aa, Dermacentor variabilis
MRVLWLSLLVAAASGFEVGKEYVYKYKGTLHVANPEQPLQASGIAFRTKLIVQPKPDGTHFKIVNFEADSFNSEQIDVAHHEFNYAANPNAAGDLEHPFAGKFDEGKLEEFSIGKNEQLWVRNLKKGVLSLFQLDLVKGRHEHHDDKGYHVKEDGLHGPCDTMYIVHEEEHDYIEVTKVKNLDKCDHEHYSFYGHQKEYQCVKCEALATYPHTATSEVYYELKGTAQHYVIGHAWGESAQLFKPHGEGKQFHVLLNRTLDLEEEHDAATTDTTLVEAGEKEHSLAQEFPETHELENPEELKRPNRLVTHFGLLPNKENFVEGLKKLAHIEYGDEDIKEIDNKESGSLLFLMLFHNFLTFSYDDINDVYQNHVLTAPEDIKDSLRHVFLDLLAAAGLNPHVTYGLNLIKHNELSVDDADRFYNKLHLNLKEVSPALLREISDSCKSDAVKSHREIWTSCKLAASAIAGGKGCKYAHDTHEEDKGTCSPEIVSHFFNYSVTPKDVEHEPEYESTVFIRSAGNLGTHKALHYLERFIYPKWHANEHKRMAALWALKQAAKHHPELARSIALPVFHNTSEPSEVRIAALLVVVVTNPDLYVLRHIGLEVLSDPSDQVVAFVISAFRALANSKYPCHKEIAQHLRYVLPLWETNPRFRKPIDRASSHLLISSGYNPKYDYGGLTLVEMIKSHDSYLPRNLYINMKDYVAGHSTDTVAFSFESWGLDKVFNRLVGPQPGSTKNLWNFMGRRRFPRDASAKERKEIEDALHIHDREYDPVYARMSLSVFGKAVDSWDFDESILDAIKSKDAPEKTAEKLLGKALRKKTFYLSHDMTYLNPTELGVPVFFDFKQAEFIYANREKIDVTHGDNAEIHLDVKRHYLYESRTQQMLGFAWTFSRSSLGSGYDARTVISWPLDLKVTLAPLEGKLSLNRPLHLPWNAINHHFHPFTFNMPYDLTQDHANAITEITANQKPLYRQDELLEFDRRYFGDVFGVAMNVKGHLIKRGLHSGLDEFYHQMTLRERLYYITINPHWHPRNVKLYFEPAGDAPTKEMDIDIAYKFLEPDDERHSHFKVHDQIGDDTEVPSTHVLNIDVNFKGDTKERKVATEFRYSFNHDLFNHKLQFFYDRTPFRSNDQEGTKICLEASAKFPKPDWSRVKNLATFYQGKHIDANLDIHYGSSCEGQSSISIHGQYTHTDKDEEQLVNAAAGKPITGNLRYNGLHRMALQCNAGREHGIPFNYYCMKFLRHSSRLAKLTADVEWKNYKPLLNKLLPLHTKYHALRPEHGGFFGIIRSHFTGENGKLHLVSQVPWWDLKDKPHTDIVITTEDGQHFKHWNVPTFSHMLEPRAFSSLGYSNIAEYAKQYRHRHCDLQKLSLRTFDGSLVQLPETDCYKVVTRDCSPNKRFLVLARSTNNPSLTKALKVFIHTTKLEILPVTEDSGLIVRVDGNKVDVVPERPYSHTDHDVELFEVRTREKWFEVTSKSYGLYLTFNGNLLFMQTAPFYRGKLCGLCGDYNLDRNHELSGPDGHLYNSTLEFAKSYVVPSPDCHPPTH

SEQ ID NO: 188 - protein, 847 aa, Dermacentor variabilis
MRLHFIALVVFAAAASLCHARSLLLHERHFADDEAYIVDTLKDIGRTLKDQFLVIGSQIQNKTQELKEAVGNHAQQVTEQLKALGEQLKKSRDALAKKLFELFSPAKYTDSNGNIFQIIASLANIRGRLENFLKEVHLATGEKWEKLKALIHELRSEIRQKVQELLHGSSAPATYTALDEFGYGDDEAYIVDTLKDIGRTLKDQFLVIGSQIQNKTQELKEAVGNHAQPVKEQLKALGEQLKKSRDALAKKLFELFSPAKYTDSNGNIFQIIASLANIRGRLENFLKEVHLATGEKWEKLKALIHELRSEIRQKVQELLHGSSAPATYTALDEFGYGDDEAYIVDTLKDIGRTLKDQFLVIGSQIQNKTQELKEAVGNHAQPVKEQLKALGEQLKKSRDALAKKLFELFSPAKYTDSNGNIFQIIASLANIRGRLENFLKEVHLATGEKWEKLKALIHELRSEIRQKVQELLSKNVHASKSGKAQDEVRRSSPLAERALSLATRMFSLRWRLAVLLKELDEGQEKFDDVKSKIERLRKEIRYQVALYFNRTKVQPTFYYASDDDDKEVYVTDTLKNVALTLKEKFLVLGEKIKTKLAELKVAVGERAVSIQEQLKVLYSQLQQAREDLKKQLFELFRPAAYASFQETAAKTYEKLNVIRQKLLKFLEDVRTLIGEKFESTKEVIAELREEIRHRVQELLVGQTGVAMYLAEDSVPKVSVINTLKETAKTLKEKFQTLGQKIKAKVAELREAVGEQAVLVKRHLESLKSQFNKAHSDLMAKLFALFRPAQYGTLNEAPEEEQHLSLLKLKLQRLLAATGASPAEEWPALDDAVYELDQEIQAYVKKYQ

SEQ ID NO: 189 - protein, 289 aa, Dermacentor variabilis
MAPRNWKRPLTLIYNDNYKYGNSLYSGALDDIEKRYQASLSRTTFRADRGDLGLSTFSSSNIGSAPEPSAGRPATTATNESDHALLSPSFSAPSRSPVRSMLDADHDLRKARSQGDFGRLRSSALRPNSWSDEEALLDDQSYTRRRRRRKQDRSDFLLSSPTVAAGENIDYYFDKCRELKNELEALHGRVLDAEARARSEAGTLRNKVHTEIAELSVQLEDTTAQNNELQKAVKRQAKQLMDLQAYCDEMQRHMQGAVDDLSTANKRCLDLQNEVDSLRSNLESGMRHI

SEQ ID NO: 190 - protein, 800 aa, Coxiella endosymbiont of Rhipicephalus microplus
MRVYKRFLCTIPLIVFCLSFNGSAYAFIIRSIHIQGLERISKSTVRSYLPIHEGQEYCPQYSQSIIEALYKTGFFDNIQLSRRGENLIINVKEYPTISLIHLNGNQVISNKKLQPIFSKFRIVEGETFNPTRLKQIEQGLEQQYDAMGYCEAHISTNIIRESHNCVALYININEGWITKVLSIQFVGNRAFSARTLRHQFKLTTPGLFTSITHTDRYSQIKLEEDLERLMIFYLDHGYLRFRVVSHELQWSQNKKSVYITVYLNEGAVYRINSYEITSKTYDLKEQFYKLITLKSGDVFSRQAIIDANIKIHNFLADRGYPFAKVTIIPTIDDPQRLVHINFIIVPNERIYVRRINFLGNQRTDQKVLRQEMRQYEGSIYSLSKIEESKRRLELLGYFSDVTYTTQSVTHSRDQVDLYYHVQEVPAGRASIQGGYSDVYGFLYGSSISEPNFMGTGKYLSIGFQNSQYQKNYSINYKNPYYTTGGLQRGFLIYYSRIKPDAKFNLTSYLENGYGADVTYGYFVSERNSIMFGYGLEDITISHVDIPNAAPSMLAFLGTTNGVQNTNVNYNEFKLIAEWVYNGLDRAIFPTKGFYRGLDLEVGVPLFKSSLNYYLATYSVKYYQPLGHKFILNVLTTFGYGEKFYHNRLPFFKNFFSGGIGSIPAFAPNSLGPKNRYPLGSDFSAIGGNLETIFGVHLILPQLNQKVRTAIVFDAGNVFQVPRFPGDIAIPARGGASDPEANIRPQIIQDDKFSIKNLRPSLGFAVEWYTSFAPIDFTLAFPLNRRVGDNFQAFQFTFVVS

SEQ ID NO: 191 - protein, 228 aa, Coxiella endosymbiont of Rhipicephalus microplus
MSVKKLITFATAGIASINVTSAAVASGLNYVPAPSYTGVYVEGNIGYVYRPWKSDVMTALSGIGTLNNLDVLSTSFQGNGGFTLGGTLGYQLNQYFAVEGSWFSFPKVKFTINSTTFMMKCGNPYAAFKATAPIYQNTYVFGKLGVAYLYNRSSHALISSNLSNFDSRHSNYWNPLFSAGFQYYFTPNWSVNAQYTFVPGCQKSVSSTRFLAPVAHLFTVAIGYKFLM

SEQ ID NO: 192 - protein, 263 aa, Coxiella endosymbiont of Rhipicephalus microplus
MKKLFFIFTFLYFCVLTSCARKDVNPYQEDHRASLIKLFRAGKKALVKKNYSEAIKNFETLDAIYPFNSYTQQTQLDIIYAYYKNEDIVSAIVAADRYIRLYPRGRHVDYAYYMRGILGFYYLGLSWVQKLVGVNPAFRDISILQQSYKAFATLVDTFPQSPYVSNALTHMKYIRNLMAKREIIIAQFYLKHCAYVAAANRAAYVVQHFQGSPEVIKGLAIMVQAYRALDLPKMADASYRLLKMNYPNSPELRKFTHLKFFAN

SEQ ID NO: 193 - protein, 166 aa, Coxiella endosymbiont of Rhipicephalus microplus
MTKRLFVAAYVLVSISWVGASIGQTVGTVDIRQIFQASPSQVKNINTQLEKEFSPQRERIINLGKSLQEDTKKFQRNEAVMSDKEKQELKNKIDQKQKEFQQAQLKFHQELYNAQNKAMFEFMTKISGVVRALSEEEKVDLVLPKDTVLYARDTKDITSDVIAKLK

SEQ ID NO: 194 - protein, 114 aa, Coxiella endosymbiont of Rhipicephalus microplus
MWGVTLILLVCLITTLEVEGCVYHPTLQQGNIITDKDLSNLNTGMTKDQVRAMFGDPVLVNIFADNQMVYIYTFQPSHLRMQSTHLIIYLHNNYVTNFWTDKNKPIEQLKLSNP

SEQ ID NO: 195 - protein, 116 aa, Coxiella endosymbiont of Rhipicephalus microplus
MDMVVNQISRRGYRDWLVQRITALLSGIYTIFIIIFLIINYPISYRQWYFLFTHLTMKFFTLLVIFSVLWHAWIGMWTIFTDYVKSKLTRLTLEIIVCLLLVTYFIWAIEFLWIAR

SEQ ID NO: 196 - protein, 406 aa, Coxiella endosymbiont of Rhipicephalus microplus
MLIIEIKVPMLPESVTDATVAKWYKKENDFISRDENLVDLETDKVMLEVVAPKDGIIKKIQVKEGGVVKASEILAILEVDNEENIIKKEKKDIDEYDIDEYEVKNSEEKKIEKQDEESESDLSPSVRRLVTEKGLDVTKIEGRGKEGRITKKDIEDYLETQKTKLSEKKIRKGELTEKRVPLSRIRQRIAERLVQVQQEAALLTTFNEINMKSVMDLRTKYRNEFEKKYKVRLGLMSFFTKASVEALKRFPIINASIDGREIIYHHYYDIGIAVGTERGLVVPVLRNTENMSMADIEKQIREYSSRAQEGRLGIDELTGGTFTITNGGTYGSLLSTPIINLPQTAILGMHKIEDRPVVVNSEIVIRPIMLVALSYDHRVIDGREAVLFLVTIKKLLEDPARLVLGI

SEQ ID NO: 197 - protein, 397 aa, Coxiella endosymbiont of Rhipicephalus microplus
MSKEKFIREKPHLNVGTIGHVDHGKTTLTAALTKVLSDKFGGEVRAFDQIDNAPEERARGITIATSHVEYQSDKRHYAHVDCPGHADYVKNMITGAAQMDGAILVVSAADGPMPQTREHILLAKQVGVPAIVVFLNKADMVDDQELLELVEIEVRDLLTSYDFPGDQTPIVVGSALKALQGDTAELGIPVILKLVETMDAYFEEPQREKEKPFLMPIEDVFSISGRGTVVTGRIERGIVKVGDEIEIVGINATTKTTCTGVEMFRKLLDQGEAGDNVGVLLRGTKREEVERGQVLAKPGTITPHTMFEGEIYVLLKEEGGRHTPFLKGYRPQFYFRTTDVTGEVLSLPADVEMVMPGDNVKVTVKLIAPVAMDEGLRFAVREGGRTVGAGVVTKIIK

SEQ ID NO: 198 - protein, 215 aa, Coxiella endosymbiont of Rhipicephalus microplus
MNTMNYKINLFFFILFLLTIPLVGISVKKPDAASQLSQLLSSFNTYQATFRQTTFDSEKRVIQKSWGRIMLKRPRNFRWETNIFTNQIIITNGKSLWIYNKDLSQAIQQPLERRKTITPVSFLSGSIKDLKPNFTIKSSLNNGTIMFELVPRIKKNLNFKWIQLKFTQKKLTELTLLNQLEERSIFRFKHIKINQPLADYLFQLKPSHDVDIIKQ

SEQ ID NO: 199 - protein, 240 aa, Coxiella-like endosymbiont
MLKKFSIGTIIIFASFALYTSALADSMNSLSFNNDNGFYVRGEGGYGWAADYKAKKVISFPVSIVTPLGVQPTLSINHKKTRGFMSRISAGYNINQYFGIETGFSMYHPAYRSLTFKSNLPLPQGSDIGTAKTSLYTFDLMGKVTTCYNNFYAFIESGAAYVHAKNSALRTNFSNNLITFLKASSKDFVQPKVGADIGYILTSNVAVDISYACIFGKGSISSPNYLPNLKTTIIGLTYKL

SEQ ID NO: 200 - protein, 581 aa, Coxiella endosymbiont of Rhipicephalus microplus
MKNYLSILSIISLFYLSFSSFAWARISPPPVHNTLEVQILGFPLTPLENVFKRLAEKQIAIQDEFTIATIMKFYREIPKEIKEAIKPYGYFKPYIHSYIQRCHSSLWYSHFVVNPGPRMYFTHVKLQIIGPGSFDKAFQQLYKNFPIKAGNFFDSQKYEQAKNDLFNVAATQGFFKAKILKSQIQINLSTYRTTVTIIFNTGPRFHFGVTHFSYTPFSPAFMNHFLQYHKGDYFSEEKVKQTREGLVNCNYFSTVIVTPEPEKAIGLYVPVTIHLHTEPKKQYSFGLGYGTDTRVRALIRTNFRWINPYGDRFNAYLRTSSVNSALVANYYIPGGNPIKDQYVLTADFLNQDQDVGKGRSERLTASYQTNLNGWQQTISLTALQERYNLTGLPRTNAKVLYPSALWQHIHADNLLNPKRGYSIIATISAAAKGFLSKTSFLQSRINSRFLFTAWDQTRFILRNSLGYTAIKNIVNLPLSFQFLAGGAQNLRGYSYNSIGPGRSFLVGSFEIQQKIMTNLYLAAFIDAGNVSNQIYKNKLKVDVGLGLVMLTPIGMFEVTIANPISEPKKRWVIQFSMGSPL

SEQ ID NO: 201 - protein, 306 aa, Coxiella endosymbiont of Rhipicephalus microplus
MKIKNLYGIIIILTSLLLIEVVALGDAPVVDITQKSNSQETSDGIPTTVTSENFNSSATEITPSDSASFLPTQPLSDTQRIARLEQQIKNLININLPQQIIDLQQQLAQVRGQLQEQQHDLQLVNKQLRTFYNNLNQRLIQLKNLNSDTLNDNSSSQKTTGNSFSNNGNIQLQDSTAYQIAINFLNKNQYDKSLAAFQNYLNNYPNGNYVANAHYWLGEIYLEQNNRKKAVLEFQIIKNKFPQSEKVLDAQLKLAMIHAENGQVIQAKQELMEIKKQHPNSTVAQLASICLQQLESPNASLTNSLL

SEQ ID NO: 202 - protein, 179 aa, Coxiella endosymbiont of Rhipicephalus microplus
MRSRFQWAILSMLLALTGCKSYIPSIEGESAATGIKDAAKSYTFSGESSYHSQVARDFEGRIINSLTAPTNQTYYFDFDSTAAQSADLQAIHIQSNYLITHPTAKVRLEGNTDNRGSREYNIGLGWRRDQAIARIFEQEGVAPSQMDMVSYGKEHPAVPSNNEYAWRLNRRVNLIYEAY

SEQ ID NO: 203 - protein, 397 aa, Coxiella endosymbiont of Rhipicephalus microplus; identical to SEQ ID NO: 197

SEQ ID NO: 204 - protein, 469 aa, Coxiella-like endosymbiont
MLSKFLVSIVSFCVTTVLNPLFAATNQQINKRIEYFQKQINQFKIQKKQSQNKLTYRYYYQKCICPNGTSIYHISHVGIGPYLNTQEAFNVSTLIINTPTIREDAWILLNQYQLAEKCSGMGIPTFPLPQITFSGKLEGQVGYKSTYSGSRTVNINFSGAELDTYLQKNLWVSGYISLDYDPNELVDGSDVFINRAFLTIGNLSRFPFYISIGQVYVPFGRYNSLMISSPVTLGLGRTRARAVTVGYQTNKNTVHAEIYGYQELTNNLSSSNQNNQWGMDIGYEFNNGGQVSGEVGASLISNLADSEGMQSTVFLHNKILDHNIQAINFYGSFSINTMVFIAEYVGALRSFDISNDIRFANQSIICRPTAFHTEANYTFHIASKVSSLGIGYDHTGQSLALGLPQERYSIFYNVNIWRDTNFALEYRHDVNYSRNSTTIRIHSIYPFSGITDDFSKSNNVVMAQFDLYF

SEQ ID NO: 205 - protein, 201 aa, Coxiella endosymbiont of Rhipicephalus microplus
MLLSAIAASTSCTLTPPTTTLHFPTQPTVVIYKAKSWQQRYHALRQYLLWNINGTFSIQQPSRTIIAEYKWQQKKSNYHIRIYSILGIYSIEIYGRPGIIMLSSSSQKYYTTLTPEKLLQKQLGWKLPVLNLFYWVRGIPAPGIYHSKFDSSGHLIFLQQNGWKIRFSRYSRINFIDLPHTLILNKKPFTVKIIIKSWKLY

SEQ ID NO: 206 - protein, 208 aa, Coxiella-like endosymbiont
MKNSLKFMLLSAIAASTSCTLTPPTTTLHFPTQPTVVIYKAKSWQQRYHALRQYLLWNINGTFSIQQPSRTIIAEYKWQQKKSNYHIRIYSILGIYSIEIYGRPGIIMLSSSSQKYYTTLTPEKLLQKQLGWKLPVLNLFYWVRGIPAPGIYHSKFDSSGHLIFLQQNGWKIRFSRYSRINFIDLPHTLILNKKPFTVKIIIKSWKLY

SEQ ID NO: 207 - protein, 492 aa, Coxiella-like endosymbiont
MKRKLINFITALLFLIMQSIYADNLIQVYYQAVEKDPTFLEAHASWLSAQQDLPIAISGSGAPGSSLLPYVTITGSLAQTHQKIAVDFTPNSGDNGDRASEYFKKNTYLVTLTQPIFNYATWKSISRAHFTVKAATARYIADTQDLILRTAQAYFNVLNAYDQLQFTLAQKKSFLHQLITAEEKFKVGLIAITGVYDAQASYDQAVAQEIQDRNNLANQLENLRAITGREYKSLAGLKNTIPLLIPHPSNINAWTIIAERQSYSIQSVLYSMLAQQELVKQTAAGWYPTLTGVITHESNQQGFPSTLSELPATTGANFINKTGTTTSGGVNLNFPIFQGGYVIHATQKEEYNYLSASQQLNLTHRQVVQQTRQAYLGVDSGISKIKADEQAIVSAENKLKATQAGYVVGTRTMVNVLDAVTNLYQAQQQWASDRYMYIINIITLKQQAGTLCPQDLLEINSWLGKSVKFDLNKPVIVSKYATELPTALSKNN

SEQ ID NO: 208 - protein, 801 aa, Coxiella-like endosymbiont
MRIHKRFLFTIPIIVFYLPFSLSVYAFVIRSIQIQGLQGISKSTVRSYLPIHEGQEYFSQRGQVIIEALYETGFFDNVQLSRRGEALIITVKERPTISLIRLSGNKAITSKKLRPILNKLGIMEGQTFDPTKLKEIEQGLEQQYGAMGYHAAHISTNVIRESRNRVALYININEDGIAKVLSIQFVGNRAFSARTLRSQFKLTTSGLFTCISHADRYSQIKLEKDLERLITFYLNHGYLRFLVVSHDVRWSRDKQSVYITVYVNEGPVYRIKGYEISGETYGFKERLYRLITLKPGDVFSRQAIIDTNTKIGNFLADRGYVFARVTIIPTIDDQQHLVHINFTIVPGERVYVRRINFLGNQRTDQEVLRREMRQYEGSTYSLSKIEESKRRLELLGYLSDVTYTPQPIPNSRDQVDLYYHVKEVSVGRASIQGGYSDVYGFLYGASISEPNFMGTGKYVSIGFQNSQYQQNYSINYNNPYYTTWGLQRGFSIYYSRIKPDAKFNLASYLEDGYGADVTYGYPVSERNSIMFGYGLKDITISHVDIANAAPSVLAFWDTTNEVQNISANYNQVKLTAGWVYNGLDRTIFPTKGPYTGLSLEVGVPLFKSSLDCYLATYAAKYYQPLGHGFILNLLVTLGYGDKFGHDRLPFFKNFFAGGIGSVPAFAPNSLGPKNRYPLGSDFSAIGGNLETIFGVHLILPQFSQKVRTAIVFDAGNVFQVPRFPGDIAIPARGGASDPEANIRPQIIQDDKFSLKNLRPSLGLFAEWYTPFAPIDFTLAFPLNRRAGDNFQAFQFSFGVSL

SEQ ID NO: 209 - protein, 231 aa, Coxiella-like endosymbiont
MSVKKIITFATAGIASVGVASAAVAGGPDYVPAPSYAGVYVEGNVGYVYRPWKSDVTTLPGTLNNLGVLSSSSRGNGGFTFGGDLGYQFNQYFAVEGGWFYLPKAKFTINPTTFTIKATTFTIKGGLPYAALKAMVPIYENTYVFGKLGLAYSYNRSSSALVSSDLTNFVSDRSSYWNPLFAVGVQYYFTPNWSVNAQYTFVPGYRSAFSTRFVAPVAHLFTVGIGYKFLM

SEQ ID NO: 210 - protein, 197 aa, Coxiella-like endosymbiont
MDAIYPFGPHAQQAQLDIIYAYYKNGDIGSAIVAADRYIRLYPRGYHVDYAYYMRGIIGFDLRLSWIQKLAGVNPASRDISTLQESYRAFATLVDMFPQSPYVPDALIRMKYIHNLMAQREIMIAQFYLKRHAYVAAANRATYVVQHFQDSPEAIKGLVIIVQAYRALDLPKMADSPSYRLLKINYLNSPKLRKLPG

SEQ ID NO: 211 - protein, 165 aa, Coxiella-like endosymbiont
MMKRLFAATYLSVAMSWAVASIGQTVGTVDMRQIFQASLQVKNINTRLEKEFSPQRERIINLGKSLQEDTKKFQRNEAVMSEKEKQYLKNKIDEKQKELQQAQVEFQQGLYNAQNKAMGEFMTKISGAVKAISEKEKVDLVLPKDTVLYARDSKDITSDVISELK

SEQ ID NO: 212 - protein, 115 aa, Coxiella-like endosymbiont
MDMVDQRSRRGYCDWFVQRITALLSGIYAVFLIIFLLIHHPISYAQWHSLFTHLAMKIFTLLVIFSVLWHAWIGMWTIFTDYVKNKPTRLALETIVCLLLVAYFIWAIEFLWIVR

SEQ ID NO: 213 - protein, 438 aa, Coxiella-like endosymbiont
MANSIEKISVPDLGGASEVDVIELLVKPGDRIAKEDGLITLEGDKASMDVPSPLAGTIKDIKVKVGDKIKEGDEILTIEVKKSEKDDKGKKEEKEDEKEAEKERSEEKEVKESPKEKKAKPEEKTKIEVSEGEGFGTTVHAGPGVRRVAREFGINLNKIKGTSQKDHRILKEDVQQYVKQQLKIAKRKGDEGLPFPPAPKIDFSKFGAIEQKALSKIKEATGVNLTRNWMSVPHVTQFGEADISELEAFRQSQKAYAEKNNVRLTPLVFIIKAVVNALKRFPHFNASLDPSWEYLILKKYFHIGVAVDTPEGLVVPVIRDADKKGLFELAKELGEISEKARTKGLSLNDMQGGCFSISSLGGIGGTAFTPIINTPEVAILGVSQMQWKSVCSKSRNCKTRLMLPLSLSYDHRVIDGADGARFIVYLAERLSDIRTLLL

SEQ ID NO: 214 - protein, 406 aa, Coxiella-like endosymbiont
MSKEKFIREKPHLNVGTIGHVDHGKTTLTAALTKVLSDKFGGEARAFDQIDNAPEERARGITIATSHVEYQSDKRHYAHVDCPGHADYVKNMITGAAQMDGAILVVSAADGPMPQTREHIVLAKQVGVPVIVVFLNKADMVDDKELLELVEIEVRDLLTSYDFPGDKTPIVVGSALKALEGDSGELGVPAVLKLVETMDAYFEEPQREKDKPFLMPIEDVFSISGRGTVVTGRIERGIVKVGDEIEIVGIKATTKTTCTGVEMFRKLLDEGQAGDNVGVLLRGTKREEVERGQVLAKPGTITPHMTFEAEIYVLSKEEGGRHTPFLKGYRPQFYFRTTDVTGEVLSLPGDVEMVMPGDNVKVTVKLIAPVAMDEGLRFAVREGGRTVGAGVVTKIIRKLLSNCGNK

SEQ ID NO: 215 - protein, 237 aa, Coxiella-like endosymbiont
MLKKFAIGATAVATFGLCADALADSMDSSGSDGTGFYVRGEGSYGWADSKANKVIGFPAGIVAPLGIQPTLRTNNKKSRGFTGRIGASYSINQYFGIETSFSVYHPAYRSLTFKGNLPLPQGTGTGAVKTSLYAFDLMGKATIPFNNFYAFVEGGGAYVHAKNSALTANFGNNPITFLKASSKGFVRPKVGAGIGYNLISNLAVDVSYSRIFGKGSISSPNYLSNLNTTMVGLTYKF

SEQ ID NO: 216 - protein, 540 aa, Coxiella-like endosymbiont
MPFIPLENVFKRLTEKQAAIKDQFTTATIMKFYQDIPKEIEEGIKPYGYFKPYIRSYIQRRHSSLWYSDFIVNPGPRMYFTHVEVQIIGPGSSDKAFQRLYENFPIKAMDFFDSEKYEQAKNDFFNVAAARGFFKAKMIKSQIQINLNTYRATLTTIFDTGPRFRFGVTHFSCTPFNEAFIHGFLQYREDHYFSQEKVKQTREGLANSNYFSTVVVTPEPEKAKGLYVPVTIYLHTESKKQYNFGLGYGTDTGVRALIRTNFRWINPYGHRFNAYFRASSVNSALIANYYIPGGNPVKGQYVLTATFLNQDQDTGKGRSGRLTASYQTDLNGWQQTISLTALQERYNLTGLPQTNARVLYPSILWQRIHADNLLNPKKGYSIMATISDAAKGFFSKTGFLQSQIDGRFLFTAWDQTRFIIRSSLGYTAIKNILNLPLSFQFFAGGAQSLRGYSYNSIGPGRGLVVGSFEIQQKILKNLYLTAFIDAGNVSDQISKNKLKVGVGPGLVMLTPIGMFELTIANAIIEPKKPWVMQFSMGSPL

SEQ ID NO: 217 - protein, 305 aa, Coxiella-like endosymbiont
MKIKILYALTGLTTLLLIEVVALGDAPVVDITQQTSGQETPAEIPITATVESSNGSATEITPPGPAASSPTQPLTDSQRIARLEQQMNNLININLPQKITDLQQQLAQVRGQLQEQQHDLQLVNNQLRSFYNDLNQRITQLKNLNSDSSNDNSSSQKTTRNSLSNNGNIQLEDSTAYQTALNFLTKNQYDKAEASFQNYLNDYPNGNYVANAHYWLGEIYLEQKNRKKAALEFQIIKNKFPKSEKVLDAQLKLAIIHAENNQVAQAERELMEIKKQHPSSTVAQLASIRLQQLEAANSPSTNPLL

SEQ ID NO: 218 - protein, 191 aa, Coxiella-like endosymbiont
MVGSTTVRGVHMGKRFQWAILSMAILGLTACKSHTPSMSGESAATGVGNGAKTYALAGQSGYHGQVMRDSKGEIINSLVAPANQTYYFDFDSTAVRSVDLQAIHIQANYLATNPKAKVRLEGNTDNRGSREYNIGLGWHRDQAVARIFEQEGVEPSQLDMVSYGKEHPVVTSNNEYAWQLNRRVNLIYEAY

SEQ ID NO: 219 - protein, 406 aa, Coxiella-like endosymbiont; identical to SEQ ID NO: 214

SEQ ID NO: 220 - protein, 465 aa, Coxiella-like endosymbiont
MSSKFLVSIVSLCVVSALTPSLAATKQQINKRIDYLQEQINQLRAHQKQAQNKPTHTHHYQEYTCQNGKCVYHTSRLGIGPYLNTQEAFDGSELIINTPTIREDARMLINQYQLEQECSEMGIPTPFLPRVTFSGKLEGQVAYGSTYAGLRTTNINFSGAELDTYIQGNLWVSGCMDLDYDPDELVNRSRVFMNRAFVTIGNLSRFPFYISIGQVYVPFGRYNSLMISSPVTLGLGRTRARALTVGYQTGKNAALHAEVYGYQELTNNLSHSNQNNRWGTDIGYEFNNGGRVSGEVGASLISNLADSQGMQATVFLHDETLHRTVEAIDFYGNIFIDPAVFIAEYVGALRSFDVADVSFANQGARPTAFHAEANYTFHTGSKPSSLGIGYGHTTQALALGLPQVRYSIFYNVNVWRDTNFALEYRHNINYSRNAISTEINLIPSGMAAYLGKSDNVLTAQFDLFF

SEQ ID NO: 221 - protein, 801 aa, Candidatus Coxiella mudrowiae
MRVHKRFLFTIPLIVFCLSFSLSAYAFVIRSIQIQGLQGISESTVRSYLPIHEGQEYFSRRGQAIIKALYKTGFFDNVQLNRRGEALIITVKERPTISLIRLSGNKPITSKKLRYILNKLGIVEGQTFDPTKLKKIEQGLEQKYGVMGYHAAHISTNVIRESCNRVALYININEGRIAKVLSIQFVGNCAFSARTLRSQFKLTTPGLFTCISHADRYSQIKLEEDLERLITFYLNHGYLRFRVVSHDVRCSQDKQSVYITVYVNEGPVYRINGYEISGETYGFKERLYRLITLKPGDIFSRQAIIDTNTKIGNFLADRGYAFARVTIIPTIDDQQHLVHINFTIVPGERVYVRRINFLGNQRTDQEVLRGEMPQYEGSIYSLSKIEESKRRLELLNYLSDVTYTPQPVPNSRDQVDLYYYVKEVAAGRASIQGGYSDVYGFLYGASISEPNFMGTGKYVSIGFQNSQYQQNYFISYNNPYYTTWGLRLGFSIYYSRIKPNAKFNLASYLEDGYGADVTYGYPVSERNSITFGYGLEHITISHVDIANAAPSVLAFCGTTNGVQNTSANYNQVKLTAGWVYNGLDRAIFPTKGLYTGLGLEVGVPLFKSSLDYYLATYAAKYYQPLGHGFILNLLETLGYGDKFGHDRLPFFKNFFAGGIGSVPAFAPNSLGPKNRYPLGSDFGAIGGNLETIFGVHLILPQFSQKVRTAIVFDAGNVFQVPRFPGDIAIPARGGVSDPEANIRPQIIQDDKFSLKNLRPSLGVSVEWYTPFAPIDFTLAFPLNRRAGDNFQAFQFSFGMSL

SEQ ID NO: 222 - protein, 224 aa, Coxiella-like endosymbiont of Rhipicephalus sanguineus
MSVKKLITFATAGIASVGVTSAVVAGGPDYVPAPSYAGVYVEGNVGYVYRPWKSDVTTLPGTLNNLGVLSSSSRENGGFTFGGDLGYQFNQYFAVEGGWFYLPKAKFTINPTTFTIKGGIPYAALKAMAPIYENTYVFGKLGVAYTYNQSSSASVTSDLINFISPRSNYWNPLFSVGVQYYFTPNWSVNAQYTFVPGYRSASSSHFVTPVADLFTVGIGYKFLM

SEQ ID NO: 223 - protein, 256 aa, Coxiella-like endosymbiont of Rhipicephalus sanguineus
MKKLFFILTFLYLCILTSCAGKDVDPYQVYRGETSIKLFTAGEKALAKKNYSEAVKNFEALDAIYPFGPHAQQAQLDIIYVYYKNGDIPSAIVAADRYIRLYPQGRHVDYAYYMRGIVGFDLGLSWVQKIAGVNPASRDISTLQQSYRAFATLVDTFPQSPYVPDALTRMKYIRNLIAQREIMIAQFYLKRHAYVAAANRATYVVQHFQGSPEVIKGLAIMVQAYRALDLPKMADSSYQLLKINYPNSPELQKLPG

SEQ ID NO: 224 - protein, 125 aa, Coxiella-like endosymbiont of Rhipicephalus sanguineus
MMKCLFVATCLSVAINWTAAAIGQTVGRVDMRQIFQVSQQVKNINTRLEKKFSPQREKIINLGKSLQEDAKKFQRNEAVMSEKEKQDLKNKIGEKQKELQQAQVEFQQGLYNAQNKAMAEFMTKI

SEQ ID NO: 225 - protein, 112 aa, Coxiella-like endosymbiont of Rhipicephalus sanguineus
MRRVTLIFLVCLITTLWGCVYHPNIQQGNIITNKDLGNLDTGMTKDQVRVALGDPVLVNIFSDNRMVYVYTFQPGHRRMQSTHLIIYLHNNHVTNFWIDKNKTIGWVNLPNL

SEQ ID NO: 226 - protein, 437 aa, Coxiella-like endosymbiont of Rhipicephalus sanguineus
MANSIEKISVPDLGGVSEVDVIELLVKPGDRVAKEDGLITLEGNKASMDVPSPLAGTIKDIKVKVGDKVKEGDEILTIEVKKGEKEDKGKKEKKEDEKEAEKERSEEKEVKEYPKEKKAKPEEKTEIEVSEGEGFGTAVHAGPGVRRIACEFSVNLTKIKGTGQKDRILKEDVQQYVKQQLKIAEKKGSEGLPFPPAPKIDFSKFGTIEEKALSKIKKATGVNLTRNWMTVPHVTQFGEADISELEAFRQSQKSYAEKNKLRLTPLVFIIKAVVNALKRFPHFNASLDASEKYLILKKYFHIGVAVDTPEGLVVPVIRDADKKGLFELAKELGEISEKACTKGLSLNDMQGGCFSISSLGGIGGTAFTPIINAPEVAILGVSQMQSKSVCSKSGDCKIRLMLPLSLSYDHRVIDGADGARFIVYLAERLSDIRTLLL

SEQ ID NO: 227 - protein, 397 aa, Coxiella-like endosymbiont of Rhipicephalus sanguineus
MSKEKFVREKPHLNVGTIGHVDHGKTTLTAALTKVLSDKFGGEVRAFDQIDNAPEERARGITIATSHVEYQSDKRHYAHVDCPGHADYVKNMITGAAQMDGAILVVSAADGPMPQTREHIVLAKQVGVPAIVVFLNKADMVDDKELLELVEMEVRDLLASYDFPSDETPIVVGSALKALEGDAGELGVSAVLKLVETMDAYFEEPQREKDKPFLMPIEDVFSISGRGTVVTGRIERGIVKVGDEIEIVGIKATTKTTCTGVEMFRKLLDEGQAGDNVGILLRGTKREEVERGQVLAKSGTITPHTTFEAEIYVLSKEEGGRHTPFFKGYRPQFYFRTTDVTGEVLSLPGDVEMVMPGDNVKVTVKLIAPVAMDEGLRFAVREGGRTVGAGVVTKIFE

SEQ ID NO: 228 - protein, 302 aa, Coxiella-like endosymbiont of Rhipicephalus sanguineus
MYFTHVELEIIGPGSSDKAFQRLYENFRIKAGDFFDSEKYEEAKNDLFNVAAARGFFKAKMLKSQIQINLSTYRAAVTIIFDTGPRFHFGVTHFSYTPFNEAFMHRFLQYPEGHYFSQEKIKQTREGLTNSNYFSIVVVTPEPEKTKGLYVPVTIHLHTEPKKQYSFGLGYGTDTGVRALIRTNFRWINPYGHRFNAYLCASSVNSALVANYYIPGRNPAKDQYVLTAAFLNQDQDTGKGRSGRLTASYQTDLNGWQQIINLTALQERYNLTGLPRTNARVLYPSILWQRIHADNLLNPKKG

SEQ ID NO: 229 - protein, 220 aa, Coxiella-like endosymbiont of Rhipicephalus sanguineus
MNNLININLPQQITDLQQQLAQVRGQLQEQQRDLQLVNNQLRSFYNDLNQRITQFKNLNSDSSNDNSSSQKTIGNSLSTNNANIQLQDSTAYQTALNFLTKNQYDKAEASFQNYLNDYPNGNYVANTHYWLGEIYLEQKDRKKAALEFQIIKNKFPKSEKVLDAQLKLAIIHAENGQVAQAKQELMEIKKQHPSSMVAQLASIRLQQLKVANSPSTNPLL

SEQ ID NO: 230 - protein, 180 aa, Coxiella-like endosymbiont of Rhipicephalus sanguineus
MPKRFQWTILSMVILGLTACKSHTAFMDGGSAATEVGDRAKTYALVGESSYHGQVMQDSQGRIINPLLTPANQTYYFDFDSTAVRVVDLQTIHIQANYLAIHPKAKVRLEGNTDNRGSREYNIGLGWRRDQAIARIFEQEGVAPSQLDMVSYGKEHPAVSSNNEYAWRLNRRVNLIYEAY

SEQ ID NO: 231 - protein, 397 aa, Coxiella-like endosymbiont of Rhipicephalus sanguineus; identical to SEQ ID NO: 227

SEQ ID NO: 232 - protein, 112 aa, Coxiella-like endosymbiont of Rhipicephalus sanguineus
MIGGKFNLGNLMKNSKKIQDMMQKAQEKLSKIEVTGESGAGMVQVTLTAQHEVINLILSDELLKESKEVIEDLIKAALNHANQKVIKITQEKVMSAGSLFGSDFGSKEESEK

SEQ ID NO: 233 - protein, 816 aa, Coxiella endosymbiont of Amblyomma americanum
MVRPLHKRLLFTIAFIVLWIPLNLEAHDFIVRFIRIRGLQEISESTVRHYLPISKGQKYTFRQGQFIIKKLYKTGFFDDIKISSCGKILVITVKERPTVSVIHLNNQSVITNKKLRLILYRYGIIEGQIFDPFKLEQVKQGLEQQYGVMGYYSVHISTNVVRKSSNRIELYIKVEKPKIAKVYSIRFIGNQSFKEHILYRQFKKLISTGFLDTWIGNHRANRYSQIRLEKDLENLTIFYLNHGYLRFRLISYDVQWSSDKSKVYITVHINEGPAYYVNMCEISSANGDVKNLLRRFITLKSGDTFSRKNVIRISNNIRNFLGNQGYAFAKVVIIPIINDKRHLVNIKFNIIPGKLVHVRWINFLGNQHTNQAVLRREMRQYEGSIYSLSKIEESKRRLALLDYLSEVAYTVQPVSNSIDQVDLCYHVREIASGRASIQGGYSDVYGFLYGVSVVDPNFMGSGKYIAIKFQNNQYQRNYSIIYNNPYYTVWGLQQGVSMYYSRVRPDKKFNLSPYLEDGYGIDIAYSYPVSDHNIVMFSYGIGHVAVNNVDVVNAAPSVLAFLGTINGVQNVNAMYNQVKLAVNWTYNKLDRTILPTTGLYVGSVLEVNIPIFFVPSLDYYLATYTMKYYRPLWKEMGRNNGFILNVLATVGYGNGFDRNSRLPFFKNFFSGGIGSLPAFAPNSLGPKNRYPLGRDFGAIGGNLEVIFGAHLIFPQFFSQNVRTAIVFDAGNVFQVPRFFGDIAIPARGGAFDPEANTRPQIIQDDRFSLKNLRSSLGLSLEWYTAFAPIDFTLAFPLSRRSGDHFQAFQFSLGMIL

SEQ ID NO: 234 - protein, 230 aa, Coxiella endosymbiont of Amblyomma americanum
MLVKKLITFVTASIASVGVTSAAVAGGPDYVSTPAGGEYSGIYIEGNAGYVYHPWGSDVTTVPGILKNFSLISSSSRGDGGSTFGGDLGYQFNKNIALEGGWFYLPKATFNTVDPGSFAIKGGVAYTALKGMASIYGNTYAFGKLGVSYTYNWSSIALASSDLNSTNNVSSCSNYWNPLFAAGMQYYLTPNWSVSTQYVFVPGYRSASSNRFVTPVVHLLTMNVGYKFVI

SEQ ID NO: 235 - protein, 272 aa, Coxiella endosymbiont of Amblyomma americanum
MKKLLIITIFLCLCVLNGCTAEDFDPYQGYRKRSEIALFAAGKRALEKRNYSEAIKNFEALNVIYPSSSHILRKARLNVICAYYKNKDISSTISAANRYIRFYPKDCHVDLAYYIRGISEYHKVTLSRIQNLMGVNVVTRDISILQQSYETFSVLVDAFPKSAYTADALTCMKHIRSLMAQHEIIIAQFYLKRHAYVAAANRAACVVQNFQDSPENVAKGLKIIDTAYHALGLINFNCQCHKTYTHSKLKTIYHTQSLSYEEMPKFKRNCSN

SEQ ID NO: 236 - protein, 165 aa, Coxiella endosymbiont of Amblyomma americanum
MKKCLFKAVYLSIMMGWAASVVGQTIGVVDIRQIFQVSPQIKDINTKLEKKFSLQREQIVDLSKCLREDIKTLQRNASVMNKKELENVRDRIQKEQKELQKVQIVFQRNLYKAQSKAVNRFMNKILSVVKTVSEKQKIDLVLPKSAILYAKDKKDITSNVIAELK

SEQ ID NO: 237 - protein, 111 aa, Coxiella endosymbiont of Amblyomma americanum
MVNQKSYRGYRNWCVQRITACLGGIYTVFVIIFLVTHHPISYAEWNNLFTHTTMKIFTLSVVLSMLWHAWIGVWTIITDYVKDKPIQLILEAAVCLLLVICFIMCISKFFK

SEQ ID NO: 238 - protein, 407 aa, Coxiella endosymbiont of Amblyomma americanum
MSIIEIKVPLLPESVTDATIAKWYKQQGDVVSQNENLVDLETDKIALEVHAPKSGIIKKTVFKEGAVVKSGDVLVALEVSTIDAVVGLEEKKGTEDTIEVIEVKKPKTLNKREKKFGPSVRRLISREDLVGINKVLVENDKEKHITQKNAESYSDNQTDKYKEEQEIIREDIKKTRIEKRVPLSRIRQRIAERLVRIQQEAALLTTFNEINMQMVVDLRKKYCDEFEKKHKVRLGFMSFFAKASVEALKNFPLVNSSIDKNDIVYHDYYDIGIAIGAKRGLVVPILRNVENMSIVNIERKIREYAVRAQEGCLSIDELTGGTFTITNGGTYGSLLSTPIINLPQTAILGMHKIEDRPIVEQGRIVIRPIMLVALSYDHRIIDGREAILFLVTIKKLLENPFRLILEI

SEQ ID NO: 239 - protein, 397 aa, Coxiella endosymbiont of Amblyomma americanum
MSKVRFIREKPHLNVGTIGHVDHGKTTLTAALTKVLSERFGGEARAFDQIDNAPEERARGITIATSHVEYQSDSRHYAHVDCPGHADYVKNMITGAAQMDGAILVVSAADGPMPQTREHIVLAKQVGVPAIVVFLNKGDIVDDEELLALVEMEVRDLLTSYDFPGDETPVVTGSALKALEGDGGALGIPSVLKLVQVMDAYFKDPKREKDKPFLMPIEDVFSISGRGTVVTGRIERGIVKAGDEVEIVGIKTTVKTTCTGVEMFRKLLDEGQAGDNVGVLLRGTKREEVERGQVLAQPKTITPHTTFEAEVYVLSKEEGGRHTPFVKGYRPQFYFRTTDVTGEVLRLPKGVEVVMPGDNVTVTVQLIAPVAMDEGLRFAVREGGRTVGAGVVTKIVE

SEQ ID NO: 240 - protein, 397 aa, Coxiella endosymbiont of Amblyomma americanum; identical to SEQ ID NO: 239

SEQ ID NO: 241 - protein, 464 aa, Coxiella endosymbiont of Amblyomma americanum
MSSRFLILISTIILYIISFFTPLLAATKSQVASHFSVQTTQQKKSQRKSCICAFREYACPNLKIGPIDSYFNVRRTLNDPSELIVNIPAIREDASILVRQYQLERECQELGISVPSFPQVTFSGKLEGQLVYGSYSGFKTSNIDFSSVELNTYVRGNPWVSGYMALNYDSNNDSLSNGRSPVFLDRAFIAIGDLNQLPVYISIGQVYIPFGHYSSLMITTPVTLELGRIRSQAITIGYQKTDDNALHAELLVYKGLTNNLLHGNKRNQWGIDFGYKFSTGSRVNSEIGASLVSNLADSQGMQAAAFSSSLLPGKILHHAVQAIDLYGNFTINPMTFTAEYVGALSSFDISDISFKGRGACPSAFHTEANYALRSVGSRSSIGISYDHTAQAMMLGFPQDRYSIFCNIGIWKDTNFALEYRHDTNYSTKITSSVNGGIKNSIPASIIIGANKNDNVITARFDLYF

SEQ ID NO: 242 - protein, 110 aa, Coxiella endosymbiont of Amblyomma americanum
MVDSKFNFDRLMQNTKKIQEIMKKAQEELSTIEVTGESGAGMVRVTVTAQHEIVKLVLSDELLKERKEIIEDLIKAALNNANHRIMQITQEKIIFSGKFFHSNNESNVKK

(19) United States
(12) Patent Application Publication
(10) Pub. No.: US 2026/0137763 A1
(43) Pub. Date: May 21, 2026

(54) Anti-Tick Vaccine Compositions and Related Methods

(71) Applicant: Arizona Board of Regents acting for and on behalf of Northern Arizona University, Flagstaff, AZ (US)

(72) Inventors: David Wagner, Flagstaff, AZ (US); Joseph Busch, Flagstaff, AZ (US); Jason Sahl, Flagstaff, AZ (US); Erik W. Settles, Flagstaff, AZ (US); Nathan Stone, Flagstaff, AZ (US)

(73) Assignee: Arizona Board of Regents acting for and on behalf of Northern Arizona University, Flagstaff, AZ (US)

(21) Appl. No.: 19/375,059

(22) Filed: Oct. 30, 2025

Related U.S. Application Data
(60) Provisional application No. 63/714,050, filed on Oct. 30, 2024.

(51) Int. Cl. A61K 39/00; A61K 39/02

(52) U.S. Cl. CPC A61K 39/0003; A61K 39/0208

(57) ABSTRACT

Implementations of an anti-tick vaccine composition may include one or more conserved tick proteins from a tick species; and one or more conserved outer membrane proteins of an endosymbiont of the tick species.

### CROSS REFERENCE TO RELATED APPLICATIONS

This document claims the benefit of the filing date of U.S. Provisional Patent Application 63/714,050, entitled “Anti-Tick Vaccine Compositions to Prevent Tick-Related Diseases” to Wagner et al. which was filed on Oct. 30, 2024, the disclosure of which is hereby incorporated entirely herein by reference.

### SEQUENCE LISTING

This document contains the material in and hereby incorporates entirely herein by reference the sequence listing file in XML format filed herewith named NAU115.xml, created Oct. 30, 2025, which is 328,316 bytes in size.

### BACKGROUND

### 1. Technical Field

Aspects of this document relate generally to vaccines such as compositions and methods for preventing animals from becoming hosts to ticks and reducing overall tick populations.

### 2. Background

Ticks and tick-borne diseases are a significant and increasing global problem for human and animal health. Human diseases like Rocky Mountain Spotted Fever and Lyme disease are transmitted via ticks. Animal diseases like babesiosis, bovine anaplasmosis, and cattle fever are transmitted through ticks as well.

### SUMMARY

Implementations of an anti-tick vaccine composition may include one or more conserved tick proteins from a tick species; and one or more conserved outer membrane proteins of an endosymbiont of the tick species.

Implementations of an anti-tick vaccine may include one, all, or any of the following:

The one or more conserved tick proteins and the one or more conserved outer membrane proteins each have a conservation level equal to or greater than 97%.

The tick species may be Rhipicephalus microplus and the one or more conserved tick proteins may be at least 75%, 80%, 85%, 90%, 95%, or 100% identical to a protein selected from the group consisting of SEQ ID NO: 1-28 and any combination or fragment thereof and the endosymbiont may be CLERM and the one or more conserved outer membrane proteins may be selected from the group consisting of SEQ ID NO: 190-207 and any combination or fragment thereof.

The tick species may be Rhipicephalus sanguineus sensu lato and the one or more conserved tick proteins may be at least 75%, 80%, 85%, 90%, 95%, or 100% identical to a protein selected from the group consisting of SEQ ID NO: 29-56 and any combination or fragment thereof and the endosymbiont may be CLERS and the one or more conserved outer membrane proteins may be selected from the group consisting of SEQ ID NO: 221-232 and any combination or fragment thereof.

The tick species may be Amblyomma americanum and the one or more conserved tick proteins may be at least 75%, 80%, 85%, 90%, 95%, or 100% identical to a protein selected from the group consisting of SEQ ID NO: 113-140 and any combination or fragment thereof and the endosymbiont may be CLEAA and the one or more conserved outer membrane proteins may be selected from the group consisting of SEQ ID NO: 233-242 and any combination or fragment thereof.

The endosymbiont may be CLERA and the one or more conserved outer membrane proteins may be at least 75%, 80%, 85%, 90%, 95%, or 100% identical to a protein selected from the group consisting of SEQ ID NO: 208-220 and any combination or fragment thereof.

The tick species may be Ixodes scapularis and the one or more conserved tick proteins may be at least 75%, 80%, 85%, 90%, 95%, or 100% identical to a protein selected from the group consisting of SEQ ID NO: 57-84 and any combination or fragment thereof.

The tick species may be Ixodes pacificus and the one or more conserved tick proteins may be at least 75%, 80%, 85%, 90%, 95%, or 100% identical to a protein selected from the group consisting of SEQ ID NO: 85-112 and any combination or fragment thereof.

The tick species may be Haemaphysalis longicornis and the one or more conserved tick proteins may be at least 75%, 80%, 85%, 90%, 95%, or 100% identical to a protein selected from the group consisting of SEQ ID NO: 141-161 and any combination or fragment thereof.

The tick species may be Dermacentor variabilis and the one or more conserved tick proteins may be at least 75%, 80%, 85%, 90%, 95%, or 100% identical to a protein selected from the group consisting of SEQ ID NO: 162-189 and any combination or fragment thereof.

Implementations of a method of vaccinating an animal against hosting a tick may include administering to the animal a pharmaceutically effective amount of a bivalent vaccine including one or more conserved tick proteins from a tick species and one or more conserved outer membrane proteins of an endosymbiont of the tick species.

Implementations of a method of vaccinating an animal may include one, all, or any of the following:

Administering may include injecting or delivering the vaccine into a body or mucosa of the animal or oral application to the animal and where the bivalent vaccine further may include where the one or more conserved tick proteins and the one or more conserved outer membrane proteins each have a conservation level equal to or greater than 97%.

The tick species may be Rhipicephalus microplus and the one or more conserved tick proteins may be at least 75%, 80%, 85%, 90%, 95%, or 100% identical to a protein selected from the group consisting of SEQ ID NO: 1-28 and any combination or fragment thereof and the endosymbiont may be CLERM and the one or more conserved outer membrane proteins may be selected from the group consisting of SEQ ID NO: 190-207 and any combination or fragment thereof.

The tick species may be Rhipicephalus sanguineus sensu lato and the one or more conserved tick proteins may be at least 75%, 80%, 85%, 90%, 95%, or 100% identical to a protein selected from the group consisting of SEQ ID NO: 29-56 and any combination or fragment thereof and the endosymbiont may be CLERS and the one or more conserved outer membrane proteins may be selected from the group consisting of SEQ ID NO: 221-232 and any combination or fragment thereof.

The tick species may be Amblyomma americanum and the one or more conserved tick proteins may be at least 75%, 80%, 85%, 90%, 95%, or 100% identical to a protein selected from the group consisting of SEQ ID NO: 113-140 and any combination or fragment thereof and the endosymbiont may be CLEAA and the one or more conserved outer membrane proteins may be selected from the group consisting of SEQ ID NO: 233-242 and any combination or fragment thereof.

The endosymbiont may be CLERA and the one or more conserved outer membrane proteins may be at least 75%, 80%, 85%, 90%, 95%, or 100% identical to a protein selected from the group consisting of SEQ ID NO: 208-220 and any combination or fragment thereof.

The tick species may be Ixodes scapularis and the one or more conserved tick proteins may be at least 75%, 80%, 85%, 90%, 95%, or 100% identical to a protein selected from the group consisting of SEQ ID NO: 57-84 and any combination or fragment thereof.

The tick species may be Ixodes pacificus and the one or more conserved tick proteins may be at least 75%, 80%, 85%, 90%, 95%, or 100% identical to a protein selected from the group consisting of SEQ ID NO: 85-112 and any combination or fragment thereof.

The tick species may be Haemaphysalis longicornis and the one or more conserved tick proteins may be at least 75%, 80%, 85%, 90%, 95%, or 100% identical to a protein selected from the group consisting of SEQ ID NO: 141-161 and any combination or fragment thereof.

The tick species may be Dermacentor variabilis and the one or more conserved tick proteins may be at least 75%, 80%, 85%, 90%, 95%, or 100% identical to a protein selected from the group consisting of SEQ ID NO: 162-189 and any combination or fragment thereof.

The foregoing and other aspects, features, and advantages will be apparent to those artisans of ordinary skill in the art from the DESCRIPTION and DRAWINGS, and from the CLAIMS.

### BRIEF DESCRIPTION OF THE DRAWINGS

Implementations will hereinafter be described in conjunction with the appended drawings, where like designations denote like elements, and:

FIG. 1 is an amino acid distance metric for various proteins from a from a study of diversity of Rhipicephalus microplus ticks from a variety of geographic locations;

FIG. 2 is a chart of the amino acid similarity percentage among a set from proteins from R. microplus and Rhipicephalus. Sanguineus ticks;

FIG. 3 is a diagram of a selection and down-selection process for a set of Ixodes scapularis tick proteins and for the corresponding endosymbiont Rickettsia buchneri proteins;

FIG. 4 is a diagram of an implementation of a process of utilizing an animal model to assess the vaccine potentials for tick proteins and endosymbiont proteins;

FIG. 5 is a diagram of an implementation of a process of utilizing a rabbit, mouse, and white-tailed deer models to evaluate tick proteins and endosymbiont proteins to form bivalent vaccine candidates;

FIG. 6 is a timeline of various experimental activities in a process for evaluating a protein in a rabbit model;

FIG. 7 is a timeline of various experimental activities in a process for evaluating a protein in a white-tailed deer model;

FIG. 8 is a timeline of various experimental activities in a process for evaluating a protein in a mouse model;

FIG. 9 is a diagram of a first portion of a process for evaluating CLERM proteins for testing for use in a vaccine composition;

FIG. 10 is a diagram of a second portion of a process for evaluating CLERM proteins for testing for use in a vaccine composition;

FIG. 11 is a diagram of a process of selecting CLERM proteins for evaluation in a cattle study;

FIG. 12 is a timeline of various experimental activities in a process for evaluating a protein in a cattle model;

FIG. 13 is a graph showing locations of field collections of R. microplus in North and Central America; and

FIG. 14 is two diagrams of the overlap genes in the genomes of three CLERS genomes and a CLERM genome, CLERS genome, and Coxiella burnetti genome.

### DESCRIPTION

This disclosure, its aspects and implementations, are not limited to the specific components, assembly procedures or method elements disclosed herein. Many additional components, assembly procedures and/or method elements known in the art consistent with the intended anti-tick vaccine compositions and related methods will become apparent for use with particular implementations from this disclosure. Accordingly, for example, although particular implementations are disclosed, such implementations and implementing components may comprise any shape, size, style, type, model, version, measurement, concentration, material, quantity, method element, step, and/or the like as is known in the art for such anti-tick vaccine compositions and related methods, and implementing components and methods, consistent with the intended operation and methods.

Anti-tick vaccines target tick proteins. Anti-tick vaccines represent an alternative to acaricide-based tick control. The strategy of an anti-tick vaccine is to immunize a host (dog, cad, cattle, deer, or native mice) with tick antigens that stimulate a strong humoral IgG antibody response. Antibodies from the vaccinated animal then enter a tick via its blood meal and bind to corresponding protein targets in the gut, salivary gland, or ovary, negatively impacting the tick's physiology and sometimes causing mortality. An important outcome is to weaken ticks and reduce their reproductive output, so that successive tick generations are reduced to the point that disease transmission breaks down or becomes minimal. In selecting the proteins, prioritizing “concealed” antigens within ticks that a host immune system normally would not encounter is an important consideration because these protein(s) occur inside the tick body (midgut, hemolymph, ovary, etc.).

IgG antibodies in host blood retain their reactivity outside of the host (inside a tick) and, although not usually bactericidal by themselves, can also neutralize endosymbiont bacteria by binding and inhibiting abundant cell surface proteins on the bacteria. For example, host antibodies induced acutely negative effects and even mortality in Ixodes ricinus ticks fed on transgenic mice vaccinated with E. coli. IgG antibodies can cross the tick midgut and enter the hemolymph where they can bind to vaccine targets and persist through metamorphosis to nymphal or adult stages. IgG antibodies can even reach tissues such as salivary glands, ovaries and eggs, and muscle.

Anti-microbiota vaccines target bacterial species (some of which are endosymbionts) that live inside of ticks. A complementary strategy for anti-tick vaccines is to target tick microbiota that provision ticks with required nutrients. This approach can weaken or even kill the ticks and thereby disrupt pathogen transmission. Obligate members of the tick microbiome include bacterial endosymbionts that are maternally inherited. These maternally inherited bacterial endosymbionts are often the most dominant species within a tick and have great potential as targets of pest control in their own right. Endosymbionts exist because ticks feed solely on blood, an incomplete source of certain nutrients, especially B vitamins and amino acids. As such, these essential nutrients must be supplied by one or more endosymbiont genera present in the gut microbiome, including Coxiella, Francisella, and Rickettsia species. The paper by Duron et al. entitled “Evolutionary changes in symbiont community structure in ticks,” Molecular Ecology V. 26, p. 2904-2921 (2017, doi: 10.1111/mec. 14094), the disclosure of which is hereby incorporated entirely by reference, discusses many of the characteristics and distributions of various endosymbionts in various tick species. Coxiella-like endosymbionts (CLEs) are important in many genera of ticks including Rhipicephalus. Indeed, the CLE in R. microplus (referred to herein as “CLERM”) is required for development past the metanymph stage, and experimental elimination of the CLERM has demonstrated downregulation of multiple genes that are important for blood feeding and likely other factors. Experimental reduction of the CLE of the highly invasive Asian longhorned tick Haemaphysalis longicornis using tetracycline led to a decline in tick feeding behavior due to decreased serotonin, as the precursor for tick serotonin is provided via a shikimate pathway for chorismate in its CLE as described in the paper to Zhong et al, “Symbiont-regulated serotonin biosynthesis modulates tick feeding activity,” Cell Host & Microbe, V. 29, p. 1545-1557 (Oct. 13, 2021, doi.org/10.1016/j.chom.2021.08.011). Other tick species may also rely on precursors provided using a shikimate pathway including those that utilize Francisella species as endosymbionts as the existence of such a pathway is disclosed in the paper to Wagner et al., “Genomic characterization of Francisella tularensis and other diverse Francisella species from complex samples,” PLOS One, p. 1-30 (Oct. 12, 2022, doi.org/10.1371/journal.pone.0273273), the disclosure of which is hereby incorporated entirely herein by reference.

The obligate bacterial endosymbionts targeted in the vaccine implementations disclosed herein are generally intracellular and live inside arthropod cells of the Malpighian tubules, midgut, and ovaries. There is a high probability that antibodies targeting endosymbiont outer membrane proteins (OMPs) can enter tick cells and attack these bacteria. Multiple studies have demonstrated that antibodies against intracellular tick targets must pass through the cell membrane to be effective and this was experimentally confirmed using ISE6 tick cell culture. In fact, of seven published vaccine candidates focused solely on intracellular tick proteins, all produced significantly negative effects in ticks that fed on vaccinated hosts and the greatest efficacy reported (90%) was conveyed by the ribosomal PO antigen. The other reported intracellular candidates include subolesin, VDAC, ferretin-1, Hc-23, interphase cytoplasm foci protein (2G7), and troponin P27/30.

Mechanisms of antibody access to tick cells include 1) direct uptake into midgut epithelial cells via phagocytosis during blood meal processing, or 2) circulation in the tick hemolymph and distribution to other tissues where antibodies can subsequently enter cells. Another mechanism that further allows additional access of IgG antibodies to intracellular endosymbionts is that vaccines such as Bm86 and other midgut antigens are known for their ability to raise a strong IgG response that can severely damage tick midgut tissue, possibly by activating the complement cascade of the innate immune response. The resulting damaged or lysed cells should release endosymbiont bacteria, thus also exposing them to circulating IgG antibodies. Finally, endosymbionts spend part of their time outside of host cells and retain the ability to re-enter host cells, which suggests they are not intracellular at all times. They can circulate in hemolymph when migrating to ovarian tissues, as observed in the Rickettsia endosymbiont of whiteflies. This translocation is critical for ensuring vertical transmission to eggs and embryos. During periods outside of host cells, endosymbionts would be vulnerable to binding by host IgG antibodies received by the tick from a bloodmeal. A dual-action or bivalent vaccine that uses one or more tick proteins in combination with and one or more endosymbiont proteins may provide maximum access for endosymbiont and tick vaccine targets inside cells. By “in combination” is meant a vaccine where the composition includes both one or more tick proteins and one or more endosymbiont proteins which are administered at the same time. It also means a vaccine where the one or more tick proteins is included in a composition that is administered first followed by administration of a composition that contains the one or more endosymbiont protein and vice versa. The goal of the combination vaccine is that the IgG antibody effects of the vaccine will be felt by the host at the same time so as to enable ticks feeding from a host to acquire IgG antibodies for both the tick protein(s) and the endosymbiont protein(s) in their next single blood meal. In this document, the focus is on outer membrane proteins (OMPs) of endosymbionts as vaccine targets, because they will be most accessible to any host IgG antibodies that are able to enter tick cells or come into contact with endosymbionts outside of tick cells. However, it is possible in other vaccine implementations that other proteins internal to the endosymbionts may also be selected.

An additional consideration for anti-tick vaccines is to utilizes highly conserved protein targets for both the tick protein(s) and endosymbiont protein(s) selected. The first anti-tick vaccines for cattle used the Rhipicephalus microplus tick protein Bm86 and were implemented in the early 1990s. Vaccines marketed under the tradenames TICKGARD® by Pastoral AG of Adelaide, South Australia and GAVAC® by the Center for Genetic Engineering and Biotechnology of Camaguey, Cuba initially provided 55-100% control of R. microplus and Rhipicephalus annulatus (depending on the tick population), thereby reducing the number of acaricide treatments needed for a herd by 60% on average. However, the molecular basis of these first-generation vaccines was poorly understood and there are geographic differences in the efficacy of Bm86-based vaccines on different tick populations (resulting in 0-91% tick control), likely due to antigenic variation within key epitopes of this locus. FIG. 1 illustrates how, using a distance metric from a study of diversity of R. microplus ticks from a variety of geographic locations the Bm86 protein is one of the least conserved, but others like VDAC, AQP1, and VgR are highly conserved. Indeed, accounting for protein variation is important to prevent “vaccine escape” by different groups of a species population a problem that achieved widespread public awareness during the SARS-COV-2 vaccination campaign. The bivalent vaccine implementations disclosed herein target proteins that are highly conserved in both the tick and endosymbiont and, thus, useful against all populations of a single (or multiple) tick species. A bivalent vaccine can also be highly robust to immune escape, requiring mutations in both the tick and endosymbiont genomes for vaccine resistance to evolve, and therefore should have greater long-term efficacy against ticks.

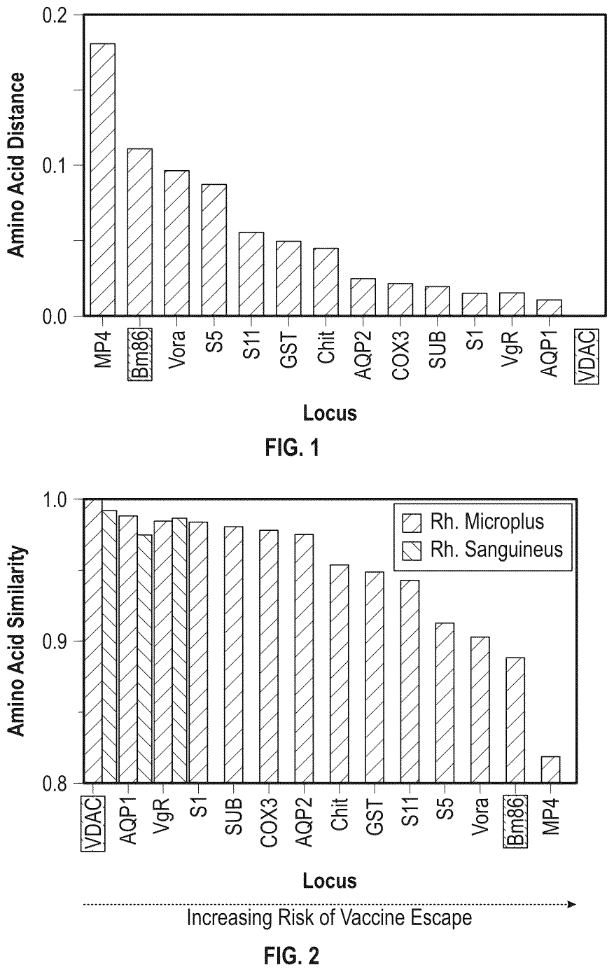

The present implementations of vaccine compositions designed for use in methods of vaccinating an animal against hosting a tick utilize at least one protein from at least one tick species at least one protein from at least one endosymbiont of the at least one tick species. Specific protein implementations for the endosymbionts utilize outer membrane proteins. Both the at least one protein from the tick and the at least one protein from the endosymbiont are selected based on how highly conserved they are among different populations of the tick species. In some implementations, the at least one protein from the tick and the at least one protein from the endosymbiont are highly conserved meaning greater than or equal to 97% amino acid similarity across many different tick populations. In some implementations, the tick protein targets may be selected based on an expectation that the protein targets will appear in 100% of ticks in the different tick populations. Referring to FIG. 2, a diagram showing the amino acid similarity between geographically dispersed tick populations of R. microplus and R. sanguineus shows a number of proteins above the 97% threshold and also indicates that the same protein is conserved across tick species to a similar degree (see VDAC). The lower the similarity percentage the lower the conservation of the protein which, as indicated in FIG. 2 also means there is a higher risk of vaccine escape for these proteins. Bm86 is illustrated to have less than a 90% amino acid conservation in the full-length protein, meaning that a vaccine based on it might have reduced efficacy in different tick populations allowing a large number of remaining ticks to successfully infest vaccinated hosts and pass on their specific genetic signature.

Examples of highly conserved tick proteins include, by non-limiting example, VDAC, AQP1, VgR, Serpin 1, Sub and Ribosomal PO. In the case of Vitellin, evidence that this gene is associated with Rickettsia endosymbionts has been observed. For Bm86, evidence that that the corresponding antibodies cause physical damage to the tick midgut has been observed. Preliminary data has also indicated three locations on the Bm86 protein where an IgG antibody can bind to it, further increasing the odds that an IgG antibody will find a positive interaction with the protein in a tick.

Non-limiting examples of outer membrane proteins (OMPs) of Coxiella and Rickettsia endosymbionts that may be utilized in a vaccine include OmpA, OmpB, OmpH, EF-tu, and YbgF. The OmpA and OmpB proteins are abundant on the outer membrane of Gram-negative bacteria. The OmpH and EF-tu proteins are expressed in the adult female life stage of R. microplus ticks. The YbgF protein is a vaccine target against the pathogen Coxiella burnetiid, and also found in endosymbiont bacteria.

Examples of tick species for which the vaccine compositions disclosed herein may be directed follow. In some implementations, a single vaccine composition may be used to target multiple tick species or a single tick species depending upon the tick protein used and the specific endosymbiont being targeted as well. Many other tick species other than those listed here that target various animals may be targeted in vaccine compositions using the principles disclosed herein.

In particular implementations, the tick species may be Rhipicephalus sanguineus. This species is commonly known as the brown dog tick, but is actually a complex of multiple different species of ticks with multiple variants in the United States and globally. In this document, the combination of species and variants referred to as brown dog ticks will be referenced as Rhipicephalus sanguineus sensu lato (s. l.) to include all of these variations. The obligate nutritional endosymbiont of R. sanguineus s. l. is a Coxiella-like bacteria referred to herein as the Coxiella-like endosymbiont of R. Sanguineus or CLERS. It is possible that an intact shikimate pathway exists with the CLERS organism. The hosts targeted with the vaccine implementation would be dogs and cats and likely would be administered as an injectable formulation or deliverable to mucosal surfaces through other mechanisms, such as a gene gun.

In particular implementations, the tick species may be Rhipicephalus microplus. This tick species is commonly referred to as a cattle fever tick. The obligate nutritional endosymbiont for R. microplus is the Coxiella-like endosymbiont of R. microplus or CLERM. It is possible that an intact shikimate pathway exists with the CLERM organism. In other implementations, the tick may be Rhipicephalus anmilatus or Rhipicephalus appendiculatus which are also commonly referred to as cattle fever ticks. The obligate nutritional endosymbiont for R. annulatus is referred to herein as the Coxiella-like endosymbiont of R. annulatus or CLERAN. The obligate nutritional endosymbiont for R. appendiculatus is referred to as the Coxiella-like endosymbiont of R. appendiculatus or CLERAP. The hosts for the vaccine implementations directed toward these ticks would be cattle and would likely be administered as an injectable formulation or deliverable to mucosal surfaces through other mechanisms, such as a gene gun.

In particular implementations, the tick species may be Ixodes scapularis. This tick species is commonly referred to as the black-legged tick or deer tick and is the primary vector of Lyme disease along with other pathogens in the United States. The obligate nutritional endosymbiont of I. scapularis is Rickettsia buchneri. No intact shikimate pathway is currently known for this endosymbiont. The target hosts for this tick species are white-tailed deer and small mammals including mice and rabbits. Because the target hosts are wildlife, edible oral baits are the method of administration of the vaccine composition for this tick species.

In particular implementations, the tick species may be Haemaphysalis longicornis. This tick species is commonly known as the Asian longhorned tick which has only recently been introduced into the United States but is spreading rapidly. While this tick species is known to transmit pathogens the primary issue experienced by hosts is that the number of ticks present on the host can become extremely high leading to weight loss, anemia, and reduced production in the case of various livestock. The obligate nutritional endosymbiont of H. longicornis is a Coxiella-like endosymbiont of H. longicornis or CLEHL. An intact shikimate pathway exists with the CLEHL organism. While cattle are the primary host target for the vaccine, there are other animals who may benefit from treatment. Administration of a vaccine directed toward this tick species would be primarily an injectable formulation or deliverable to mucosal surfaces through other mechanisms, such as a gene gun.

In particular implementations, the tick species may be Amblyomma americanum. This tick species is commonly known as the lone star tick. While this tick species can transmit diseases, the main health risk to humans of this tick is that it is the primary source of alpha-gal syndrome in humans. Alpha-gal syndrome is induced in a human host following a bite from a lone star tick because galactose-α-1,3-galactose or alpha-gal is produced in the saliva of the tick. The human body then produces antibodies to alpha-gal. Since alpha-gal is also found in red meat products, this means that the host's antibodies can cause a serious and potentially life-threatening allergy to the red meat products going forward (which is why the allergy is also called the red meat allergy or tick bite meat allergy). As the vaccine compositions disclosed herein are not directed specifically for human use, the ability to target the animals who host the lifecycle of A. americanum will be key. The obligate nutritional endosymbiont of A. americanum is a Coxiella-like endosymbiont of A. Americanum or CLEAA. It is possible that an intact shikimate pathway exists in the CLEAA organism. The administration method for the vaccine preparations is oral through baits distributed so wildlife can consume them.

In particular implementations, the tick species may be either Dermacentor variabilis or D. similis. These ticks are commonly known as the American dog tick. Historically the D. similis population on the west coast of the United States was regarded as a distinct population of D. variabilis but was recently reclassified as a separate species. However, past and current publications lump the two species together. The obligate nutritional endosymbiont for both tick species is a Francisella-like endosymbiont of D. variabilis or FLEDV. The host targets for the vaccine would be dogs and cats so the vaccine composition would be administered using an injectable formulation or deliverable to mucosal surfaces through other mechanisms, such as a gene gun.

In particular implementations, the tick species may be Ixodes pacificus. This tick is commonly known as the western black-legged tick and is the primary vector of Lyme disease on the west coast of the United States. The obligate nutritional endosymbiont for I. pacificus is a Rickettsia endosymbiont of I. pacificus or REIP. This endosymbiont is also known as Rickettsia monacensis str. Humboldt according to the paper by Alowaysi et al. entitled “Isolation and Characterization of a Rickettsia from the Ovary of a Western Black-legged Tick, Ixodes pacificus” Ticks Tick Borne Dis. V. 10, No. 4, p. 918-923 (June 2019, doi: 10.1016/j.ttbdis.2019.04.017) the disclosure of which is hereby incorporated entirely herein by reference. REIP is also known as Rickettsia species phylotype G021 according to the paper by Hunter et al., “The Rickettsia Endosymbiont of Ixodes pacificus Contains All the Genes of De Novo Folate Biosynthesis,” PLOS ONE V. 10, No. 12, p. 1-15 (Dec. 9, 2015, DOI: 10.1371/journal.pone.0144552, the disclosure of which is hereby incorporated entirely herein by reference. It is not believed that an intact shikimate pathway exists for this organism. A number of different hosts are used by this tick and so vaccination compositions may be administered orally via baits or perhaps via injections depending on the host animal targeted or deliverable to mucosal surfaces through other mechanisms, such as a gene gun.

Various compositions of vaccines that contain one or more tick proteins and one or more corresponding endosymbiont proteins can be constructed using the principles disclosed in this document. The various compositions may include one, all, or any of the following tick proteins or fragments thereof from Rhipicephalus microplus listed in Table 1 below with their corresponding SEQ ID NOs. These tick proteins are taken from strain Deutch F79, genome WOVZ00000000.1, with a mitochondrial genome of KP143546.

TABLE 1

| | | GenBank | |
|---|---|---|---|
| Protein | | Accession | SEQ ID |
| Symbol | Protein | Number | NO |
| AQP1 | Aquaporin-1 | AIT69684 | SEQ ID |
| | | | NO: 1 |
| AQP2 | Aquaporin-2 | ALJ75650.1 | SEQ ID |
| | | | NO: 2 |
| Bm86 | Glycoprotein | AAA30098 | SEQ ID |
| | (Bm86/Bm95) | | NO: 3 |
| Chit | Chitinase-1 | JAC58962 | SEQ ID |
| | | | NO: 4 |
| COX3 | mitochondrial | YP_008999531.1 | SEQ ID |
| | Cytochrome | | NO: 5 |
| | oxidase III | | |
| GST | Glutathione | AHH29555 | SEQ ID |
| | S-transferase | | NO: 6 |
| MP4 | Metalloprotease 4 | AAZ39660 | SEQ ID |
| | (reprolysin) | | NO: 7 |
| S-1 | Serine protease | AHC98652 | SEQ ID |
| | inhibitor 1 | | NO: 8 |
| S-5 | Serine protease | AHC98656 | SEQ ID |
| | inhibitor 5 | | NO: 9 |
| S-11 | Serine protease | AHC98662 | SEQ ID |
| | inhibitor 11 | | NO: 10 |
| Sub | Subolesin | AIS66740 | SEQ ID |
| | (akirin) | | NO: 11 |
| VDAC | Voltage-dependent | GU994210.1 | SEQ ID |
| | anion channel | | NO: 12 |
| Vt | Vitellin | XP_037287934.2 | SEQ ID |
| | | | NO: 13 |
| VgR | Vitellogenin | AUQ44344 | SEQ ID |
| | receptor | | NO: 14 |
| Vora | Voraxin | JX502818 | SEQ ID |
| | | | NO: 15 |
| P0 | Ribosomal P0 | UTH79929 | SEQ ID |
| | antigen | | NO: 16 |
| S-17 | Serine protease | AHC98668 | SEQ ID |
| | inhibitor 17 | | NO: 17 |
| FER2 | Ferretin-2 | XP_037282418 | SEQ ID |
| | | | NO: 18 |
| Ixochymostatin | Ixodes | XP_075726169.1 | SEQ ID |
| | scapularis | | NO: 19 |
| | cysteine-rich | | |
| | venom | | |
| | protein 6-like | | |
| PIXR2 | Reeler domain- | XP_037279600.1 | SEQ ID |
| | containing gut | | NO: 20 |
| | protein | | |
| TRO | Troponin I | XP_075732555.1 | SEQ ID |
| | | | NO: 21 |
| LIPF | gastric | XP_075743938.1 | SEQ ID |
| | triacylglycerol | | NO: 22 |
| | lipase | | |
| | (LOC120842767) | | |
| PM-CBP | Periotrophic | XP_037271582.2 | SEQ ID |
| | membrane | | NO: 23 |
| | binding | | |
| | chitin protein | | |
| CHIT | Chitinase | XP_037268021.2 | SEQ ID |
| | | | NO: 24 |
| CST | Cystatin | XP_037290527.1 | SEQ ID |
| | | | NO: 25 |
| Vg | Vitellogenin | XP_075731229.1 | SEQ ID |
| | | | NO: 26 |
| Pmy1 | Paramyosin 1 | XP_037284316.1 | SEQ ID |
| | | | NO: 27 |
| PmyX1 | Paramyosin | XP_037283529.1 | SEQ ID |
| | isoform X1 | | NO: 28 |

The various compositions may include one, all, or any of the following tick proteins or fragments thereof from Rhipicephalus sanguineus sensu lato listed in Table 2 below with their corresponding SEQ ID NOs. These tick proteins are taken from strain Rsan-2018, genome JABSTV010000000, with a mitochondrial genome of OQ184024.

TABLE 2

| Protein | | GenBank Accession | SEQ ID |
|---|---|---|---|
| Symbol | Protein | Number | NO |
| AQP1 | Aquaporin-1 | XP_037510823.1 | SEQ ID |
| | | | NO: 29 |
| AQP2 | Aquaporin-2 | XP_037518224.1 | SEQ ID |
| | | | NO: 30 |
| Bm86 | Glycoprotein | XP_037516405.1 | SEQ ID |
| | (Bm86/Bm95) | | NO: 31 |
| Chit | Chitinase-1 | XP_049270163.1 | SEQ ID |
| | | | NO: 32 |
| COX3 | mitochondrial | NP_008515.1 | SEQ ID |
| | Cytochrome oxidase | | NO: 33 |
| | III | | |
| GST | Glutathione S- | XP_037514402.1 | SEQ ID |
| | transferase | | NO: 34 |
| MP4 | Metalloprotease 4 | XP_037524104.1 | SEQ ID |
| | (reprolysin) | | NO: 35 |
| S-1 | Serine protease | XP_037500311.1 | SEQ ID |
| | inhibitor 1 | | NO: 36 |
| S-5 | Serine protease | XP_037527710.1 | SEQ ID |
| | inhibitor 5 | | NO: 37 |
| S-11 | Serine protease | XP_037500310.1 | SEQ ID |
| | inhibitor 11 | | NO: 38 |
| Sub | Subolesin (akirin) | XP_037520396.1 | SEQ ID |
| | | | NO: 39 |
| VDAC | Voltage-dependent | XP_037498097.1 | SEQ ID |
| | anion channel | | NO: 40 |
| Vt | Vitellin | XP_037527682.1 | SEQ ID |
| | | | NO: 41 |
| VgR | Vitellogenin | XP_037521270.1 | SEQ ID |
| | receptor | | NO: 42 |
| Vora | Voraxin | XP_037520117.2 | SEQ ID |
| | | | NO: 43 |
| P0 | Ribosomal P0 | XP_037506996.1 | SEQ ID |
| | antigen | | NO: 44 |
| S-17 | Serine protease | XP_037506918.1 | SEQ ID |
| | inhibitor 17 | | NO: 45 |
| FER2 | Ferretin-2 | XP_037504755.1 | SEQ ID |
| | | | NO: 46 |
| Ixochymostatin | Ixodes scapularis | XP_049270926.1 | SEQ ID |
| | cysteine-rich venom | | NO: 47 |
| | protein 6-like | | |
| PIXR2 | Reeler domain- | XP_037517706.1 | SEQ ID |
| | containing gut | | NO: 48 |
| | protein | | |
| TRO | Troponin I | XP_037507447.1 | SEQ ID |
| | | | NO: 49 |
| LIPF | gastric | XP_037514762.2 | SEQ ID |
| | triacylglycerol lipase | | NO: 50 |
| | (LOC120842767) | | |
| PM-CBP | Periotrophic | XP_037529905.1 | SEQ ID |
| | membrane binding | | NO: 51 |
| | chitin protein | | |
| CHIT | Chitinase | XP_037510731.1 | SEQ ID |
| | | | NO: 52 |
| CST | Cystatin | XP_037499815.1 | SEQ ID |
| | | | NO: 53 |
| Vg | Vitellogenin | XP_037527417.1 | SEQ ID |
| | | | NO: 54 |
| Pmy1 | Paramyosin 1 | XP_037499082.1 | SEQ ID |
| | | | NO: 55 |
| PmyX1 | Paramyosin isoform | XP_037503154.1 | SEQ ID |
| | X1 | | NO: 56 |

The various compositions may include one, all, or any of the following tick proteins or fragments thereof from Ixodes scapularis listed in Table 3 below with their corresponding SEQ ID NOs. These tick proteins are taken from strain PalLabHiFi, genome ABJB010000000, with a mitochondrial genome of MZ645749.

TABLE 3

| | | GenBank | |
|---|---|---|---|
| Protein | | Accession | SEQ ID |
| Symbol | Protein | Number | NO |
| AQP1 | Aquaporin-1 | XP_029845132.1 | SEQ ID |
| | | | NO: 57 |
| AQP2 | Aquaporin-2 | XP_029851087.2 | SEQ ID |
| | | | NO: 58 |
| Bm86 | Glycoprotein | XP_040358615.1 | SEQ ID |
| | (Bm86/Bm95) | | NO: 59 |
| Chit | Chitinase-1 | XP_040067705.1 | SEQ ID |
| | | | NO: 60 |
| COX3 | mitochondrial | YP_010535459 | SEQ ID |
| | Cytochrome oxidase | | NO: 61 |
| | III | | |
| GST | Glutathione S- | XP_029832933.2 | SEQ ID |
| | transferase | | NO: 62 |
| MP4 | Metalloprotease 4 | XP_042149676.1 | SEQ ID |
| | (reprolysin) | | NO: 63 |
| S-1 | Serine protease | XP_029832058.2 | SEQ ID |
| | inhibitor 1 | | NO: 64 |
| S-5 | Serine protease | XP_002402925.4 | SEQ ID |
| | inhibitor 5 | | NO: 65 |
| S-11 | Serine protease | XP_029832058.2 | SEQ ID |
| | inhibitor 11 | | NO: 66 |
| Sub | Subolesin (akirin) | XP_002414493.1 | SEQ ID |
| | | | NO: 67 |
| VDAC | Voltage-dependent | XP_040071973.1 | SEQ ID |
| | anion channel | | NO: 68 |
| Vt | Vitellin | XP_029835878.3 | SEQ ID |
| | | | NO: 69 |
| VgR | Vitellogenin | XP_029830196.2 | SEQ ID |
| | receptor | | NO: 70 |
| Vora | Voraxin | XP_040068701.2 | SEQ ID |
| | | | NO: 71 |
| P0 | Ribosomal P0 | XP_002399611.1 | SEQ ID |
| | antigen | | NO: 72 |
| S-17 | Serine protease | XP_040356992.2 | SEQ ID |
| | inhibitor 17 | | NO: 73 |
| FER2 | Ferretin-2 | XP_029844301 | SEQ ID |
| | | | NO: 74 |
| Ixochymostatin | Ixodes scapularis | XP_040077180 | SEQ ID |
| | cysteine-rich venom | | NO: 75 |
| | protein 6-like | | |
| PIXR2 | Reeler domain- | XP_040066572.1 | SEQ ID |
| | containing gut | | NO: 76 |
| | protein | | |
| TRO | Troponin I | XP_029849015.1 | SEQ ID |
| | | | NO: 77 |
| LIPF | gastric | XP_040069876.1 | SEQ ID |
| | triacylglycerol lipase | | NO: 78 |
| | (LOC120842767) | | |
| PM-CBP | Periotrophic | XP_029849845.2 | SEQ ID |
| | membrane binding | | NO: 79 |
| | chitin protein | | |
| CHIT | Chitinase | XP_040070242.3 | SEQ ID |
| | | | NO: 80 |
| CST | Cystatin | XP_042144652.1 | SEQ ID |
| | | | NO: 81 |
| Vg | Vitellogenin | XP_029826419.3 | SEQ ID |
| | | | NO: 82 |
| Pmy1 | Paramyosin 1 | XP_029836081.1 | SEQ ID |
| | | | NO: 83 |
| PmyX1 | Paramyosin isoform | XP_029834466.1 | SEQ ID |
| | X1 | | NO: 84 |

The various compositions may include one, all, or any of the following tick proteins or fragments thereof from Ixodes pacificus listed in Table 4 below with their corresponding SEQ ID NOs. These tick proteins are taken from strain CFI_29930, genome CAXMZB000000000, with a mitochondrial genome of PQ557592.

TABLE 4

| | | GenBank | |
|---|---|---|---|
| Protein | | Accession | SEQ ID |
| Symbol | Protein | Number | NO |
| AQP1 | Aquaporin-1 | CAN8011687.1 | SEQ ID |
| | | | NO: 85 |
| AQP2 | Aquaporin-2 | CAN7944931.1 | SEQ ID |
| | | | NO: 86 |
| Bm86 | Glycoprotein | CAN7980454.1 | SEQ ID |
| | (Bm86/Bm95) | | NO: 87 |
| Chit | Chitinase-1 | CAN7996797.1 | SEQ ID |
| | | | NO: 88 |
| COX3 | mitochondrial | WKW95282 | SEQ ID |
| | Cytochrome oxidase | | NO: 89 |
| | III | | |
| GST | Glutathione S- | CAN7985791.1 | SEQ ID |
| | transferase | | NO: 90 |
| MP4 | Metalloprotease 4 | CAN7993299.1 | SEQ ID |
| | (reprolysin) | | NO: 91 |
| S-1 | Serine protease | CAN8009594.1 | SEQ ID |
| | inhibitor 1 | | NO: 92 |
| S-5 | Serine protease | CAN8012911.1 | SEQ ID |
| | inhibitor 5 | | NO: 93 |
| S-11 | Serine protease | CAN8009594.1 | SEQ ID |
| | inhibitor 11 | | NO: 94 |
| Sub | Subolesin (akirin) | CAN7982237.1 | SEQ ID |
| | | | NO: 95 |
| VDAC | Voltage-dependent | CAN7946821.1 | SEQ ID |
| | anion channel | | NO: 96 |
| Vt | Vitellin | CAN8010991.1 | SEQ ID |
| | | | NO: 97 |
| VgR | Vitellogenin | CAN7990764.1 | SEQ ID |
| | receptor | | NO: 98 |
| Vora | Voraxin | CAN8008983.1 | SEQ ID |
| | | | NO: 99 |
| P0 | Ribosomal P0 | CAN8003031.1 | SEQ ID |
| | antigen | | NO: 100 |
| S-17 | Serine protease | CAN7942686.1 | SEQ ID |
| | inhibitor 17 | | NO: 101 |
| FER2 | Ferretin-2 | CAN7999213.1 | SEQ ID |
| | | | NO: 102 |
| Ixochymostatin | Ixodes scapularis | CAN8007949.1 | SEQ ID |
| | cysteine-rich venom | | NO: 103 |
| | protein 6-like | | |
| PIXR2 | Reeler domain- | CAN7942192.1 | SEQ ID |
| | containing gut | | NO: 104 |
| | protein | | |
| TRO | Troponin I | OZ285911.1 | SEQ ID |
| | | | NO: 105 |
| LIPF | gastric | CAN7985603.1 | SEQ ID |
| | triacylglycerol lipase | | NO: 106 |
| | (LOC120842767) | | |
| PM-CBP | Periotrophic | CAN8006181.1 | SEQ ID |
| | membrane binding | | NO: 107 |
| | chitin protein | | |
| CHIT | Chitinase | CAN8011681.1 | SEQ ID |
| | | | NO: 108 |
| CST | Cystatin | CAN8009801.1 | SEQ ID |
| | | | NO: 109 |
| Vg | Vitellogenin | CAN8010286.1 | SEQ ID |
| | | | NO: 110 |
| Pmy1 | Paramyosin 1 | CAN8001284.1 | SEQ ID |
| | | | NO: 111 |
| PmyX1 | Paramyosin isoform | CAN8000994.1 | SEQ ID |
| | X1 | | NO: 112 |

The various compositions may include one, all, or any of the following tick proteins or fragments thereof from Amblyomma americanum listed in Table 5 below with their corresponding SEQ ID NOs. These tick proteins are taken from strain F_SG_1, genome JBQHYB000000000.1, with a mitochondrial genome of NC_027609.

TABLE 5

| | | GenBank | |
|---|---|---|---|
| Protein | | Accession | SEQ ID |
| Symbol | Protein | Number | NO |
| AQP1 | Aquaporin-1 | KAK8762314.1 | SEQ ID |
| | | | NO: 113 |
| AQP2 | Aquaporin-2 | KAK8779666.1 | SEQ ID |
| | | | NO: 114 |
| Bm86 | Glycoprotein | KAK8757350.1 | SEQ ID |
| | (Bm86/Bm95) | | NO: 115 |
| Chit | Chitinase-1 | KAK8784849.1 | SEQ ID |
| | | | NO: 116 |
| COX3 | mitochondrial | YP_009159622 | SEQ ID |
| | Cytochrome oxidase | | NO: 117 |
| | III | | |
| GST | Glutathione S- | KAK8786077.1 | SEQ ID |
| | transferase | | NO: 118 |
| MP4 | Metalloprotease 4 | KAK8762780.1 | SEQ ID |
| | (reprolysin) | | NO: 119 |
| S-1 | Serine protease | KAK8762419.1 | SEQ ID |
| | inhibitor 1 | | NO: 120 |
| S-5 | Serine protease | KAK8768860.1 | SEQ ID |
| | inhibitor 5 | | NO: 121 |
| S-11 | Serine protease | KAK8762420.1 | SEQ ID |
| | inhibitor 11 | | NO: 122 |
| Sub | Subolesin (akirin) | KAK8786609.1 | SEQ ID |
| | | | NO: 123 |
| VDAC | Voltage-dependent | KAK8764890.1 | SEQ ID |
| | anion channel | | NO: 124 |
| Vt | Vitellin | KAK8768027.1 | SEQ ID |
| | | | NO: 125 |
| VgR | Vitellogenin | KAK8777062.1 | SEQ ID |
| | receptor | | NO: 126 |
| Vora | Voraxin | KAK8765932.1 | SEQ ID |
| | | | NO: 127 |
| P0 | Ribosomal P0 | KAK8758369.1 | SEQ ID |
| | antigen | | NO: 128 |
| S-17 | Serine protease | KAK8782247.1 | SEQ ID |
| | inhibitor 17 | | NO: 129 |
| FER2 | Ferretin-2 | KAK8780765.1 | SEQ ID |
| | | | NO: 130 |
| Ixochymostatin | Ixodes scapularis | KAK8770537.1 | SEQ ID |
| | cysteine-rich venom | | NO: 131 |
| | protein 6-like | | |
| PIXR2 | Reeler domain- | KAK8772316.1 | SEQ ID |
| | containing gut | | NO: 132 |
| | protein | | |
| TRO | Troponin I | KAK8766028.1 | SEQ ID |
| | | | NO: 133 |
| LIPF | gastric | KAK8765252.1 | SEQ ID |
| | triacylglycerol lipase | | NO: 134 |
| | (LOC120842767) | | |
| PM-CBP | Periotrophic | KAK8761278.1 | SEQ ID |
| | membrane binding | | NO: 135 |
| | chitin protein | | |
| CHIT | Chitinase | KAK8763642.1 | SEQ ID |
| | | | NO: 136 |
| CST | Cystatin | KAK8786974.1 | SEQ ID |
| | | | NO: 137 |
| Vg | Vitellogenin | KAK8769642.1 | SEQ ID |
| | | | NO: 138 |
| Pmy1 | Paramyosin 1 | KAK8780977.1 | SEQ ID |
| | | | NO: 139 |
| PmyX1 | Paramyosin isoform | KAK8787461.1 | SEQ ID |
| | X1 | | NO: 140 |

The various compositions may include one, all, or any of the following tick proteins or fragments thereof from Haemaphysalis longicornis listed in Table 6 below with their corresponding SEQ ID NOs. These tick proteins are taken from strain HaeL-2018, genome JABSTR000000000.1, with a mitochondrial genome of PQ380096.1.

TABLE 6

| | | GenBank | |
|---|---|---|---|
| Protein | | Accession | SEQ ID |
| Symbol | Protein | Number | NO |
| AQP1 | Aquaporin-1 | KAH9370733.1 | SEQ ID |
| | | | NO: 141 |
| AQP2 | Aquaporin-2 | KAH9380169.1 | SEQ ID |
| | | | NO: 142 |
| Bm86 | Glycoprotein | Not available | |
| | (Bm86/Bm95) | | |
| Chit | Chitinase-1 | KAH9370322.1 | SEQ ID |
| | | | NO: 143 |
| COX3 | mitochondrial | YP_009479059 | SEQ ID |
| | Cytochrome oxidase | | NO: 144 |
| | III | | |
| GST | Glutathione S- | KAH9377772.1 | SEQ ID |
| | transferase | | NO: 145 |
| MP4 | Metalloprotease 4 | Not available | |
| | (reprolysin) | | |
| S-1 | Serine protease | KAH9374893.1 | SEQ ID |
| | inhibitor 1 | | NO: 146 |
| S-5 | Serine protease | KAH9359844.1 | SEQ ID |
| | inhibitor 5 | | NO: 147 |
| S-11 | Serine protease | KAH9374893.1 | SEQ ID |
| | inhibitor 11 | | NO: 148 |
| Sub | Subolesin (akirin) | KAH9366378.1 | SEQ ID |
| | | | NO: 149 |
| VDAC | Voltage-dependent | KAH9364022.1 | SEQ ID |
| | anion channel | | NO: 150 |
| Vt | Vitellin | KAH9366454.1 | SEQ ID |
| | | | NO: 151 |
| VgR | Vitellogenin | KAH9360829.1 | SEQ ID |
| | receptor | | NO: 152 |
| Vora | Voraxin | Not available | |
| P0 | Ribosomal P0 | KAH9373747.1 | SEQ ID |
| | antigen | | NO: 153 |
| S-17 | Serine protease | Not available | |
| | inhibitor 17 | | |
| FER2 | Ferretin-2 | Not available | |
| Ixochymostatin | Ixodes scapularis | Not available | |
| | cysteine-rich venom | | |
| | protein 6-like | | |
| PIXR2 | Reeler domain- | KAH9369276.1 | SEQ ID |
| | containing gut | | NO: 154 |
| | protein | | |
| TRO | Troponin I | KAH9374109.1 | SEQ ID |
| | | | NO: 155 |
| LIPF | gastric | KAH9377209.1 | SEQ ID |
| | triacylglycerol lipase | | NO: 156 |
| | (LOC120842767) | | |
| PM-CBP | Periotrophic | KAH9363441.1 | SEQ ID |
| | membrane binding | | NO: 157 |
| | chitin protein | | |
| CHIT | Chitinase | KAH9369731.1 | SEQ ID |
| | | | NO: 158 |
| CST | Cystatin | Not available | |
| Vg | Vitellogenin | KAH9366389.1 | SEQ ID |
| | | | NO: 159 |
| Pmy1 | Paramyosin 1 | KAH9362511.1 | SEQ ID |
| | | | NO: 160 |
| PmyX1 | Paramyosin isoform | KAH9362371.1 | SEQ ID |
| | X1 | | NO: 161 |

The various compositions may include one, all, or any of the following tick proteins or fragments thereof from Dermacentor variabilis listed in Table 7 below with their corresponding SEQ ID NOs. These tick proteins are taken from strain Ectoservices, genome JBNOKL000000000, with a mitochondrial genome of NC_061217.

TABLE 7

| Protein | | GenBank Accession | SEQ ID |
|---|---|---|---|
| Symbol | Protein | Number | NO |
| AQP1 | Aquaporin-1 | XP_075536528.1 | SEQ ID |
| | | | NO: 162 |
| AQP2 | Aquaporin-2 | XP_075536657.1 | SEQ ID |
| | | | NO: 163 |
| Bm86 | Glycoprotein | XP_075548502.1 | SEQ ID |
| | (Bm86/Bm95) | | NO: 164 |
| Chit | Chitinase-1 | XP_075537933.1 | SEQ ID |
| | | | NO: 165 |
| COX3 | mitochondrial | YP_010286584.1 | SEQ ID |
| | Cytochrome oxidase | | NO: 166 |
| | III | | |
| GST | Glutathione S- | XP_075542452.1 | SEQ ID |
| | transferase | | NO: 167 |
| MP4 | Metalloprotease 4 | XP_075532073.1 | SEQ ID |
| | (reprolysin) | | NO: 168 |
| S-1 | Serine protease | XP_075555086.1 | SEQ ID |
| | inhibitor 1 | | NO: 169 |
| S-5 | Serine protease | XP_075525284.1 | SEQ ID |
| | inhibitor 5 | | NO: 170 |
| S-11 | Serine protease | XP_075555086.1 | SEQ ID |
| | inhibitor 11 | | NO: 171 |
| Sub | Subolesin (akirin) | XP_075553368.1 | SEQ ID |
| | | | NO: 172 |
| VDAC | Voltage-dependent | XP_075525107.1 | SEQ ID |
| | anion channel | | NO: 173 |
| Vt | Vitellin | XP_075528747.1 | SEQ ID |
| | | | NO: 174 |
| VgR | Vitellogenin | XP_075551372.1 | SEQ ID |
| | receptor | | NO: 175 |
| Vora | Voraxin | XP_075552980.1 | SEQ ID |
| | | | NO: 176 |
| P0 | Ribosomal P0 | XP_075545439.1 | SEQ ID |
| | antigen | | NO: 177 |
| S-17 | Serine protease | XP_075545345.1 | SEQ ID |
| | inhibitor 17 | | NO: 178 |
| FER2 | Ferretin-2 | XP_075533111.1 | SEQ ID |
| | | | NO: 179 |
| Ixochymostatin | Ixodes scapularis | XP_075534759.1 | SEQ ID |
| | cysteine-rich venom | | NO: 180 |
| | protein 6-like | | |
| PIXR2 | Reeler domain- | XP_075549897.1 | SEQ ID |
| | containing gut | | NO: 181 |
| | protein | | |
| TRO | Troponin I | XP_075545947.1 | SEQ ID |
| | | | NO: 182 |
| LIPF | gastric | XP_075540811.1 | SEQ ID |
| | triacylglycerol lipase | | NO: 183 |
| | (LOC120842767) | | |
| PM-CBP | Periotrophic | XP_075525552.1 | SEQ ID |
| | membrane binding | | NO: 184 |
| | chitin protein | | |
| CHIT | Chitinase | XP_075536725.1 | SEQ ID |
| | | | NO: 185 |
| CST | Cystatin | XM_075698355 | SEQ ID |
| | | | NO: 186 |
| Vg | Vitellogenin | XP_075527953.1 | SEQ ID |
| | | | NO: 187 |
| Pmy1 | Paramyosin 1 | XP_075536085.1 | SEQ ID |
| | | | NO: 188 |
| PmyX1 | Paramyosin isoform | XP_075530577.1 | SEQ ID |
| | X1 | | NO: 189 |

The various compositions may include one, all, or any of the following endosymbiont proteins or fragments thereof from CLERM listed in Table 8 below with their corresponding SEQ ID NOs. These tick proteins are taken from strain Deutch F79, genome DLUJ00000000.

TABLE 8

| | | GenBank | |
|---|---|---|---|
| Protein | | Accession | SEQ ID |
| Symbol | Protein | Number | NO |
| BamA | Outer membrane protein | WP_102156595.1 | SEQ ID |
| | assembly factor BamA | | NO: 190 |
| Omp-A like | Outer membrane beta-barrel | WP_102156608.1 | SEQ ID |
| | protein | | NO: 191 |
| BamD | Outer membrane protein | WP_102156616.1 | SEQ ID |
| | assembly factor BamD | | NO: 192 |
| OmpH | OmpH family outer | WP_102156618.1 | SEQ ID |
| | membrane protein | | NO: 193 |
| BamE/omlA | Outer membrane protein | WP_102156693.1 | SEQ ID |
| | assembly factor BamE | | NO: 194 |
| sdhD | Succinate dehydrogenase, | WP_102156742.1 | SEQ ID |
| | hydrophobic membrane | | NO: 195 |
| | anchor protein | | |
| SucB | 2-oxoglutarate dehydrogenase | WP_102156745.1 | SEQ ID |
| | complex dihydrolipoyllysine- | | NO: 196 |
| | residue succinyltransferase | | |
| EF-Tu | Elongation factor Tu | WP_102156778.1 | SEQ ID |
| | | | NO: 197 |
| LolA | Outer membrane lipoprotein | WP_408936549.1 | SEQ ID |
| | chaperone LolA | | NO: 198 |
| OmpA-like | Outer membrane beta-barrel | WP_157939808.1 | SEQ ID |
| | protein | | NO: 199 |
| BamA/TamA | BamA/TamA family outer | WP_102157017.1 | SEQ ID |
| | membrane protein | | NO: 200 |
| YbgF | Tol-pal system protein YbgF | WP_102157062.1 | SEQ ID |
| | | | NO: 201 |
| OmpA-like | OmpA family protein | WP_102157063.1 | SEQ ID |
| | | | NO: 202 |
| EF-Tu | Elongation factor Tu | WP_102156778.1 | SEQ ID |
| | | | NO: 203 |
| LbtU family, | Family siderophore porin, | WP_102157200.1 | SEQ ID |
| T4SS | partial | | NO: 204 |
| LolB | Lipoprotein insertase outer | WP_157939818.1 | SEQ ID |
| | membrane protein LolB | | NO: 205 |
| T1SS, TolC | TolC family outer membrane | WP_145996295.1 | SEQ ID |
| | protein | | NO: 206 |
| YbaB/EbfC | YbaB/EbfC_family_nucleoid- | WP_102156677.1 | SEQ ID |
| | associated_protein | | NO: 207 |

The various compositions may include one, all, or any of the following endosymbiont proteins or fragments thereof from CLERA listed in Table 9 below with their corresponding SEQ ID NOs. These tick proteins are taken from strain Klein Grass.

TABLE 9

| | | GenBank | |
|---|---|---|---|
| Protein | | Accession | SEQ ID |
| Symbol | Protein | Number | NO |
| BamA | Outer membrane protein | WP_304985729.1 | SEQ ID |
| | assembly factor BamA | | NO: 208 |
| Omp-A like | Outer membrane beta-barrel | WP_304986037.1 | SEQ ID |
| | protein | | NO: 209 |
| BamD | Outer membrane protein | WP_304985812.1 | SEQ ID |
| | assembly factor BamD | | NO: 210 |
| OmpH | OmpH family outer | WP_304985730.1 | SEQ ID |
| | membrane protein | | NO: 211 |
| BamE/omlA | Outer membrane protein | Not available | |
| | assembly factor BamE | | |
| sdhD | Succinate dehydrogenase, | WP_304985406.1 | SEQ ID |
| | hydrophobic membrane | | NO: 212 |
| | anchor protein | | |
| SucB | 2-oxoglutarate dehydrogenase | WP_304985005.1 | SEQ ID |
| | complex dihydrolipoyllysine- | | NO: 213 |
| | residue succinyltransferase | | |
| EF-Tu | Elongation factor Tu | WP_304985986.1 | SEQ ID |
| | | | NO: 214 |
| LolA | Outer membrane lipoprotein | Not available | |
| | chaperone LolA | | |
| OmpA-like | Outer membrane beta-barrel | WP_304985049.1 | SEQ ID |
| | protein | | NO: 215 |
| BamA/TamA | BamA/TamA family outer | WP_304985962.1 | SEQ ID |
| | membrane protein | | NO: 216 |
| YbgF | Tol-pal system protein YbgF | WP_304985902.1 | SEQ ID |
| | | | NO: 217 |
| OmpA-like | OmpA family protein | WP_304985901.1 | SEQ ID |
| | | | NO: 218 |
| EF-Tu | Elongation factor Tu | WP_304985986.1 | SEQ ID |
| | | | NO: 219 |
| LbtU family, | Family siderophore porin, | WP_304985093.1 | SEQ ID |
| T4SS | partial | | NO: 220 |
| LolB | Lipoprotein insertase outer | Not available | |
| | membrane protein LolB | | |
| T1SS, TolC | TolC family outer membrane | Not available | |
| | protein | | |
| YbaB/EbfC | YbaB/EbfC_family_nucleoid- | Not available | |
| | associated_protein | | |

The various compositions may include one, all, or any of the following endosymbiont proteins or fragments thereof from CLERS listed in Table 10 below with their corresponding SEQ ID NOs. These tick proteins are taken from strain Rsan-2018 with genome NIQB00000000.

TABLE 10

| | | GenBank | |
|---|---|---|---|
| Protein | | Accession | SEQ ID |
| Symbol | Protein | Number | NO |
| BamA | Outer membrane protein | AKQ33371.1 | SEQ ID |
| | assembly factor BamA | | NO: 221 |
| Omp-A like | Outer membrane beta-barrel | WP_251365494.1 | SEQ ID |
| | protein | | NO: 222 |
| BamD | Outer membrane protein | WP_251366098.1 | SEQ ID |
| | assembly factor BamD | | NO: 223 |
| OmpH | OmpH family outer | WP_251366371.1 | SEQ ID |
| | membrane protein | | NO: 224 |
| BamE/omlA | Outer membrane protein | WP_251366289.1 | SEQ ID |
| | assembly factor BamE | | NO: 225 |
| sdhD | Succinate dehydrogenase, | not available | |
| | hydrophobic membrane | | |
| | anchor protein | | |
| SucB | 2-oxoglutarate dehydrogenase | WP_251365959.1 | SEQ ID |
| | complex dihydrolipoyllysine- | | NO: 226 |
| | residue succinyltransferase | | |
| EF-Tu | Elongation factor Tu | WP_251365558.1 | SEQ ID |
| | | | NO: 227 |
| LolA | Outer membrane lipoprotein | not available | |
| | chaperone LolA | | |
| OmpA-like | Outer membrane beta-barrel | not available | |
| | protein | | |
| BamA/TamA | BamA/TamA family outer | WP_285896390.1 | SEQ ID |
| | membrane protein | | NO: 228 |
| YbgF | Tol-pal system protein YbgF | WP_251365919.1 | SEQ ID |
| | | | NO: 229 |
| OmpA-like | OmpA family protein | WP_251365917.1 | SEQ ID |
| | | | NO: 230 |
| EF-Tu | Elongation factor Tu | WP_251365558.1 | SEQ ID |
| | | | NO: 231 |
| LbtU family, | Family siderophore porin, | not available | |
| T4SS | partial | | |
| LolB | Lipoprotein insertase outer | not available | |
| | membrane protein LolB | | |
| T1SS, TolC | TolC family outer membrane | not available | |
| | protein | | |
| YbaB/EbfC | YbaB/EbfC_family_nucleoid- | WP_251366385.1 | SEQ ID |
| | associated_protein | | NO: 232 |

The various compositions may include one, all, or any of the following endosymbiont proteins or fragments thereof from CLEAA listed in Table 11 below with their corresponding SEQ ID NOs. These tick proteins are taken from strain F_SG_1 with genome NZ_CP007541.1.

TABLE 11

| | | GenBank | |
|---|---|---|---|
| Protein | | Accession | SEQ ID |
| Symbol | Protein | Number | NO |
| BamA | Outer membrane protein | WP_039669859.1 | SEQ ID |
| | assembly factor BamA | | NO: 233 |
| Omp-A like | Outer membrane beta-barrel | WP_039669982.1 | SEQ ID |
| | protein | | NO: 234 |
| BamD | Outer membrane protein | WP_052246319.1 | SEQ ID |
| | assembly factor BamD | | NO: 235 |
| OmpH | OmpH family outer | WP_039669860.1 | SEQ ID |
| | membrane protein | | NO: 236 |
| BamE/omlA | Outer membrane protein | not available | |
| | assembly factor BamE | | |
| sdhD | Succinate dehydrogenase, | WP_039669845.1 | SEQ ID |
| | hydrophobic membrane | | NO: 237 |
| | anchor protein | | |
| SucB | 2-oxoglutarate dehydrogenase | WP_039669848.1 | SEQ ID |
| | complex dihydrolipoyllysine- | | NO: 238 |
| | residue succinyltransferase | | |
| EF-Tu | Elongation factor Tu | WP_039669934.1 | SEQ ID |
| | | | NO: 239 |
| LolA | Outer membrane lipoprotein | not available | |
| | chaperone LolA | | |
| OmpA-like | Outer membrane beta-barrel | not available | |
| | protein | | |
| BamA/TamA | BamA/TamA family outer | not available | |
| | membrane protein | | |
| YbgF | Tol-pal system protein YbgF | not available | |
| OmpA-like | OmpA family protein | not available | |
| EF-Tu | Elongation factor Tu | WP_039669934.1 | SEQ ID |
| | | | NO: 240 |
| LbtU family, | Family siderophore porin, | WP_156962725.1 | SEQ ID |
| T4SS | partial | | NO: 241 |
| LolB | Lipoprotein insertase outer | not available | |
| | membrane protein LolB | | |
| TISS, TolC | TolC family outer membrane | not available | |
| | protein | | |
| YbaB/EbfC | YbaB/EbfC_family_nucleoid- | WP_052246316.1 | SEQ ID |
| | associated_protein | | NO: 242 |

While the sequences in the foregoing tables have included the whole proteins, in various implementations, specific peptide sequences or fragments from the whole proteins may be what are ultimately used in various vaccine compositions. As used herein, the term “fragment” refers to a portion of a protein substantially identical to any of the reference proteins disclosed herein (whether through sequence listing or otherwise) that retains the biological activity of the reference protein. In various implementations, the fragment may retain at least 50%, 75%, 80%, 90%, 95%, or 99% of the biological activity of the reference protein. Thus, this document contemplates that proteins from ticks and endosymbionts may be selected from peptides that are at least 75%, 80%, 85%, 90%, 95%, or 100% identical to the sequences disclosed herein or fragments thereof. Furthermore, in various implementations, the tick and/or endosymbiont proteins can be purified proteins or encoded by nucleic acids (DNA or RNA) introduced into the cells of the animal.

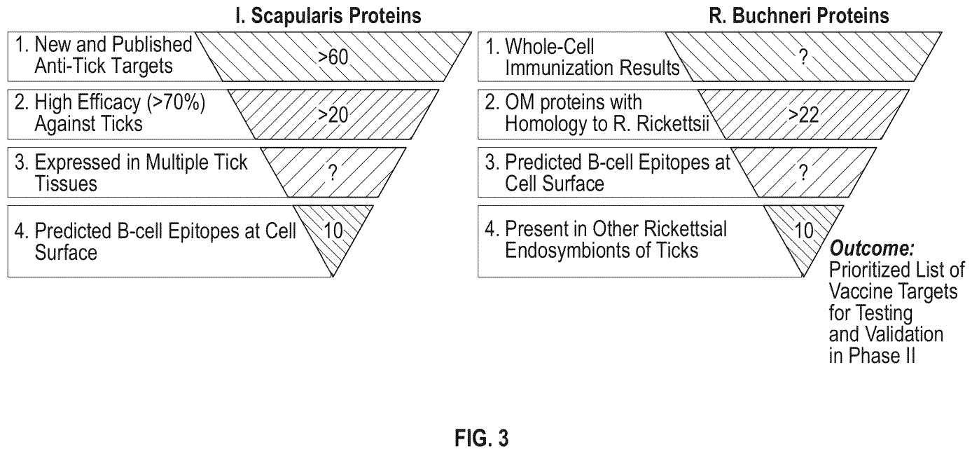

Various techniques and methods may be used to develop and determine the particular tick protein(s) and endosymbiont protein(s) selected for use in a vaccine composition like those disclosed herein. A diagram of the selection and down-selection process for the tick I. scapularis and the corresponding endosymbiont R. buchneri is illustrated in FIG. 3. In FIG. 3, the tick protein process is illustrated on the left and involves examining new and published anti-tick protein targets to come up with an initial list of proteins (here over 60 potential targets). The list of protein targets is then analyzed using various techniques disclosed later herein including animal testing to identify those targets that have greater than 70% efficacy in affecting a tick using various metrics including death, inability to reproduce, etc. This reduced list is then analyzed to pick those tick proteins that are identified as being expressed in multiple tick tissues, which significantly increases the odds that IgG antibodies from a blood meal will affect the tick. In various method implementations, an analysis of predicted B-cell epitopes at cell surfaces is also carried out to help refine the list of proteins to those who can attack the cell membrane structures directly and avoid selecting proteins that are mainly effective once they have entered a tick cell. Referring to the down-selection process on the right, a similar process is used to identify proteins for the endosymbiont R. buchneri with the difference that since less is known about what proteins from this organism will generate an IgG antibody in the first place, the process begins with whole cell immunization into a model animal followed by identifying the antigens produced. The process then involves linking the identified antigens to outer membrane (OM) proteins with homology to the related organism R. rickettsii (the organism that causes Rocky Mountain spotted fever in humans and is more well understood). Since outer membrane proteins are being targeted, the next step is to perform predictions of B-cell epitopes at the cell surface to screen out any proteins that do not appear to target a protein on the cell surface. The resulting list of proteins is then compared to a list of other Rickettsial endosymbionts in other tick species to evaluate the conservation of each protein to pick one with the highest likelihood of reaching a maximum number of ticks and tick species to reduce the odds of vaccine escape.

A screening of 30 adult female I. scapularis ticks obtained from various locations in the United States for the presence of R. buchneri indicated that this bacterium was present in all of the 30 ticks (100%). Thus, a focus on selecting proteins from R. buchneri for I. scapularis ticks would be a way to ensure conservation of the endosymbiont proteins.

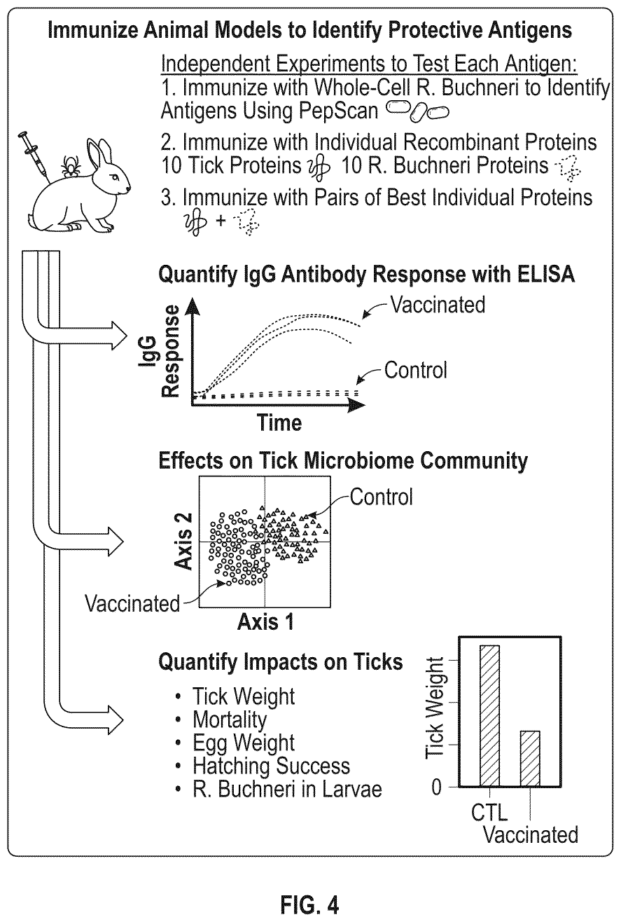

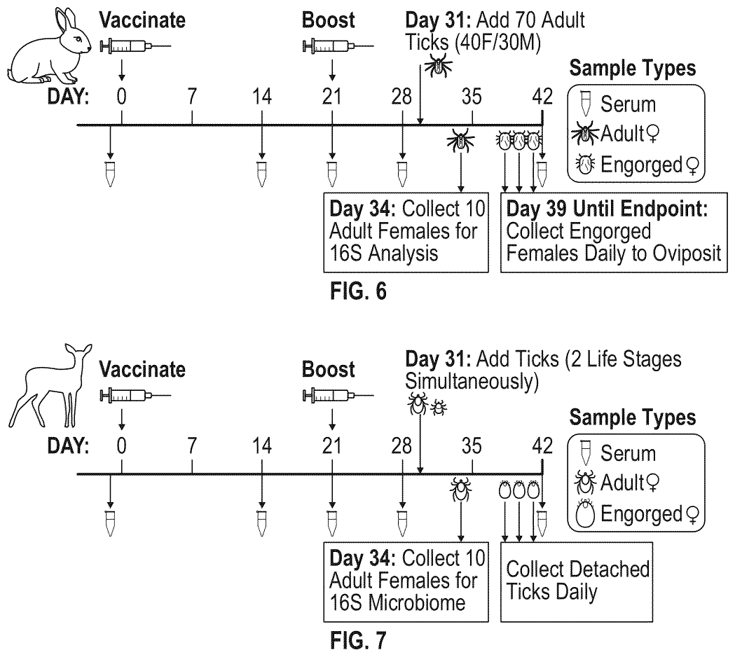

Further detail with respect to the R. buchneri process follows. The New Zealand white rabbit is a leading model for anti-tick vaccine studies. In a first trial, rabbits purchased from a commercial supplier are placed into one control group and two experimental groups (n=3 each) will be used to test two recombinant proteins (I. scapularis TnI and R. buchneri OmpB) using an adjuvant marketed under the tradename TITERMAX GOLD by TiterMax USA, Inc. of Norcross, Georgia. Intramuscular vaccinations with 50 micrograms of antigen and tick infestations will follow the timeline in FIG. 6 including evaluating immunization with whole-cell R. buchneri. Live adult I. scapularis can be obtained from the Oklahoma State University tick rearing facility. Serum from the rabbits can be drawn to quantify the IgG titer to each antigen, and 16S analysis can be performed using 10 partially fed females from each rabbit. Sera monitoring during the experiment ensures that vaccinated rabbits have raised a significantly higher level of IgG antibody compared to control animals. A suitable cutoff point in a particular implementation is an OD 450 nm reading of 0.35 for a 1/500 dilution of serum. With the adult ticks, the effects on the reproductive fitness of females based on weight, egg mass weight, and egg hatching success are then quantified including statistical comparisons between control vs. vaccinated groups. The foregoing process is summarized in FIG. 4.

If each antigen demonstrates effectiveness by itself (i.e., a significant reduction in the aforementioned measurements of female reproductive fitness for ticks feeding on vaccinated hosts compared to control group ticks), a second trial can be performed to vaccinate rabbits (n=3) using both vaccine protein targets (i.e., tick TnI+endosymbiont OmpB) concurrently. The two antigens will be delivered at the same time on opposite sides of the body together with TITERMAX GOLD adjuvant. All other methods for trial #2 can be identical to the first trial.

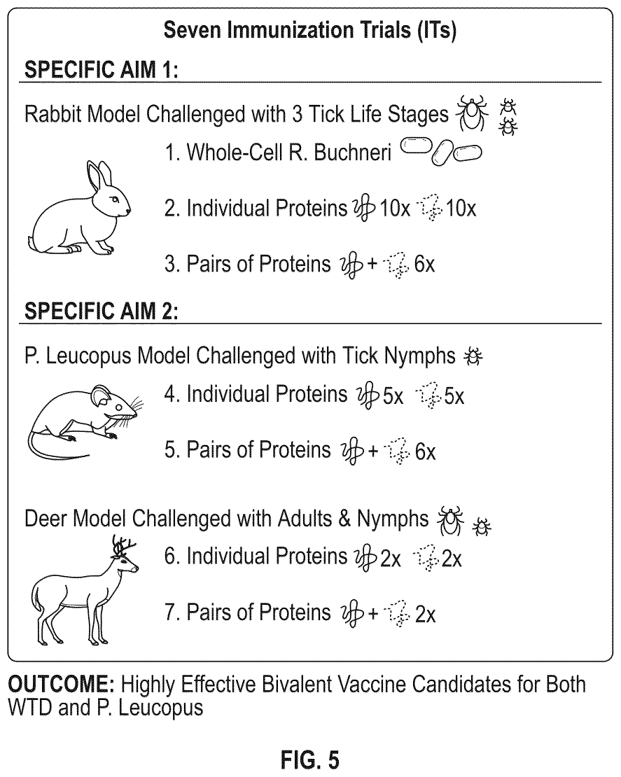

This process can then be adapted to testing the resulting tick and endosymbiont paired proteins for effectiveness in additional animal models. As illustrated in FIG. 5, following identification of one or more pairs of proteins in the rabbit model using the preceding approach, a mouse model and then a deer model are challenged. The mouse model focuses specifically on observing the effects of the vaccine on tick nymphs feeding on mice because the life cycle of I. scapularis involves the ticks feeding on small mammals first and then moving to deer as they age and become adults.

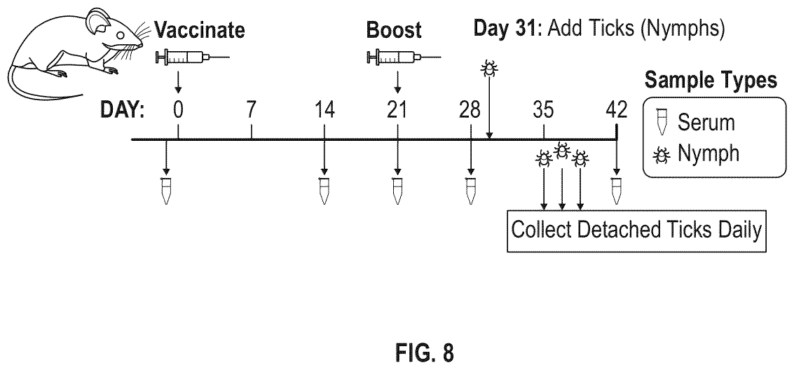

A representative testing approach and schedule for the mice (P. leucopus) is illustrated in FIG. 8. In this testing approach, up to five I. scapularis and five R. buchneri proteins can be tested in Peromyscus leucopus mice using the most protective proteins from IT2 and IT3. A small pilot study with one full-length protein can be used to determine an appropriate subcutaneous dose for mice (˜20 micrograms of antigen) and verify immunogenicity using TiterMax Gold adjuvant. Mice can then be placed into 10 experimental and 4 control groups (n=3/group; 42 total). Individual recombinant proteins are used to immunize each subject, with a booster at day 21 (FIG. 8); control animals will receive 1×PBS in TITERMAX GOLD adjuvant. A small blood sample (100 microliters) can be collected at five time periods (FIG. 8) during the experiment to quantify antibody responses through time using enzyme-linked immunosorbent assay (ELISA). At the end of the experiment, the mice are euthanized and serum collected via a terminal blood draw. On day 31, each mouse can be challenged with 20 I. scapularis nymphs. Mice will be sedated briefly with isoflurane so ticks can be applied to the dorsal neck area; ticks will be attached for the next 4 hours while mice are kept in wire-mesh tubes wrapped with stockinette. Ticks will be allowed to feed until they engorge and/or drop from the host, about 4 days for the nymphal stage. Detached ticks are collected daily and weighed. The effects of immunization on bloodmeal processing efficiency by measuring tick mortality, days to detachment, and weight upon detachment are then quantified for the various proteins being tested.

Following the testing in mice, testing of the best performing tick and endosymbiont proteins can then be conducted using vaccinations of the tick proteins and endosymbiont proteins individually followed by testing using vaccinations of the tick proteins and endosymbiont proteins combined. Young deer white-tailed deer (WTD) can be placed into 3 experimental and 1 control groups (n=3/group; 12 total). Deer will be given intramuscular vaccinations (200-400 micrograms of protein) and tick infestations will follow the timeline in FIG. 7 using TITERMAX GOLD adjuvant. A 10 mL blood sample will be obtained at five time points during the experiment to quantify antibody responses through time using ELISA. Sera monitoring during the experiment is used to determine if vaccinated WTD raise a significantly higher level of IgG antibody than control animals; a suitable cutoff is an OD 450 nm of 0.35 for a 1/500 dilution of serum. A second booster will be given if needed, which will extend the timeline by 3 weeks.

On day 31, each deer can be challenged with 100 I. scapularis nymphs and 100 adults (50 females/50 males). Ten of the adult female I. scapularis from each WTD are collected after three days of feeding to analyze their microbiome community, leaving 40 females/50 males for the reproduction study. Host animals will be prepared for infestation by shaving hair from a small dorsal area of the thorax and gluing pouches to the skin. Ticks will be introduced via the resealable pouch opening and allowed to feed until they engorge and/or drop from the host, about 4 days for juvenile stages and about 6-10 days for adult females. Detached ticks are removed daily and weighed. Adult female ticks are incubated for a study of reproductive fitness as the same as previously described. DNA is then extracted from pooled samples of newly hatched I. scapularis larvae to perform PCR-based relative quantification of R. buchneri (CKP3) in larvae originating from females fed on immunized vs. control hosts. If individual antigens demonstrate strong effects (i.e., a significant reduction in the aforementioned measurements of female reproductive fitness for ticks feeding on vaccinated hosts compared to control group ticks), a second trial using vaccination of pairs of protein targets concurrently (combined vaccine) is conducted. Deer are placed into 3 experimental and 1 control groups (n=3/group; 12 total). Intramuscular vaccinations with an appropriate amount of each antigen (200-400 micrograms) are delivered at the same time on opposite sides of the body using TITERMAX GOLD adjuvant (half volume/site). Tick infestations and 10 mL blood draws are then conducted using the timeline in FIG. 7. Sera monitoring continues during the experiment to determine if vaccinated WTD raise a significantly higher level of IgG antibody than control animals and whether an additional booster is needed. The timeline of the trial of the combined vaccine is then extended to perform repeated tick infestations (3 total) on vaccinated and control deer, with each infestation separated by 1 month (not shown in FIG. 7) to evaluate the ability of the combined vaccine to protect against repeated tick infestations.

The foregoing study begins with rabbit models and proceeds to mouse and then white-tailed deer models. However, in other implementations, the study can begin with the mouse model as outlined to test the tick bacterial endosymbiont R. buchneri for its potential efficacy as an anti-tick vaccine. Specifically, the study determines whether whole R. buchneri cells are immunoreactive in a laboratory mouse model and followed by a challenge of mice with I. scapularis ticks to quantify any negative effects on the ticks (i.e., mortality, low rate of weight increase, and/or changes in feeding duration) during blood feeding.

R. buchneri can be grown in tick cell culture using existing methods. After growing R. buchneri in I. scapularis ISE6 cell culture, live bacteria are released from the eukaryotic ISE6 cells. Whole R. buchneri cells can then either be used intact (heat killed) or lysed in preparation for mouse vaccinations. The target immunization for each mouse is the equivalent of approximately 1×10⁶ R. buchneri cells. The number of R. buchneri cells is estimated using a real-time polymerase chain-reaction (PCR) assay to quantify a single copy locus from the R. buchneri core genome. The same locus is placed in a synthetic “G-block” DNA strand that serves as a PCR control with a known copy number (1 copy per synthetic G-block). DNA from the R. buchneri whole-cell preparation is diluted serially and analyzed in multiple PCR replicates, which can be compared against the G-block control to obtain a relative quantification of the number of R. buchneri core genome copies. The number of genome copies is directly correlated with the number of R. buchneri cells per given volume.

In advance of the mouse study, a verification is conducted on the laboratory colony of I. scapularis ticks to prove that they actually carry the R. buchneri endosymbiont. This is done by extracting DNA from a sample of I. scapularis ticks from the lab colony and using the same real-time PCR assay described above.

Ten experimental mice (5 females and 5 males) can be given a subcutaneous immunization with the equivalent of approximately 1×10⁶ R. buchneri cells in 1×PBS using TITERMAX GOLD adjuvant; six control animals (5 females/5 males) will receive only 1×PBS with TITERMAX GOLD adjuvant (10 mice total). The experimental timeline is as shown in FIG. 8; a single booster is given on day 21. Blood samples (<100 microliters) from each mouse will be collected at five timepoints to quantify total IgG titer to each antigen. Sera monitoring continues during the experiment to determine if vaccinated animals raise a significantly higher level of IgG antibody compared to controls; a suitable cutoff point is an OD 450 nm reading of 0.35 for a 1/500 dilution of serum. Each mouse can then be challenged with ticks to evaluate any negative effects to the ticks. At the end of the experiment, mice will be euthanized and serum collected from a final blood draw (1-5 mL per mouse). On day 31, each mouse is challenged with the nymph stage of I. scapularis (n=20 ticks per mouse). Host animals are prepared for infestation by shaving hair from a small dorsal area of the thorax and gluing pouches to the skin. Ticks are introduced via the resealable pouch opening and allowed to feed until they engorge and/or drop from the host, about 4 days for typical nymphs. Detached ticks are removed daily and weighed. Ticks will be incubated at 22 C under 85% humidity in a 16:8 hr light/dark cycle to evaluate the molting rate of nymphs. This incubation takes about 2 months and is a good predictor of vaccine efficacy.

Data collected during the study includes total IgG ELISA. The sera collected at 1-2 week intervals are analyzed with ELISA to evaluate total IgG responses. Additional data includes mouse skin reactions at the bite site (photos) and spleens will be saved to attempt recovering antibodies specific to individual R. buchneri OMPs (i.e., OmpB, BamD, etc.). The effects of tick feeding from vaccinated vs control mice with the statistical analysis of the following measurements is also calculated including tick mortality, tick feeding duration, tick weight upon detachment, and the molt rate of nymphs. Any impacts to the R. buchneri symbionts are also assessed by extracting DNA from I. scapularis nymphs upon completion of the study. Using these DNA samples, the real-time PCR described above (including the synthetic G-block) is used to perform relative quantification of the R. buchneri symbiont in whole ticks after feeding on immunized vs. control hosts.

Additional testing can be carried out including screening against whole-cell lysates of R. buchneri in Western blots to confirm total IgG antibody reactivity with endosymbiont proteins. This analysis includes Western blots containing several types of negative controls. Pre-vaccination mouse serum (day 0) is screened against whole-cell lysates of R. buchneri in Western blots to examine the expected lack of IgG antibodies that are reactive with endosymbiont proteins. Serum from non-vaccinated control mice is screened against whole-cell lysates of R. buchneri in Western blots to examine the expected lack of IgG antibodies binding to endosymbiont proteins. Eukaryotic cell lysate from pure ISE6 tick cell culture is prepared to reveal if any tick proteins from cell culture inadvertently contaminated the R. buchneri whole cells during the isolation procedure and thus were included in the vaccine formulation. Single whole-length recombinant protein (i.e., OmpB) is tested against mouse sera using ELISA to evaluate specific IgG reactivity. For ticks that show obvious external signs of distress (discoloration, bloating), microscopy of the midgut is used to identify gross negative effects. Microscopy using immunofluorescence assay (IFA) to image antibody attachment on the outer membrane of R. buchneri cells is also used. Live, intact cells of R. buchneri isolated from ISE6 cell culture is exposed to mouse antibodies. If mouse antibodies are capable of attaching to endosymbiont OMPs they may neutralize the activity of these proteins. If neutralization is highly effective, the R. buchneri cells can be expected to have a decreased ability to enter uninfected ISE6 tick cells. This can be tested by adding antibody-treated R. buchneri cells to a culture of live ISE6 cells and looking at the entry rate of tick cells through time. This is then compared to a baseline control that uses R. buchneri cells not exposed to antibodies that will retain their ability to enter ISE6 tick cells at a high rate.

The main outcomes of the study are 1) ELISA data from IgG antibodies evaluating the immunogenicity of R. buchneri cells, 2) data to determine if vaccination reduces I. scapularis feeding efficiency (i.e., feeding time and weight) and reproductive fitness of females (i.e., egg mass weight and hatching success). Because bacterial endosymbionts are essential for tick bloodmeal processing, immunization with R. buchneri whole cells should have a strong chance of being protective against ticks. Success criteria for the study include any or all of the following: significant tick mortality after feeding on vaccinated hosts versus control hosts; significantly lower mean weight of ticks after feeding on vaccinated hosts versus control hosts; any statistically significant changes to tick feeding behavior, such as early detachment or prolonged feeding that are significantly different from ticks fed on vaccinated hosts versus control hosts; an ELISA reading (OD 450 nm) that is significantly greater in the serum of vaccinated hosts versus control hosts; a reduction in the quantity of R. buchneri cells remaining in ticks that fed on vaccinated hosts versus control hosts; a reduction in the rate at which antibody-treated R. buchneri cells can enter live ISE6 tick cells through time, as compared to a baseline control using untreated R. buchneri cells added to ISE6 tick cells.

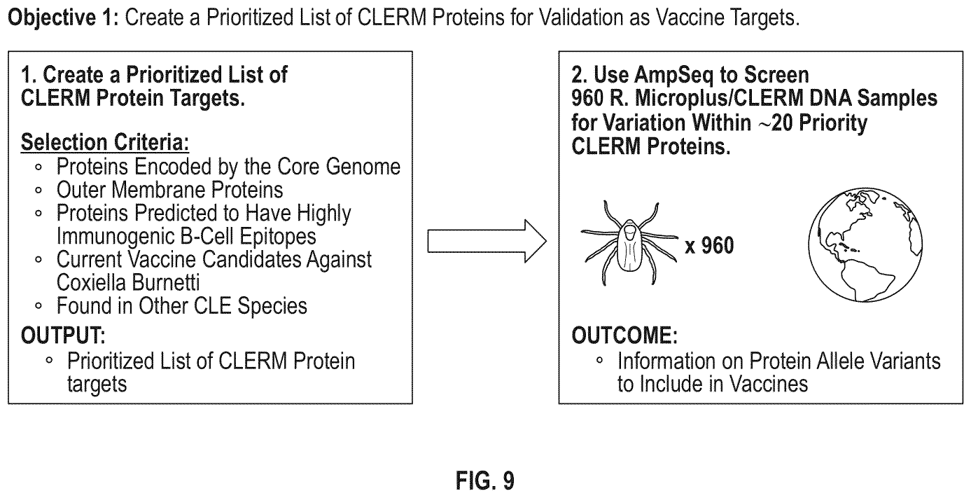

The foregoing analyses have applied to I. scapularis ticks. Similar application of the principles disclosed can be applied to those ticks that carry cattle fever (cattle fever ticks like any previously disclosed). Because the CLERM proteins are not as well understood, the analysis is laid out in FIG. 9 where a first step is to create a prioritized list of CLERM protein targets. The selection criteria may include, by non-limiting example, proteins encoded by the core genome, outer membrane proteins, proteins predicted to have highly immunogenic B-cell epitopes, current vaccine candidates against Coxiella burnetii, or highly conserved proteins found in other CLE species in other ticks. With this prioritized list of CLERM protein targets amino acid variation within 20 of these proteins will be examined across a large R. microplus/CLERM dataset (n=960) using AmpSeq. AmpSeq is a PCR-based, high-throughput sequencing approach facilitating inexpensive, multiplexed sequencing at many genomic locations (several hundred) across multiple samples simultaneously. Because the output is nucleotide sequences from DNA templates, it provides a means to identify both known and unknown mutations at genomic regions of interest. For example, it can be leveraged to assess certain mutations (i.e., single nucleotide polymorphisms (SNPs)) associated with specific strains, species, or phenotypes. Multiplexed PCRs for each sample are normalized and sequenced, providing hundreds to thousands of reads per PCR target, and reads are then aligned to a reference using the Amplicon Sequence Analysis Pipeline (ASAP). The outcome of the AmpSeq analysis is information on protein allele variants for the about 20 proteins being considered to be included in a vaccine composition.

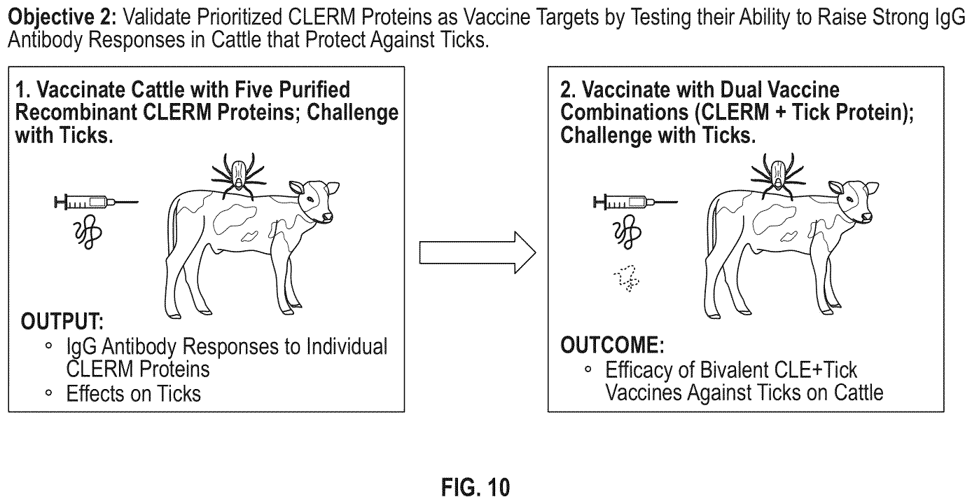

With about 5 top CLERM proteins identified from the previous analysis, the ability of the CLERM proteins to induce an IgG antibody response in cattle is assessed as illustrated in FIG. 10. The use of recombinant proteins rather than native proteins from cultured cells may be used because of the difficulty in culturing CLEs outside of their hosts. As illustrated in FIG. 10, a second round of vaccinations and tick trials is carried out in cattle using bivalent formulations that contain both the selected CLERM protein(s) and, in this implementation, the tick VDAC protein, which has high efficacy against tick infestation. This work will provide a foundational understanding of the immunogenic potential of the selected CLERM proteins in concert with a tick protein and evaluate efficacy of R. microplus control on cattle. Importantly, because CLEs like or related to CLERM are widespread across many tick species, and often ubiquitous within them, a CLERM-based vaccine (or a vaccine using a conserved protein from any CLE type disclosed herein) have great potential as a tool to control multiple tick species. This includes other tick species with CLEs that also transmit livestock pathogens, including R. annulatus and H. longicornis, and ticks that transmit important human pathogens, such as R. sanguineus, which transmits Rickettsia rickettsii (Rocky Mountain Spotted Fever) and other pathogens to humans and animals.

The process of selecting the CLERM proteins is provided in greater detail in the diagram illustrated in FIG. 11. Starting from the CLERM core genome a prioritized list of anti-CLERM protein vaccine targets is created using the process summarized in FIG. 11. First, proteins are considered which are encoded by the CLERM core genome. All intact proteins encoded by genes present in the CLERM core genome are first identified. As pseudogenes can accumulate during CLE evolution, genes encoding for partial length proteins are filtered out to ensure only full-length open reading frames are retained for consideration.

Outer membrane proteins encoded by the CLERM core genome are then identified along with modeling protein folding and 3D structure using AlphaFold1 to identify cell surface domains. Epitope prediction tools are then used (IEDB131, ABCpred132, PSORTb128, and VaxiJen113) to identify CLE OMPs that likely contain highly immunogenic B-cell epitopes. Epitopes are then mapped onto 3D models to prioritize those that are surface exposed. As a quality control step, CLE proteins that have low allergenicity values for cattle are then screened and selected.

As part of selection, prioritizing vaccine candidates currently under development against C. burnetii that are also found in the CLERM is also done. Leading C. burnetii vaccine targets include proteins such as OmpA, OmpH, YbgF, and translation elongation factor Tu, all of which are encoded in the CLERM genome and share reasonable levels of amino acid identity with C. burnetii. During the selection process, a comparison with available genomes of CLEs from other tick species to the CLERM genomes in the study is carried out to evaluate conservation of target proteins across CLE species. This will help identify targets that may be useful against multiple tick species.

After doing the foregoing, a prioritized list of about 20 proteins to screen across a large (n=960) R. microplus/CLERM DNA collection using AmpSeq is then selected. The resulting data provides an essential understanding of variation in the CLERM protein candidates across diverse R. microplus populations in different geographies in the US and around the world. In this analysis, the best five CLE proteins will be moved along to cattle/tick trials.

The next step is to test the five best CLERM OMP candidates for their ability to raise a strong IgG antibody response in a bovine model and protect against tick infestations. The vaccination trials in cattle that will allow us to: 1) compare antigenicity across individual CLERM proteins, and 2) evaluate the ability of single CLERM antigens combined with the VDAC4 tick antigen to protect vaccinated cattle against ticks. Recombinant E. coli expression plasmids that encode the CLERM proteins and a histidine tag (his6) on the N-terminus of each protein using a cloning system marketed under the tradename GATEWAY by ThermoFisher of Waltham, Massachusetts. Proteins are expressed in E. coli Rosetta cells from Millipore Sigma, lysed, and purified using a nickel affinity column marketed under the tradename HISPREP FF 16/10 by GE Life Sciences of Marlborough, Massachusetts; the latter captures any proteins via the his6 tag. Bound proteins are eluted using an imidazole concentration gradient. Eluted fractions containing the protein of interest are identified by SDS-PAGE and pooled, dialyzed, quantified with a BCA assay, and confirmed with Western blot analysis using a monoclonal antibody specific to the his6 tag. Because the analysis focuses on CLERM OMPs (OmpA, etc.), some or all of the target proteins may be insoluble. Insoluble proteins may be successfully purified by adding an E. coli thioredoxin solubility tag paired with the his6 tag, or by chemical solubilization using N-Lauroylsarcosine. Proteins purified using both methods are amenable for mammal immunization.

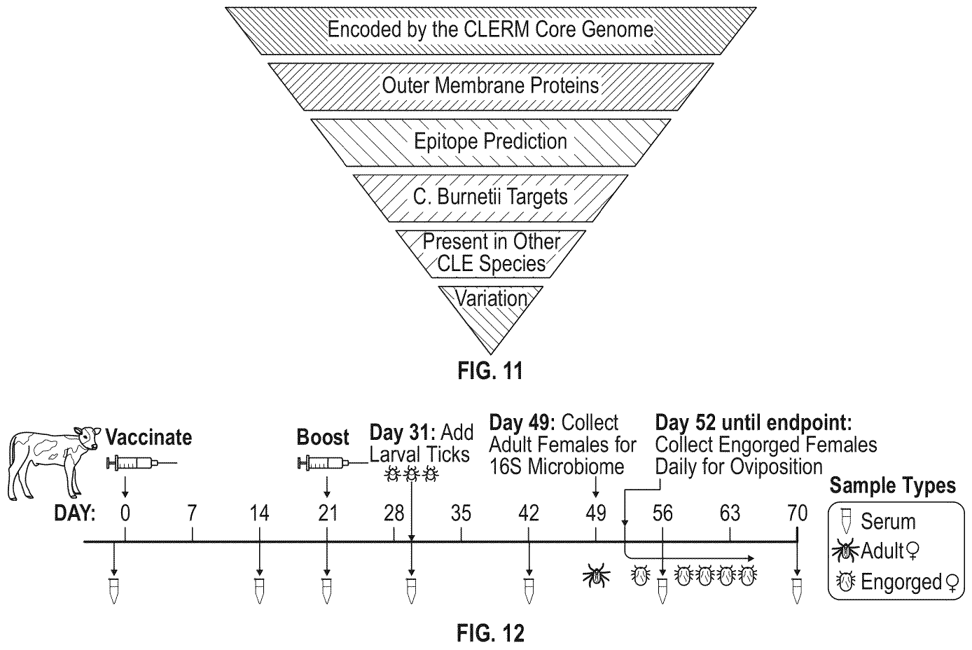

In the first round of vaccine/tick trials and referring to FIG. 12, five individual CLERM recombinant proteins' ability to stimulate an IgG antibody response in cattle and protect against experimental tick infestation is evaluated. Each of the five antigens will be tested separately (sequentially, not parallel) in groups of five experimental cattle (Bos taurus outbred European breeds), for a total of 25 experimental cows plus one set of five control cows for comparison to all experimental groups (30 animals total in trial 1). To form the vaccine, 100 micrograms of purified recombinant protein is mixed in 1 mL of a VG adjuvant marketed under the tradename MONTANIDE ISA 201 by Seppic of Colombes, France. This adjuvant is recognized for its ability to rapidly stimulate an IgG response in cattle. The vaccination/tick challenge experiment will take up to 70 days as indicated in FIG. 12, with initial vaccination on Day 0 and a booster on Day 21. Larval ticks of R. microplus are added 10 days later (Day 31) to coincide with high IgG levels. Adult females are collected after engorgement (Day 52+); engorgement is anticipated to take longer for ticks fed on vaccinated hosts and this timeline accounts for this. At multiple timepoints, 10 mL blood samples are collected as illustrated in FIG. 12 for antibody testing with ELISA.

For tick infestations on cattle, 0.5 g of larval R. microplus (˜10,000 ticks) from a specified lab colony are separated into two cloth chambers glued to shaved skin patches. Ticks remain on cattle until adult females are engorged (˜3 weeks), at which time females that drop from cattle will be collected daily (˜21-25 days post infestation). Multiple parameters of female tick fitness in control versus. vaccinated groups are collected during this time, including the number of females that survive, weight, egg mass weight, egg hatching success, larval weight, and larval survival. All live engorged female ticks (typically <1,000 for a control) will be disinfected, counted, weighed, and placed in petri dishes to oviposit for thirteen days at 26 C and 80% humidity. The total combined tick egg mass collected from each single cow being tested will be collected and weighed. Egg viability is then estimated using 0.2 g of total egg mass from each bovine, combined across the five cows in each group (i.e., 1.0 g total per group), with triplicate samples from each cow. Eggs are then incubated until 15 days after larval groups hatch and the number of unhatched eggs remaining will be estimated by weight. A 0.1 g sample of larvae (˜2,000) is counted and sorted as alive or dead to evaluate early survival. We will then perform statistical comparisons between control versus vaccinated groups to evaluate the effect on the ticks throughout. Finally, vaccine efficacy is calculated using a standard formula that is widely implemented in cattle fever tick studies, which incorporates the number of surviving females, egg mass weight, and larval weight per gram of eggs.

As part of the testing, a quantification of IgG antibody responses is carried out. The immunogenicity of each CLERM protein is evaluated using ELISA containing the purified protein and cattle-specific secondary antibodies to quantify protein-specific IgG levels in vaccinated versus control animals (n=5 in each group). ELISAs are also used to quantify IgG levels of CLERM and tick proteins used in subsequent bivalent vaccine trials discussed later. This immunological data provides key information on the capacity of CLERM proteins to stimulate an IgG antibody response that protects against ticks.

To perform Western blot analyses and verify tissue localization, intact midgut with Malpighian tubules is harvested from 30 adult female ticks (not used in the vaccination experiment) from the lab colony. The tissues are then placed into two pools, flash-frozen in 1×PBS, and shipped for laboratory analysis, where one pool is used to make tissue homogenates and the other used for localization studies. Antibodies from vaccinated cattle sera are then used to confirm the presence of the final five target CLERM proteins in the homogenized tissues via Western blot analysis. Antibodies also are used to perform immunochemical staining of intact tissues to verify that: 1) the targeted VDAC and CLERM proteins are indeed present and expressed in the expected locations, and 2) IgG antibodies are able to access and react with target proteins in situ. This is an important step for validating protein targets and evaluating reactivity with IgG antibodies. Finally, DNA will be extracted from a small amount of tissue to test with the CLERM-specific qPCR assay previously discussed to broadly assess CLERM abundance.

During the challenge experiments, adult female ticks are collected and surface disinfected from control and vaccinated animals (n=10 ticks per cow; 50 ticks per group of five cows) to extract DNA and perform 16S community analysis, as described previously. This approach is used to reveal broad effects on tick microbiome composition in adult females that result from specific IgG antibodies reacting with OM proteins of the obligate endosymbiont. Insight into the relative contribution of each CLERM target is also obtained by making comparisons across the five vaccinated groups. Because the CLERM is vertically transmitted from females to eggs and thus to larvae, DNA is extracted from pooled egg mass samples and pooled larval samples to perform real-time PCR for relative CLERM quantification using the real-time PCR assay previously described. With this analysis, the amount of CLERM in eggs and larvae is evaluated to determine if it is reduced after females feed on vaccinated bovine hosts.

Following completion of the first trial experiment, the efficacy of bivalent vaccines containing at least one CLERM protein and the tick VDAC protein will be evaluated. Two bivalent vaccines will be tested in groups of five cattle and one control group of five cattle (15 animals total in trial 2). The two CLERM proteins utilized in the second trial are selected based upon their efficacy in trial 1. This trial will follow the same methods as trial 1 summarized in FIG. 12, including evaluating tick fitness and estimating vaccine efficacy.

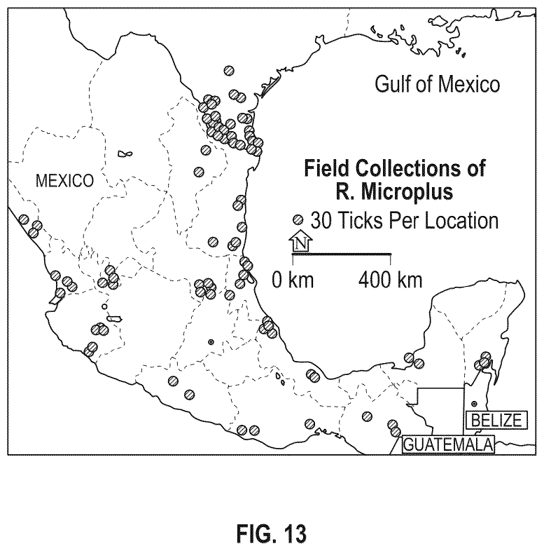

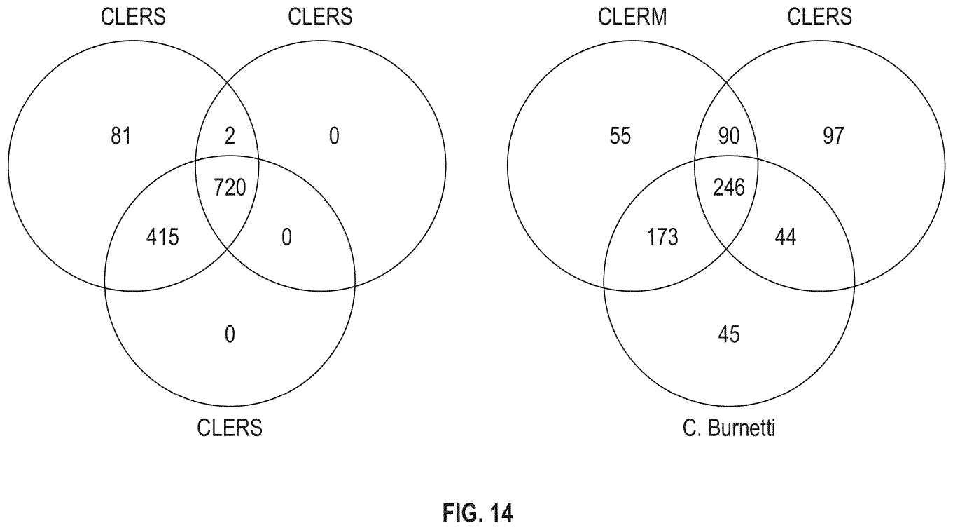

The genomic analysis of the R. microplus tick vaccines and the corresponding CLERM endosymbiont proteins also includes conservation analysis based on an extensive database of geographically dispersed tick collections over a significant period of time that include tools that allow for accurate data collection. Among these tools are microsatellite-based genotyping systems for R. microplus and R. annulatus and additional assays to detect genetic mutations associated with resistance to several classes of acaricides. Applying these tools has important insights about CFTs in Texas and Mexico, including: 1) CFT infestations are shared freely between wildlife and cattle, 2) short-distance tick dispersal typically results from infested wildlife movements, whereas long-distance dispersal always results from transport of infested cattle, and 3) resistance mutations for synthetic pyrethroids frequently spread from Mexico into Texas. Because of this long-term partnership with USDA and TAHC, and also with other CFT scientists, an extensive collection of DNA extracts from >8,000 individual R. microplus ticks is used the conservation analysis. The majority are from geographically diverse field collections in Texas and Mexico as illustrated in FIG. 13, but DNA from CFT collections from Brazil, Colombia, and Puerto Rico is also included. Importantly, all DNA extractions utilized whole or half tick bodies and, thus, include DNA from the entire microbiome community, including the CLERM. Because these extracts have already been genotyped, representative subsets for various conservation studies can be prepared that are both genetically and geographically diverse.

The need for careful evaluation of the conservation of the genes that encode targeted endosymbiont proteins across multiple representatives of that species is illustrated by the diagrams in FIG. 14. On the left the shared genes (translating into shared proteins) between three different CLERS genomes collected from brown dog ticks are represented by circles with the intersecting regions containing the number of shared genes between the genomes. This diagram indicates that while most of the genes are shared, there are a significant number that are not shared at all between genomes. Thus, ensuring that the analyses only include genes/proteins actually shared among CLERS genomes is an important filtering step to ensure that vaccine escape does not happen. In the diagram on the right, the genes from a CLERM genome, a CLERS genome, and from Coxiella burnetii are each displayed using a circle with the total common genes represented by the overlapping regions. Notwithstanding the multispecies comparison, a majority of the genes in each genome are common, which indicates that a common conserved protein between the three species could be used to create a vaccine that would attach to each bacterium relatively equally and thus increase the universality of the vaccine.

One of the strategies for endosymbiont protein selection involves considering proteins involved in the shikimate pathway. As previously mentioned, the shikimate pathway has been observed in the Asian longhorned tick and through this pathway, the endosymbiont produces the serotonin that the tick responds to that creates the interest and desire to feed on the host. The paper to Zhong previously incorporated by reference demonstrated that when the endosymbiont CLEHL was killed using tetracycline, the feeding behavior of the ticks changed causing them to not have the desire to feed. The inability to feed can significantly impact female ticks as, while they may not die and drop themselves as a result, their ability to create and lay eggs may be significantly hindered or eliminated meaning that no new larval ticks can be created in the next generation. Thus, where the shikimate pathway is known to exist in a tick endosymbiont, utilizing a protein that causes IgG antibodies to significantly reduce the total number of bacteria may have the effect of causing the ticks to starve even if they are otherwise able to process blood meals.

The shikimate pathway exists in various tick endosymbionts. It is believed to be conserved in the CLERM based on analysis of 23 CLERM genomes which recovered 721 genes of this endosymbiont with 669 genes found in all 23 CLERM genomes. While the aroD protein in Francisella tularensis is not homologous to the gene in CLE, a type I 3-dehydroquinate dehydratase, it is believed that sufficient genes exist in the CLERM for the shikimate pathway to exist. With respect to the CLERS, the genetic assembly is missing the shikimate dehydrogenase gene aroE and subsequent enrichments are also missing this gene which possibly indicates that a full shikimate pathway is not present in CLERS. With respect to the CLEAA, the shikimate pathway is believed to exist, but a similar issue with the aroD is present as with the CLERM. An analysis of 9 adult female ticks that included genome enrichment to capture the transcriptome of CLERM identified 6 outer membrane proteins that were expressed in all 9 ticks, indicating conservation that means these could be used as vaccine target proteins.

In various vaccine implementations, the tick protein and endosymbiont protein can be selected to enable multispecies effectiveness. For example, a combined vaccine administered orally using baits that targets both I. scapularis and A. americanum and their corresponding endosymbionts could be constructed. This combined vaccine would target the carriers of the most important vector-borne disease in the United States, Lyme disease and the increasing problem of alpha gal previously discussed. Individual landowners would buy oral baits and leave them out on their properties to treat the local deer and mouse populations to correspondingly reduce local tick populations and simultaneously reducing the risk of tick feeding and pathogen transmission to humans.

In another implementation, a combined vaccine composition in an injectable form, or deliverable to mucosal surfaces through other mechanisms such as a gene gun, could be prepared that targets D. variablis/D. similis, I. scapularis, A. americanum, and/or R. sanguineus and all of their respective endosymbionts could be developed using the principles disclosed herein. This vaccine would cover all of the most common ticks found on pet dogs and cats in the United States. The vaccine could be given during routine veterinary examinations like the rabies vaccine. This vaccine has the similar benefit of protecting humans from these ticks as well as the dogs and cats from pathogens transmitted from these ticks.

The following references are each hereby incorporated entirely herein by reference:
Abbas, M. N., M. A. Jmel, I. Mekki, I. Dijkgraaf, and M. Kotsyfakis. 2023. Recent advances in tick antigen discovery and anti-tick vaccine development. International Journal of Molecular Sciences 24:4969.
Ackerman, S., F. B. Clare, T. W. McGill, and D. E. Sonenshine. 1981. Passage of host serum components, including antibody, across the digestive tract of Dermacentor variabilis (Say). J Parasitol 67:737-740.
Allen, J. R., and S. J. Humphreys. 1979. Immunization of Guinea pigs and cattle against ticks. Nature 280:491-493.
Ben-Yakir, D., C. J. Fox, J. T. Homer, and R. W. Barker. 1987. Quantification of host immunoglobulin in the hemolymph of ticks. J Parasitol 73:669-671.
Ben-Yosef, M., A. Rot, M. Mahagna, E. Kapri, A. Behar, and Y. Gottlieb. 2020. Coxiella-like endosymbiont of Rhipicephalus sanguineus is required for physiological processes during ontogeny. Front Microbiol 11:493.
Bonnet, S. I., F. Binetruy, A. M. Hernandez-Jarguin, and O. Duron. 2017. The tick microbiome: Why non-pathogenic microorganisms matter in tick biology and pathogen transmission. Front Cell Infect Microbiol 7:236.
Chakraborty, C., A. R. Sharma, M. Bhattacharya, and S. S. Lee. 2022. A detailed overview of immune escape, antibody escape, partial vaccine escape of SARS-COV-2 and their emerging variants with escape mutations. Front Immunol 13:801522.
Chinzei, Y., and H. Minoura. 1987. Host immunoglobulin G titre and antibody activity in haemolymph of the tick, Ornithodoros moubata. Med Vet Entomol 1:409-416.
Cowdry, E. V. 1925. A group of microorganisms transmitted hereditarily in ticks and apparently unassociated with disease. The Journal of experimental medicine 41:817-830.
de la Fuente, J., C. Almazan, M. Canales, J. M. Perez de la Lastra, K. M. Kocan, and P. Willadsen. 2007. A ten-year review of commercial vaccine performance for control of tick infestations on cattle. Anim Health Res Rev 8:23-28.
de La Fuente, J., M. Rodriguez, and J. C. Garcia-Garcia. 2000. Immunological control of ticks through vaccination with Boophilus microplus gut antigens. Ann N Y Acad Sci 916:617-621.
de la Fuente, J., M. Rodriguez, C. Montero, M. Redondo, J. C. Garcia-Garcia, L. Mendez, E. Serrano, M. Valdes, A. Enriquez, M. Canales, E. Ramos, O. Boue, H. Machado, and R. Lleonart. 1999. Vaccination against ticks (Boophilus spp.): the experience with the Bm86-based vaccine Gavac™. Genetic Analysis-Biomolecular Engineering 15:143-148.
Duron, O., F. Binetruy, V. Noel, J. Cremaschi, K. D. McCoy, C. Arnathau, O. Plantard, J. Goolsby, A. A. Perez de Leon, D. J. A. Heylen, A. R. Van Oosten, Y. Gottlieb, G. Baneth, A. A. Guglielmone, A. Estrada-Pena, M. N. Opara, L. Zenner, F. Vavre, and C. Chevillon. 2017. Evolutionary changes in symbiont community structure in ticks. Mol Ecol 26:2905-2921.
Duron, O., P. Doublet, F. Vavre, and D. Bouchon. 2018. The importance of revisiting Legionellales diversity. Trends Parasitol 34:1027-1037.
Forthal, D. N. 2014. Functions of Antibodies. Microbiol Spectr 2: AID-0019-2014.
Freeman, J. M., R. B. Davey, L. S. Kappmeyer, D. M. Kammlah, and P. U. Olafson. 2010. Bm86 midgut protein sequence variation in South Texas cattle fever ticks. Parasites & Vectors 3.
Galay, R. L., T. Matsuo, E. P. Hernandez, M. R. Talactac, K. Kusakisako, R. Umemiya-Shirafuji, M. Mochizuki, K. Fujisaki, and T. Tanaka. 2018. Immunofluorescent detection in the ovary of host antibodies against a secretory ferritin injected into female Haemaphysalis longicornis ticks. Parasitol Int 67:119-122.
Garcia-Garcia, J. C., I. L. Gonzalez, D. M. Gonzalez, M. Valdes, L. Mendez, J. Lamberti, B. D'Agostino, D. Citroni, H. Fragoso, M. Ortiz, M. Rodriguez, and J. De La Fuente. 1999. Sequence variations in the Boophilus microplus Bm86 locus and implications for immunoprotection in cattle vaccinated with this antigen. Experimental and Applied Acarology 23:883-895.
Guizzo, M. G., L. F. Parizi, R. D. Nunes, R. Schama, R. M. Albano, L. Tirloni, D. P. Oldiges, R. P. Vieira, W. H. C. Oliveira, M. S. Leite, S. A. Gonzales, M. Farber, O. Martins, I. D. S. Vaz, Jr., and P. L. Oliveira. 2017. A Coxiella mutualist symbiont is essential to the development of Rhipicephalus microplus. Sci Rep 7:17554.
Guizzo, M. G., L. Tirloni, S. A. Gonzalez, M. D. Farber, G. Braz, L. F. Parizi, E. S. L. A. Dedavid, I. da Silva Vaz, Jr., and P. L. Oliveira. 2022. Coxiella endosymbiont of Rhipicephalus microplus modulates tick physiology with a major impact in blood feeding capacity. Front Microbiol 13:868575.
Hussain, S., N. Perveen, A. Hussain, B. Song, M. U. Aziz, J. Zeb, J. Li, D. George, A. Cabezas-Cruz, and O. Sparagano. 2022. The symbiotic continuum within ticks: Opportunities for disease control. Frontiers in Microbiology 13.
Kasaija, P. D., M. Contreras, H. Kirunda, A. Nanteza, F. Kabi, S. Mugerwa, and J. de la Fuente. 2023. Inspiring anti-tick vaccine research, development and deployment in tropical Africa for the control of cattle ticks: Review and insights. Vaccines (Basel) 11.
Kemp, D. H., R. D. Pearson, J. M. Gough, and P. Willadsen. 1989. Vaccination against Boophilus microplus: localization of antigens on tick gut cells and their interaction with the host immune system. Exp Appl Acarol 7:43-58.
Lalzar, I., Y. Friedmann, and Y. Gottlieb. 2014. Tissue tropism and vertical transmission of Coxiella in Rhipicephalus sanguineus and Rhipicephalus turanicus ticks. Environ Microbiol 16:3657-3668.
Maitre, A., A. Wu-Chuang, J. Azelyte, V. Palinauskas, L. Mateos-Hernandez, D. Obregon, A. Hodzic, C. Valiente Moro, A. Estrada-Pena, J. C. Paoli, A. Falchi, and A. Cabezas-Cruz. 2022. Vector microbiota manipulation by host antibodies: the forgotten strategy to develop transmission-blocking vaccines. Parasit Vectors 15:4.
Manzano-Román, R., A. Encinas-Grandes, and R. Pérez-Sánchez. 2006. Antigens from the midgut membranes of Ornithodoros erraticus induce lethal anti-tick immune responses in pigs and mice. Vet Parasitol 135:65-79.
Mateos-Hernandez, L., D. Obregon, J. Maye, J. Borneres, N. Versille, J. de la Fuente, A. Estrada-Pena, A. Hodzic, L. Simo, and A. Cabezas-Cruz. 2020. Anti-tick microbiota vaccine impacts Ixodes ricinus performance during feeding. Vaccines (Basel) 8.
Mateos-Hernandez, L., D. Obregon, A. Wu-Chuang, J. Maye, J. Borneres, N. Versille, J. de la Fuente, S. Diaz-Sanchez, L. G. Bermudez-Humaran, E. Torres-Maravilla, A. Estrada-Pena, A. Hodzic, L. Simo, and A. Cabezas-Cruz. 2021. Anti-microbiota vaccines modulate the tick microbiome in a taxon-specific manner. Front Immunol 12:704621.
Merino, O., P. Alberdi, J. M. Perez de la Lastra, and J. de la Fuente. 2013. Tick vaccines and the control of tick-borne pathogens. Front Cell Infect Microbiol 3:30.
Noda, H., U. G. Munderloh, and T. J. Kurtti. 1997. Endosymbionts of ticks and their relationship to Wolbachia spp. and tick-borne pathogens of humans and animals. Appl Environ Microbiol 63:3926-3932.
Ortega-Sánchez, R., M. Camacho-Nuez, E. J. Castaneda-Ortiz, M. B. Martinez-Benitez, D. J. Hernandez-Silva, G. Aguilar-Tipacamu, and J. Mosqueda. 2020. Vaccine efficacy of recombinant BmVDAC on Rhipicephalus microplus fed on Babesia bigemina-infected and uninfected cattle. Vaccine 38:3618-3625.
Parthasarathi, B. C., B. Kumar, S. K. Bhure, A. K. Sharma, Manisha, G. Nagar, S. Kumar, A. Nandi, H. V. Manjunathachar, G. M. Chigure, M. Shakya, M. Sankar, J. Fuente, and S. Ghosh. 2023. Co-immunization efficacy of recombinant antigens against Rhipicephalus microplus and Hyalomma anatolicum tick infestations. Pathogens 12.
Qian, L., X. Lin, X. Gao, R. U. Khan, J. Y. Liao, S. Du, J. Ge, S. Zeng, and S. Q. Yao. 2023. The dawn of a new era: Targeting the “undruggables” with antibody-based therapeutics. Chem Rev.
Rodríguez-Durán, A., S. Ullah, L. F. Parizi, A. Ali, and I. da Silva Vaz Junior. 2023. Rabbits as animal models for anti-tick vaccine development: A global scenario. Pathogens 12:1117.
Shan, H., Y. Liu, J. Luan, and S. Liu. 2021. New insights into the transovarial transmission of the symbiont Rickettsia in whiteflies. Sci China Life Sci 64:1174-1186.
Smith, T. A., T. Driscoll, J. J. Gillespie, and R. Raghavan. 2015. A Coxiella-like endosymbiont is a potential vitamin source for the Lone Star tick. Genome Biol Evol 7:831-838.
Tellam, R. L., D. Kemp, G. Riding, S. Briscoe, D. Smith, P. Sharp, D. Irving, and P. Willadsen. 2002. Reduced oviposition of Boophilus microplus feeding on sheep vaccinated with vitellin. Veterinary parasitology 103:141-156.
Thorpe, C. J., X. R. Wang, U. G. Munderloh, and T. J. Kurtti. 2021. Tick cell culture analysis of growth dynamics and cellular tropism of Rickettsia buchneri, an endosymbiont of the blacklegged tick, Ixodes scapularis. Insects 12.
Vaughan, J. A., D. E. Sonenshine, and A. F. Azad. 2002. Kinetics of ingested host immunoglobulin G in hemolymph and whole body homogenates during nymphal development of Dermacentor variabilis and Ixodes scapularis ticks (Acari: Ixodidae). Exp Appl Acarol 27:329-340.
Wang, H., and P. A. Nuttall. 1994. Excretion of host immunoglobulin in tick saliva and detection of IgG-binding proteins in tick haemolymph and salivary glands. Parasitology 109 (Pt 4): 525-530.
Webster, R. G., W. G. Laver, G. M. Air, and G. C. Schild. 1982. Molecular mechanisms of variation in influenza viruses. Nature 296:115-121.
Willadsen, P., and D. H. Kemp. 1989. Novel vaccination for control of the Babesia vector, Boophilus microplus. Trans R Soc Trop Med Hyg 83 Suppl: 107.
Wu-Chuang, A., L. Mateos-Hernandez, A. Maitre, R. O. M. Rego, R. Šíma, S. Porcelli, S. Rakotobe, A. Foucault-Simonin, S. Moutailler, V. Palinauskas, J. Aželytė, L. Simo, D. Obregon, and A. Cabezas-Cruz. 2023. Microbiota perturbation by anti-microbiota vaccine reduces the colonization of Borrelia afzelii in Ixodes ricinus. Microbiome 11:151.
Wu-Chuang, A., D. Obregon, L. Mateos-Hernandez, and A. Cabezas-Cruz. 2022. Anti-tick microbiota vaccines: how can this actually work? Biologia 77:1555-1562.
Zhong, J. 2012. Coxiella-like endosymbionts. Pages 365-379 Coxiella burnetii: Recent advances and new perspectives in research of the Q fever bacterium. Adv Exp Med Biol.
Zhong, Z., T. Zhong, Y. Peng, X. Zhou, Z. Wang, H. Tang, and J. Wang. 2021. Symbiont-regulated serotonin biosynthesis modulates tick feeding activity. Cell Host Microbe 29:1545-1557 e1544.
Zindel, R., Y. Gottlieb, and A. Aebi. 2011. Arthropod symbioses: a neglected parameter in pest- and disease-control programmes. Journal of Applied Ecology 48:864-872.
Bian, Y., W. Chen, G. Yang, X. Gu, S. Wang, J. Sun, K. Li, and H. Wei. 2011. Cloning, expression and evaluation of the efficacy of a recombinant Haemaphysalis concinna Hc-23 antigen in rabbits. Vaccine 29:1041-1044.
de la Fuente, J., R. Manzano-Roman, V. Naranjo, K. M. Kocan, Z. Zivkovic, E. F. Blouin, M. Canales, C. Almazan, R. C. Galindo, D. L. Step, and M. Villar. 2010. Identification of protective antigens by RNA interference for control of the lone star tick, Amblyomma americanum. Vaccine 28:1786-1795.
Galay, R. L., T. Miyata, R. Umemiya-Shirafuji, H. Maeda, K. Kusakisako, N. Tsuji, M. Mochizuki, K. Fujisaki, and T. Tanaka. 2014. Evaluation and comparison of the potential of two ferritins as anti-tick vaccines against Haemaphysalis longicornis. Parasit Vectors 7:482.
Moreno-Cid, J. A., J. M. Perez de la Lastra, M. Villar, M. Jimenez, R. Pinal, A. Estrada-Pena, R. Molina, J. Lucientes, C. Gortazar, J. de la Fuente, and S. A. V. S. Group. 2013. Control of multiple arthropod vector infestations with subolesin/akirin vaccines. Vaccine 31:1187-1196.
Ortega-Sánchez, R., M. Camacho-Nuez, E. J. Castaneda-Ortiz, M. B. Martinez-Benitez, D. J. Hernandez-Silva, G. Aguilar-Tipacamu, and J. Mosqueda. 2020. Vaccine efficacy of recombinant BmVDAC on Rhipicephalus microplus fed on Babesia bigemina-infected and uninfected cattle. Vaccine 38:3618-3625.
Rodríguez-Mallon, A., E. Fernandez, P. E. Encinosa, Y. Bello, L. Mendez-Perez, L. C. Ruiz, D. Perez, M. Gonzalez, H. Garay, O. Reyes, L. Mendez, and M. P. Estrada. 2012. A novel tick antigen shows high vaccine efficacy against the dog tick, Rhipicephalus sanguineus. Vaccine 30:1782-1789.
You, M. J. 2005. Immunization of mice with recombinant P27/30 protein confers protection against hard tick Haemaphysalis longicornis (Acari: Ixodidae) infestation. J Vet Sci 6:47-51.

In places where the description above refers to particular implementations of anti-tick vaccine compositions and related methods and implementing components, sub-components, methods and sub-methods, it should be readily apparent that a number of modifications may be made without departing from the spirit thereof and that these implementations, implementing components, sub-components, methods and sub-methods may be applied to other anti-tick vaccine compositions and related methods.

What is claimed is:

1. An anti-tick vaccine composition comprising:
one or more conserved tick proteins from a tick species; and
one or more conserved outer membrane proteins of an endosymbiont of the tick species.

2. The composition of claim 1, wherein the one or more conserved tick proteins and the one or more conserved outer membrane proteins each have a conservation level equal to or greater than 97%.

3. The composition of claim 1, wherein the tick species is Rhipicephalus microplus and the one or more conserved tick proteins is at least 75%, 80%, 85%, 90%, 95%, or 100% identical to a protein selected from the group consisting of SEQ ID NO: 1-28 and any combination or fragment thereof and the endosymbiont is CLERM and the one or more conserved outer membrane proteins is selected from the group consisting of SEQ ID NO: 190-207 and any combination or fragment thereof.

4. The composition of claim 1, wherein the tick species is Rhipicephalus sanguineus sensu lato and the one or more conserved tick proteins is at least 75%, 80%, 85%, 90%, 95%, or 100% identical to a protein selected from the group consisting of SEQ ID NO: 29-56 and any combination or fragment thereof and the endosymbiont is CLERS and the one or more conserved outer membrane proteins is selected from the group consisting of SEQ ID NO: 221-232 and any combination or fragment thereof.

5. The composition of claim 1, wherein the tick species is Amblyomma americanum and the one or more conserved tick proteins is at least 75%, 80%, 85%, 90%, 95%, or 100% identical to a protein selected from the group consisting of SEQ ID NO: 113-140 and any combination or fragment thereof and the endosymbiont is CLEAA and the one or more conserved outer membrane proteins is selected from the group consisting of SEQ ID NO: 233-242 and any combination or fragment thereof.

6. The composition of claim 1, wherein the endosymbiont is CLERA and the one or more conserved outer membrane proteins is at least 75%, 80%, 85%, 90%, 95%, or 100% identical to a protein selected from the group consisting of SEQ ID NO: 208-220 and any combination or fragment thereof.

7. The composition of claim 1, wherein the tick species is Ixodes scapularis and the one or more conserved tick proteins is at least 75%, 80%, 85%, 90%, 95%, or 100% identical to a protein selected from the group consisting of SEQ ID NO: 57-84 and any combination or fragment thereof.

8. The composition of claim 1, wherein the tick species is Ixodes pacificus and the one or more conserved tick proteins is at least 75%, 80%, 85%, 90%, 95%, or 100% identical to a protein selected from the group consisting of SEQ ID NO: 85-112 and any combination or fragment thereof.

9. The composition of claim 1, wherein the tick species is Haemaphysalis longicornis and the one or more conserved tick proteins is at least 75%, 80%, 85%, 90%, 95%, or 100% identical to a protein selected from the group consisting of SEQ ID NO: 141-161 and any combination or fragment thereof.

10. The composition of claim 1, wherein the tick species is Dermacentor variabilis and the one or more conserved tick proteins is at least 75%, 80%, 85%, 90%, 95%, or 100% identical to a protein selected from the group consisting of SEQ ID NO: 162-189 and any combination or fragment thereof.

11. A method of vaccinating an animal against hosting a tick, the method comprising:
administering to the animal a pharmaceutically effective amount of a bivalent vaccine comprising one or more conserved tick proteins from a tick species and one or more conserved outer membrane proteins of an endosymbiont of the tick species.

12. The method of claim 11, wherein administering comprises injecting or delivering the vaccine into a body or mucosa of the animal or oral application to the animal and where the bivalent vaccine further comprises where the one or more conserved tick proteins and the one or more conserved outer membrane proteins each have a conservation level equal to or greater than 97%.

13. The method of claim 11, wherein the tick species is Rhipicephalus microplus and the one or more conserved tick proteins is at least 75%, 80%, 85%, 90%, 95%, or 100% identical to a protein selected from the group consisting of SEQ ID NO: 1-28 and any combination or fragment thereof and the endosymbiont is CLERM and the one or more conserved outer membrane proteins is selected from the group consisting of SEQ ID NO: 190-207 and any combination or fragment thereof.

14. The method of claim 11, wherein the tick species is Rhipicephalus sanguineus sensu lato and the one or more conserved tick proteins is at least 75%, 80%, 85%, 90%, 95%, or 100% identical to a protein selected from the group consisting of SEQ ID NO: 29-56 and any combination or fragment thereof and the endosymbiont is CLERS and the one or more conserved outer membrane proteins is selected from the group consisting of SEQ ID NO: 221-232 and any combination or fragment thereof.

15. The method of claim 11, wherein the tick species is Amblyomma americanum and the one or more conserved tick proteins is at least 75%, 80%, 85%, 90%, 95%, or 100% identical to a protein selected from the group consisting of SEQ ID NO: 113-140 and any combination or fragment thereof and the endosymbiont is CLEAA and the one or more conserved outer membrane proteins is selected from the group consisting of SEQ ID NO: 233-242 and any combination or fragment thereof.

16. The method of claim 11, wherein the endosymbiont is CLERA and the one or more conserved outer membrane proteins is at least 75%, 80%, 85%, 90%, 95%, or 100% identical to a protein selected from the group consisting of SEQ ID NO: 208-220 and any combination or fragment thereof.

17. The method of claim 11, wherein the tick species is Ixodes scapularis and the one or more conserved tick proteins is at least 75%, 80%, 85%, 90%, 95%, or 100% identical to a protein selected from the group consisting of SEQ ID NO: 57-84 and any combination or fragment thereof.

18. The method of claim 11, wherein the tick species is Ixodes pacificus and the one or more conserved tick proteins is at least 75%, 80%, 85%, 90%, 95%, or 100% identical to a protein selected from the group consisting of SEQ ID NO: 85-112 and any combination or fragment thereof.

19. The method of claim 11, wherein the tick species is Haemaphysalis longicornis and the one or more conserved tick proteins is at least 75%, 80%, 85%, 90%, 95%, or 100% identical to a protein selected from the group consisting of SEQ ID NO: 141-161 and any combination or fragment thereof.

20. The method of claim 11, wherein the tick species is Dermacentor variabilis and the one or more conserved tick proteins is at least 75%, 80%, 85%, 90%, 95%, or 100% identical to a protein selected from the group consisting of SEQ ID NO: 162-189 and any combination or fragment thereof.
